(19) United States
(12) Patent Application Publication
(10) Pub. No.: US 2024/0287022 A1
(43) Pub. Date: Aug. 29, 2024

(54) IL4I1 INHIBITORS AND METHODS OF USE

(71) Applicant: Merck Sharp & Dohme LLC, Rahway, NJ (US)

(72) Inventors: BRANDON D. CASH, Natick, MA (US); GEORGE MADALIN GIAMBASU, Natick, MA (US); ANDREW M. HAIDLE, SOMERVILLE, MA (US); BRETT A. HOPKINS, BROWNSBURG, IN (US); MATTHEW A. LARSEN, TEMPLETON, CA (US); CHARLES A. LESBURG, WABAN, MA (US); PING LIU, Westfield, MA (US); RYAN QUIROZ, SAN FRANCISCO, CA (US); SULAGNA SANYAL, BELMONT, MA (US); CATHERINE M. WHITE, Canton, MA (US); XIN YAN, NEWTON, MA (US); XIAO MEI ZHENG, Dover, MA (US)

(73) Assignee: Merck Sharp & Dohme LLC, Rahway, NJ (US)

(21) Appl. No.: 18/569,352

(22) PCT Filed: Jun. 23, 2022
(86) PCT No.: PCT/US2022/034622

§ 371 Date: Dec. 12, 2023

Related U.S. Application Data
(60) Provisional application No. 63/215,535, filed on Jun. 28, 2021.

(51) Int. Cl. C07D 401/06; A61K 31/4375; A61K 31/4704; A61K 31/4709; A61K 31/4725; A61K 31/4745; A61K 31/496; A61K 31/497; A61K 31/498; A61K 31/5025; A61K 31/519; A61K 31/538; A61K 31/5383; C07D 215/227; C07D 401/10; C07D 401/12; C07D 405/10; C07D 405/14; C07D 409/06; C07D 413/06; C07D 413/14; C07D 417/06; C07D 417/14; C07D 471/04; C07D 471/06; C07D 487/04; C07D 498/04; C07D 513/04

(52) U.S. Cl. CPC C07D 401/06; A61K 31/4375; A61K 31/4704; A61K 31/4709; A61K 31/4725; A61K 31/4745; A61K 31/496; A61K 31/497; A61K 31/498; A61K 31/5025; A61K 31/519; A61K 31/538; A61K 31/5383; C07D 215/227; C07D 401/10; C07D 401/12; C07D 405/10; C07D 405/14; C07D 409/06; C07D 413/06; C07D 413/14; C07D 417/06; C07D 417/14; C07D 471/04; C07D 471/06; C07D 487/04; C07D 498/04; C07D 513/04

(57) ABSTRACT

Described herein are compounds of Formula I or a pharmaceutically acceptable salt thereof wherein A, E, U, X, Y, Z, R¹, R² and n are as defined herein. The compounds of Formula I act as IL4I1 inhibitors and can be useful in preventing, treating or acting as a remedial agent for IL4I1-related diseases. Also provided herein are pharmaceutical compositions comprising the compounds of the invention, or their pharmaceutically acceptable salts, and a pharmaceutically acceptable carrier and methods of treatment with the compounds of the invention.

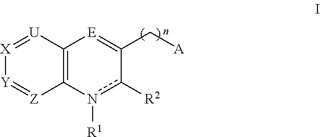

### FIELD OF THE INVENTION

The present invention is directed to IL4I1 inhibitors. Specifically, the IL4I1 inhibitors described herein can be useful in preventing, treating or acting as a remedial agent for IL4I1-related diseases.

### BACKGROUND OF THE INVENTION

IL4I1 is a glycosylated protein that belongs to the L-amino-acid oxidase (LAAO) family of flavin adenine dinucleotide (FAD)-bound enzymes. IL4I1 is secreted from certain cells and performs oxidative deamination of phenylalanine into phenylpyruvate, liberating H₂O₂ and NH₃.

The highest production of IL4I1 is found in cells of myeloid origin (monocyte/macrophages and dendritic cells) of the human immune system, particularly after stimulation with inflammatory and T helper type 1 (Th1) stimuli. Accordingly, IL4I1 is strongly produced by dendritic cell and macrophage populations from chronic Th1 granulomas of sarcoidosis and tuberculosis, but not Th2 granulomas (schistosomiasis). Moreover, tumor-infiltrating macrophages from various histological types of tumors strongly produce IL4I1. Molinier-Frenkel V., Prévost-Blondel A. and Castellano F., The IL4I1 Enzyme: A New Player in the Immunosuppressive Tumor Microenvironment, Cells, 2019, 8, 757-765.

The presence of IL4I1-producing cells in the tumor cell microenvironment restrains the anti-tumor immune response by directly limiting the proliferation and functionality of cytotoxic T cells and Th1 cells, or indirectly by facilitating the accumulation of Treg cells. Analyses of human tumor and normal tissue biopsies have identified increased expression of both IL4I1 mRNA and protein in tumor infiltrating myeloid cells. The Cancer Genome Atlas (TCGA) indicate that, among solid tumors, endometrial carcinoma contains the highest levels of IL4I1 mRNA expression, followed by serious ovarian and triple negative breast cancers. Phenylpyruvic acid, the product of phenylalanine oxidation by IL4I1, is elevated in endometrial and ovarian tumor samples relative to matched adjacent tissue from the same patients. Furthermore, accumulation of detectable phenylpyruvic acid in the tumor samples is dependent on the presence of IL4I1 itself.

Currently there are no commercially available specific inhibitors against IL4I1. Some molecules have been shown to inhibit the related LAAOs found in snake venom, but they are generally non-selective and have little activity. Therefore there is a need for specific inhibitors of IL4I1. More specifically there is a need for compounds that specifically inhibit IL4I1 and can be useful for the treatment of indications where IL4I1 is most expressed and/or active, including endometrial, ovarian and triple negative breast cancers.

### BRIEF SUMMARY OF THE INVENTION

Described herein are compounds of Formula I.

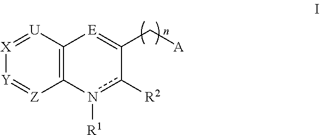

wherein A, E, U, X, Y, Z, R¹, R² and n are described in further detail below.

The compounds described herein are IL4i1 inhibitors, which can be useful in the prevention, treatment or amelioration of IL4i1-related diseases.

Also described are methods of treating cancer comprising administering to a patient in need thereof a compound described herein, or a pharmaceutically acceptable salt thereof.

Also described are uses of a compound described herein, or a pharmaceutically acceptable salt thereof, to treat cancer in a patient in need thereof.

Also described are pharmaceutical compositions comprising a compound described herein, or a pharmaceutically acceptable salt thereof and a pharmaceutically acceptable carrier.

Also described are pharmaceutical compositions comprising a compound described herein and a pharmaceutically acceptable carrier.

Also described are methods of preventing, treating or ameliorating the symptoms of cancer comprising administering to a patient in need thereof a compound described herein, or a pharmaceutically acceptable salt thereof and at least one additional therapeutic agent.

Also described are uses of a compound described herein, or a pharmaceutically acceptable salt thereof, in combination with at least one additional therapeutic agent to prevent, treat or ameliorate the conditions of cancer in a patient in need thereof.

Also described are pharmaceutical compositions comprising a compound described herein, or a pharmaceutically acceptable salt thereof, at least one additional therapeutic agent and a pharmaceutically acceptable carrier.

Also described are pharmaceutical compositions comprising a compound described herein, at least one additional therapeutic agent and a pharmaceutically acceptable carrier.

### DETAILED DESCRIPTION OF THE INVENTION

Described herein are compounds of Formula I:

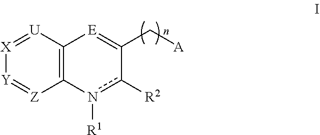

or a pharmaceutically acceptable salt thereof, wherein:
A is aryl, C₃-C₁₀cycloalkyl, heteroaryl or cycloheteroalkyl, wherein the aryl, C₃-C₁₀cycloalkyl, heteroaryl or cycloheteroalkyl is unsubstituted or substituted with 1 to 4 substituents independently selected from the group consisting of halogen, C₁-C₆alkyl, haloC₁-C₆alkyl, —OH, C₁-C₆alkylOH, haloC₁-C₆alkylOH, —COC₁-C₆alkyl, —CN, C₁-C₆alkylCN, alkoxy, haloalkoxy, C₃-C₁₀cycloalkyl, C₁-C₆alkylC₃-C₁₀cycloalkyl, —OC₃-C₁₀cycloalkyl, —O(C₁-C₆)C₃-C₁₀cycloalkyl, aryl, C₁-C₆alkylaryl, heteroaryl, C₁-C₆alkylheteroaryl, C₁-C₆alkylcycloheteroalkyl, cycloheteroalkyl, —Ocycloheteroalkyl, —COcycloheteroalkyl, —NHcycloheteroalkyl, —SO₂NH₂, C₁-C₆alkylSO₂NH₂, C₁-C₆alkylNHSO₂C₁-C₆alkyl, —NHSO₂C₁-C₆alkyl, —NHCOOC₁-C₆alkyl, —NHCOC₁-C₆alkyl, C₁-C₆alkylNHCOOC₁-C₆alkyl, C₁-C₆alkylNHCOC₁-C₆alkyl and

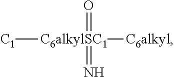

wherein the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, —NHcycloheteroalkyl, cycloheteroalkyl, —COcycloheteroalkyl, C₁-C₆alkylcycloheteroalkyl, is unsubstituted or substituted with 1 to 3 substituents independently selected from the group consisting of halogen, C₁-C₆alkyl, C₃-C₁₀cycloalkyl, haloC₁-C₆alkyl, NH₂, alkoxy, —OH, C₁-C₆alkylOH, —CN, C₁-C₆alkylCN, COC₁-C₆alkyl and oxo;
E is N, O or CR³;
U is N, S or CR⁴, wherein when U is N, X, Y and Z are CR⁵, CR⁶ and CR⁷ respectively;
X is a bond, N or CR⁵, wherein when X is N, U, Y and Z are CR⁴, CR⁶ and CR⁷ respectively;
Y is N or CR⁶, wherein when Y is N, X, U and Z are CR⁵, CR⁴ and CR⁷ respectively;
Z is N, S or CR⁷, wherein when Z is N, X, Y and U are CR⁵, CR⁶ and CR⁴ respectively;
R¹ is a bond between the nitrogen R¹ is attached and the carbon R² is attached, hydrogen or C₁-C₆alkyl, or when taken with R⁷ forms a C₅-C₇cycloalkyl;
R² is oxo or —OH, wherein when R² is oxo R¹ is not a bond between the nitrogen R¹ is attached and the carbon R² is attached;
R³ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁴ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁵ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁶ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁷ is hydrogen, halogen, —CN, —OH, C₁-C₆alkyl, C₁-C₆alkylNH₂, C₁-C₆alkylNHC₁-C₆alkyl, alkoxy or C₁-C₆alkylOH or when taken with R¹ forms a C₅-C₇cycloalkyl; and
n is 1 or 2.

With regard to the compounds described herein, R² is oxo or —OH. In certain embodiments, R² is —OH. In certain embodiments, when R² is —OH, R¹ is a bond between the nitrogen R¹ is attached and the carbon R² is attached. In other embodiments, R² is oxo. When R² is oxo, R¹ is not a bond between the nitrogen R¹ is attached and the carbon R² is attached In certain embodiments, when R² is oxo, R¹ is hydrogen or C₁-C₆alkyl, or when taken with R⁷ forms a C₅-C₇cycloalkyl. Such embodiments are shown in Formula Ia and Formula Ib:

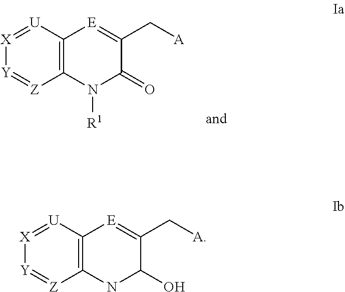

Also described herein are compounds of Formula Ia:

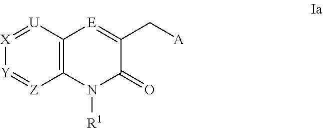

or a pharmaceutically acceptable salt thereof, wherein:
A is aryl, C₃-C₁₀cycloalkyl, heteroaryl or cycloheteroalkyl, wherein the aryl, C₃-C₁₀cycloalkyl, heteroaryl or cycloheteroalkyl is unsubstituted or substituted with 1 to 4 substituents independently selected from the group consisting of halogen, C₁-C₆alkyl, haloC₁-C₆alkyl, —OH, C₁-C₆alkylOH, haloC₁-C₆alkylOH, COC₁-C₆alkyl, —CN, C₁-C₆alkylCN, alkoxy, haloalkoxy, C₃-C₁₀cycloalkyl, C₁-C₆alkylC₃-C₁₀cycloalkyl, —OC₃-C₁₀cycloalkyl, —O(C₁-C₆)C₃-C₁₀cycloalkyl, aryl, C₁-C₆alkylaryl, heteroaryl, C₁-C₆alkylheteroaryl, C₁-C₆alkylcycloheteroalkyl, cycloheteroalkyl, —Ocycloheteroalkyl, COcycloheteroalkyl, NHcycloheteroalkyl, SO₂NH₂, C₁-C₆alkylSO₂NH₂, C₁-C₆alkylNHSO₂C₁-C₆alkyl, NHSO₂C₁-C₆alkyl, NHCOOC₁-C₆alkyl, NHCOC₁-C₆alkyl, C₁-C₆alkylNHCOOC₁-C₆alkyl, C₁-C₆alkylNHCOC₁-C₆alkyl and

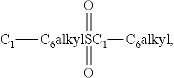

wherein the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, and C₁-C₆alkylcycloheteroalkyl, is unsubstituted or substituted with 1 to 3 substituents independently selected from the group consisting of halogen, C₁-C₆alkyl, C₃-C₁₀cycloalkyl, haloC₁-C₆alkyl, NH₂, alkoxy, —OH, C₁-C₆alkylOH, —CN, C₁-C₆alkylCN, COC₁-C₆alkyl and oxo;
E is N, O or CR³;
U is N, S or CR⁴, wherein when U is N, X, Y and Z are CR⁵, CR⁶ and CR⁷ respectively;
X is a bond, N or CR⁵, wherein when X is N, U, Y and Z are CR⁴, CR⁶ and CR⁷ respectively;
Y is N or CR⁶, wherein when Y is N, X, U and Z are CR⁵, CR⁴ and CR⁷ respectively;
Z is N, S or CR⁷, wherein when Z is N, X, Y and U are CR⁵, CR⁶ and CR⁴ respectively;
R¹ is hydrogen or C₁-C₆alkyl, or when taken with R⁷ forms a C₅-C₇cycloalkyl;
R³ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁴ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁵ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁶ is hydrogen, halogen, —CN, —OH, C₁-C₆alkyl, C₁-C₆alkylNH₂, C₁-C₆alkylNHC₁-C₆alkyl, alkoxy or C₁-C₆alkylOH; and
R⁷ is hydrogen, halogen, —CN, —OH, C₁-C₆alkyl, C₁-C₆alkylNH₂, C₁-C₆alkylNHC₁-C₆alkyl, alkoxy or C₁-C₆alkylOH or when taken with R¹ forms a C₅-C₇cycloalkyl.

With regard to the compounds described herein, E is N, O or CR³. In certain embodiments, E is N. In certain embodiments, E is N or CR³. In other embodiments, E is O. In still other embodiments, E is CR³.

With regard to the compounds described herein, R³ is hydrogen, halogen, —CN, —OH, C₁-C₆alkyl, C₁-C₆alkylNH₂, C₁-C₆alkylNHC₁-C₆alkyl, alkoxy or C₁-C₆alkylOH. In certain embodiments, R³ is hydrogen. In certain embodiments, R³ is halogen. Suitable halogens include, but are not limited to, a fluorine, a chlorine, a bromine or an iodine radical. In certain embodiments, R³ is —CN. In certain embodiments, R³ is —OH. In certain embodiments, R³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R³ is C₁-C₆alkylNH₂. Suitable C₁-C₆alkylNH₂ include but are not limited to, CH₂NH₂ and CH₂CH₂NH₂. In certain embodiments, R³ is C₁-C₆alkylNHC₁-C₆alkyl. Suitable C₁-C₆alkylNH₂C₁-C₆alkyls include but are not limited to, CH₂NHCH₃ and CH₂CH₂NH CH₂CH₃. In certain embodiments, R³ is alkoxy. Suitable alkoxys include, but are not limited to, methoxy, ethoxy, n-propoxy, isopropoxy and n-butoxy. In certain embodiments, R³ is C₁-C₆alkylOH. Suitable alcohols, but are not limited to, methanol, ethanol, propanol, butanol and isopropanol.

In certain embodiments, E is CR³, wherein R³ is hydrogen or methyl.

With regard to the compounds described herein, U is N, S or CR⁴. In certain embodiments, U is N. In certain embodiments, U is N, and, X, Y, and Z are CR⁵, CR⁶ and CR⁷, respectively. In certain embodiments, U is N or CR⁴. In certain embodiments, U is S. In certain embodiments, U is CR⁴.

With regard to the compounds described herein, R⁴ is hydrogen, halogen, —CN, —OH, C₁-C₆alkyl, C₁-C₆alkylNH₂, C₁-C₆alkylNHC₁-C₆alkyl, alkoxy or C₁-C₆alkylOH. In certain embodiments, R⁴ is hydrogen. In certain embodiments, R⁴ is halogen. Suitable halogens include, but are not limited to, a fluorine, a chlorine, a bromine or an iodine radical. In certain embodiments, R⁴ is —CN. In certain embodiments, R⁴ is —OH. In certain embodiments, R⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁴ is C₁-C₆alkylNH₂. Suitable C₁-C₆alkylNH₂ include but are not limited to, CH₂NH₂ and CH₂CH₂NH₂. In certain embodiments, R⁴ is C₁-C₆alkylNHC₁-C₆alkyl. Suitable C₁-C₆alkylNH₂C₁-C₆alkyls include but are not limited to, CH₂NHCH₃ and CH₂CH₂NH CH₂CH₃. In certain embodiments, R⁴ is alkoxy. Suitable alkoxys include, but are not limited to, methoxy, ethoxy, n-propoxy, isopropoxy and n-butoxy. In certain embodiments, R⁴ is C₁-C₆alkylOH. Suitable alcohols include, but are not limited to, methanol, ethanol, propanol, butanol and isopropanol.

In certain embodiments, U is CR⁴, wherein R⁴ is hydrogen.

With regard to the compounds described herein, X is a bond, N or CR⁵. In certain embodiments X is a bond. In certain embodiments, X is a bond when R² is —OH. In certain embodiments, X is N. In certain embodiments, X is N, and U, Y and Z are CR⁴, CR⁶ and CR⁷, respectively. In certain embodiments, X is CR⁵.

With regard to the compounds described herein, R⁵ is hydrogen, halogen, —CN, —OH, C₁-C₆alkyl, C₁-C₆alkylNH₂, C₁-C₆alkylNHC₁-C₆alkyl, alkoxy or C₁-C₆alkylOH. In certain embodiments, R⁵ is hydrogen. In certain embodiments, R⁵ is halogen. Suitable halogens include, but are not limited to, a fluorine, a chlorine, a bromine or an iodine radical. In certain embodiments, R⁵ is —CN. In certain embodiments, R⁵ is —OH. In certain embodiments, R⁵ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁵ is C₁-C₆alkylNH₂. Suitable C₁-C₆alkylNH₂ include but are not limited to, CH₂NH₂ and CH₂CH₂NH₂. In certain embodiments, R⁵ is C₁-C₆alkylNHC₁-C₆alkyl. Suitable C₁-C₆alkylNH₂C₁-C₆alkyls include but are not limited to, CH₂NHCH₃ and CH₂CH₂NH CH₂CH₃. In certain embodiments, R⁵ is alkoxy. Suitable alkoxys include, but are not limited to, methoxy, ethoxy, n-propoxy, isopropoxy and n-butoxy. In certain embodiments, R⁵ is C₁-C₆alkylOH. Suitable hydroxyalkyls include, but are not limited to, hydroxymethyl, hydroxyethyl, hydroxypropyl, hydroxybutyl, and hydroxy-isopropyl.

In certain embodiments, X is N or CR⁵, wherein R⁵ is hydrogen, fluorine, methoxy, —CN or hydroxy-isopropyl. In certain embodiments, X is CR⁵, wherein R⁵ is hydrogen, fluorine, methoxy, —CN or hydroxy-isopropyl.

With regard to the compounds described herein, Y is N or CR⁶. In certain embodiments, Y is N. In certain embodiments, Y is N, and X, U and Z are CR⁵, CR⁴ and CR⁷, respectively. In certain embodiments, Y is CR⁶.

With regard to the compounds described herein, R⁶ is hydrogen, halogen, —CN, —OH, C₁-C₆alkyl, C₁-C₆alkylNH₂, C₁-C₆alkylNHC₁-C₆alkyl, alkoxy or C₁-C₆alkylOH. In certain embodiments, R⁶ is hydrogen. In certain embodiments, R⁶ is halogen. Suitable halogens include, but are not limited to, a fluorine, a chlorine, a bromine or an iodine radical. In certain embodiments, R⁶ is —CN. In certain embodiments, R⁶ is —OH. In certain embodiments, R⁶ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁶ is C₁-C₆alkylNH₂. Suitable C₁-C₆alkylNH₂ include but are not limited to, CH₂NH₂ and CH₂CH₂NH₂. In certain embodiments, R⁶ is C₁-C₆alkylNHC₁-C₆alkyl. Suitable C₁-C₆alkylNH₂C₁-C₆alkyls include but are not limited to, CH₂NHCH₃ and CH₂CH₂NH CH₂CH₃. In certain embodiments, R⁶ is alkoxy. Suitable alkoxys include, but are not limited to, methoxy, ethoxy, n-propoxy, isopropoxy and n-butoxy. In certain embodiments, R⁶ is C₁-C₆alkylOH. Suitable alcohols include, but are not limited to, methanol, ethanol, propanol, butanol and isopropanol.

In certain embodiments, Y is N or CR⁶, wherein R⁶ is hydrogen. In certain embodiments, Y is CR⁶, wherein R⁶ is hydrogen.

With regard to the compounds described herein, Z is N, S or CR⁷. In certain embodiments, Z is N. In certain embodiments, Z is N, and X, Y and U are CR⁵, CR⁶ and CR⁴, respectively. In certain embodiments, Z is N or CR⁷. In certain embodiments, Z is S. In certain embodiments, Z is CR⁷.

With regard to the compounds described herein, R⁷ is hydrogen, halogen, —CN, —OH, C₁-C₆alkyl, C₁-C₆alkylNH₂, C₁-C₆alkylNHC₁-C₆alkyl, alkoxy or C₁-C₆alkylOH or when taken with R¹ forms a C₅-C₇cycloalkyl. In certain embodiments, R⁷ is hydrogen. In certain embodiments, R⁷ is halogen. Suitable halogens include, but are not limited to, a fluorine, a chlorine, a bromine or an iodine radical. In certain embodiments, R⁷ is —CN. In certain embodiments, R⁷ is —OH. In certain embodiments, R⁷ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁷ is C₁-C₆alkylNH₂. Suitable C₁-C₆alkylNH₂ include but are not limited to, CH₂NH₂ and CH₂CH₂NH₂. In certain embodiments, R⁷ is C₁-C₆alkylNHC₁-C₆alkyl. Suitable C₁-C₆alkylNH₂C₁-C₆alkyls include but are not limited to, CH₂NHCH₃ and CH₂CH₂NH CH₂CH₃. In certain embodiments, R⁷ is alkoxy. Suitable alkoxys include, but are not limited to, methoxy, ethoxy, n-propoxy, isopropoxy and n-butoxy. In certain embodiments, R⁷ is C₁-C₆alkylOH. Suitable alcohols include, but are not limited to, methanol, ethanol, propanol, butanol and isopropanol.

In certain embodiments, R⁷, when taken with R¹ forms a C₅-C₇cycloalkyl. In certain embodiments, R⁷, when taken with R¹ forms a cyclopentane ring. In certain embodiments, R⁷, when taken with R¹ forms a cyclohexane ring. In certain embodiments, R⁷, when taken with R¹ forms a cycloheptane ring.

In certain embodiments, Z is N or CR⁷, wherein R⁷ is hydrogen, hydroxyethyl, CH₂NH₂ or CH₂NHCH₃ or when taken with R¹ forms a cyclohexane ring.

With regard to the compounds described herein, R¹ is a bond between the nitrogen R¹ is attached and the carbon R² is attached, hydrogen or C₁-C₆alkyl. In certain embodiments, R¹ is a bond between the nitrogen R¹ is attached and the carbon R² is attached. In certain embodiments, when R² is oxo, R¹ is not a bond between the nitrogen R¹ is attached and the carbon R² is attached. In certain embodiments, R¹ is hydrogen. In other embodiments, R¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments described herein, R¹ is hydrogen or C₁-C₆alkyl, or when taken with R⁷ forms a C₅-C₇cycloalkyl. In certain embodiments, R¹ is hydrogen. In other embodiments, R¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

With regard to the compounds described herein, n is 1 or 2. In certain embodiments, n is 1. In other embodiments, n is 2.

In certain embodiments, described herein are compounds having the following formulas:

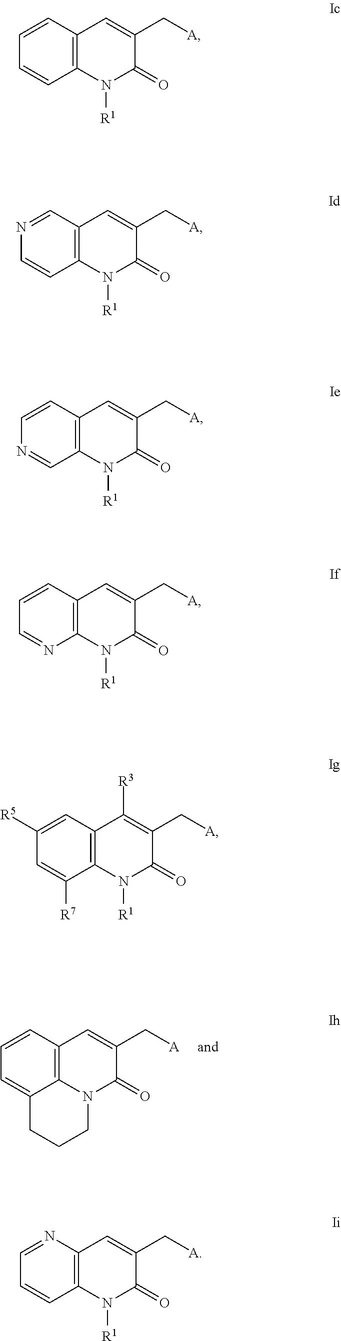

With regard to compounds described herein, A is aryl, C₃-C₁₀cycloalkyl, heteroaryl or cycloheteroalkyl, wherein the aryl, C₃-C₁₀cycloalkyl, heteroaryl or cycloheteroalkyl is unsubstituted or substituted with 1 to 4 substituents independently selected from the group consisting of halogen, C₁-C₆alkyl, haloC₁-C₆alkyl, —OH, C₁-C₆alkylOH, haloC₁-C₆alkylOH, COC₁-C₆alkyl, —CN, C₁-C₆alkylCN, alkoxy, haloalkoxy, C₃-C₁₀cycloalkyl, C₁-C₆alkylC₃-C₁₀cycloalkyl, —OC₃-C₁₀cycloalkyl, —O(C₁-C₆)C₃-C₁₀cycloalkyl, aryl, C₁-C₆alkylaryl, heteroaryl, C₁-C₆alkylheteroaryl, C₁-C₆alkylcycloheteroalkyl, cycloheteroalkyl, —Ocycloheteroalkyl, COcycloheteroalkyl, NHcycloheteroalkyl, SO₂NH₂, C₁-C₆alkylSO₂NH₂, C₁-C₆alkylNHSO₂C₁-C₆alkyl, NHSO₂C₁-C₆alkyl, NHCOOC₁-C₆alkyl, NHCOC₁-C₆alkyl, C₁-C₆alkylNHCOOC₁-C₆alkyl, C₁-C₆alkylNHCOC₁-C₆alkyl and

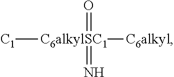

wherein the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, C₁-C₆alkylcycloheteroalkyl, is unsubstituted or substituted with 1 to 3 substituents selected from the group consisting of halogen, C₁-C₆alkyl, C₃-C₁₀cycloalkyl, haloC₁-C₆alkyl, NH₂, alkoxy, —OH, C₁-C₆alkylOH, —CN, C₁-C₆alkylCN, COC₁-C₆alkyl and oxo.

With regard to compounds described herein, A is aryl, C₃-C₁₀cycloalkyl, heteroaryl or cycloheteroalkyl. In certain embodiments, A is aryl. In certain embodiments, A is a monocyclic aryl. In other embodiments, A is a bicyclic aryl. In other embodiments, A is a multicyclic aryl. Suitable aryls include, but are not limited to, phenyl and naphthyl. In certain embodiments, A is aryl, wherein the aryl is phenyl. In certain embodiments, A is aryl, wherein the aryl is naphthyl. In certain embodiments, the aryl is

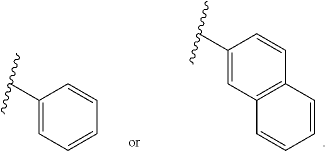 [SMILES: *c1ccccc1 ; *c1ccc2ccccc2c1]

In other embodiments, A is C₃-C₁₀cycloalkyl. In certain embodiments, A is a monocyclic cycloalkyl. In other embodiments, A is a bicyclic cycloalkyl. In other embodiments, A is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, A is C₃-C₁₀cycloalkyl, wherein the C₃-C₁₀cycloalkyl is:

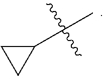 [SMILES: *C1CC1]

In certain embodiments, A is heteroaryl. In certain embodiments, A is a nitrogen-containing heteroaryl. In certain embodiments, A is a monocyclic heteroaryl. In other embodiments, A is a bicyclic heteroaryl. In other embodiments, A is a multicyclic heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, A is heteroaryl, wherein the heteroaryl is:

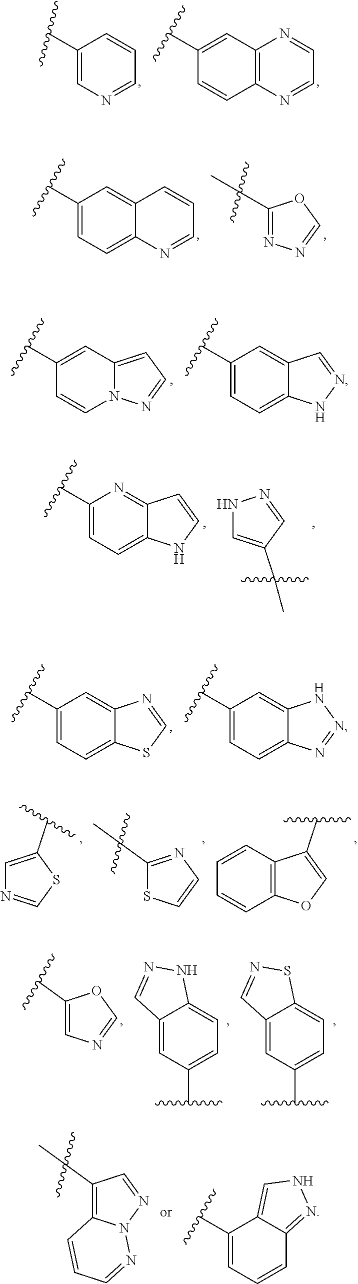

In certain embodiments, A is heteroaryl, wherein the heteroaryl is:

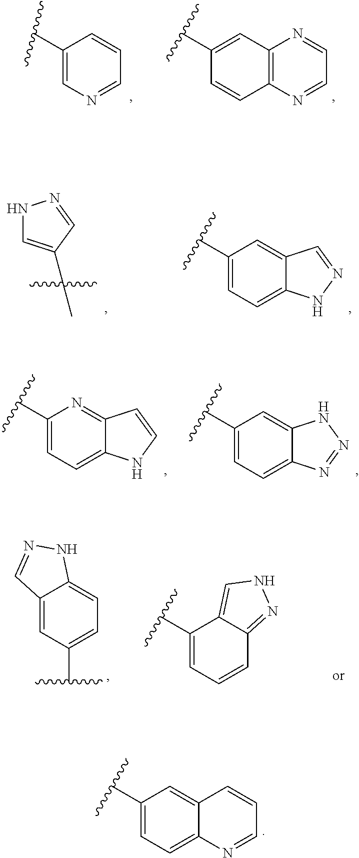

In certain embodiments, A is cycloheteroalkyl. In certain embodiments, A is a monocyclic cycloheteroalkyl. In other embodiments, A is a multicyclic cycloheteroalkyl. In still other embodiments, A is a bicyclic cycloheteroalkyl. In certain embodiments, A is a nitrogen-containing cycloheteroalkyl. In other embodiments, A is an oxygen-containing cycloheteroalkyl. In other embodiments, A is a sulfur-containing cycloheteroalkyl. Suitable cycloheteroalkyls include, but are not limited to, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, tetrahydropyran, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl.

In certain embodiments, A is a cycloheteroalkyl, wherein the cycloheteroalkyl is:

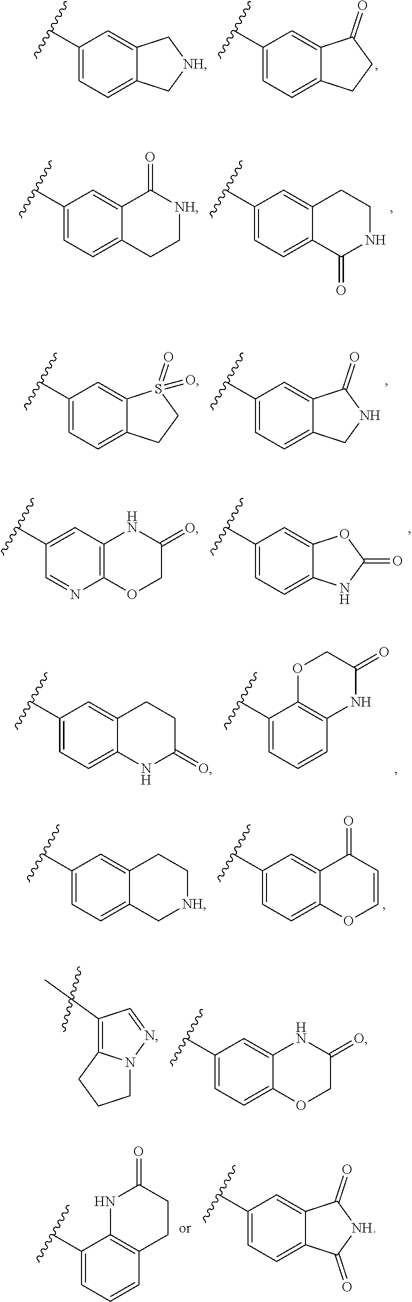

With regard to compounds described herein, A is aryl, C₃-C₁₀cycloalkyl, heteroaryl or cycloheteroalkyl, wherein the aryl, C₃-C₁₀cycloalkyl, heteroaryl or cycloheteroalkyl is unsubstituted or substituted with 1 to 4 substituents. In certain embodiments, A is unsubstituted. In other embodiments, A is substituted with 1 to 4 substituents. In certain embodiments, A is substituted with 1 substituent. In certain embodiments, A is substituted with 2 substituents. In certain embodiments, A is substituted with 3 substituents. In certain embodiments, A is substituted with 4 substituents.

In certain embodiments, A is substituted with 1 to 4 substituents independently selected from the group consisting of halogen, C₁-C₆alkyl, haloC₁-C₆alkyl, —OH, C₁-C₆alkylOH, haloC₁-C₆alkylOH, COC₁-C₆alkyl, —CN, C₁-C₆alkylCN, alkoxy, haloalkoxy, C₃-C₁₀cycloalkyl, C₁-C₆alkylC₃-C₁₀cycloalkyl, —OC₃-C₁₀cycloalkyl, —O(C₁-C₆)C₃-C₁₀cycloalkyl, aryl, C₁-C₆alkylaryl, heteroaryl, C₁-C₆alkylheteroaryl, C₁-C₆alkylcycloheteroalkyl, cycloheteroalkyl, —Ocycloheteroalkyl, COcycloheteroalkyl, NHcycloheteroalkyl, SO₂NH₂, C₁-C₆alkylSO₂NH₂, C₁-C₆alkylNHSO₂C₁-C₆alkyl, NHSO₂C₁-C₆alkyl, NHCOOC₁-C₆alkyl, NHCOC₁-C₆alkyl, C₁-C₆alkylNHCOOC₁-C₆alkyl, and C₁-C₆alkylNHCOC₁-C₆alkyl.

In certain embodiments, A is substituted with a halogen. Suitable halogens include, but are not limited to, a fluorine, a chlorine, a bromine or an iodine radical. In certain embodiments, A is substituted with fluorine, bromine or chlorine.

In certain embodiments, A is substituted with a C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, A is substituted with methyl, ethyl or

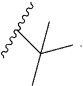 [SMILES: *C(C)(C)C]

In certain embodiments, A is substituted with a haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, A is substituted with difluoromethyl. In certain embodiments, A is substituted with trifluoromethyl. In certain embodiments, A is substituted with difluoromethyl or trifluoromethyl.

In certain embodiments, A is substituted with —OH.

In certain embodiments, A is substituted with a C₁-C₆alkylOH. Suitable alcohols include, but are not limited to, methanol, ethanol, propanol and butanol. In certain embodiments, A is substituted with

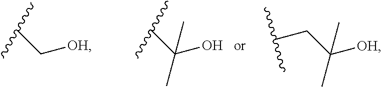

In certain embodiments, A is substituted with a haloC₁-C₆alkylOH. In certain embodiments, A is substituted with

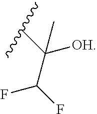 [SMILES: *C(C)(O)C(F)F]

In certain embodiments, A is substituted with a COC₁-C₆alkyl. In certain embodiments, A is substituted with COCH₃.

In certain embodiments, A is substituted with —CN.

In certain embodiments, A is substituted with a C₁-C₆alkylCN. In certain embodiments, A is substituted with

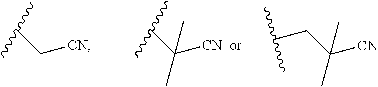

In certain embodiments, A is substituted with a alkoxy. Suitable alkoxys include, but are not limited to, methoxy, ethoxy, n-propoxy, isopropoxy and n-butoxy. In certain embodiments, A is substituted with methoxy.

In certain embodiments, A is substituted with a haloalkoxy. Suitable haloalkoxys include, but are not limited to, trifluoromethoxy, difluoromethoxy and monofluoromethoxy. In certain embodiments, A is substituted with trifluoromethoxy.

In certain embodiments, A is substituted with a C₃-C₁₀cycloalkyl. In certain embodiments, A is a monocyclic cycloalkyl. In other embodiments, A is a bicyclic cycloalkyl.

In other embodiments, A is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, A is substituted with

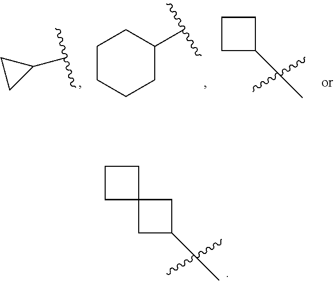

In certain embodiments, A is substituted with a C₁-C₆alkylC₃-C₁₀cycloalkyl. In certain embodiments, A is substituted with

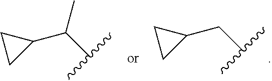

In certain embodiments, A is substituted with a —OC₃-C₁₀cycloalkyl. In certain embodiments, A is substituted with

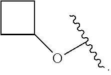 [SMILES: *OC1CCC1]

In certain embodiments, A is substituted with a —O(C₁-C₆)C₃-C₁₀cycloalkyl. In certain embodiments, A is substituted with

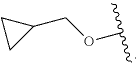 [SMILES: *OCC1CC1]

In certain embodiments, A is substituted with an aryl. Suitable aryls include, but are not limited to, phenyl and naphthyl. In certain embodiments, A is substituted with phenyl.

In certain embodiments, A is substituted with a C₁-C₆alkylaryl. In certain embodiments, A is substituted with

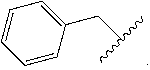 [SMILES: *Cc1ccccc1]

In certain embodiments, A is substituted with a heteroaryl. In certain embodiments, A is substituted with

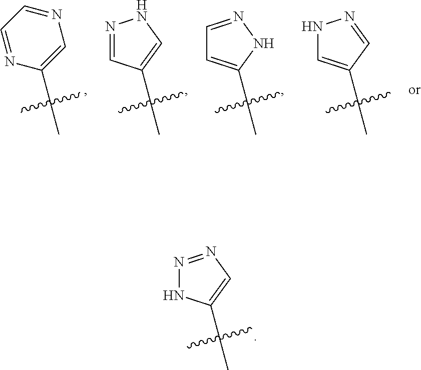 [SMILES: *c1cnccn1 ; *c1cn[nH]c1 ; *c1ccn[nH]1 ; *c1cn[nH]c1 ; *c1cnn[nH]1]

In certain embodiments, A is substituted with a C₁-C₆alkylheteroaryl. In certain embodiments, A is substituted with

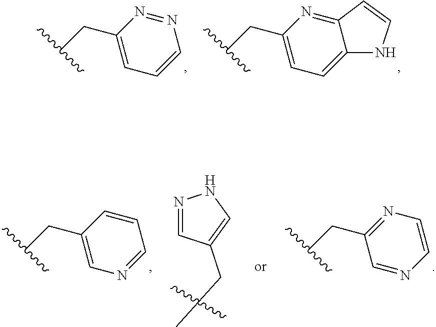

In certain embodiments, A is substituted with a C₁-C₆alkylcycloheteroalkyl. In certain embodiments, A is substituted with

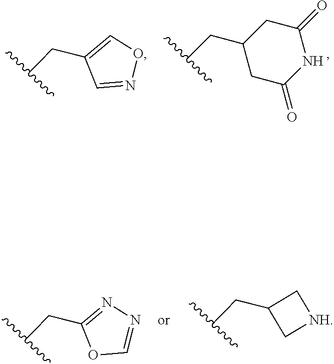 [SMILES: *Cc1cnoc1 ; *CC1CC(=O)NC(=O)C1 ; *Cc1nnco1 ; *CC1CNC1]

In certain embodiments, A is substituted with a cycloheteroalkyl. In certain embodiments, A is substituted with

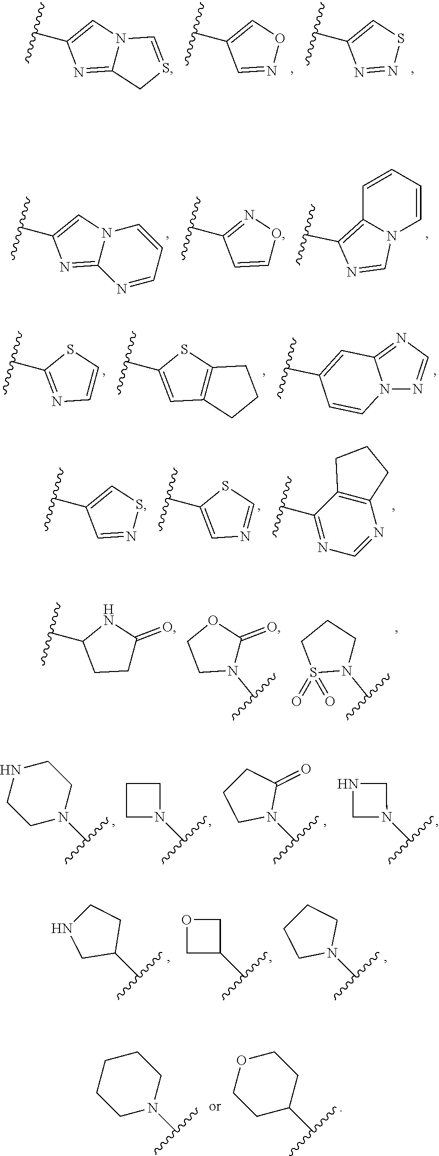

In certain embodiments, A is substituted with a —Ocycloheteroalkyl. In certain embodiments, A is substituted with

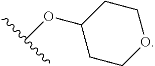 [SMILES: *OC1CCOCC1]

In certain embodiments, A is substituted with a —COcycloheteroalkyl. In certain embodiments, A is substituted with

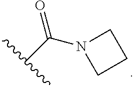 [SMILES: *C(=O)N1CCC1]

In certain embodiments, A is substituted with a NHcycloheteroalkyl. In certain embodiments, A is substituted with

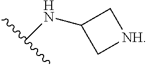 [SMILES: *NC1CNC1]

In certain embodiments, A is substituted with a SO₂NH₂.

In certain embodiments, A is substituted with a C₁-C₆alkylSO₂NH₂. In certain embodiments, A is substituted with a CH₂SO₂NH₂.

In certain embodiments, A is substituted with a C₁-C₆alkylNHSO₂C₁-C₆alkyl. In certain embodiments, A is substituted with CH₂NHSO₂CH₂CH₃ or CH₂NHSO₂CH₃.

In certain embodiments, A is substituted with a NHSO₂C₁-C₆alkyl. In certain embodiments, A is substituted with NHSO₂CH₂CH₃, NHSO₂CH₃.

In certain embodiments, A is substituted with a NHCOOC₁-C₆alkyl. In certain embodiments, A is substituted with NHCOOCH₃.

In certain embodiments, A is substituted with a NHCOC₁-C₆alkyl. In certain embodiments, A is substituted with NHCOCH₃.

In certain embodiments, A is substituted with a C₁-C₆alkylNHCOOC₁-C₆alkyl. In certain embodiments, A is substituted with CH₂NHCOOCH₃.

In certain embodiments, A is substituted with a C₁-C₆alkylNHCOC₁-C₆alkyl. In certain embodiments, A is substituted with CH₂NHCOCH₃.

In certain embodiments, A is substituted with

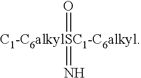

In certain embodiments, A is substituted with

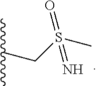 [SMILES: *CS(C)(=N)=O]

In certain embodiments, when A is substituted with a C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, C₁-C₆alkylcycloheteroalkyl, as defined above, the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, C₁-C₆alkylcycloheteroalkyl is unsubstituted or substituted with 1 to 3 substituents selected from the group consisting of halogen, C₁-C₆alkyl, C₃-C₁₀cycloalkyl, haloC₁-C₆alkyl, NH₂, alkoxy, —OH, C₁-C₆alkylOH, —CN, C₁-C₆alkylCN, COC₁-C₆alkyl and oxo.

In certain embodiments, when A is substituted with a C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, C₁-C₆alkylcycloheteroalkyl, as defined above, the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, NHcycloheteroalkyl, cycloheteroalkyl, COcycloheteroalkyl, C₁-C₆alkylcycloheteroalkyl is unsubstituted or substituted with 1 to 3 substituents selected from the group consisting of chlorine, fluorine, bromine, methyl, isopropyl, ethyl, cyclopropyl, trifluoromethyl, difluoromethyl, NH₂, methoxy, —OH, methanol, ethanol, —CN, CH₂CN, and COCH₃.

In other embodiments, A is

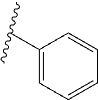 [SMILES: *c1ccccc1]

independently unsubstituted or substituted with 1 to 3 substituents selected from the group consisting of chlorine, fluorine, methoxy, methyl, —CN, —CH₂NHCOCH₃, —SO₂NH₂, —CH₂SO₂NH₂, —CH₂NHSO₂CH₃, C(CH₃)₂OH, —COCH₃, —OCF₃, —NHCOOCH₃, —NHC(O)CH₃, —CH₂NHCOOCH₃,

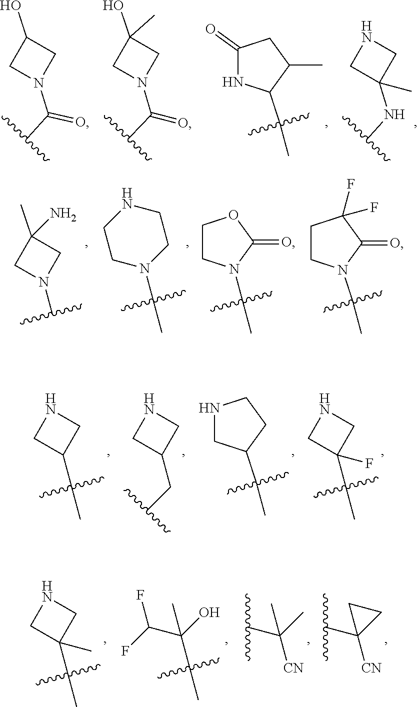

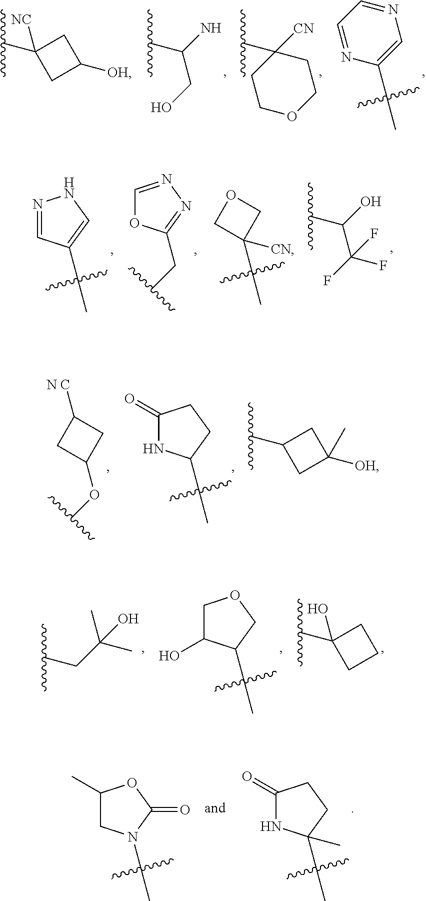

In certain embodiments, A is C₃-C₁₀cycloalkyl. In certain embodiments, A is cyclopropyl substituted with phenyl.

In certain embodiments, A is heteroaryl, wherein the heteroaryl is

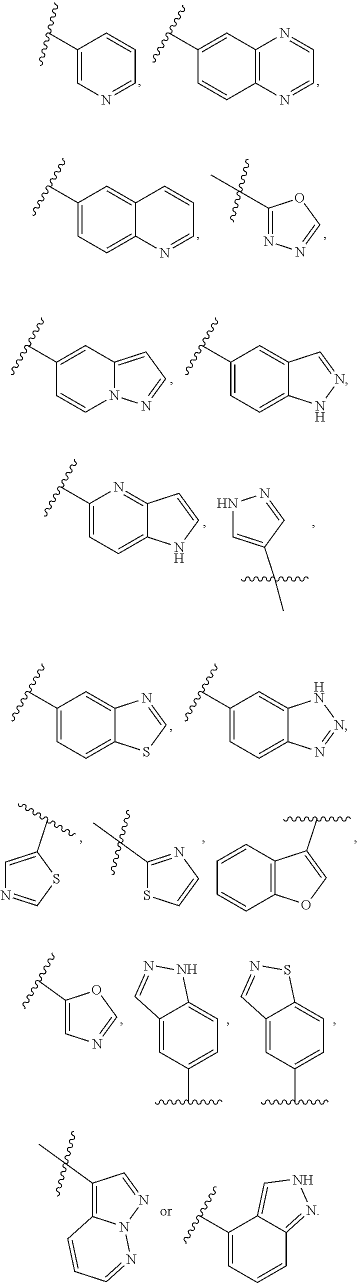

In certain embodiments, A is heteroaryl, wherein the heteroaryl is

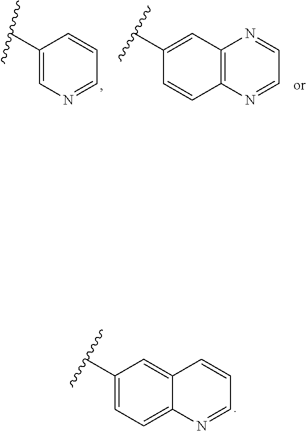

In certain embodiments, A is heteroaryl, wherein the heteroaryl is

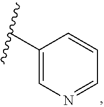 [SMILES: *c1cccnc1]

wherein the heteroaryl is unsubstituted or substituted with 1 to 3 substituents selected from the group consisting of chlorine, fluorine, —CH₂CN, —NHSO₂CH₂CH₃, —NHSO₂CH₃, —CH₂NHCOCH₃, —CH₂NHSO₂CH₃, —NHCOCH₃,

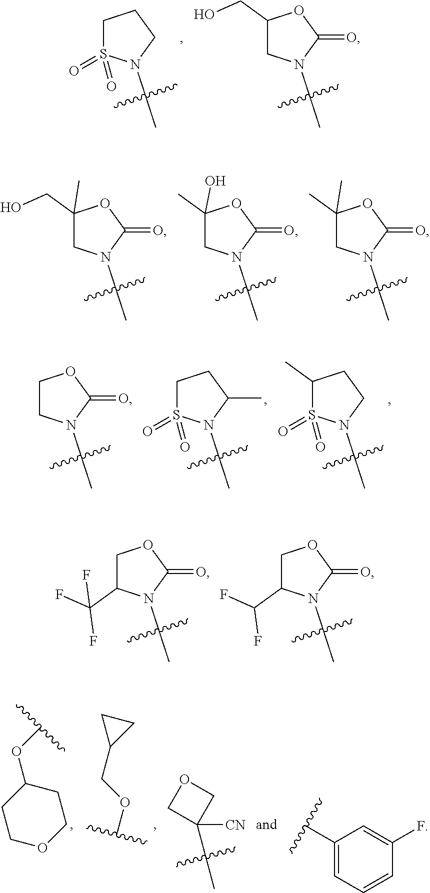

In certain embodiments, A is cycloheteroalkyl, wherein the cycloheteroalkyl is

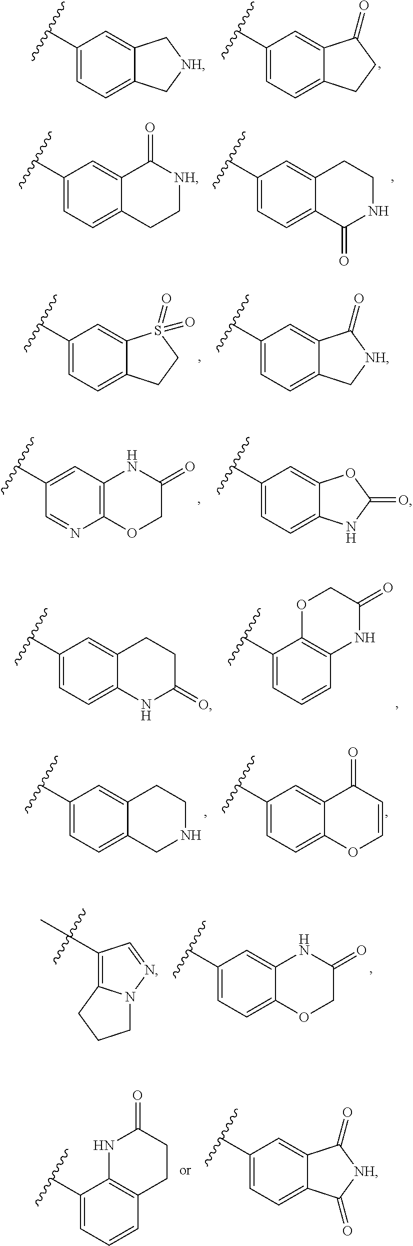

wherein the cycloheteroalkyl is unsubstituted or substituted with 1 to 3 substituents independently selected from the group consisting of fluorine, chlorine, COCH₃, —CN, methyl, ethyl, isobutyl, CH₂NHCOOCH₃,

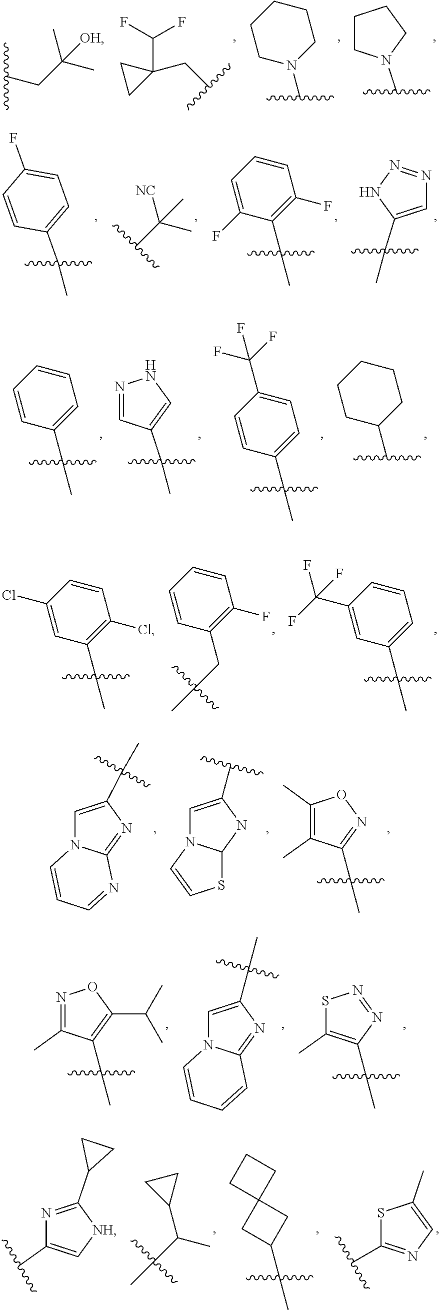

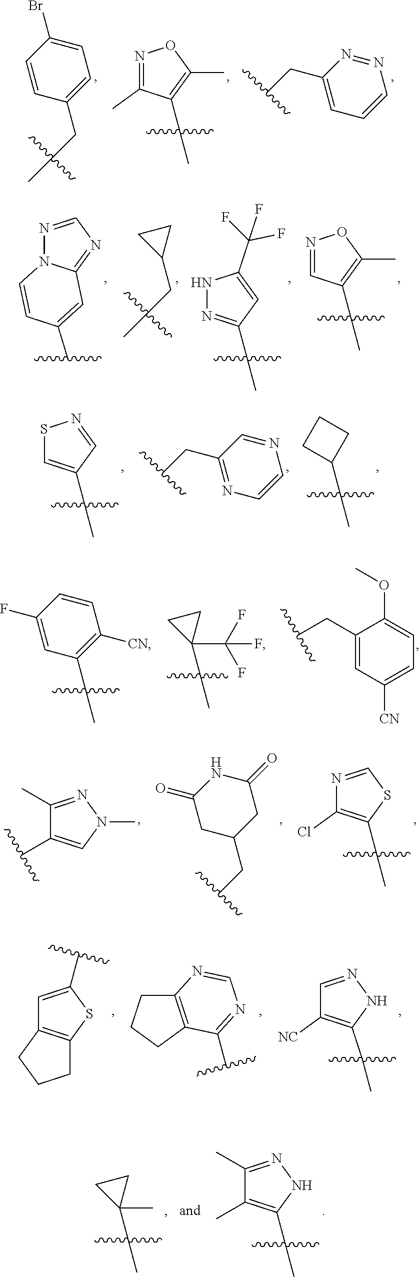

In certain embodiments, A is a heteroaryl, wherein the heteroaryl is

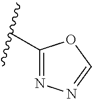 [SMILES: *c1nnco1]

wherein the heteroaryl is unsubstituted or substituted with 1 to 3 substituents selected from the group consisting of methyl, CH₂NHCOOCH₃, NHCOOCH₃,

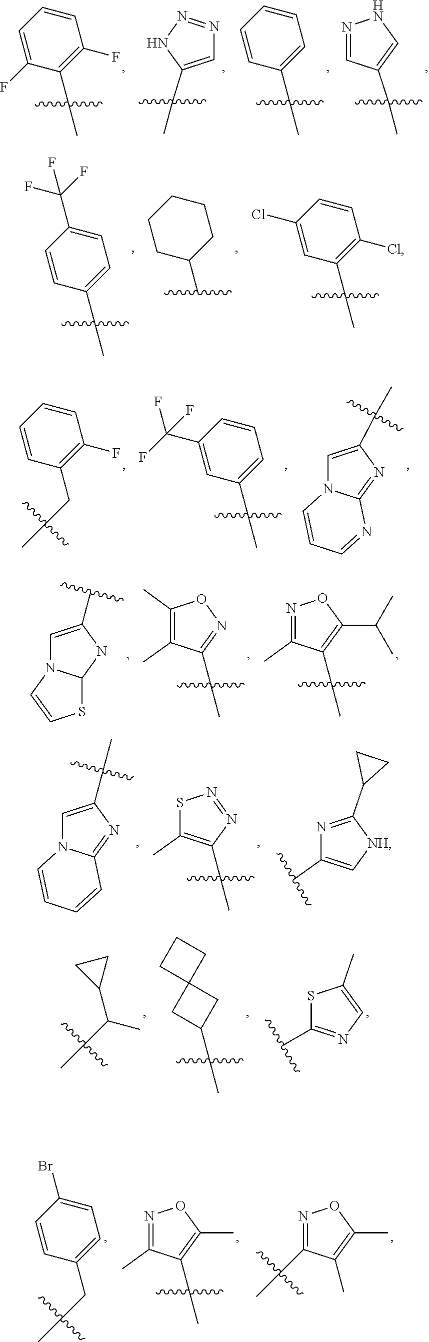

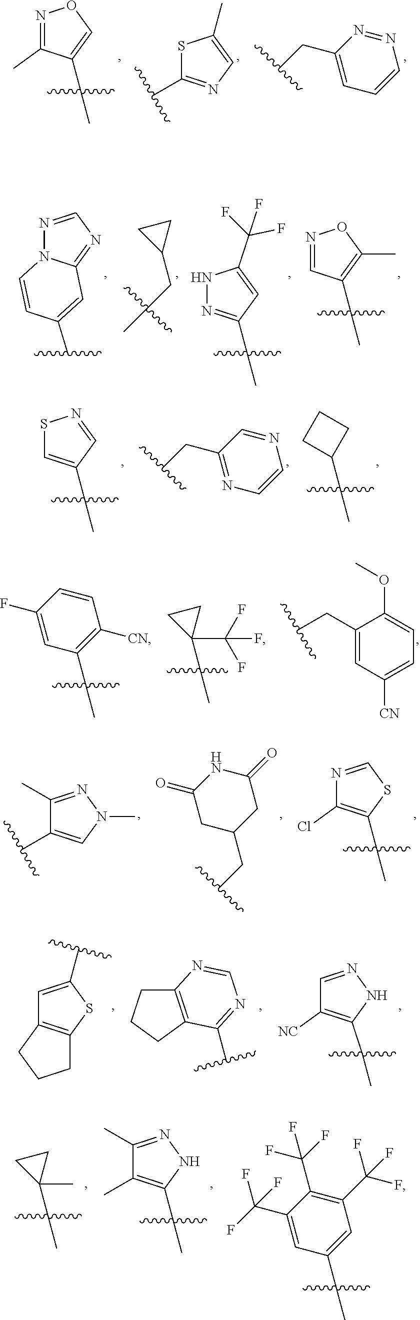

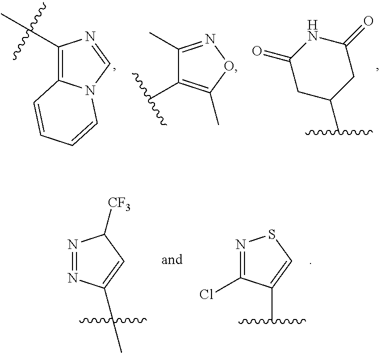

Also described herein are compounds having the following structures:

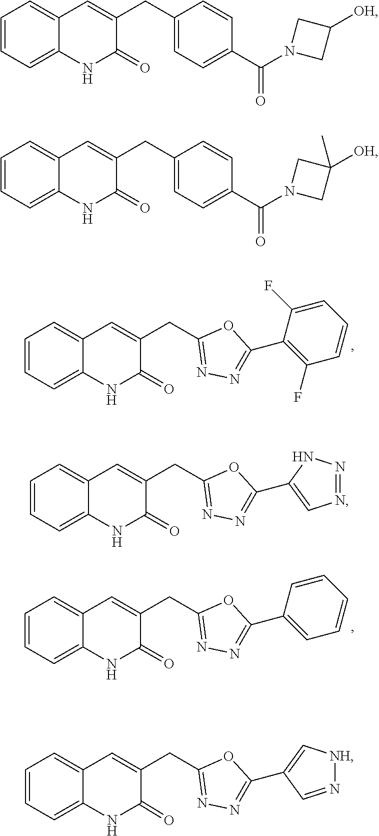

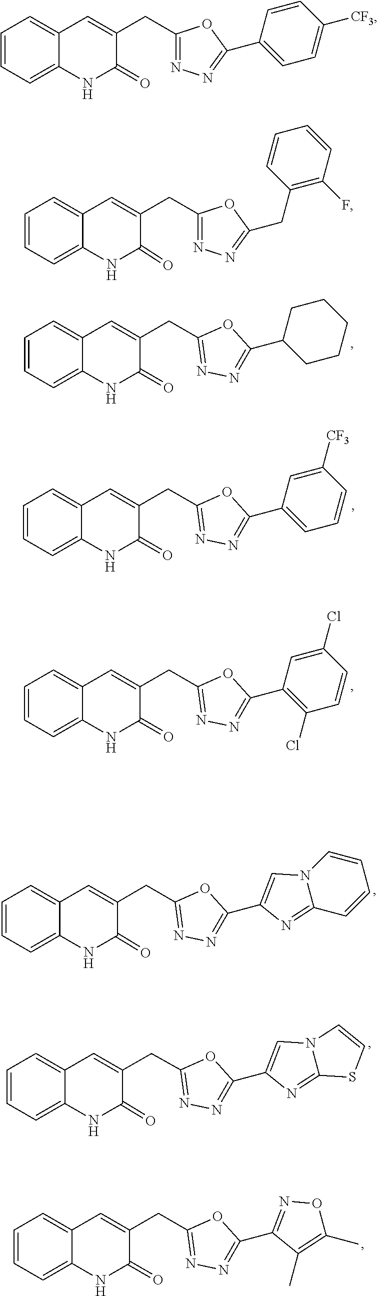

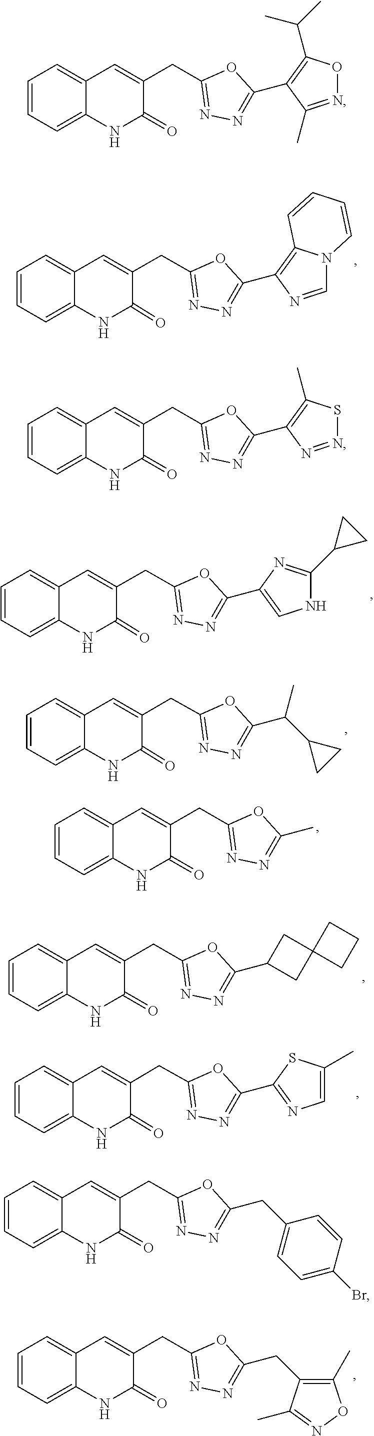

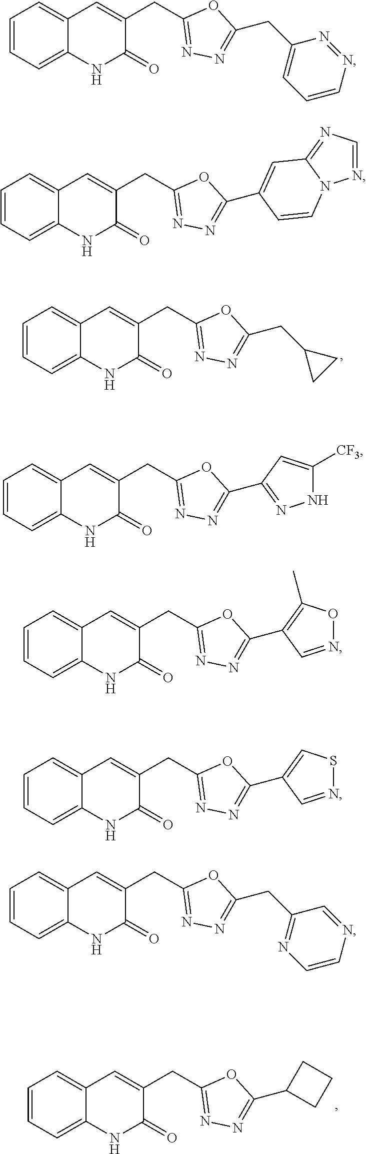

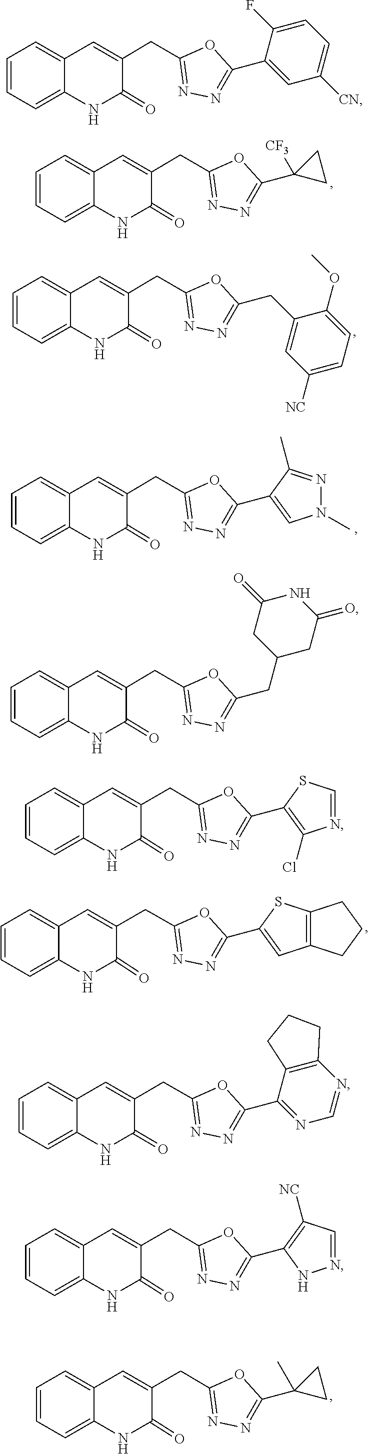

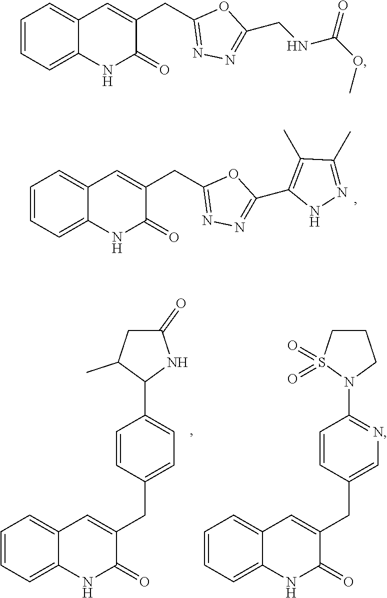

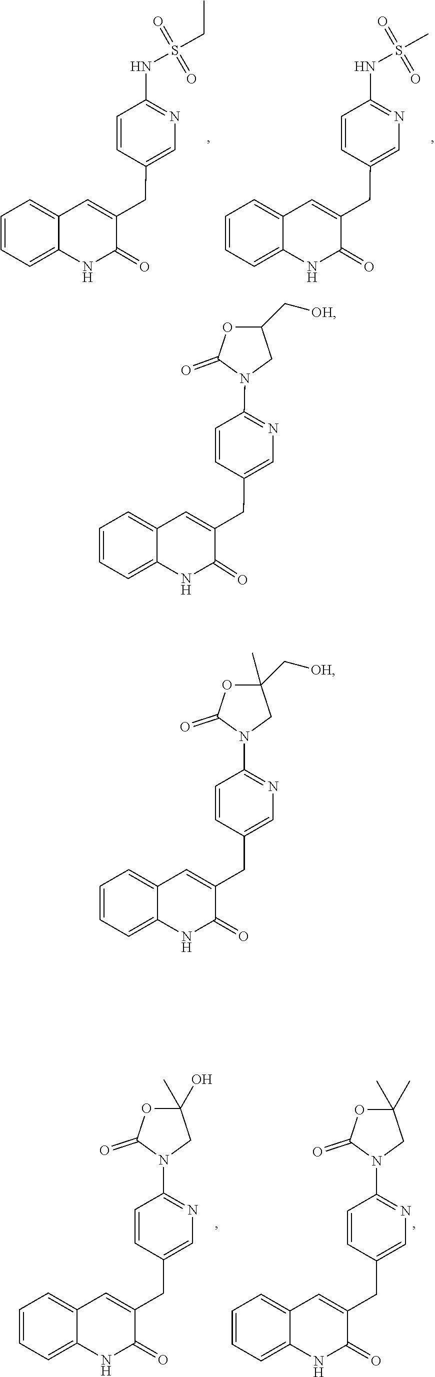

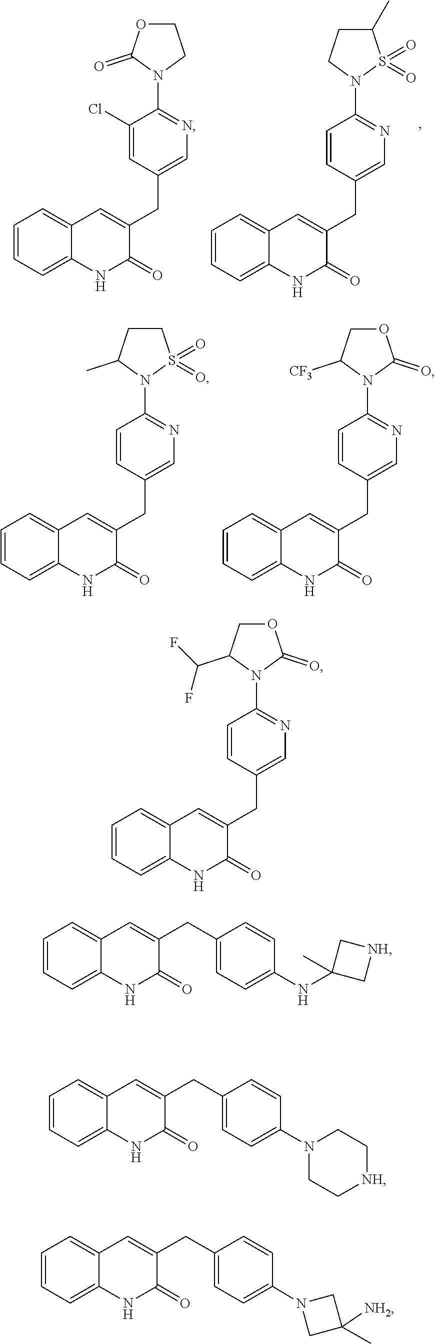

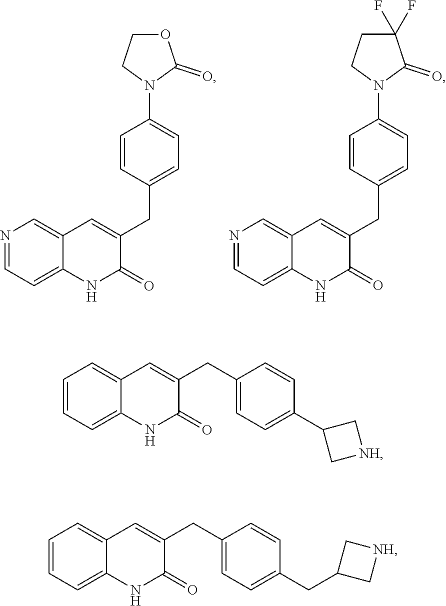 [SMILES: O=C1OCCN1c1ccc(Cc2cc3cnccc3[nH]c2=O)cc1 ; O=C1N(c2ccc(Cc3cc4cnccc4[nH]c3=O)cc2)CCC1(F)F ; O=c1[nH]c2ccccc2cc1Cc1ccc(C2CNC2)cc1 ; O=c1[nH]c2ccccc2cc1Cc1ccc(CC2CNC2)cc1]

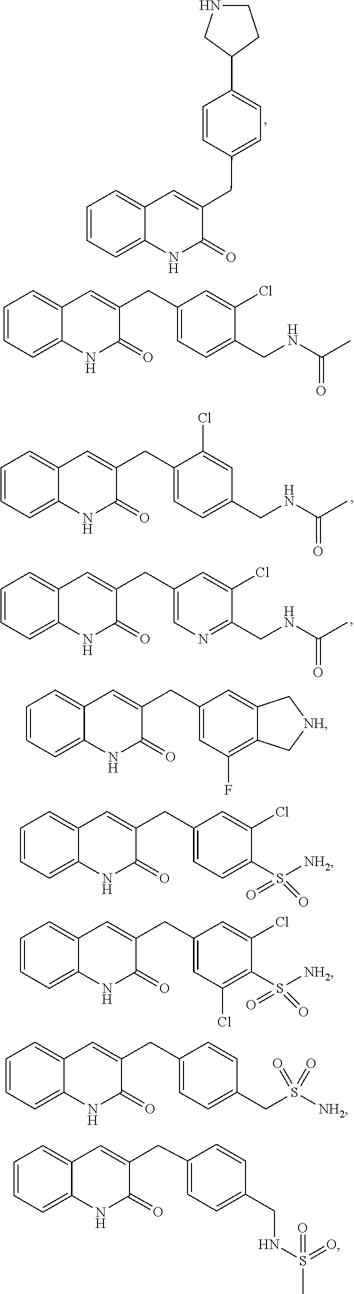

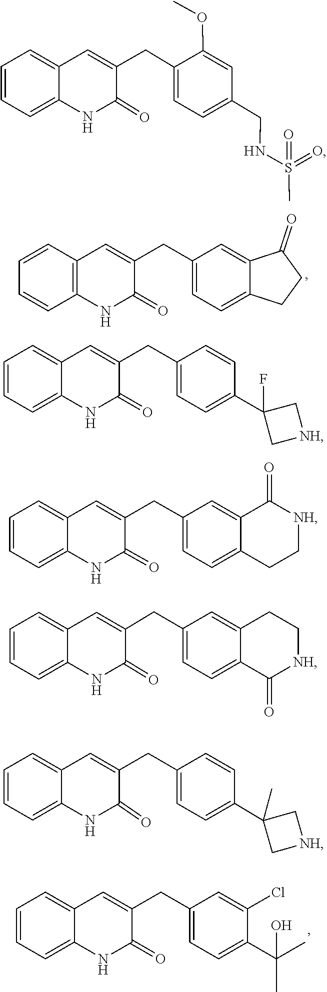

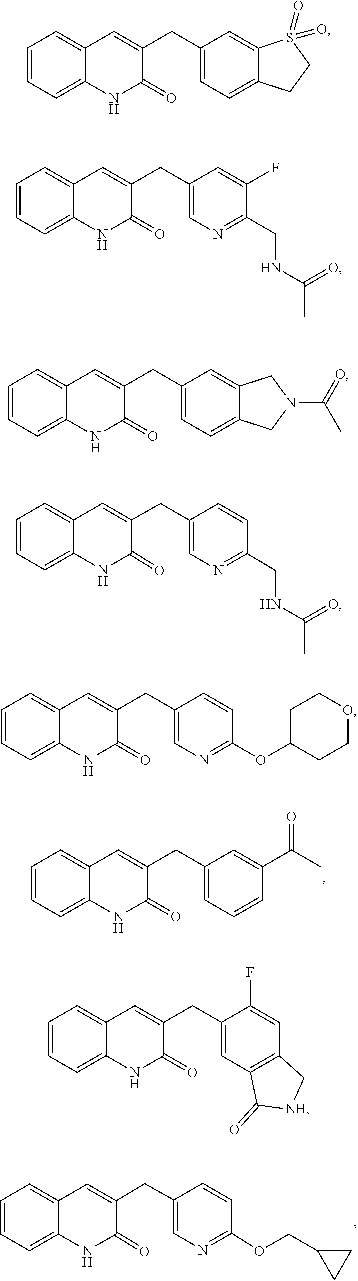

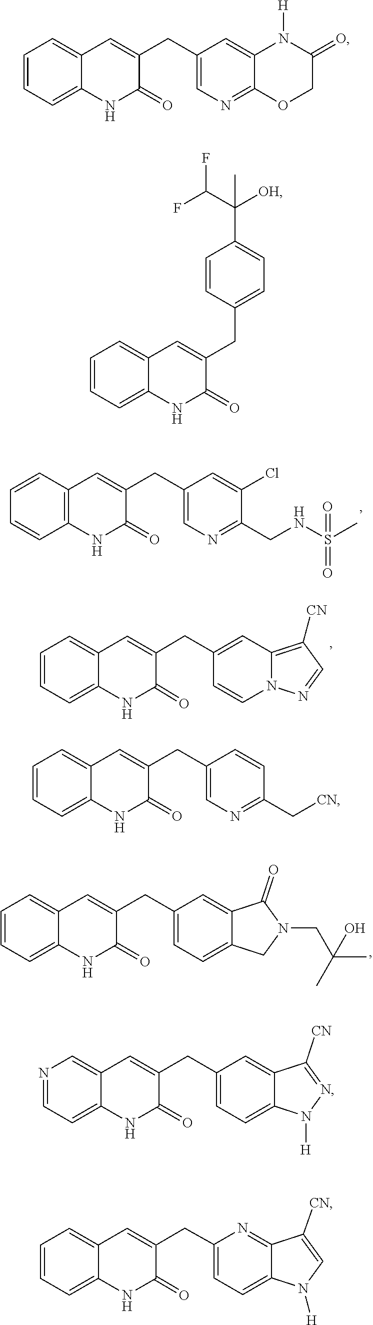

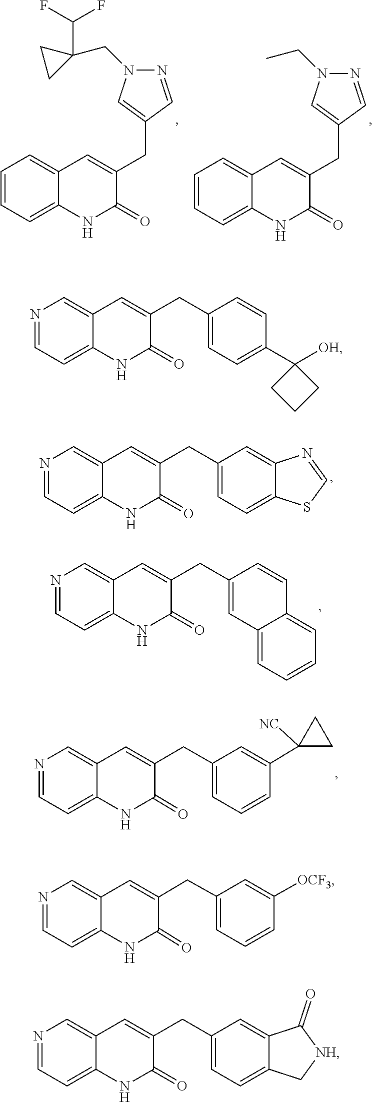

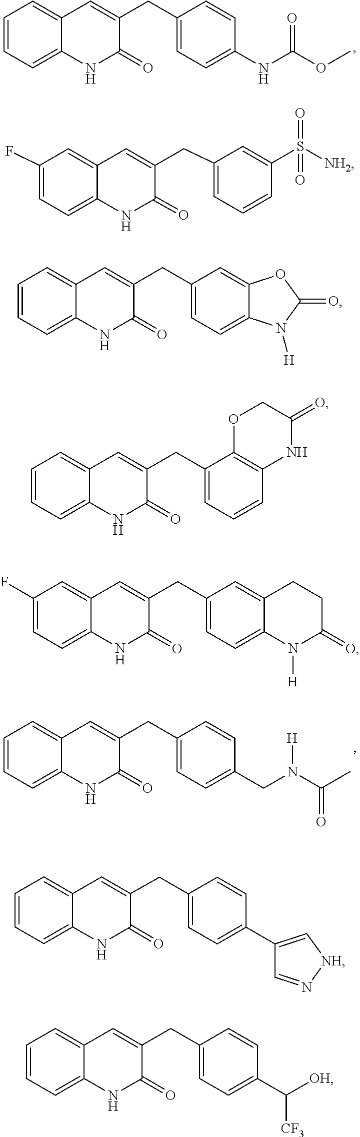

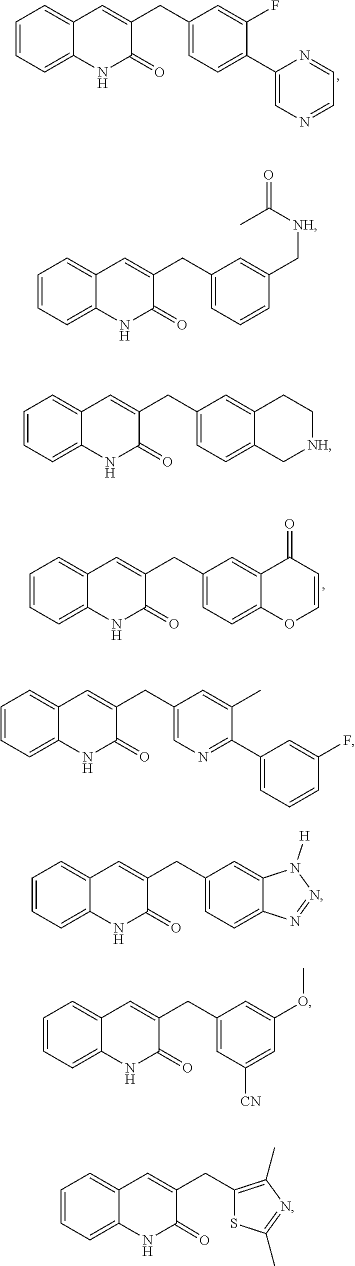

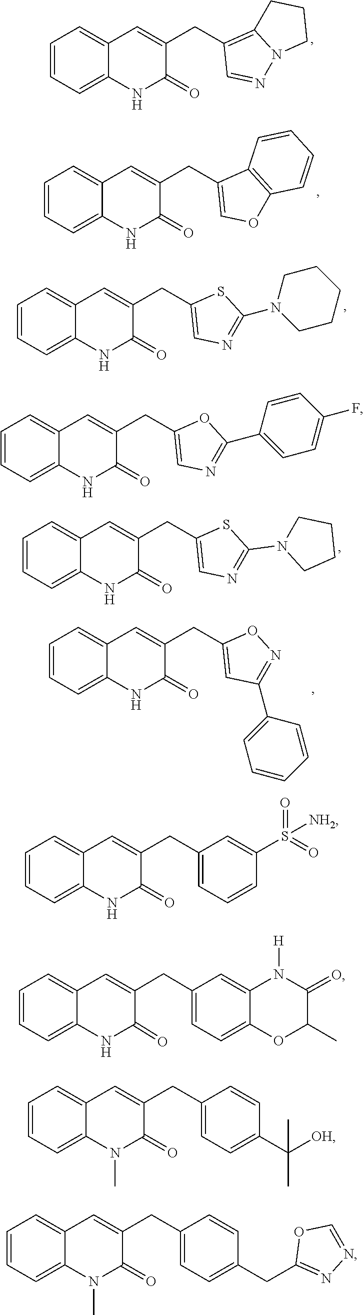

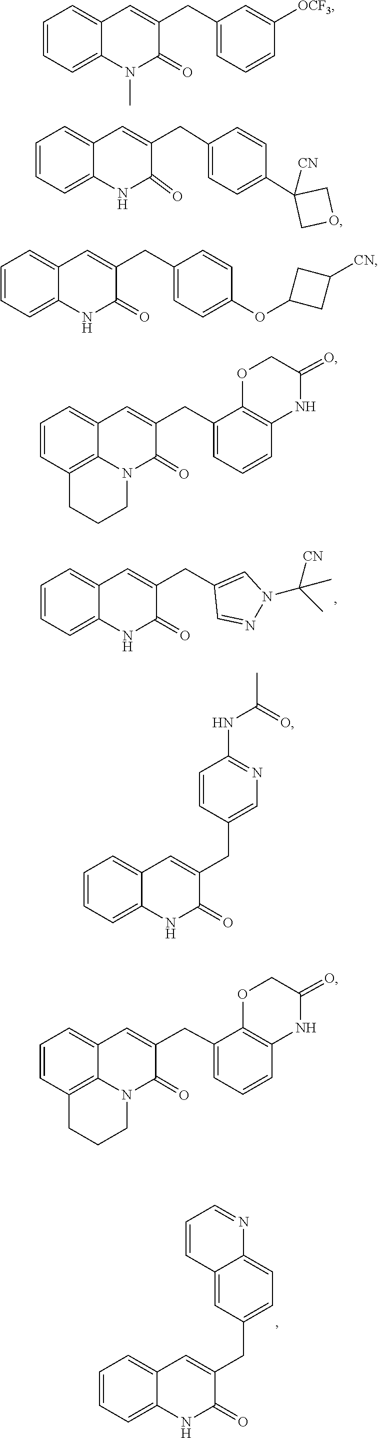

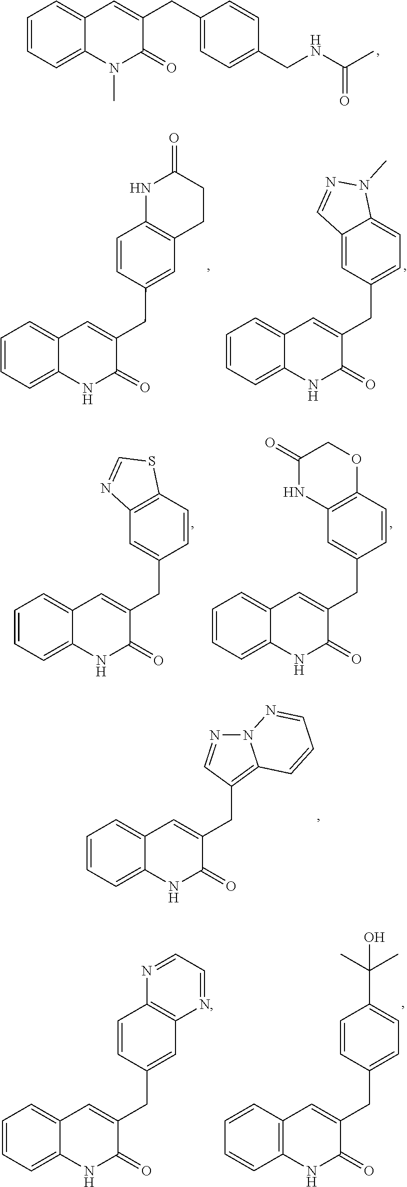

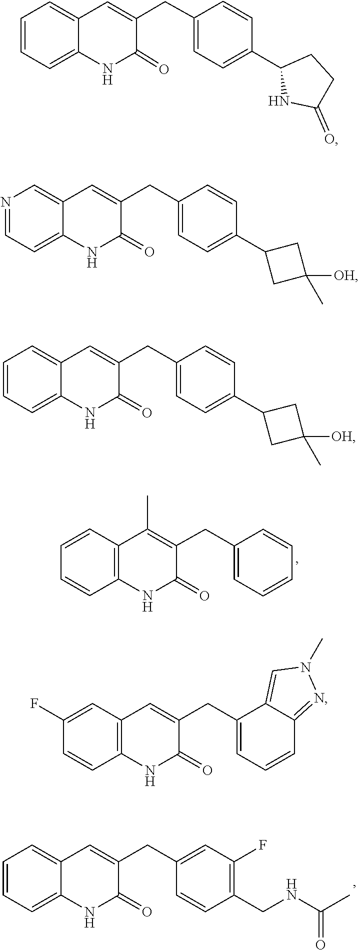 [SMILES: O=C1CC[C@@H](c2ccc(Cc3cc4ccccc4[nH]c3=O)cc2)N1 ; CC1(O)CC(c2ccc(Cc3cc4cnccc4[nH]c3=O)cc2)C1 ; CC1(O)CC(c2ccc(Cc3cc4ccccc4[nH]c3=O)cc2)C1 ; Cc1c(Cc2ccccc2)c(=O)[nH]c2ccccc12 ; Cn1cc2c(Cc3cc4cc(F)ccc4[nH]c3=O)cccc2n1 ; CC(=O)NCc1ccc(Cc2cc3ccccc3[nH]c2=O)cc1F]

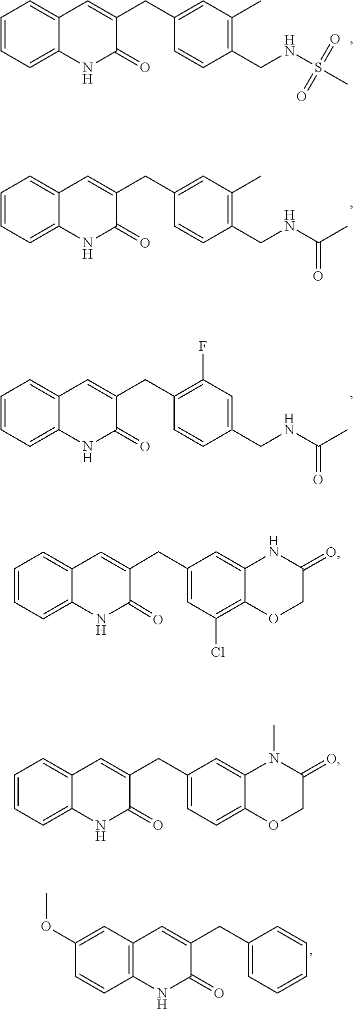 [SMILES: Cc1cc(Cc2cc3ccccc3[nH]c2=O)ccc1CNS(C)(=O)=O ; CC(=O)NCc1ccc(Cc2cc3ccccc3[nH]c2=O)cc1C ; CC(=O)NCc1ccc(Cc2cc3ccccc3[nH]c2=O)c(F)c1 ; O=C1COc2c(Cl)cc(Cc3cc4ccccc4[nH]c3=O)cc2N1 ; CN1C(=O)COc2ccc(Cc3cc4ccccc4[nH]c3=O)cc21 ; COc1ccc2[nH]c(=O)c(Cc3ccccc3)cc2c1]

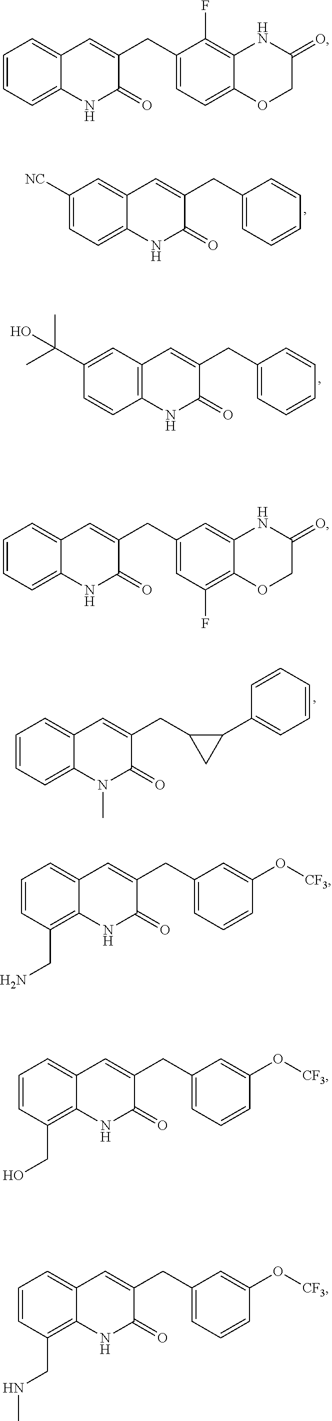

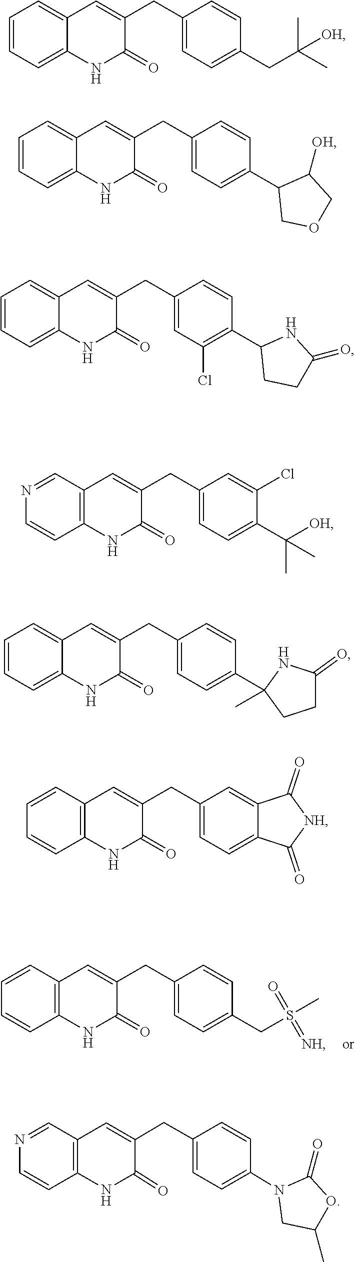

or pharmaceutically acceptable salt thereof.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

Under nomenclature used throughout this disclosure, the terminal portion of the designated side chain is described last preceded by the adjacent functionality toward the point of attachment.

The term “halogen” includes a fluorine, a chlorine, a bromine or an iodine radical.

The term “oxo” means the functional group “═O”, such as, for example, (1) “C═(O)”, that is a carbonyl group; (2) “S═(O)”, that is, a sulfoxide group; and (3) “N═(O)”, that is, an N-oxide group, such as pyridyl-N-oxide.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “alkoxy” or “—OC₁-C₆alkyl” refers to an alkyl group having 1 to 6 carbons linked to oxygen. Examples include methoxy, ethoxy, butoxy, isopropoxy and propoxy.

The term “haloalkoxy” or “—OC₁-C₆halogen-substituted alkyl” refers to an alkoxy group, wherein one or more hydrogens is replaced with a halogen. Examples include trifluoromethoxy.

The term “COOC₁-C₆alkyl” refers to a —COOH group wherein the —OH is replaced with an alkoxy group as defined above. Examples include methoxycarbonyl, ethoxycarbonyl, isopropylcarbonyl and butoxycarbonyl.

The term “COC₁-C₆alkyl” refers to a —COH group wherein the —OH is replaced with an alkoxy group as defined above.

The term “haloC₁-C₆alkyl” encompasses C₁-C₆alkyl with the hydrogen atoms thereof being partially or completely substituted with halogen, examples thereof including fluoromethyl, difluoromethyl, trifluoromethyl, 2-fluoroethyl, 1,2-difluoroethyl, 2,2-difluoroethyl and the like.

The term “aryl” means mono- or bicyclic aromatic rings containing only carbon atoms. Examples include phenyl and naphthyl.

The term “C₃-C₁₀cycloalkyl” encompasses bridged, saturated or unsaturated cycloalkyl groups having 3 to 8 carbons. “Cycloalkyl” also includes non-aromatic rings, monocyclic, non-aromatic rings fused to a saturated cycloalkyl group and aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Additional examples include

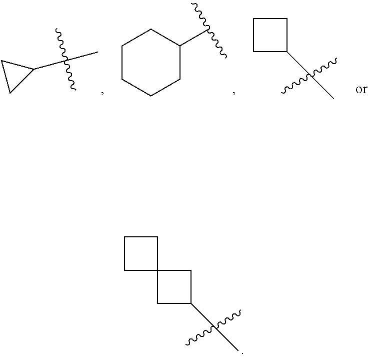

The term “heteroaryl” means an aromatic cycloheteroalkyl that contains at least one ring heteroatom selected from O, S and N. Examples of heteroaryl groups include pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, oxadiazolyl, quinoxalinyl, purinyl, benzimidazolyl, quinolyl, isoquinolyl, and the like. Additional examples include

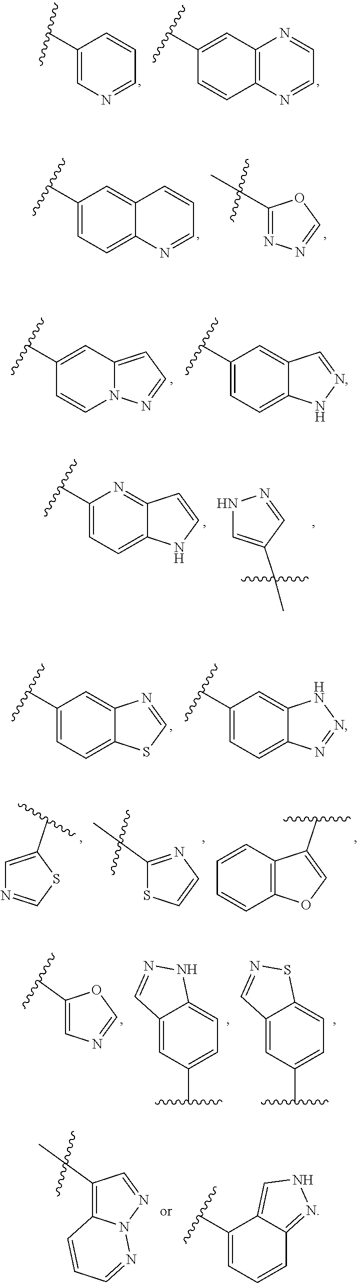

The term “cycloheteroalkyl” means mono- or bicyclic or bridged partially unsaturated and saturated rings containing at least one heteroatom selected from N, S and O, each of said rings having from 3 to 10 atoms in which the point of attachment may be carbon or nitrogen. Examples include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, tetrahydropyran, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Additional examples include

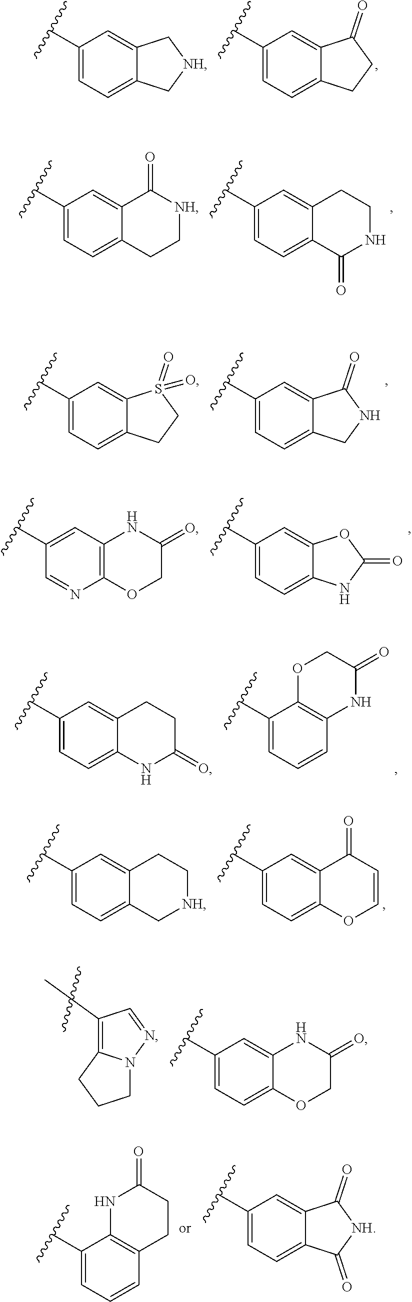

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, including a human, canine, feline, bovine, or porcine patient, preferably a human patient, receiving or about to receive medical treatment.

The term “treat” or “treatment” means to administer an agent, such as a composition containing any of the compounds described herein, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein (i.e. Formula I and Formulas 1a-1i) are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein (i.e. Formula I and Formulas 1a-1i). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents or Intermediates.

It should be noted that chemically unstable compounds are excluded from the embodiments contained herein.

### Methods of Treatment

Also encompassed by the present invention are methods of preventing, treating or ameliorating IL4I1-related diseases. The compounds described herein can be effective in preventing, treating or ameliorating various IL4I1-related diseases, such as cancer. Described herein are methods for treatment of cancer displaying IL4I1-expressing cells in a patient comprising administration of a compound of the invention, or a pharmaceutical salt thereof, to the patient. Described herein are methods for prevention of cancer displaying IL4I1-expressing cells in a patient comprising administration of a compound of the invention, or a pharmaceutical salt thereof, to the patient. Described herein are methods for ameliorating the symptoms or clinical effects of cancer displaying IL4I1-expressing cells in a patient comprising administration of a compound of the invention, or a pharmaceutical salt thereof, to the patient.

In one embodiment described herein, the cancer to be treated is selected from the group consisting of cancers displaying IL4I1-expressing cells and lymphomas displaying IL4I1-expressing cells. In certain embodiment, the cancers to be treated are solid tumors. In certain embodiments, the cancers to be treated are selected from carcinomas, sarcomas, mesotheliomas, blastomas and germ cell tumors. In another particular embodiment, the cancer to be treated is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be treated is a lymphoma displaying IL4I1-expressing cells such as a B-cell lymphoma displaying IL4I1-expressing cells.

In certain embodiments, the cancer to be treated is a cancer displaying IL4I1-expressing cells selected from the group consisting of PMBL (Primary Mediastinal large B-cell Lymphoma), classical Hodgkin lymphomas (cHL), NLPHL (Nodular lymphocyte predominant Hodgkin's lymphoma), non-mediastinal Diffuse Large B-Cell Lymphoma (DLBCL) and SLL/CLL (Small Lymphocytic Lymphoma/Chronic Lymphocytic Leukemia). In another specific embodiment, the cancer to be treated is a lymphoma displaying IL4I1-expressing cells.

In one embodiment described herein, the cancer to be prevented is selected from the group consisting of cancers displaying IL4I1-expressing cells and lymphomas displaying IL4I1-expressing cells. In a certain embodiment, the cancer to be prevented is a solid tumor. In certain embodiments, the cancer to be prevented is selected from carcinomas, sarcomas, mesotheliomas, blastomas and germ cell tumors. In another particular embodiment, the cancer to be prevented is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be prevented is a lymphoma displaying IL4I1-expressing cells such as a B-cell lymphoma displaying IL4I1-expressing cells.

In certain embodiments, the cancer to be prevented is a cancer displaying IL4I1-expressing cells selected from the group consisting of PMBL, cHL, NLPHL, DLBCL and SLL/CLL. In another specific embodiment, the cancer to be treated is a lymphoma displaying IL4I1-expressing cells.

In one embodiment described herein, the cancer to be ameliorated is selected from the group consisting of cancers displaying IL4I1-expressing cells and lymphomas displaying IL4I1-expressing cells. In a certain embodiment, the cancer to be ameliorated is a solid tumor. In certain embodiments, the cancer to be ameliorated is selected from carcinomas, sarcomas, mesotheliomas, blastomas and germ cell tumors. In another particular embodiment, the cancer to be ameliorated is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be ameliorated is a lymphoma displaying IL4I1-expressing cells such as a B-cell lymphoma displaying IL4I1-expressing cells.

In certain embodiments, the cancer to be ameliorated is a cancer displaying IL4I1-expressing cells selected from the group consisting of PMBL, cHL, NLPHL, DLBCL and SLL/CLL. In another specific embodiment, the cancer to be treated is a lymphoma displaying IL4I1-expressing cells.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

Accordingly, the invention relates to pharmaceutical compositions comprising a therapeutically effective amount of a compound of the invention (i.e. a compound of Formula I or any of Formulas 1a-1i, or any IL4i1 inhibitor compound described herein), or a pharmaceutically acceptable salt thereof, and a pharmaceutically acceptable carrier. As used herein, a “therapeutically effective amount” is an amount sufficient to produce the desired clinical outcome, e.g. treatment or prevention of cancer displaying Il4i1-expressing cells or amelioration of the clinical effects or presentation thereof. Such a therapeutically effective amount may be contained in a single dosage form (e.g. one tablet or injection) or split into more than one of the dosage form (e.g. more than one tablet or injection, which together contain a therapeutically effective amount).

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropyl cellulose, sorbitol, sorbitan fatty acid ester, polysorbate, sucrose fatty acid ester, polyoxymethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day, or from about 0.001 to about 50 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

Examples of other active ingredients that may be administered in combination with a compound of any of the Formulas described herein (e.g. Formula I or any of Formulas 1a-1i) or a pharmaceutically acceptable salt thereof and either administered separately or in the same pharmaceutical composition, include, but are not limited to pain relieving agents, anti-angiogenic agents, anti-neoplastic agents, anti-diabetic agents, anti-infective agents, or gastrointestinal agents, or combinations thereof.

Suitable compounds that may be used in combination with a compound according to the present invention include without limitation sildenafil, vardenafil, tadalafil and alprostadil, epoprostenol, iloprost, bosentan, amlodipine, diltiazem, nifedipine, ambrisentan and warfarin, fluticasone, budesonide, mometasone, flunisolide, beclomethasone, montelukast, zafirlukast, zileuton, salmeterol, formoterol, theophylline, albuterol, levalbuterol, pirbuterol, ipratropium, prednisone, methylprednisolone, omalizumab, corticosteroid and cromolyn, atorvastatin, lovastatin, simvastatin, pravastatin, fluvastatin, rosuvastatin, gemfibrozil, fenofibrate, nicotinic acid, and clopidogrel and pharmaceutically acceptable salts thereof.

Additionally, a compound of any of the Formulas disclosed herein (e.g. Formula I or any of Formulas 1a-1i) may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present invention.

In one embodiment, the other active agent is selected from the group consisting of vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines, CTLA-4, LAG-3 and PD-1 antagonists.

PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Higashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in particular embodiments, the human constant region is an IgG1 or IgG4 constant region. In some embodiments, the antigen binding fragment is selected from the group consisting of Fab, Fab′-SH, F(ab′)₂, scFv and Fv fragments. Examples of PD-1 antagonists include, but are not limited to, pembrolizumab (KEYTRUDA®, Merck & Co., Inc., Rahway, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMFINZI®, Astra Zeneca Pharmaceuticals, LP, Wilmington, DE, avelumab (BAVENCIO®, Merck KGaA, Darmstadt, Germany and Pfizer, Inc., New York, NY), cemiplimab (LIBTAYO®, Regeneron Pharmaceuticals, Inc., Tarrytown, NY, and Sanofi-Aventis LLC, Bridgewater, NJ), dostarlimab (JEMPERLI®, GlaxoSmithKline LLC, Philadelphia, PA), Sasanlimab (PF-06801591), Retifanlimab (MGA012), Cetrelimab (JNJ-63723283), Tebotelimab (MGD013), Cadonilimab, (AK104), Ezabenlimab (BI754091), Budigalimab (ABBV-181), Spartalizumab (PDR001), Zimberelimab (AB122), Serplulimab (HLX10), Cosibelimab (CK-301), Sugemalimab (CS1001), Camrelizumab, Sintilimab, Tislelizumab, Toripalimab (TAB001), Penpulimab (AK105) and Adebrelimab (SHR-1316).

Examples of monoclonal antibodies (mAbs) that bind to human PD-1, which are useful in the treatment methods, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, which are useful in the treatment methods, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein that binds to human PD-1.

Thus, one embodiment provides a method of treating cancer comprising administering an effective amount of a compound of the invention, or a pharmaceutically acceptable salt thereof, in combination with a PD-1 antagonist to a subject in need thereof. In such embodiments, a compound of the invention, or a pharmaceutically acceptable salt thereof, and a PD-1 antagonist are administered concurrently or sequentially.

Specific non-limiting examples of such cancers which can be treated in accordance with this embodiment include melanoma (including unresectable or metastatic melanoma), head & neck cancer (including recurrent or metastatic head and neck squamous cell cancer), classical Hodgkin lymphoma, urothelial carcinoma, gastric cancer, cervical cancer, primary mediastinal large-B-cell lymphoma, microsatellite instability-high cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, recurrent or metastatic head and neck squamous cell cancer, classical Hodgkin lymphoma, urothelial carcinoma, gastric cancer, cervical cancer, primary mediastinal large-B-cell lymphoma, microsatellite instability-high or mismatch repair deficient cancer, esophageal cancer, renal cancer, endometrial carcinoma, tumor mutational burden-high cancer, triple negative breast cancer, non-small cell lung cancer, and hepatocellular carcinoma. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab, or any of the anti-PD-1 and anti-PD-L1 antibodies disclosed herein. In one such embodiment, the agent is pembrolizumab. In another such embodiment, the agent is nivolumab. In another such embodiment, the agent is atezolizumab.

Pembrolizumab is approved by the U.S. FDA for the treatment of patients with unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), microsatellite instability-high or mismatch repair deficient cancer, microsatellite instability-high or mismatch repair deficient colorectal cancer (CRC), primary mediastinal large B-cell lymphoma, gastric cancer, urothelial cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, triple-negative breast cancer (TNBC), as described in the Prescribing Information for KEYTRUDA™ (Merck & Co., Inc., Rahwayg, NJ USA; initial U.S. approval 2014, updated February 2022). In another embodiment, there is provided a method of treating cancer comprising administering an effective amount of a compound of the invention, or a pharmaceutically acceptable salt thereof, to a person in need thereof, in combination with pembrolizumab, wherein said cancer is selected from unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), microsatellite instability-high or mismatch repair deficient cancer, microsatellite instability-high or mismatch repair deficient colorectal cancer (CRC), gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), triple-negative breast cancer (TNBC).

In another embodiment, there is provided a method of treating cancer comprising administering an effective amount of a compound of the invention, or a pharmaceutically acceptable salt thereof in combination with a PD-1 antagonist, to a person in need thereof, wherein said cancer is selected from unresectable or metastatic melanoma, Stage IIB, IIC, or III melanoma following complete resection, non-small cell lung cancer, head and neck squamous cell cancer, classical Hodgkin Lymphoma, microsatellite instability-high or mismatch repair deficient cancer, microsatellite instability-high or mismatch repair deficient colorectal cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, and triple-negative breast cancer. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab, or any of the anti-PD-1 and anti-PD-L1 antibodies disclosed herein. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab.

In another embodiment, there is provided a method of treating cancer comprising administering an effective amount of a compound of the invention, or a pharmaceutically acceptable salt thereof, in combination with a PD-1 antagonist, to a person in need thereof, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab.

In one embodiment, there is provided a method of treating unresectable or metastatic melanoma comprising administering an effective amount of a compound of the invention, or a pharmaceutically acceptable salt thereof, in combination with a PD-1 antagonist to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating recurrent or metastatic head and neck squamous cell cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating classical Hodgkin lymphoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating triple-negative breast cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating urothelial carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating gastric cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating cervical cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating primary mediastinal large-B-cell lymphoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating microsatellite instability-high (MSI-H) cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating non-small cell lung cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating hepatocellular carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating Merkel cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating renal cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating endometrial cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating cutaneous squamous cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating tumor mutational burden-high cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

Examples of vascular endothelial growth factor (VEGF) receptor inhibitors include, but are not limited to, bevacizumab (sold under the trademark AVASTIN by Genentech/Roche), axitinib, (N-methyl-2-[[3-[([pound])-2-pyridin-2-ylethenyl]-1H-indazol-6-yl]sulfanyl]benzamide, also known as AG013736, and described in PCT Publication No. WO01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)₂-aminopropanoate, also known as BMS-582664), motesanib (N-(2,3-dihydro-3,3-dimethyl-1H-indoi-6-yl)-2-[(4-pyridinyimethy)amino]-3-pyridinecarboxamide. and described in PCT Publication No. WO 02/068470), pasireotide (also known as SO 230, and described in PCT Publication No. WO02/010192), and sorafenib (sold under the tradename NEXAVAR).

Examples of topoisomerase II inhibitors include but are not limited to, etoposide (also known as VP-16 and Etoposide phosphate, sold under the tradenames TOPOSAR, VEPESID and ETOPOPHOS), and teniposide (also known as VM-26, sold under the tradename VUMON).

Examples of alkylating agents include but are not limited to, 5-azacytidine (sold under the trade name VIDAZA), decitabine (sold under the trade name of DECOGEN), temozolomide (sold under the trade names TEMODAR and TEMODAL by Merck & Co., Inc., Rahway, NJ, USA), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX and MYLERAN), carboplatin (sold under the tradename PARAPLATIN), lomustine (also known as CCNU, sold under the tradename CeeNU), cisplatin (also known as CDDP, sold under the tradenames PLATINOL and PLATINOL-AQ), chlorambucil (sold under the tradename LEUKERAN), cyclophosphamide (sold under the tradenames CYTOXAN and NEOSAR), dacarbazine (also known as DTIC, DIC and imidazole carboxamide, sold under the tradename DTIC-DOME), altretamine (also known as hexamethylmelamine (HMM) sold under the tradename HEXALEN), ifosfamide (sold under the tradename IFEX), procarbazine (sold under the tradename MATULANE), mechlorethamine (also known as nitrogen mustard, mustine and mechloroethamine hydrochloride, sold under the tradename MUSTARGEN), streptozocin (sold under the tradename ZANOSAR), thiotepa (also known as thiophosphoamide, TESPA and TSPA, and sold under the tradename THIOPLEX).

Examples of anti-tumor antibiotics include, but are not limited to, doxorubicin (sold under the tradenames ADRIAMYCIN and RUB EX), bleomycin (sold under the tradename LENOXANE), daunorubicin (also known as dauorubicin hydrochloride, daunomycin, and rubidomycin hydrochloride, sold under the tradename CERUBIDINE), daunorubicin liposomal (daunorubicin citrate liposome, sold under the tradename DAUNOXOME), mitoxantrone (also known as DHAD, sold under the tradename NOVANTRONE), epirubicin (sold under the tradename ELLENCE), idarubicin (sold under the tradenames IDAMYCIN, IDAMYCIN PFS), and mitomycin C (sold under the tradename MUTAMYCIN).

Examples of anti-metabolites include, but are not limited to, claribine (2-chlorodeoxyadenosine, sold under the tradename LEUSTATIN), 5-fluorouracil (sold under the tradename ADRUCIL), 6-thioguanine (sold under the tradename PURINETHOL), pemetrexed (sold under the tradename ALIMTA), cytarabine (also known as arabinosylcytosine (Ara-C), sold under the tradename CYTOSAR-U), cytarabine liposomal (also known as Liposomal Ara-C, sold under the tradename DEPOCYT), decitabine (sold under the tradename DACOGEN), hydroxyurea (sold under the tradenames HYDREA, DROXIA and MYLOCEL), fludarabine (sold under the tradename FLUDARA), floxuridine (sold under the tradename FUDR), cladribine (also known as 2-chlorodeoxyadenosine (2-CdA) sold under the tradename LEUSTATIN), methotrexate (also known as amethopterin, methotrexate sodium (MTX), sold under the tradenames RHEUMATREX and TREXALL), and pentostatin (sold under the tradename NIPENT).

Examples of retinoids include, but are not limited to, alitretinoin (sold under the tradename PANRETIN), tretinoin (all-trans retinoic acid, also known as ATRA, sold under the tradename VESANOID), Isotretinoin (13-c/s-retinoic acid, sold under the tradenames ACCUTANE, AMNESTEEM, CLARAVIS, CLARUS, DECUTAN, ISOTANE, IZOTECH, ORATANE, ISOTRET, and SOTRET), and bexarotene (sold under the tradename TARGRETIN).

In such combinations the compound of the present invention and other active agents may be administered separately or in conjunction. In addition, the administration of one agent may be prior to, concurrent to, or subsequent to the administration of other agent(s).

### EXAMPLES

### Schemes

In general, compounds of Formula I were synthesized according to one of Schemes 1-15.

### Scheme 1

Certain compounds of Formula I were synthesized by converting alkyl chloride 1 to 2 under palladium catalyzed Suzuki conditions with the corresponding aryl boronic acid(ester) or under nickel catalyzed reductive coupling conditions with the corresponding aryl bromide. Then a deprotection completed the synthesis.

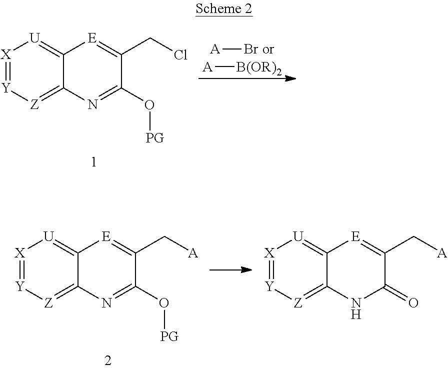

Certain compounds of Formula I were synthesized from alkyl chloride 3 under palladium catalyzed Suzuki conditions with the corresponding aryl/benzyl boronic acid(ester) or under nickel catalyzed reductive coupling conditions with the corresponding aryl bromide.

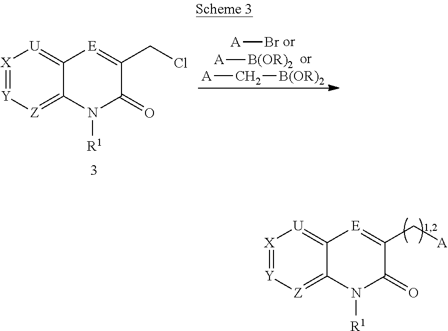

Certain compounds of Formula I were synthesized from aryl bromide 3 under palladium catalyzed Negishi conditions with the corresponding benzyl zinc reagent.

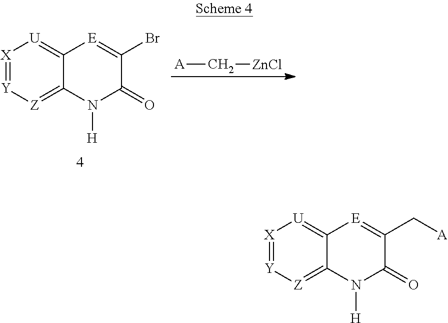

Certain compounds of Formula I were synthesized from aryl acetic acid 5 with the corresponding hydrazide under amide coupling/dehydration conditions (oxadiazole formation).

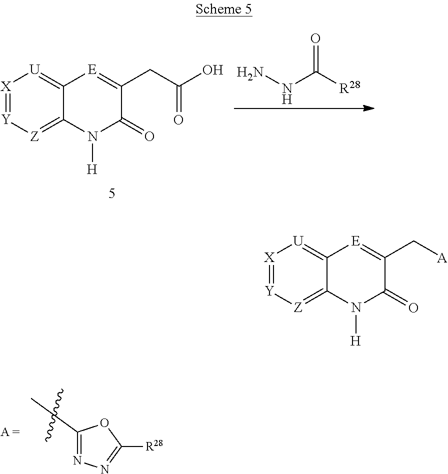

Certain compounds of Formula I were synthesized from aryl acetyl hydrazide 6 with the corresponding carboxylic acid under amide coupling/dehydration conditions (oxadiazole formation).

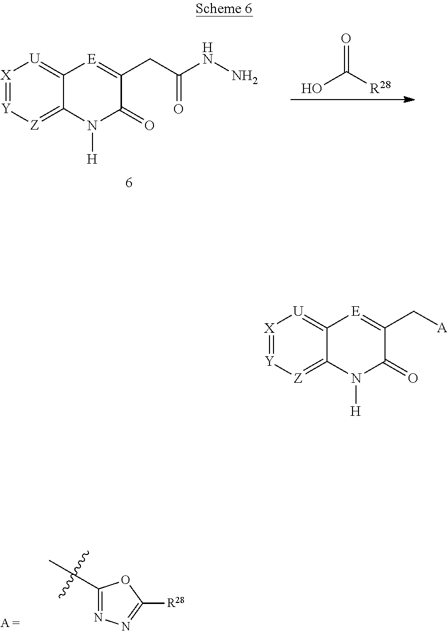

Certain compounds of Formula I were synthesized by converting, alkyl chloride 1 to aryl halide 7 under palladium catalyzed Suzuki conditions with the corresponding haloaryl boronic acid(ester). Then 7 was converted to 8 under Cu or Pd catalyzed N-arylation conditions with the corresponding nitrogen-centered nucleophile, Ni/Ir catalyzed C—H arylation conditions with the corresponding alkane, or Ni catalyzed reductive coupling with the corresponding alkyl halide. Then a deprotection completed the synthesis.

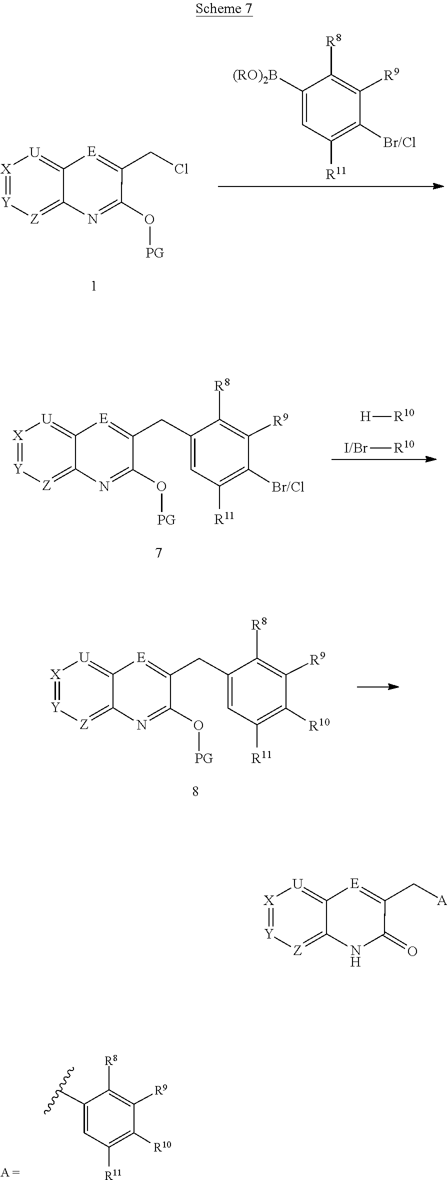

Certain compounds of Formula I were synthesized by converting alkyl chloride 1 to aryl halide 9 under palladium catalyzed Suzuki conditions with the corresponding halopyridyl boronic acid(ester). Then 9 was converted to 10 under Cu or Pd catalyzed N-arylation conditions with the corresponding nitrogen-centered nucleophile or Ni catalyzed reductive coupling with the corresponding alkyl halide. Then a deprotection completed the synthesis.

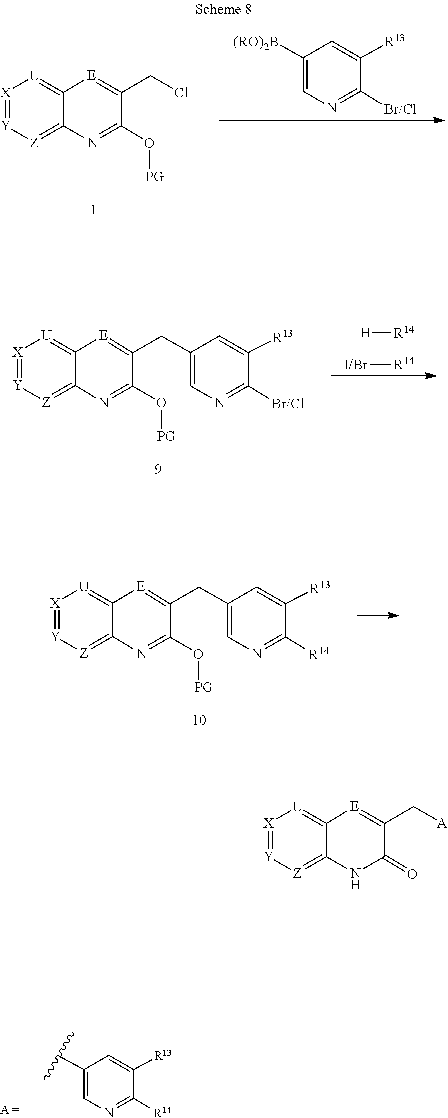

Certain compounds of Formula I were synthesized by converting alkyl chloride 1 to carboxylic acid 10 under palladium catalyzed Suzuki conditions with the corresponding aryl boronic acid and a deprotection step conducted in the same pot. Then the synthesis was completed with an amide coupling reaction.

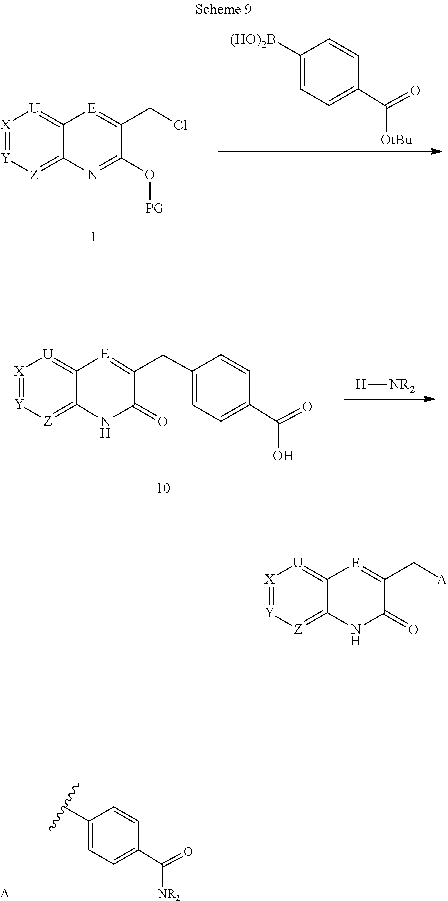

Certain compounds of Formula I were synthesized by converting alkyl chloride 3 was converted to alkene 11 under palladium catalyzed Suzuki conditions with the corresponding vinyl boronic acid. Then the synthesis was completed with a Pd catalyzed cyclopropanation with 1-methyl-nitrosourea as the methylene source.

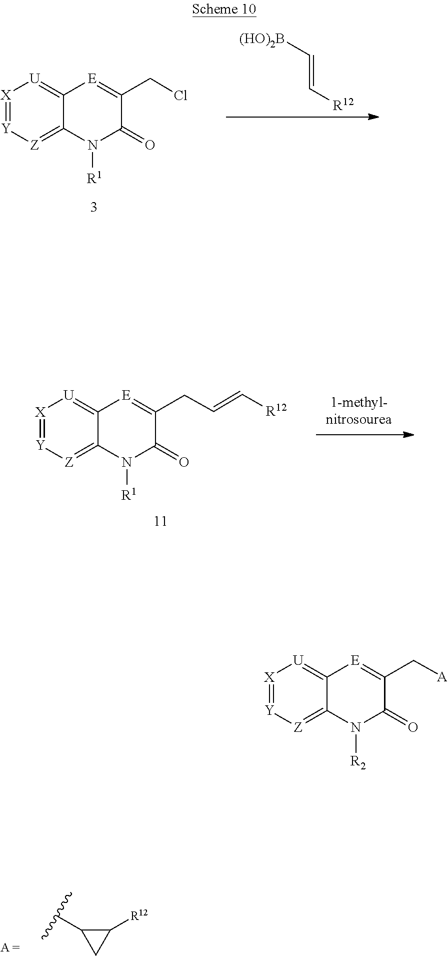

Certain compounds of Formula I were synthesized by converting alkyl chloride 1 to methyl carboxylate 12 under palladium catalyzed Suzuki conditions with the corresponding aryl boronic acid, followed by a separate deprotection step. Then the synthesis was completed with reaction with methyl magnesium bromide to afford the tertiary alcohol.

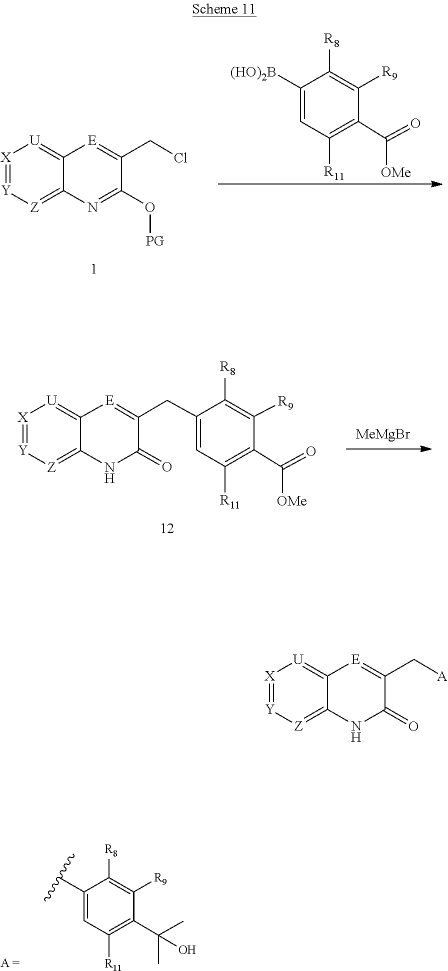

Certain compounds of Formula I were synthesized by reacting amides 13 with functionalized aryl aldehydes 14 using palladium catalyzed conditions.

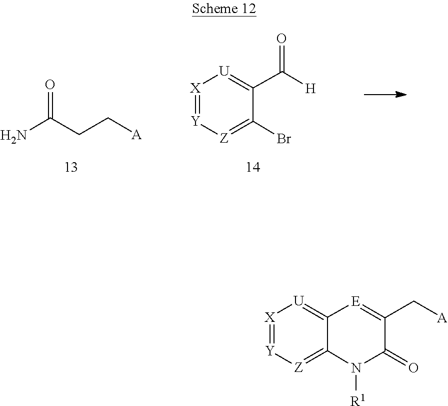

Certain compounds of Formula I were synthesized by reacting bromides 15 with n-BuLi followed by quench with acetone.

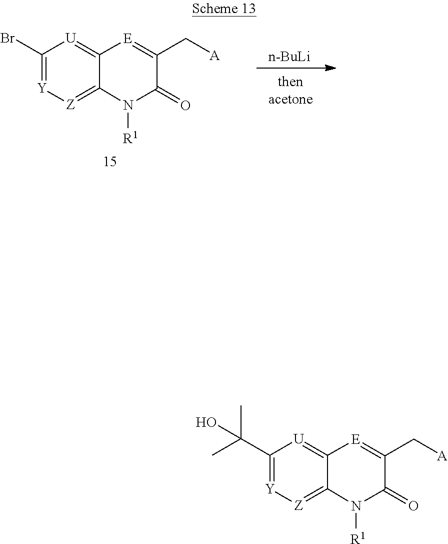

Certain compounds of Formula I were synthesized by reacting bromides 16 with copper cyanide under palladium catalyzed conditions to afford the aryl cyanides. Then a deprotection completed the synthesis.

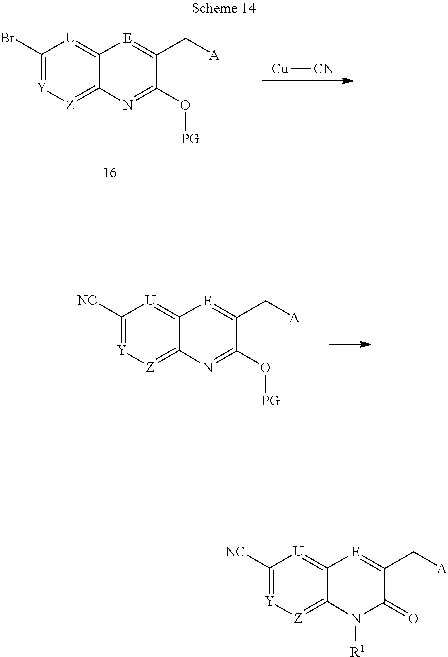

Certain compounds of Formula I were synthesized by compounds 16 with aldehydes in the presence of LDA. Subsequent reaction with TFA followed by Stille coupling under palladium catalyzed conditions completed the synthesis.

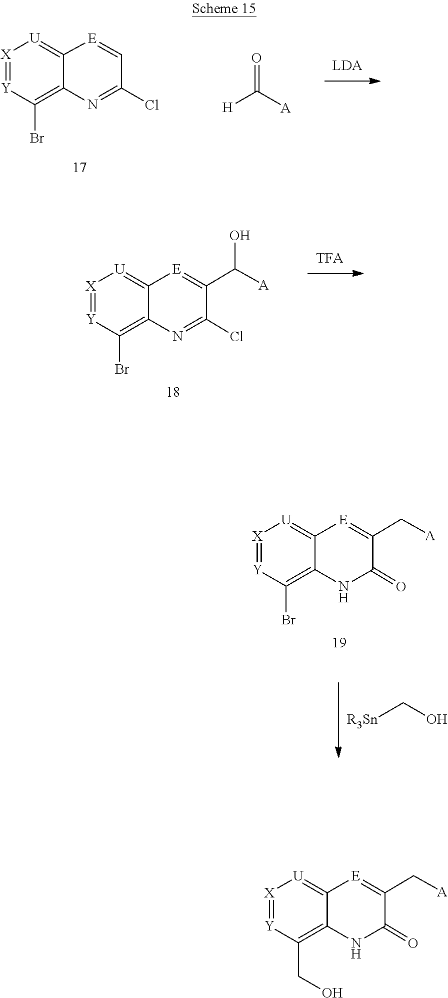

Certain compounds of Formula I were synthesized by reacting aryl bromides 19 with alkyl fluoroborates under palladium catalyzed conditions. Deprotection with acid afforded alkylamino compounds 20. Subsequent methylation completed the synthesis.

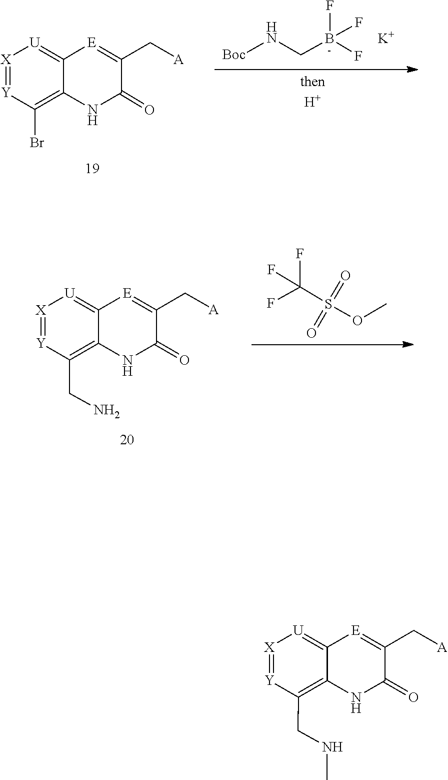

### General Experimental Information:

Unless otherwise noted, all reactions were magnetically stirred and performed under an inert atmosphere such as nitrogen or argon.

Unless otherwise noted, diethyl ether used in the experiments described below was Fisher ACS certified material and stabilized with BHT.

Unless otherwise noted, “concentrated” means evaporating the solvent from a solution or mixture using a rotary evaporator or vacuum pump.

Unless otherwise noted, “evaporated” means evaporating using a rotary evaporator or vacuum pump.

Unless otherwise noted, silica gel chromatography was carried out on an ISCO®, Analogix®, or Biotage® automated chromatography system using a commercially available cartridge as the column. Columns were usually filled with silica gel as the stationary phase.

Unless otherwise noted, proton nuclear magnetic resonance (¹H NMR) spectra and proton-decoupled carbon nuclear magnetic resonance (¹³C{¹H} NMR) spectra were recorded on 400, 500, or 600 MHz Bruker or Varian NMR spectrometers at ambient temperature. All chemical shifts (δ) were reported in parts per million (ppm). Proton resonances were referenced to residual protium in the NMR solvent, which can include, but is not limited to, CDCl₃, DMSO-d₆, and MeOD-d₄. Carbon resonances are referenced to the carbon resonances ofthe NMR solvent. Data are represented as follows: chemical shift, multiplicity (br=broad, br s=broad singlet, s=singlet, d=doublet, dd=doublet of doublets, ddd=doublet of doublet of doublets, t=triplet, q=quartet, mn=multiplet), coupling constants OJ in Hertz (Hz), integration.

Abbreviations used in the experimentals may include, but are not limited to, the following:

| | |
|---|---|
| AcOH | acetic acid |
| Aq | Aqueous |
| Boc | tert-butoxycarbonyl |
| Celite | diatomaceous earth |
| DCM | dichloromethane |
| DIPEA | N,N-diisopropylethylamine |
| DMA | N,N-dimethylacetamide |
| DMAP | 4-(dimethylamino)pyridine |
| DME | 1,2-dimethoxyethane |
| DMF | N,N-dimethylformamide |
| DMSO | dimethylsulfoxide |
| Dppf | 1,1′-bis(diphenylphosphino)ferrocene |
| Dtbpf | 1,1′-Bis(di-tert-butylphosphino)ferrocene |
| EtOAc | ethyl acetate |
| H | Hours |
| HATU | (1-[bis(dimethylamino)methylene]-1H-1,2,3-triazolo[4,5-b]pyridinium |
| | 3-oxide hexafluorophosphate |
| HPLC | high performance liquid chromatography |
| Ir[dF(CF3)ppy] 2(dtbbpy)PF6 | 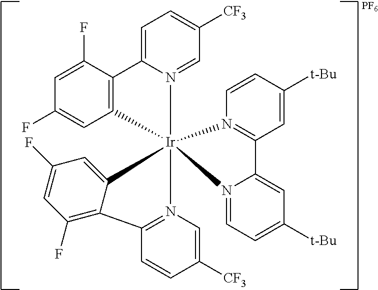 |
| LC/MS | liquid chromatography mass spectrometry |
| M | molarity |
| MeCN | Acetonitrile |
| MeOH | Methanol |
| MP-TMT | macroporous polystyrene-bound 2,4,6-mercaptotriazine |
| MTBE | methyl tert-butyl ether |
| NaHMDS | sodium(bistrimethylsilyl)amide |
| NBS | N-bromosuccinimide |
| n-BuLi | n-butyllithium |
| NCS | N-chlorosuccinimide |
| [Ni(dtbbpy) (H₂O)₄]Cl₂ | 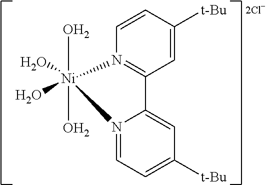 |
| NMP | N-methyl-2-pyrrolidinone |
| Pd(dppf)Cl₂ | Bis(diphenylphosphino)ferrocene]dichloropalladium(II) |
| PS-TPP | triphenylphosphine, polymer-bound |
| RuPhos-Pd G4 | 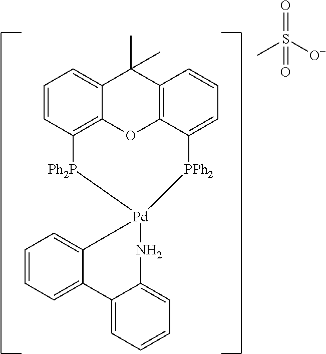 |
| SFC | super-critical fluid chromatograpy (CO₂) |
| Si-DPP-Pd | SiliaCat DDP-Pd (R) (silica-bound palladium catalyst) |
| T3P | propanephosphonic anhydride |
| TEA | Triethylamine |
| TFA | 2,2,2-trifluoroacetic acid |
| THF | tetrahydrofuran |
| TMS-Cl | chlorotrimethylsilane |
| TMSI | Iodotrimethylsilane |
| w/v | weight/volume |
| Xantphos-Pd G2 | 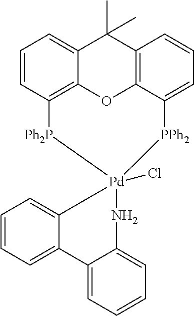 |
| Xantphos-Pd G3 | 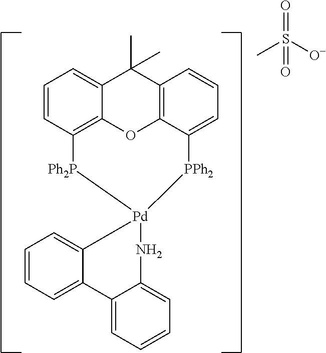 |
| Xphos-Pd G3 | 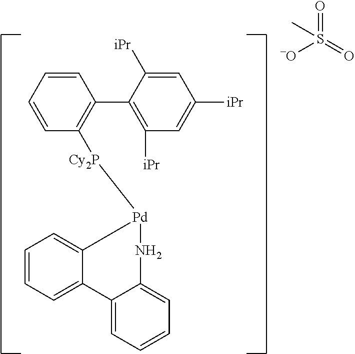 |

### Intermediate 1

### Preparation of 2-(2-oxo-1,2-dihydroquinolin-3-yl)acetohydrazide

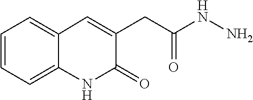 [SMILES: NNC(=O)Cc1cc2ccccc2[nH]c1=O]

A mixture of 2-(2-oxo-1,2-dihydroquinolin-3-yl)acetic acid (2.5 g, 12 mmol) and p-toluenesulfonic acid (0.47 g, 2.5 mmol) in MeOH (50 ml) was stirred and heated to reflux for 2 hours. Hydrazine (60% (w/v) in water, 20.0 mL, 380 mmol) was then added to the reaction mixture. The reaction mixture was stirred at reflux for an additional 1.5 hours. The reaction mixture was cooled to room temperature and concentrated under reduced pressure. The residue was suspended in water and filtered. The collected solids were washed with water and dried under vacuum to afford 2-(2-oxo-1,2-dihydroquinolin-3-yl)acetohydrazide. ¹H NMR (600 MHz, DMSO-d₆) δ 9.10 (s, 1H), 7.80 (s, 1H), 7.64-7.60 (m, 1H), 7.48-7.43 (m, 1H), 7.30 (d, J=8.2 Hz, 1H), 7.19-7.13 (m, 1H), 4.22 (s, 2H). LC/MS (m/z): 218 (M+H)⁺

### Intermediate 2

### Preparation of 4-((2-oxo-1,2-dihydroquinolin-3-yl)methyl)benzoic acid

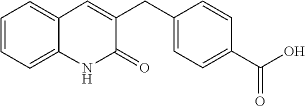 [SMILES: O=C(O)c1ccc(Cc2cc3ccccc3[nH]c2=O)cc1]

### Step A: tert-Butyl 4-((2-methoxyquinolin-3-yl)methyl)benzoate

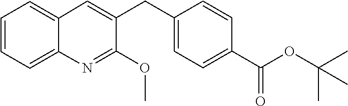 [SMILES: COc1nc2ccccc2cc1Cc1ccc(C(=O)OC(C)(C)C)cc1]

DME (15 mL) and water (5 mL) were added to a mixture of (4-(tert-butoxycarbonyl)phenyl)boronic acid (1.34 g, 6.05 mmol), 3-(chloromethyl)-2-methoxyquinoline (1.14 g, 5.50 mmol), tetrakis(triphenylphosphine)palladium(0) (0.13 g, 0.11 mmol), and sodium carbonate (1.28 g, 12.1 mmol) under a nitrogen sparge at room temperature. The reaction mixture was stirred and heated at 90° C. for 16 hours. The reaction mixture was cooled to room temperature, diluted with DCM (200 mL), and filtered through a phase separator. The filtrate was concentrated under reduced pressure, and the residue was purified by silica gel chromatography (ethyl acetate in hexanes) to afford tert-butyl 4-((2-methoxyquinolin-3-yl)methyl)benzoate. LC/MS (m/z): 350 (M+H)⁺

### Step B: 4-((2-Oxo-1,2-dihydroquinolin-3-yl)methyl)benzoic acid

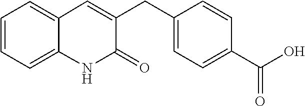 [SMILES: O=C(O)c1ccc(Cc2cc3ccccc3[nH]c2=O)cc1]

A mixture of tert-butyl 4-((2-methoxyquinolin-3-yl)methyl)benzoate in HCl (4.0 M in dioxane, 10 mL, 40 mmol) and HCl (1 M in water, 1 mL, 1 mmol) was stirred and heated to 60° C. for 2 hours. The reaction mixture was cooled to room temperature and concentrated under reduced pressure to afford 4-((2-oxo-1,2-dihydroquinolin-3-yl)methyl)benzoic acid, which was used without purification. LC/MS (m/z): 280 (M+H)⁺

### Intermediate 3

### Preparation of 3-(4-bromobenzyl)-2-methoxy-1,6-naphthyridine

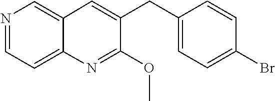 [SMILES: COc1nc2ccncc2cc1Cc1ccc(Br)cc1]

DME (20 mL) and water (10 mL) were added to a mixture of (4-bromophenyl)boronic acid (1.55 g, 7.70 mmol), 3-(chloromethyl)-2-methoxy-1,6-naphthyridine (1.46 g, 7.00 mmol), tetrakis(triphenylphosphine)palladium(0) (160 mg, 0.140 mmol), and sodium carbonate (1.63 g, 15.4 mmol) under a sparge of nitrogen at room temperature. The reaction mixture was stirred and heated at 90° C. for 16 hours. The reaction mixture was cooled to room temperature, diluted with DCM (200 mL), filtered through a phase separator, and concentrated under reduced pressure. The residue was purified by silica gel chromatography (ethyl acetate in hexanes) to afford 3-(4-bromobenzyl)-2-methoxy-1,6-naphthyridine. LC/MS (m/z): 329, 331 (M+H)⁺

### Intermediate 4

### Preparation of tert-butyl (3-(chloromethyl)quinolin-2-yl) carbonate

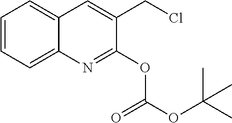 [SMILES: CC(C)(C)OC(=O)Oc1nc2ccccc2cc1CCl]

A mixture of 3-(chloromethyl)quinolin-2(1H)-one (1.00 g, 5.16 mmol), di-tert-butyl dicarbonate (1.32 mL, 5.68 mmol), and DMAP (0.032 g, 0.26 mmol) in CH₂Cl₂ (30.0 mL) was stirred at room temperature for 1 hour. The mixture was diluted with water and extracted with CH₂Cl₂ (3×). The organic layers were combined, dried over magnesium sulfate, filtered, and concentrated under reduced pressure. The residue was purified by silica gel chromatography (ethyl acetate in hexanes) to afford tert-butyl (3-(chloromethyl)quinolin-2-yl) carbonate. LC/MS (m/z): 194 (M+H-Boc)⁺

### Intermediate 5

### Preparation of (S)-5-(4-(4,4,5,5-tetramethyl-1,3,2-dioxaborolan-2-yl)phenyl)pyrrolidin-2-one

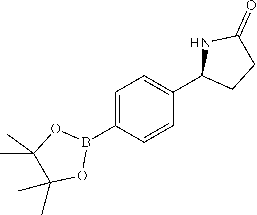 [SMILES: CC1(C)OB(c2ccc([C@@H]3CCC(=O)N3)cc2)OC1(C)C]

A mixture of [1,1′-bis(diphenylphosphino)ferrocene]dichloropalladium(II) (3.96 g, 5.41 mmol), bis(pinacolato)diboron (35.7 g, 141 mmol), potassium acetate (31.9 g, 325 mmol), and (S)-5-(4-bromophenyl)pyrrolidin-2-one (26.0 g, 108 mmol) in dioxane (260 mL) was sparged with nitrogen at room temperature. The reaction mixture was stirred and heated to 75° C. for 14 hours. The mixture was cooled to room temperature, filtered through Celite (washing with ethyl acetate), and concentrated under reduced pressure. The residue was purified by silica gel chromatography (methanol in dichloromethane) to afford (S)-5-(4-(4,4,5,5-tetramethyl-1,3,2-dioxaborolan-2-yl)phenyl)pyrrolidin-2-one. LC/MS (m/z): 288 (M+H)⁺

### Intermediate 6

### Preparation of 3-(chloromethyl)-2-methoxyquinoline

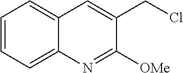 [SMILES: COc1nc2ccccc2cc1CCl]

### Step A: 2-Chloroquinoline-3-carbaldehyde

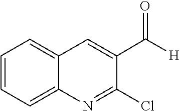 [SMILES: O=Cc1cc2ccccc2nc1Cl]

POCl₃ (3500 mL, 38 mol) was added dropwise via an addition funnel to DMF (1060 mL, 13.4 mol) under a nitrogen atmosphere at 0-5° C. The mixture was stirred for 5 minutes at 0° C. Acetanilide (550 g, 4.06 mol) was added, and the reaction mixture was heated to 75-80° C. for 8 hours. The reaction mixture was cooled to room temperature and poured into a stirred mixture of crushed ice. The mixture was filtered, and the collected solids were washed with water and dried under vacuum. The residue was recrystallized from ethyl acetate to afford 2-chloroquinoline-3-carbaldehyde.

### Step B: 2-Methoxyquinoline-3-carbaldehyde

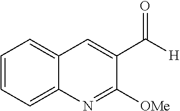 [SMILES: COc1nc2ccccc2cc1C=O]

2-Chloroquinoline-3-carboxaldehyde (521 g, 2.71 mol) was added to a mixture of KOH (230 g, 4.1 mol) in MeOH (13 L). The reaction mixture was stirred and refluxed for 10 hours. The reaction mixture was cooled to room temperature and diluted with water (20 L). The reaction mixture was filtered, and the collected solids were washed with water and Et₂O, and dried under vacuum to afford 2-methoxyquinoline-3-carbaldehyde.

### Step C: (2-Methoxyquinolin-3-yl)methanol

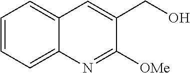 [SMILES: COc1nc2ccccc2cc1CO]

Sodium borohydride (142 g, 3.73 mol) was added to a mixture of 2-methoxyquinoline-3-carbaldehyde (410 g, 2.19 mmol) in THF (6150 mL) and MeOH (6150 mL) under a nitrogen atmosphere at 0° C. The reaction mixture was stirred at 0° C. for 30 minutes. The reaction mixture was quenched with water at 0° C. and diluted with Et₂O. The organic layer was separated, washed with brine, dried over anhydrous sodium sulfate, and concentrated under reduced pressure to afford (2-methoxyquinolin-3-yl)methanol.

### Step D: 3-(Chloromethyl)-2-methoxyquinoline

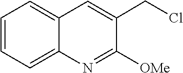 [SMILES: COc1nc2ccccc2cc1CCl]

A mixture of (2-methoxyquinolin-3-yl)methanol (367 g, 1.94 mol) and triphenylphosphine (560 g, 2.13 mol) in CH₂Cl₂ (7340 mL) was cooled to 0° C. CCl₄ (206 ml, 2.13 mol) was slowly added to the reaction mixture at 0° C. The reaction mixture was stirred for 15 hours at room temperature. An additional mixture of triphenylphosphine (50 g, 0.21 mol) and CCl₄ (50 mL, 0.52 mol) in DMF 1000 mL was added, and the reaction mixture was heated to 40° C. (with reflux condenser) and stirred for 15 hours. The reaction mixture was concentrated under reduced pressure. The residue was purified by silica gel chromatography (ethyl acetate in petroleum ether) to afford 3-(chloromethyl)-2-methoxyquinoline. ¹H NMR (400 MHz, chloroform-d) δ 8.10 (s, 1H), 7.90 (d, J=8.4 Hz, 1H), 7.75 (dd, J=8, 1.2 Hz, 1H), 7.68-7.64 m, 1H), 7.44-7.40 (m, 1H), 4.75 (s, 2H), 4.16 (s, 3H). LC/MS (m/z): 208 (M+H)⁺

### Intermediate 7

### Preparation of 3-(chloromethyl)quinolin-2(1H)-one

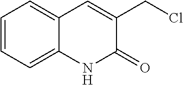 [SMILES: O=c1[nH]c2ccccc2cc1CCl]

### Step A: 3-Chloro-N-phenylpropanamide

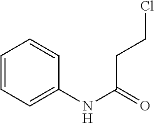 [SMILES: O=C(CCCl)Nc1ccccc1]

3-chloropropanoyl chloride (313 g, 2.47 mol) was added dropwise to a mixture of aniline (200 g, 2.15 mol) and potassium carbonate (163 g, 1.18 mol) in water (1.0 L) at room temperature. The reaction mixture was stirred at room temperature for 2 hours. The mixture was filtered and the collected solids were washed with water (2×1.0 L). The solids were dried under vacuum to afford 3-chloro-N-phenylpropanamide. ¹H NMR (400 MHz, chloroform-d) δ 7.43-7.56 (m, 2H), 7.23-7.43 (m, 3H), 7.13 (t, J=7.0 Hz, 1H), 3.88 (t, J=6.3 Hz, 2H), 2.81 (t, J=6.4 Hz, 2H). LC/MS (m/z): 184 (M+H)⁺

### Step B: 2-Chloro-3-(chloromethyl)quinoline

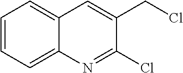 [SMILES: ClCc1cc2ccccc2nc1Cl]

POCl₃ (1.46 kg, 9.53 mol) was added dropwise to DMF (261 mL, 3.4 mol) at 0° C. The internal reaction temperature was maintained below 10° C. during the addition. The mixture was stirred at 0° C. for an additional 10 minutes after the addition was complete. 3-Chloro-N-phenylpropanamide (250 g, 1.36 mol) was added to the reaction mixture portion wise (maintaining the internal reaction temperature below 45° C.) The reaction mixture was stirred at 45° C. for 50 minutes, and then heated to 80° C. and stirred for an additional 3 hours. The reaction mixture was cooled to room temperature and concentrated under reduced pressure. The residue was triturated with water and the mixture was filtered. The collected solids were purified by silica gel chromatography (ethyl acetate in petroleum ether) to afford 2-chloro-3-(chloromethyl)quinoline. ¹H NMR (400 MHz, chloroform-d) δ 8.21-8.31 (m, 1H), 8.02 (d, J=8.4 Hz, 1H), 7.80-7.87 (m, 1H), 7.76-7.74 (m, 1H), 7.50-7.67 (m, 1H), 4.83 (s, 2H). LC/MS (m/z): 212 (M+H)⁺

### Step C: 3-(Chloromethyl)quinolin-2(1H)-one

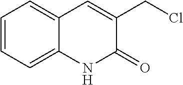 [SMILES: O=c1[nH]c2ccccc2cc1CCl]

A mixture of 2-chloro-3-(chloromethyl)quinoline (130 g, 612 mmol) in acetic acid (900 mL) was stirred and heated to 120° C. for 6 hours. The reaction mixture was cooled to room temperature, added in a single portion to water (3.0 L), and filtered. The collected solids were added to methanol (3.0 L) and the mixture was stirred and heated to 60° C. for 2 hours. The mixture was cooled to room temperature and stirred for an additional 16 hours. The mixture was filtered, and the filtrate was concentrated under reduced pressure to afford 3-(chloromethyl)quinolin-2(1H)-one. ¹H NMR (400 MHz, DMSO-d₆) δ 11.96 (br s, 1H), 8.10 (s, 1H), 7.67 (d, J=8.0 Hz, 1H), 7.46-7.53 (m, 1H), 7.30 (d, J=8.4 Hz, 1H), 7.19-7.17 (m, 1H), 4.62 (s, 2H). LC/MS (m/z): 194 (M+H)⁺

### Intermediate 8

### Preparation of 3-(4-bromobenzyl)-2-methoxyquinoline

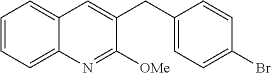 [SMILES: COc1nc2ccccc2cc1Cc1ccc(Br)cc1]

A mixture of 3-(chloromethyl)-2-methoxyquinoline (1.00 g, 4.82 mmol), (4-bromophenyl)boronic acid (1.16 g, 5.78 mmol), sodium carbonate (1.07 g, 10.1 mmol), and tetrakis(triphenylphosphine)palladium(0) (0.111 g, 0.096 mmol) in DME (20 mL) and water (10 mL) was sparged with argon for 5 minutes at room temperature. The reaction mixture was stirred and heated to 100° C. under an argon atmosphere (with reflux condenser attached). The reaction mixture was cooled to room temperature and concentrated under reduced pressure. The residue was purified by silica gel chromatography (ethyl acetate in hexanes) to afford 3-(4-bromobenzyl)-2-methoxyquinoline. ¹H NMR (300 MHz, chloroform-d) δ 7.83 (d, J=8.4 Hz, 1H), 7.63-7.54 (m, 3H), 7.43-7.31 (m, 3H), 7.11 (d, J=8.4 Hz, 2H), 4.08 (s, 3H), 3.98 (s, 2H). LC/MS (m/z): 328, 330 (M+H)⁺

### Intermediate 9

### Preparation of 3-(chloromethyl)-2-methoxy-1,6-naphthyridine

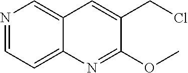

### Step A: tert-Butyl (3-formylpyridin-4-yl)carbamate

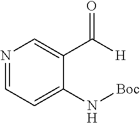 [SMILES: CC(C)(C)OC(=O)Nc1ccncc1C=O]

A mixture of tert-butyl pyridin-4-ylcarbamate (100 g, 515 mmol) in THF was sparged with nitrogen for 5 minutes and then cooled to −70° C. A solution of n-BuLi (2.5 M in hexanes, 412 mL, 1.0 mol) was added dropwise at −70° C. over a period of 1 hour. The reaction mixture was then warmed to −20° C. and stirred for an additional 2 hours. DMF (79 mL, 1.0 mol) was added dropwise over a period of 1 hour at −20° C. The reaction mixture was warmed to room temperature and stirred for an additional 12 hours. The reaction mixture was quenched with saturated aqueous ammonium chloride (1.5 L) and extracted with ethyl acetate (3×1.0 L). The organic layer was separated, dried over anhydrous sodium sulfate, filtered, and concentrated under reduced pressure to afford tert-butyl (3-formylpyridin-4-yl)carbamate, which was used without purification. ¹H NMR (400 MHz, DMSO-d₆) δ 10.41 (s, 1H), 10.01 (s, 1H), 8.93 (s, 1H), 8.63 (d, J=6.0 Hz, 1H), 8.15 (d, J=6.0 Hz, 1H), 1.51 (s, 9H)

### Step B: 4-aminonicotinaldehyde

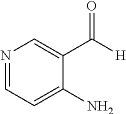

A mixture of tert-butyl (3-formylpyridin-4-yl)carbamate in HCl (37% in water, 75 mL) was sparged with nitrogen for 5 minutes and then stirred and heated to 100° C. for 5 hours. The reaction mixture was cooled to 0° C. Saturated aqueous sodium carbonate was added until the pH˜8. The mixture was extracted with ethyl acetate (3×1 L). The organic layers were combined, dried over anhydrous sodium sulfate, filtered, and concentrated under reduced pressure to afford 4-aminonicotinaldehyde, which was used without purification. ¹H NMR (400 MHz, DMSO-d₆) δ 9.87 (s, 1H), 8.53 (s, 1H), 8.09 (d, J=6.0 Hz, 1H), 7.65 (br s, 2H), 6.66 (d, J=6.0 Hz, 1H).

### Step C: Methyl 2-oxo-1,2-dihydro-1,6-naphthyridine-3-carboxylate

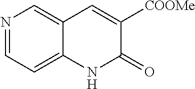 [SMILES: COC(=O)c1cc2cnccc2[nH]c1=O]

Dimethyl propanedioate (270 g, 2.05 mol) was added to a mixture of 4-aminonicotinaldehyde (125 g, 1.02 mol) in methanol (2.5 L) at room temperature. Piperidine (25.3 mL, 259 mmol) was added and the reaction mixture was stirred and heated to 80° C. for 12 hours. The reaction mixture was cooled to room temperature, concentrated under reduced pressure, and triturated with MTBE (2.0 L). The mixture was filtered, and the collected solids were dried under vacuum to afford methyl 2-oxo-1,2-dihydro-1,6-naphthyridine-3-carboxylate, which was used without purification. ¹H NMR (400 MHz, DMSO-d₆) δ 12.30 (br s, 1H) 8.97 (s, 1H) 8.61 (s, 1H) 8.53 (d, J=5.6 Hz, 1H) 7.20 (d, J=5.6 Hz, 1H) 3.82 (s, 3H). LC/MS (m/z): 205 (M+H)⁺

### Step D: Methyl 2-chloro-1,6-naphthyridine-3-carboxylate

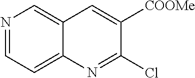 [SMILES: COC(=O)c1cc2cnccc2nc1Cl]

POCl₃ (380 g, 2.48 mol) was added to a mixture of methyl 2-oxo-1,2-dihydro-1,6-naphthyridine-3-carboxylate (100 g, 490 mmol) in acetonitrile (1.5 L) at room temperature. The reaction mixture was stirred and heated to 100° C. for 12 hours. The reaction mixture was cooled to room temperature and concentrated under reduced pressure to afford methyl 2-chloro-1,6-naphthyridine-3-carboxylate, which was used without purification. LC/MS (m/z): 223 (M+H)⁺

### Step E: Methyl 2-methoxy-1,6-naphthyridine-3-carboxylate

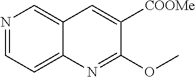 [SMILES: COC(=O)c1cc2cnccc2nc1OC]

A mixture of methyl 2-chloro-1,6-naphthyridine-3-carboxylate (110 g, 494 mmol) in methanol (1.5 L) was stirred and heated to 80° C. for 5 hours. The reaction mixture was cooled to room temperature and concentrated under reduced pressure to afford methyl 2-methoxy-1,6-naphthyridine-3-carboxylate, which was used without purification. LC/MS (m/z): 219 (M+H)⁺

### Step E: 2-Methoxy-1,6-naphthyridine-3-carboxylic acid

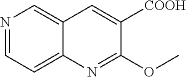

Trimethylsilanolate (129 g, 1.01 mol) was added to a mixture of methyl 2-methoxy-1,6-naphthyridine-3-carboxylate (110 g, 504 mmol) in 1,4-dioxane (1.0 L) at room temperature. The reaction mixture was stirred at room temperature for 2 hours. The reaction mixture was then cooled to 0° C. and diluted with HCl (4.0 M in dioxane, 100 mL, 400 mmol). The mixture was warmed to room temperature and concentrated under reduced pressure to afford 2-methoxy-1,6-naphthyridine-3-carboxylic acid, which was used without purification. LC/MS (m/z): 205 (M+H)⁺

### Step F: (2-Methoxy-1,6-naphthyridin-3-yl)methanol

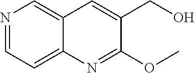

Borane-THF (1.0 M in THF, 519 mL, 519 mmol) was added to a mixture of 2-methoxy-1,6-naphthyridine-3-carboxylic acid (25.0 g 104 mmol) in THF (100 mL) at room temperature. The reaction mixture was stirred at room temperature for 2 hours. The reaction mixture was cooled to 0° C. and diluted with HCl (1.0 M in water, 500 mL, 500 mmol). The mixture was warmed to room temperature and stirred for 2 hours. Saturated aqueous sodium bicarbonate was added until the pH˜8. The mixture was extracted with ethyl acetate (3×3 L), and the organic layers were combined, dried over anhydrous sodium sulfate, filtered, and concentrated under reduced pressure to afford (2-methoxy-1,6-naphthyridin-3-yl)methanol, which was used without purification. LC/MS (m/z): 191 (M+H)⁺

### Step G: 3-(Chloromethyl)-2-methoxy-1,6-naphthyridine

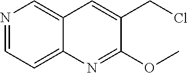

Thionyl chloride (138 g, 1.16 mol) was added slowly to a mixture of (2-methoxy-1,6-naphthyridin-3-yl)methanol (70.0 g, 368 mmol) and DMF (0.50 mL, 6.5 mmol) in DCM (1.00 L) at 0° C. The reaction mixture was warmed to room temperature and stirred for an additional 2 hours. The reaction mixture was concentrated under reduced pressure, and the residue was suspended between ethyl acetate (1.0 L) and saturated aqueous sodium bicarbonate (1.2 L). The organic layer was separated, and the aqueous layer was washed with additional ethyl acetate (2×800 mL). The organic layers were combined, dried over anhydrous sodium sulfate, filtered, and concentrated under reduced pressure. The residue was purified by silica gel chromatography (ethyl acetate in petroleum ether) to afford 3-(chloromethyl)-2-methoxy-1,6-naphthyridine. ¹H NMR (400 MHz, DMSO-d₆) δ 9.22 (s, 1H), 8.64 (d, J=5.6 Hz, 1H), 8.52 (s, 1H), 7.67 (d, J=6.0 Hz, 1H), 4.86 (s, 2H), 4.10 (s, 3H). LC/MS (m/z): 209 (M+H)⁺

### Intermediate 10

### Preparation of 2-methyl-2-(4-(4,4,5,5-tetramethyl-1,3,2-dioxaborolan-2-yl)-1H-pyrazol-1-yl)propanenitrile

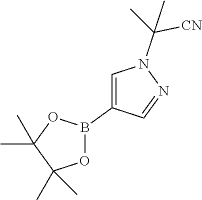 [SMILES: CC(C)(C#N)n1cc(B2OC(C)(C)C(C)(C)O2)cn1]

Iodomethane (0.147 ml, 2.36 mmol) was added to a mixture of 2-(4-(4,4,5,5-tetramethyl-1,3,2-dioxaborolan-2-yl)-1H-pyrazol-1-yl)acetonitrile (250 mg, 1.07 mmol) in THF (5 ml) at 0° C. NaHMDS (1.0 M in THF, 3.0 ml, 3.0 mmol) was added to the reaction mixture over a period of 10 minutes at 0° C. The mixture was warmed to room temperature. Saturated aqueous ammonium chloride (15 mL) was added to the mixture. The mixture was extracted with DCM (2×25 mL). The organic layer was dried over anhydrous sodium sulfate, filtered, and concentrated under reduced pressure. The residue was purified by silica gel chromatography (0-50% [EtOAc:EtOH (3:1)] in hexanes) to afford 2-methyl-2-(4-(4,4,5,5-tetramethyl-1,3,2-dioxaborolan-2-yl)-1H-pyrazol-1-yl)propanenitrile. LC/MS (m/z): 262 (M+H)+.

### Intermediate 11

### Preparation of 6-bromo-2-(2-hydroxy-2-methylpropyl)isoindolin-1-one

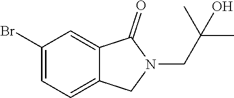 [SMILES: CC(C)(O)CN1Cc2ccc(Br)cc2C1=O]

A mixture of 6-bromoisoindolin-1-one (100 mg, 0.472 mmol), cesium carbonate (307 mg, 0.943 mmol), NMP (1.5 ml), and 2,2-dimethyloxirane (0.126 ml, 1.42 mmol) was stirred and heated in a microwave reactor at 150° C. for 1 hour. The reaction mixture was cooled to room temperature, diluted with diethyl ether (30 mL), and washed with water (2×30 mL). The organic layer was dried over magnesium sulfate, filtered, and concentrated under reduced pressure. The residue was purified by silica gel chromatography (0-60% [EtOAc:EtOH (3:1)] in hexanes) to afford 6-bromo-2-(2-hydroxy-2-methylpropyl)isoindolin-1-one. LC/MS (m/z): 284, 286 (M+H)+.

### Intermediate 12

### Preparation of 3-(4-bromobenzyl)quinolin-2(1H)-one

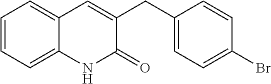 [SMILES: O=c1[nH]c2ccccc2cc1Cc1ccc(Br)cc1]

Chlorotrimethylsilane (2.2 mL, 17 mmol) was added to a mixture of 3-(4-bromobenzyl)-2-methoxyquinoline (1.44 g, 4.39 mmol) and sodium iodide (2.63 g, 17.6 mmol) in acetonitrile (20 mL) at room temperature. The reaction mixture was stirred at room temperature for 16 hours. The mixture was diluted with methanol and concentrated under reduced pressure. The residue was purified by silica gel chromatography (ethyl acetate in hexanes) to afford 3-(4-bromobenzyl)quinolin-2(1H)-one. LC/MS (m/z): 314, 316 (M+H)⁺

### Intermediate 13

### Preparation of 3-(4-bromo-3-chlorobenzyl)quinolin-2(1H)-one

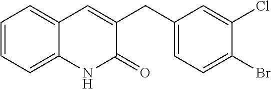 [SMILES: O=c1[nH]c2ccccc2cc1Cc1ccc(Br)c(Cl)c1]

### Step A: 3-(4-Bromo-3-chlorobenzyl)-2-methoxyquinoline

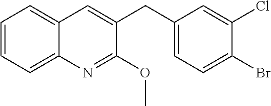 [SMILES: COc1nc2ccccc2cc1Cc1ccc(Br)c(Cl)c1]

A mixture of 3-(chloromethyl)-2-methoxyquinoline (1.00 g, 4.82 mmol), (4-bromo-3-chlorophenyl)boronic acid (1.25 g, 5.30 mmol), sodium carbonate (1.07 g, 10.1 mmol), and tetrakis(triphenylphosphine)palladium(0) (0.11 g, 0.096 mmol) was sparged with argon for 5 minutes. DME (20 mL) and water (10 mL) were added, and the mixture was stirred and heated at 150° C. under an argon atmosphere for 3.5 hours (attached to reflux condenser). The mixture was cooled to room temperature, diluted with DCM and water, passed through a phase separator, and concentrated under reduced pressure to afford 3-(4-bromo-3-chlorobenzyl)-2-methoxyquinoline, which was used without purification. LC/MS (m/z): 362, 364 (M+H)⁺

### Step B: 3-(4-Bromo-3-chlorobenzyl)quinolin-2(1H)-one

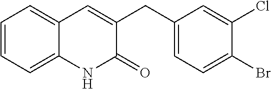 [SMILES: O=c1[nH]c2ccccc2cc1Cc1ccc(Br)c(Cl)c1]

Chlorotrimethylsilane (2.5 mL, 20 mmol) was added to a mixture of 3-(4-bromo-3-chlorobenzyl)-2-methoxyquinoline (1.75 g, 4.82 mmol) and sodium iodide (2.89 g, 19.3 mmol) in acetonitrile (20 mL). The mixture was stirred at room temperature for three days. The mixture was diluted with water and filtered. The collected solids were washed with hexanes followed by 20% EtOAc/hexanes. The solids were dried under reduced pressure to afford 3-(4-bromo-3-chlorobenzyl)quinolin-2(1H)-one, which was used without purification. LC/MS (m/z): 348, 350 (M+H)⁺

### Intermediate 14

### Preparation of 3-((6-chloropyridin-3-yl)methyl)-2-methoxyquinoline

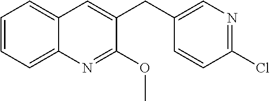 [SMILES: COc1nc2ccccc2cc1Cc1ccc(Cl)nc1]

A mixture of (6-chloropyridin-3-yl)boronic acid (0.834 g, 5.30 mmol), 3-(chloromethyl)-2-methoxyquinoline (1.00 g, 4.82 mmol), tetrakis(triphenylphosphine)palladium (0) (0.083 g, 0.072 mmol), and sodium carbonate (1.07 g, 10.1 mmol) in 1,4-dioxane (10.7 mL) and water (5.4 mL) was sparged with argon at room temperature, and then stirred and heated at 100° C. for 2 hours. The reaction mixture was cooled to room temperature, diluted with water, and extracted with DCM. The organic layer was separated, dried over magnesium sulfate, filtered, and concentrated under reduced pressure. The residue was purified by silica gel chromatography (ethyl acetate in hexanes) to afford 3-((6-chloropyridin-3-yl)methyl)-2-methoxyquinoline.

LC/MS (m/z): 285 (M+H)⁺

### Intermediate 15

### Preparation of 3-((5,6-dichloropyridin-3-yl)methyl)-2-methoxyquinoline

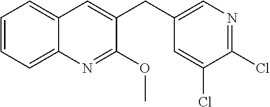 [SMILES: COc1nc2ccccc2cc1Cc1cnc(Cl)c(Cl)c1]

A mixture of 2,3-dichloropyridine-5-boronic acid (508 mg, 2.65 mmol), 3-(chloromethyl)-2-methoxyquinoline (500 mg, 2.41 mmol), tetrakis(triphenylphosphine)palladium (0) (42 mg, 0.036 mmol), and sodium carbonate (536 mg, 5.06 mmol) in 1,4-dioxane (7.2 mL) and water (2.4 mL) was sparged with argon at room temperature, and then stirred and heated to 100° C. for 10 minutes. The reaction mixture was cooled to room temperature, diluted with water, and extracted with DCM. The organic layer was separated, dried over magnesium sulfate, filtered, and concentrated under reduced pressure. The residue was purified by silica gel chromatography (ethyl acetate in hexanes) to afford 3-((5,6-dichloropyridin-3-yl)methyl)-2-methoxyquinoline. LC/MS (m/z): 319, 321 (M+H)⁺

### Intermediate 16

### Preparation of 3-(chloromethyl)-1-methylquinolin-2(1H)-one

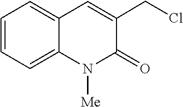 [SMILES: Cn1c(=O)c(CCl)cc2ccccc21]

### Step A: 2-Oxo-1,2-dihydroquinoline-3-carbaldehyde

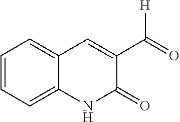 [SMILES: O=Cc1cc2ccccc2[nH]c1=O]

A mixture of 2-chloroquinoline-3-carbaldehyde (110 g, 574 mmol) in acetic acid (2200 mL) was stirred and heated to 100° C. for 6 hours. The reaction mixture was cooled to 0° C. The mixture was triturated with ice water. The mixture was filtered and the solids were dried under vacuum to afford 2-oxo-1,2-dihydroquinoline-3-carbaldehyde, which was used without purification.

### Step B: 1-Methyl-2-oxo-1,2-dihydroquinoline-3-carbaldehyde

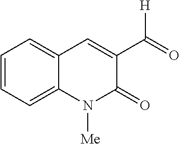 [SMILES: Cn1c(=O)c(C=O)cc2ccccc21]

A mixture of 2-oxo-1,2-dihydroquinoline-3-carbaldehyde (82.0 g, 474 mmol) and potassium carbonate (198 g, 1.43 mol) in N,N-dimethylformamide (2460 mL was stirred at room temperature for 20 minutes. Iodomethane (87.5 g, 616 mmol) was added at room temperature, and the reaction mixture was stirred for 16 hours at room temperature. The mixture was triturated with ice water and filtered. The collected solids were dried under vacuum to afford 1-methyl-2-oxo-1,2-dihydroquinoline-3-carbaldehyde, which was used without purification.

### Step C: 3-(Hydroxymethyl)-1-methylquinolin-2(1H)-one

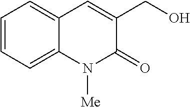 [SMILES: Cn1c(=O)c(CO)cc2ccccc21]

A mixture of 1-methyl-2-oxo-1,2-dihydroquinoline-3-carbaldehyde (62.0 g, 331 mmol) in methanol (1240 mL) was cooled to 0° C. NaBH₄ (12.7 g, 336 mmol) was added to the mixture at 0° C. The reaction mixture was stirred at 0° C. for 2 hours. The mixture was quenched with water and extracted with ethyl acetate (3×3 L). The organic layers were combined, dried over anhydrous sodium sulfate, filtered, and concentrated under reduced pressure. The residue was purified by recrystallization from n-hexane. The solids were collected by filtration and dried under vacuum to afford 3-(hydroxymethyl)-1-methyl-1,2-dihydroquinolin-2-one. ¹H NMR (400 MHz, methanol-d₄) δ 7.96 (d, J=1.6 Hz, 1H), 7.74 (dd, J=7.9, 1.5 Hz, 1H), 7.65 (ddd, J=8.6, 6.9, 1.5 Hz, 1H), 7.60 (d, J=8.5 Hz, 1H), 7.34 (ddd, J=8.1, 7.0, 1.2 Hz, 1H), 4.62 (d, J=1.6 Hz, 2H), 3.78 (s, 3H). LC/MS (m/z): 190 (M+H)⁺

### Step D: 3-(Chloromethyl)-1-methylquinolin-2(1H)-one

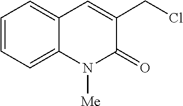 [SMILES: Cn1c(=O)c(CCl)cc2ccccc21]

Thionyl chloride (5.0 mL, 16 mmol) was added to mixture of 3-(hydroxymethyl)-1-methylquinolin-2(1H)-one (3.0 g, 16 mmol) in dichloromethane (100 mL) at 0° C. After the addition was complete, the reaction mixture was warmed to room temperature and stirred for 15 hours. The reaction mixture was concentrated under reduced pressure and the residue was purified by silica gel chromatography (methanol in dichloromethane) to afford 3-(chloromethyl)-1-methylquinolin-2(1H)-one. ¹H NMR (400 MHz, DMSO-d₆) δ 8.11 (s, 1H), 7.76-7.70 (m, 1H), 7.67-7.58 (m, 1H), 7.55-7.47 (m, 1H), 7.32-7.19 (m, 1H), 4.65 (s, 2H), 3.63 (s, 3H). LC/MS (m/z): 208 (M+H)⁺

### Intermediate 17

### Preparation of 3-(bromomethyl)-2-methoxyquinoline

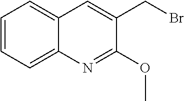

Triphenylphosphine (9.98 g, 38.1 mmol) was added to a mixture of carbon tetrabromide (12.6 g, 38.1 mmol) and (2-methoxyquinolin-3-yl)methanol (6.0 g, 32 mmol) in DCM (150 mL) at 0° C. The mixture was sparged with nitrogen, and then warmed to room temperature and stirred for 3 hours. The mixture was concentrated under reduced pressure. The residue was purified by silica gel chromatography (ethyl acetate in petroleum ether) to afford 3-(bromomethyl)-2-methoxyquinoline. ¹H NMR (500 MHz, chloroform-d) δ 8.03 (s, 1H), 7.84 (d, J=8.4 Hz, 1H), 7.71 (dd, J=8.0, 1.1 Hz, 1H), 7.62 (ddd, J=8.4, 7.0, 1.5 Hz, 1H), 7.38 (ddd, J=8.0, 7.0, 1.1 Hz, 1H), 4.62 (s, 2H), 4.15 (s, 3H). LC/MS (m/z): 252, 254 (M+H)⁺

### Intermediate 18

### Preparation of tert-butyl (methyl(oxo)(4-(4,4,5,5-tetramethyl-1,3,2-dioxaborolan-2-yl)benzyl)-λ⁶-sulfanylidene)carbamate

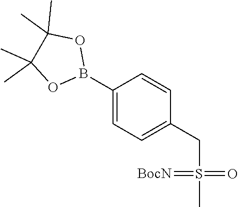

### Step A: (4-bromobenzyl)(methyl)sulfane

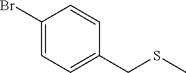

Triethylamine (1.23 mL, 8.80 mmol) was added to a mixture of 1-bromo-4-(bromomethyl)benzene (1.1 g, 4.4 mmol) and sodium methanethiolate (410 mg, 5.9 mmol) in DMF (4 mL) at 0° C. The mixture was stirred and heated at 30° C. for 12 h. The mixture was quenched with water (40 mL) and extracted with EtOAc (10 mL×3). The organic layers were combined, washed with brine (30 mL), dried over anhydrous Na₂SO₄, filtered, and concentrated under reduced pressure to afford (4-bromobenzyl)(methyl)sulfane, which was used in the next step without purification. ¹H NMR (500 MHz, chloroform-d) δ 7.47-7.41 (m, 2H), 7.18 (d, J=8.2 Hz, 2H), 3.62 (s, 2H), 1.98 (s, 3H).

### Step B: 1-bromo-4-((methylsulfinyl)methyl)benzene

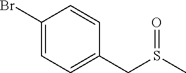

A mixture of meta-Chloroperoxybenzoic acid (75% w/w, 674 mg, 3.9 mmol) in DCM (17.5 mL) was slowly added to a mixture of (4-bromobenzyl)(methyl)sulfane (848 mg, 3.91 mmol) in DCM (12.5 mL) at 0° C. The mixture was stirred at 20° C. for 2 h. The mixture was washed sequentially with solutions of saturated aqueous Na₂SO₃ (20 mL), saturated aqueous NaHCO₃ (20 mL), and water (20 mL). The organic layer was dried over anhydrous Na₂SO₄, filtered, and concentrated under reduced pressure. The residue was purified by silica gel chromatography (eluting ethyl acetate in petroleum ether) to afford 1-bromo-4-((methylsulfinyl)methyl)benzene. ¹H NMR (500 MHz, chloroform-d) (7.53 (d, J=8.4 Hz, 2H), 7.18 (d, J=8.4 Hz, 2H), 3.93 (d, J=2.7 Hz, 2H), 2.47 (s, 3H). LC/MS (m/z): 233, 235 (M+H)⁺

### Step C: tert-butyl ((4-bromobenzyl)(methyl)(oxo)-λ⁶-sulfanylidene)carbamate

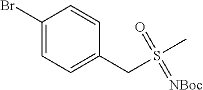

Iodosobenzene diacetate (1.29 g, 3.99 mmol) was added to a mixture of 1-bromo-4-((methylsulfinyl)methyl)benzene (620 mg, 2.66 mmol), BocNH₂ (467 mg, 3.99 mmol), magnesium oxide (429 mg, 10.6 mmol), and Rh₂(OAc)₄ (29 mg, 66 μmol) in DCM (2 mL) at 20° C. The mixture was stirred and heated at 40° C. for 8 h. The mixture was filtered through Celite and the filtrate was concentrated under reduced pressure. The residue was purified by silica gel chromatography (eluting ethyl acetate in petroleum ether) to afford tert-butyl ((4-bromobenzyl)(methyl)(oxo)-λ⁶-sulfaneylidene)carbamate. ¹H NMR (500 MHz, chloroform-d) δ 7.56 (d, J=8.2 Hz, 2H), 7.30 (d, J=8.2 Hz, 2H), 4.71 (s, 2H), 2.94 (s, 3H), 1.51 (s, 9H). LC/MS (m/z): 348, 350 (M+H)⁺

### Step D: tert-butyl (methyl(oxo)(4-(4,4,5,5-tetramethyl-1,3,2-dioxaborolan-2-yl)benzyl)-λ⁶-sulfanylidene)carbamate

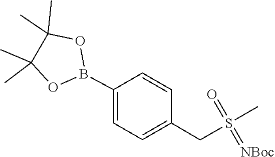

A mixture of tert-butyl ((4-bromobenzyl)(methyl)(oxo)-λ⁶-sulfaneylidene)carbamate (300 mg, 861 μmol), bis(pinacolato)diboron (328 mg, 1.29 mmol), Pd(dppf)Cl₂ (32 mg, 43 μmol), potassium acetate (254 mg, 2.58 mmol), and 1,4-dioxane (5 mL) was degassed with nitrogen for 5 min. The mixture was stirred and heated at 80° C. for 5 h. The mixture was filtered and the filtrate was concentrated under reduced pressure. The residue was purified by silica gel chromatography (eluting ethyl acetate in petroleum ether) to afford tert-butyl (methyl(oxo)(4-(4,4,5,5-tetramethyl-1,3,2-dioxaborolan-2-yl)benzyl)-λ⁶-sulfaneylidene)carbamate. ¹H NMR (500 MHz, chloroform-d) δ 7.84 (d, J=7.8 Hz, 2H), 7.41 (d, J=7.8 Hz, 2H), 4.87-4.79 (m, 1H), 4.77-4.69 (m, 1H), 2.88 (s, 3H), 1.51 (s, 9H), 1.34 (s, 12H). LC/MS (m/z): 396 (M+H)⁺

### Intermediate 19

### Preparation of (R and S)-5-methyl-3-(4-(4,4,5,5-tetramethyl-1,3,2-dioxaborolan-2-yl)phenyl)oxazolidin-2-one

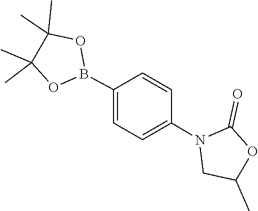 [SMILES: CC1CN(c2ccc(B3OC(C)(C)C(C)(C)O3)cc2)C(=O)O1]

### Step A: (R and S)-3-(4-bromophenyl)-5-methyloxazolidin-2-one

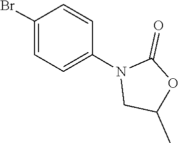

A mixture of (+/−)-5-methyloxazolidin-2-one (0.500 g, 4.95 mmol), TEA (2.07 mL, 14.8 mmol), 4-bromophenylboronic acid (1.49 g, 7.42 mmol), and copper(II) acetate (1.80 g, 9.89 mmol) in chloroform (10 mL) was degassed and backfilled with oxygen (three times). The mixture was stirred at 25° C. for 16 h. The mixture was concentrated under reduced pressure and the residue was partitioned between water (35 mL) and DCM (35 mL). The organic layer was separated and the aqueous layer was re-extracted with DCM (35 mL×3). The organic layers were combined, washed with brine (35 mL), dried over anhydrous Na₂SO₄, filtered, and concentrated under reduced pressure. The residue was purified by silica gel chromatography (eluting ethyl acetate in petroleum ether) to afford (R and S)-3-(4-bromophenyl)-5-methyloxazolidin-2-one. ¹H NMR (400 MHz, methanol-d₄) δ 7.57-7.53 (m, 2H), 7.46-7.37 (m, 2H), 4.67-4.60 (m, 2H), 4.11-4.04 (m, 1H), 1.30-1.28 (m, 3H). LC/MS (m/z): 258, 260 (M+H)⁺

### Step B: (R and S)-5-methyl-3-(4-(4,4,5,5-tetramethyl-1,3,2-dioxaborolan-2-yl)phenyl)oxazolidin-2-one

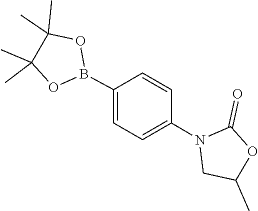 [SMILES: CC1CN(c2ccc(B3OC(C)(C)C(C)(C)O3)cc2)C(=O)O1]

A mixture of (R and S)-3-(4-bromophenyl)-5-methyloxazolidin-2-one (0.460 g, 1.80 mmol), 4,4,4′,4′,5,5,5′,5′-octamethyl-2,2′-bi(1,3,2-dioxaborolane) (684 mg, 2.69 mmol), Pd(dppf)Cl₂ (329 mg, 0.449 mmol), and potassium acetate (529 mg, 5.39 mmol) in 1,4-dioxane (15 mL) at 25° C. was bubbled with a stream of N2 for 5 minutes. The mixture was stirred and heated at 80° C. for 12 h. The mixture was concentrated under reduced pressure and the residue was partitioned between water (20 mL) and EtOAc (20 mL). The organic layer was separated and the aqueous layer was re-extracted with EtOAc (20 mL×3). The organic layers were combined, washed with brine (20 mL), dried over anhydrous Na₂SO₄, filtered, and concentrated under reduced pressure. The residue was purified by silica gel chromatography (eluting ethyl acetate in petroleum ether) to afford (R and S)-5-methyl-3-(4-(4,4,5,5-tetramethyl-1,3,2-dioxaborolan-2-yl)phenyl)oxazolidin-2-one. ¹H NMR (400 MHz, methanol-d₄) δ 7.78 (d, J=8.6 Hz, 2H), 7.50 (d, J=8.6 Hz, 2H), 4.75-4.66 (m, 1H), 4.17-4.04 (m, 2H), 1.35 (s, 12H), 1.30 (d, J=6.1 Hz, 3H). LC/MS (m/z): 304 (M+H)⁺

### Example 1

### Preparation of 3-(4-(3-hydroxyazetidine-1-carbonyl)benzyl)quinolin-2(1H)-one

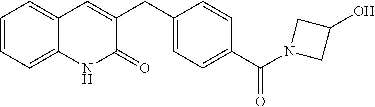 [SMILES: O=C(c1ccc(Cc2cc3ccccc3[nH]c2=O)cc1)N1CC(O)C1]

4-((2-oxo-1,2-dihydroquinolin-3-yl)methyl)benzoic acid (21.0 mg, 0.0750 mmol) and HATU (28.5 mg, 0.0750 mmol) and azetidin-3-ol, hydrochloride (34.6 mg, 0.150 mmol) were added to a 8 mL vial. Then, DMF (1.5 mL) was added followed by DIPEA (0.090 mL, 0.52 mmol). The reaction mixture was allowed to stir at room temperature for 16 h, and then it was filtered and purified by reversed phase HPLC (MeCN/water w/0.1% TFA). ¹H NMR (500 MHz, DMSO-d₆) δ 11.82 (s, 1H), 7.76 (s, 1H), 7.60 (d, J=7.8 Hz, 1H), 7.54 (d, J=8.1 Hz, 2H), 7.45 (t, J=7.3 Hz, 1H), 7.36 (d, J=8.1 Hz, 2H), 7.30 (d, J=8.2 Hz, 1H), 7.16 (t, J=7.2 Hz, 1H), 5.80 (d, J=6.0 Hz, 1H), 4.51-4.38 (m, 2H), 4.27-4.18 (m, 1H), 4.05-3.96 (m, 1H), 3.88 (s, 2H), 3.79-3.73 (m, 1H). LC/MS (m/z): 335 (M+H)+.

Examples in the Table 1 below were prepared in a similar manner to Example 1 using the appropriate starting materials.

TABLE 1

| | Structure | LCMS (m/z) |
|---|---|---|
| Example | name | (M + H)+ |
| 2 | 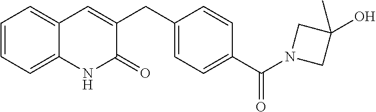 [SMILES: CC1(O)CN(C(=O)c2ccc(Cc3cc4ccccc4[nH]c3=O)cc2)C1] | 349 |
| | 3-(4-(3-hydroxy-3-methylazetidine-1-carbonyl)benzyl)- | |
| | quinolin-2(1H)-one | |

### Example 3

### Preparation of 3-((5-(2,6-difluorophenyl)-1,3,4-oxadiazol-2-yl)methyl)quinolin-2(1H)-one

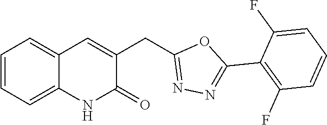 [SMILES: O=c1[nH]c2ccccc2cc1Cc1nnc(-c2c(F)cccc2F)o1]

To a vial containing 2,6-difluorobenzohydrazide (34.4 mg, 0.200 mmol), PS-TPP (126 mg, 0.300 mmol, 2.38 mmol/g), and 2-(2-oxo-1,2-dihydroquinolin-3-yl)acetic acid (20.3 mg, 0.100 mmol) was added trichloroacetonitrile (0.100 ml, 1.00 mmol) and MeCN (2.5 ml). The reaction was stirred and heated at 160° C. for 15 minutes. The mixture was filtered, washed with MeCN, and concentrated. The residue was purified by reversed-phase HPLC (MeCN/water w/0.1% TFA) to afford 3-((5-(2,6-difluorophenyl)-1,3,4-oxadiazol-2-yl)methyl)quinolin-2(1H)-one. ¹H NMR (600 MHz, DMSO-d₆) δ 11.97 (s, 1H), 8.04 (s, 1H), 7.79-7.72 (m, 1H), 7.69 (d, J=7.6 Hz, 1H), 7.54-7.48 (m, 1H), 7.38 (t, J=8.7 Hz, 2H), 7.34 (d, J=8.2 Hz, 1H), 7.24-7.18 (m, 1H), 4.26 (s, 2H). LC/MS (m/z): 340 (M+H)+

Examples shown in Table 2 below, were prepared according to procedures analogous to those outlined in Example 2 above using the appropriate starting materials, described in the Preparations or Intermediates above, or as obtained from commercial sources.

TABLE 2

| | Structure | LCMS (m/z) |
|---|---|---|
| Example | name | (M + H)+ |
| 4 | 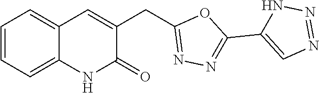 [SMILES: O=c1[nH]c2ccccc2cc1Cc1nnc(-c2cnn[nH]2)o1] | 295 |
| | 3-((5-(1H-1,2,3-triazol-5-yl)-1,3,4-oxadiazol-2- | |
| | yl)methyl)quinolin-2(1H)-one | |
| 5 | 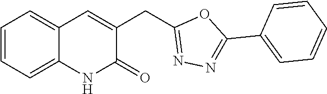 [SMILES: O=c1[nH]c2ccccc2cc1Cc1nnc(-c2ccccc2)o1] | 304 |
| | 3-((5-phenyl-1,3,4-oxadiazol-2-yl)methyl)- | |
| | quinolin-2(1H)-one | |
| 6 | 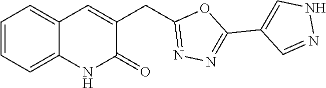 [SMILES: O=c1[nH]c2ccccc2cc1Cc1nnc(-c2cn[nH]c2)o1] | 294 |
| | 3-((5-(1H-pyrazol-4-yl)-1,3,4-oxadiazol-2-yl)- | |
| | methyl)quinolin-2(1H)-one | |
| 7 | 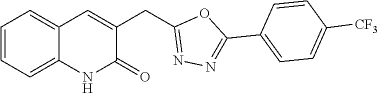 [SMILES: O=c1[nH]c2ccccc2cc1Cc1nnc(-c2ccc(C(F)(F)F)cc2)o1] | 372 |
| | 3-((5-(4-(trifluoromethyl)phenyl)-1,3,4-oxadiazol- | |
| | 2-yl)methyl)quinolin-2(1H)-one | |
| 8 | 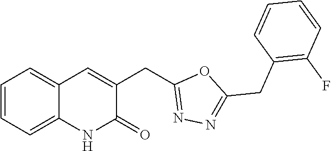 [SMILES: O=c1[nH]c2ccccc2cc1Cc1nnc(Cc2ccccc2F)o1] | 336 |
| | 3-((5-(2-fluorobenzyl)-1,3,4-oxadiazol-2-yl)- | |
| | methyl)quinolin-2(1H)-one | |
| 9 | 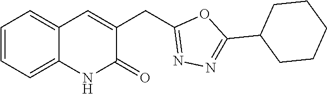 [SMILES: O=c1[nH]c2ccccc2cc1Cc1nnc(C2CCCCC2)o1] | 310 |
| | 3-((5-cyclohexyl-1,3,4-oxadiazol-2-yl)methyl)- | |
| | quinolin-2(1H)-one | |
| 10 | 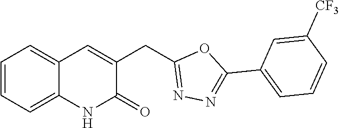 [SMILES: O=c1[nH]c2ccccc2cc1Cc1nnc(-c2cccc(C(F)(F)F)c2)o1] | 372 |
| | 3-((5-(3-(trifluoromethyl)phenyl)-1,3,4-oxadiazol- | |
| | 2-yl)methyl)quinolin-2(1H)-one | |

### Example 11

### Preparation of 3-((5-(2,5-dichlorophenyl)-1,3,4-oxadiazol-2-yl)methyl)quinolin-2(1H)-one

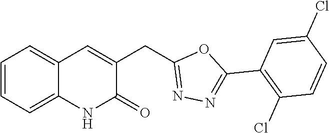 [SMILES: O=c1[nH]c2ccccc2cc1Cc1nnc(-c2cc(Cl)ccc2Cl)o1]

2-(2-oxo-1,2-dihydroquinolin-3-yl)acetohydrazide (0.0220 g, 0.100 mmol), 2,5-dichlorobenzoic acid (38.0 mg, 0.200 mmol), DMA (1 ml) T3P 50% wt in DMF (200 μL, 0.100 mmol) and DIPEA (0.300 ml, 1.72 mmol) were added to a vial. The mixture was stirred at room temperature for 16 h. Additional DMA (1 ml), T3P 50% wt in DMF (200 μL, 0.100 mmol), and DIPEA (0.300 ml, 1.72 mmol) were again added, and the mixture was heated at 110° C. for 6 hours. The mixture was cooled to room temperature and purified by reversed phase HPLC (MeCN/water w/0.1% TFA) to afford 3-((5-(2,5-dichlorophenyl)-1,3,4-oxadiazol-2-yl)methyl)quinolin-2(1H)-one. ¹H NMR (500 MHz, DMSO-d₆) δ 11.98 (s, 1H), 8.04 (s, 1H), 7.77-7.66 (m, 4H), 7.52 (t, J=7.7 Hz, 1H), 7.34 (d, J=8.3 Hz, 1H), 7.21 (t, J=7.5 Hz, 1H), 4.25 (s, 2H). LC/MS (m/z): 372 (M+H)+

Examples as shown in Table 3 below, were prepared according to procedures analogous to those outlined in Example 11 above using the appropriate starting materials, described in the Preparations or Intermediates above, or as obtained from commercial sources.

TABLE 3

| | Structure | LCMS (m/z) |
|---|---|---|
| Example | Name | (M + H)+ |
| 12 | 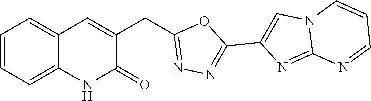 [SMILES: O=c1[nH]c2ccccc2cc1Cc1nnc(-c2cn3cccnc3n2)o1] | 345 |
| | 3-((5-(imidazo[1,2-a]pyrimidin-2-yl)-1,3,4-oxadiazol- | |
| | 2-yl)methyl)quinolin-2(1H)-one | |
| 13 | 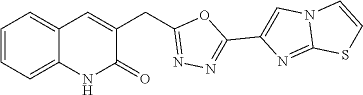 [SMILES: O=c1[nH]c2ccccc2cc1Cc1nnc(-c2cn3ccsc3n2)o1] | 350 |
| | 3-((5-(imidazo[2,1-b]thiazol-6-yl)-1,3,4-oxadiazol-2- | |
| | yl)methyl)quinolin-2(1H)-one | |
| 14 | 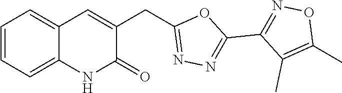 [SMILES: Cc1onc(-c2nnc(Cc3cc4ccccc4[nH]c3=O)o2)c1C] | 323 |
| | 3-((5-(4,5-dimethylisoxazol-3-yl)-1,3,4-oxadiazol-2- | |
| | yl)methyl)quinolin-2(1H)-one | |
| 15 | 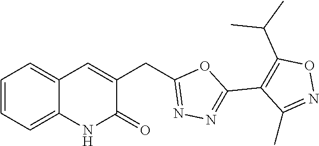 [SMILES: Cc1noc(C(C)C)c1-c1nnc(Cc2cc3ccccc3[nH]c2=O)o1] | 351 |
| | 3-((5-(5-isopropyl-3-methylisoxazol-4-yl)-1,3,4- | |
| | oxadiazol-2-yl)methyl)quinolin-2(1H)-one | |
| 16 | 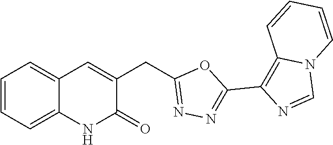 [SMILES: O=c1[nH]c2ccccc2cc1Cc1nnc(-c2ncn3ccccc23)o1] | 344 |
| | 3-((5-(imidazo[1,5-a]pyridin-1-yl)-1,3,4-oxadiazol- | |
| | 2-yl)methyl)quinolin-2(1H)-one | |
| 17 | 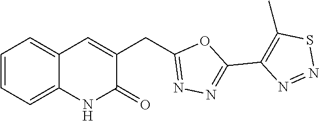 [SMILES: Cc1snnc1-c1nnc(Cc2cc3ccccc3[nH]c2=O)o1] | 326 |
| | 3-((5-(5-methyl-1,2,3-thiadiazol-4-yl)-1,3,4- | |
| | oxadiazol-2-yl)methyl)quinolin-2(1H)-one | |
| 18 | 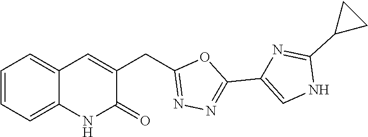 [SMILES: O=c1[nH]c2ccccc2cc1Cc1nnc(-c2c[nH]c(C3CC3)n2)o1] | 334 |
| | 3-((5-(2-cyclopropyl-1H-imidazol-4-yl)-1,3,4-oxadiazol- | |
| | 2-yl)methyl)quinolin-2(1H)-one | |
| 19 | 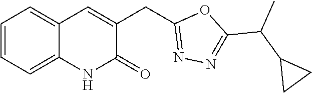 [SMILES: CC(c1nnc(Cc2cc3ccccc3[nH]c2=O)o1)C1CC1] | 296 |
| | rac-3-((5-(1-cyclopropylethyl)-1,3,4-oxadiazol-2- | |
| | yl)methyl)quinolin-2(1H)-one | |
| 20 | 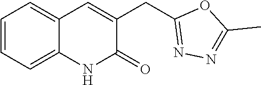 [SMILES: Cc1nnc(Cc2cc3ccccc3[nH]c2=O)o1] | 242 |
| | 3-((5-methyl-1,3,4-oxadiazol-2-yl)meth- | |
| | yl)quinolin-2(1H)-one | |
| 21 | 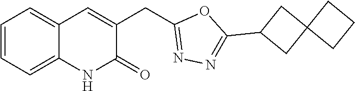 [SMILES: O=c1[nH]c2ccccc2cc1Cc1nnc(C2CC3(CCC3)C2)o1] | 322 |
| | 3-((5-(spiro[3.3]heptan-2-yl)-1,3,4-oxadiazol-2-yl)- | |
| | methyl)quinolin-2(1H)-one | |
| 22 | 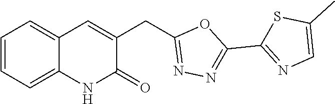 [SMILES: Cc1cnc(-c2nnc(Cc3cc4ccccc4[nH]c3=O)o2)s1] | 325 |
| | 3-((5-(5-methylthiazol-2-yl)-1,3,4-oxadiazol-2-yl)- | |
| | methyl)quinolin-2(1H)-one | |
| 23 | 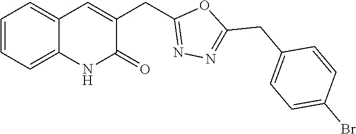 [SMILES: O=c1[nH]c2ccccc2cc1Cc1nnc(Cc2ccc(Br)cc2)o1] | 396 |
| | 3-((5-(4-bromobenzyl)-1,3,4-oxadiazol-2-yl)methyl)- | |
| | quinolin-2(1H)-one | |
| 24 | 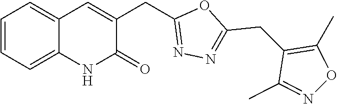 [SMILES: Cc1noc(C)c1Cc1nnc(Cc2cc3ccccc3[nH]c2=O)o1] | 337 |
| | 3-((5-((3,5-dimethylisoxazol-4-yl)methyl)-1,3,4- | |
| | oxadiazol-2-yl)methyl)quinolin-2(1H)-one | |
| 25 | 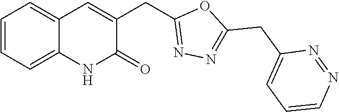 [SMILES: O=c1[nH]c2ccccc2cc1Cc1nnc(Cc2cccnn2)o1] | 320 |
| | 3-((5-(5-isopropyl-3-methylisoxazol-4-yl)-1,3,4- | |
| | oxadiazol-2-yl)methyl)quinolin-2(1H)-one | |
| 26 | 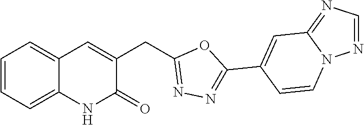 [SMILES: O=c1[nH]c2ccccc2cc1Cc1nnc(-c2ccn3ncnc3c2)o1] | 345 |
| | 3-((5-([1,2,4]triazolo[1,5-a]pyridin-7-yl)-1,3,4- | |
| | oxadiazol-2-yl)methyl)quinolin-2(1H)-one | |
| 27 | 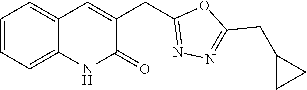 [SMILES: O=c1[nH]c2ccccc2cc1Cc1nnc(CC2CC2)o1] | 282 |
| | 3-((5-(cyclopropylmethyl)-1,3,4-oxadiazol-2- | |
| | yl)methyl)quinolin-2(1H)-one | |
| 28 | 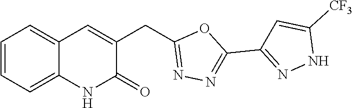 [SMILES: O=c1[nH]c2ccccc2cc1Cc1nnc(-c2cc(C(F)(F)F)[nH]n2)o1] | 362 |
| | 3-((5-(5-(trifluoromethyl)-1H-pyrazol-3-yl)-1,3,4- | |
| | oxadiazol-2-yl)methyl)quinolin-2(1H)-one | |
| 29 | 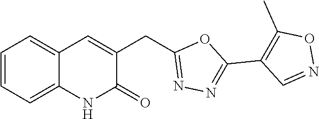 [SMILES: Cc1oncc1-c1nnc(Cc2cc3ccccc3[nH]c2=O)o1] | 309 |
| | 3-((5-(5-methylisoxazol-4-yl)-1,3,4-oxadiazol-2- | |
| | yl)methyl)quinolin-2(1H)-one | |
| 30 | 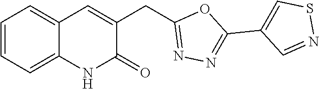 [SMILES: O=c1[nH]c2ccccc2cc1Cc1nnc(-c2cnsc2)o1] | 311 |
| | 3-((5-(isothiazol-4-yl)-1,3,4-oxadiazol-2-yl)- | |
| | methyl)quinolin-2(1H)-one | |
| 31 | 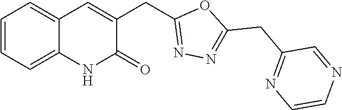 [SMILES: O=c1[nH]c2ccccc2cc1Cc1nnc(Cc2cnccn2)o1] | 320 |
| | 3-((5-(pyrazin-2-ylmethyl)-1,3,4-oxadiazol-2-yl)- | |
| | methyl)quinolin-2(1H)-one | |
| 32 | 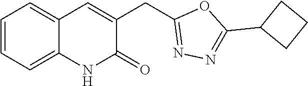 [SMILES: O=c1[nH]c2ccccc2cc1Cc1nnc(C2CCC2)o1] | 282 |
| | 3-((5-cyclobutyl-1,3,4-oxadiazol-2-yl)methyl)- | |
| | quinolin-2(1H)-one | |
| 33 | 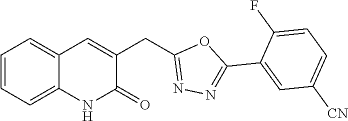 [SMILES: N#Cc1ccc(F)c(-c2nnc(Cc3cc4ccccc4[nH]c3=O)o2)c1] | 347 |
| | 4-fluoro-3-(5-((2-oxo-1,2-dihydroquinolin-3-yl)- | |
| | methyl)-1,3,4-oxadiazol-2-yl)benzonitrile | |
| 34 | 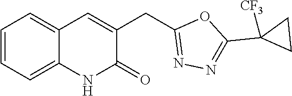 [SMILES: O=c1[nH]c2ccccc2cc1Cc1nnc(C2(C(F)(F)F)CC2)o1] | 336 |
| | 3-((5-(1-(trifluoromethyl)cyclopropyl)-1,3,4- | |
| | oxadiazol-2-yl)methyl)quinolin-2(1H)-one | |
| 35 | 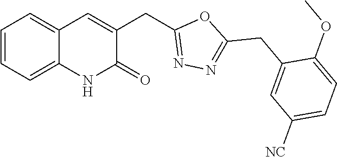 [SMILES: COc1ccc(C#N)cc1Cc1nnc(Cc2cc3ccccc3[nH]c2=O)o1] | 373 |
| | 4-methoxy-3-((5-((2-oxo-1,2-dihydroquinolin-3-yl)- | |
| | methyl)-1,3,4-oxadiazol-2-yl)methyl)benzonitrile | |
| 36 | 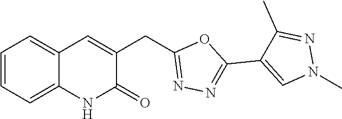 [SMILES: Cc1nn(C)cc1-c1nnc(Cc2cc3ccccc3[nH]c2=O)o1] | 322 |
| | 3-((5-(1,3-dimethyl-1H-pyrazol-4-yl)-1,3,4-oxadiazol- | |
| | 2-yl)methyl)quinolin-2(1H)-one | |
| 37 | 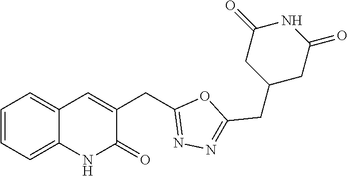 [SMILES: O=C1CC(Cc2nnc(Cc3cc4ccccc4[nH]c3=O)o2)CC(=O)N1] | 353 |
| | 4-((5-((2-oxo-1,2-dihydroquinolin-3-yl)methyl)-1,3,4- | |
| | oxadiazol-2-yl)methyl)piperidine-2,6-dione | |
| 38 | 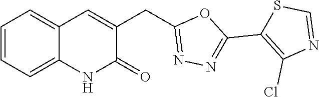 [SMILES: O=c1[nH]c2ccccc2cc1Cc1nnc(-c2scnc2Cl)o1] | 345 |
| | 3-((5-(4-chlorothiazol-5-yl)-1,3,4-oxadiazol-2-yl)- | |
| | methyl)quinolin-2(1H)-one | |
| 39 | 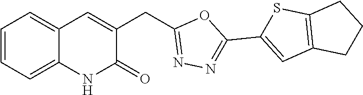 [SMILES: O=c1[nH]c2ccccc2cc1Cc1nnc(-c2cc3c(s2)CCC3)o1] | 350 |
| | 3-((5-(5,6-dihydro-4H-cyclopenta[b]thiophen-2-yl)- | |
| | 1,3,4-oxadiazol-2-yl)methyl)quinolin-2(1H)-one | |
| 40 | 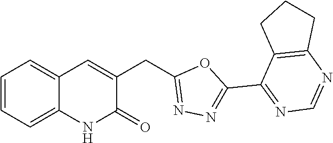 [SMILES: O=c1[nH]c2ccccc2cc1Cc1nnc(-c2ncnc3c2CCC3)o1] | 346 |
| | 3-((5-(6,7-dihydro-5H-cyclopenta[d]pyrimidin-4-yl)- | |
| | 1,3,4-oxadiazol-2-yl)methyl)quinolin-2(1H)-one | |
| 41 | 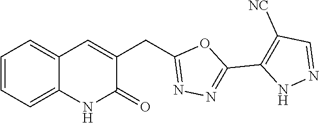 [SMILES: N#Cc1cn[nH]c1-c1nnc(Cc2cc3ccccc3[nH]c2=O)o1] | 319 |
| | 5-(5-((2-oxo-1,2-dihydroquinolin-3-yl)methyl)- | |
| | 1,3,4-oxadiazol-2-yl)-1H-pyrazole-4-carbonitrile | |
| 42 | 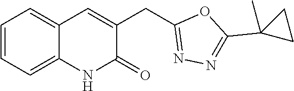 [SMILES: CC1(c2nnc(Cc3cc4ccccc4[nH]c3=O)o2)CC1] | 282 |
| | 3-((5-(1-methylcyclopropyl)-1,3,4-oxadiazol-2- | |
| | yl)methyl)quinolin-2(1H)-one | |
| 43 | 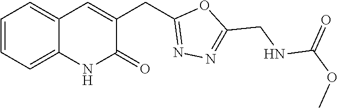 [SMILES: COC(=O)NCc1nnc(Cc2cc3ccccc3[nH]c2=O)o1] | 315 |
| | methyl ((5-((2-oxo-1,2-dihydroquinolin-3-yl)methyl)- | |
| | 1,3,4-oxadiazol-2-yl)methyl)carbamate | |
| 44 | 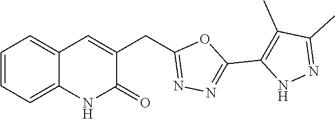 [SMILES: Cc1n[nH]c(-c2nnc(Cc3cc4ccccc4[nH]c3=O)o2)c1C] | 322 |
| | 3-((5-(3,4-dimethyl-1H-pyrazol-5-yl)-1,3,4-oxadiazol- | |
| | 2-yl)methyl)quinolin-2(1H)-one | |

### Example 45

### Preparation of 3-(4-(3-methyl-5-oxopyrrolidin-2-yl)benzyl)quinolin-2(1H)-one

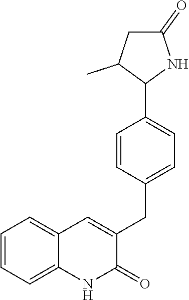 [SMILES: CC1CC(=O)NC1c1ccc(Cc2cc3ccccc3[nH]c2=O)cc1]

4-methylpyrrolidin-2-one (37 mg, 0.38 mmol), Ir[dF(CF₃)ppy]₂(dtbbpy)PF₆ (4.5 mg, 4.5 μmol), 3-(4-bromobenzyl)-2-methoxyquinoline (49 mg, 0.15 mmol) and [Ni(dtbbpy)(H₂O)₄]Cl₂ (0.4 mg, 1 μmol) were added to a 8 mL vial. To this was added DMSO (1.25 mL), quinuclidine-3yl-acetate (0.013 mL, 0.083 mmol) and water (0.32 ml, 18.0 mmol). The mixture was sparged with argon for 5 minutes. The mixture was stirred and irradiated in a penn photoreactor for 2 hours (450 nm, 100% light, full fan and half stir rate). Additional quinuclidine-3yl-acetate (0.013 mL, 0.083 mmol) was added by micro syringe, and the mixture was stirred and irradiated for 3 more hours. The mixture was diluted with water (1.5 mL) and extracted with EtOAc (3×6 mL). The combined organic layer was concentrated. To this crude material was added sodium iodide (90 mg, 0.60 mmol) and MeCN (2 mL) followed by TMS-Cl (0.077 mL, 0.60 mmol). The mixture was stirred at room temperature for 1 hour. The mixture was concentrated, and the residue was purified by reversed phase HPLC (MeCN/water w/0.1% NH₄OH). ¹H NMR (600 MHz, DMSO-d₆) δ 11.78 (s, 1H), 7.94 (s, 1H), 7.73 (s, 1H), 7.58 (d, J=7.4 Hz, 1H), 7.46-7.42 (m, 1H), 7.32-7.28 (m, 3H), 7.23 (d, J=8.1 Hz, 2H), 7.16-7.13 (m, 1H), 4.11 (d, J=7.1 Hz, 1H), 3.83 (s, 2H), 2.37 (dd, J=16.2, 8.2 Hz, 1H), 2.12-2.03 (m, 1H), 1.95 (dd, J=16.2, 9.2 Hz, 1H), 1.04 (d, J=6.7 Hz, 3H). LC/MS (m/z): 333 (M+H)+.

### Example 46

### Preparation of 3-((6-(1,1-dioxidoisothiazolidin-2-yl)pyridin-3-yl)methyl)quinolin-2(1H)-one

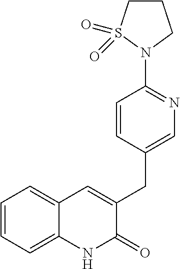 [SMILES: O=c1[nH]c2ccccc2cc1Cc1ccc(N2CCCS2(=O)=O)nc1]

### Step A: 2-(5-((2-methoxyquinolin-3-yl)methyl)pyridin-2-yl)isothiazolidine 1,1-dioxide

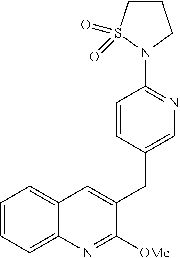 [SMILES: COc1nc2ccccc2cc1Cc1ccc(N2CCCS2(=O)=O)nc1]

3-((6-chloropyridin-3-yl)methyl)-2-methoxyquinoline (150 mg, 0.527 mmol), isothiazolidine 1,1-dioxide (96.0 mg, 0.790 mmol), XantPhos-Pd G2 (46.8 mg, 0.0530 mmol) and Cs₂CO₃ (515 mg, 1.58 mmol) were added to a 8 mL vial in 1,4-dioxane (2.6 mL). The reaction mixture was purged with Ar. The mixture was stirred and heated at 80° C. for 8 h. The mixture was quenched with water, and extracted with DCM. The organic layer was dried, filtered, and the solvents were evaporated. The crude material was purified on silica gel with 40-60% EtOAc in hexanes as eluent to afford 2-(5-((2-methoxyquinolin-3-yl)methyl)pyridin-2-yl)isothiazolidine 1,1-dioxide LC/MS (m/z): 392 (M+Na)⁺

### Step C: 3-((6-(1,1-dioxidoisothiazolidin-2-yl)pyridin-3-yl)methyl)quinolin-2(1H)-one

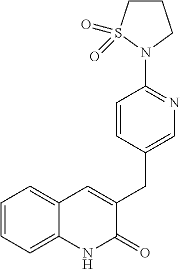 [SMILES: O=c1[nH]c2ccccc2cc1Cc1ccc(N2CCCS2(=O)=O)nc1]

To a 8 mL vial containing product from step A were added sodium iodide (316 mg, 2.11 mmol) and chlorotrimethylsilane (267 μl, 2.11 mmol) in 3 mL of MeCN. The reaction mixture was stirred at room temperature for 16 h. The mixture was quenched with water and saturated NaHCO₃ aq. The precipitate was collected by filtration, washed with water several times and dried under high vacuum to afford 3-((6-(1,1-dioxidoisothiazolidin-2-yl)pyridin-3-yl)methyl)quinolin-2(1H)-one. ¹H NMR (600 MHz, DMSO-d₆) δ 11.82 (s, 1H), 8.29 (d, J=2.0 Hz, 1H), 7.79-7.67 (m, 2H), 7.60 (d, J=7.8 Hz, 1H), 7.46-7.43 (m, 1H), 7.29 (d, J=8.2 Hz, 1H), 7.17-7.10 (m, 2H), 3.88 (t, J=6.6 Hz, 2H), 3.80 (s, 2H), 3.55 (t, J=7.3 Hz, 2H), 2.38 (p, J=6.9 Hz, 2H). LC/MS (m/z): 356 (M+H)+

Examples shown in Table 4 below, were prepared according to procedures analogous to those outlined in Example 46 above using the appropriate starting materials, described in the Preparations or Intermediates above, or as obtained from commercial sources.

TABLE 4

| | Structure | LCMS (m/z) |
|---|---|---|
| Example | Name | (M + H)+ |
| 47 | 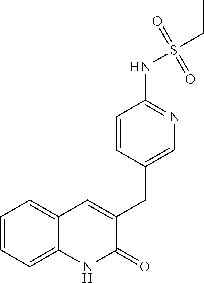 [SMILES: CCS(=O)(=O)Nc1ccc(Cc2cc3ccccc3[nH]c2=O)cn1] | 344 |
| | N-(5-((2-oxo-1,2-dihydroquinolin- | |
| | 3-yl)methyl)pyridin-2-yl)ethane- | |
| | sulfonamide | |
| 48 | 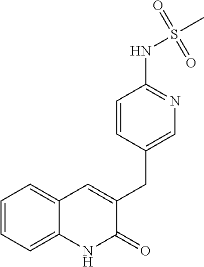 [SMILES: CS(=O)(=O)Nc1ccc(Cc2cc3ccccc3[nH]c2=O)cn1] | 330 |
| | N-(5-((2-oxo-1,2-dihydroquin- | |
| | olin-3-yl)methyl)pyridin- | |
| | 2-yl)methanesulfonamide | |
| 49 | 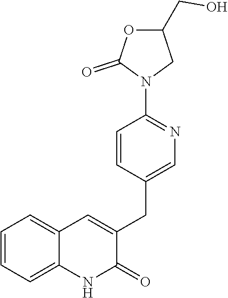 [SMILES: O=C1OC(CO)CN1c1ccc(Cc2cc3ccccc3[nH]c2=O)cn1] | 352 |
| | rac-5-(hydroxymethyl)-3-(5-((2-oxo- | |
| | 1,2-dihydroquinolin-3-yl)methyl)- | |
| | pyridin-2-yl)oxazolidin-2-one | |
| 50 | 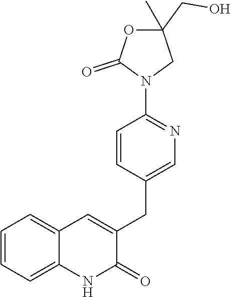 [SMILES: CC1(CO)CN(c2ccc(Cc3cc4ccccc4[nH]c3=O)cn2)C(=O)O1] | 366 |
| | rac-5-(hydroxymethyl)-5-methyl-3-(5- | |
| | ((2-oxo-1,2-dihydroquinolin-3-yl)- | |
| | methyl)pyridin-2-yl)oxazolidin-2-one | |
| 51 | 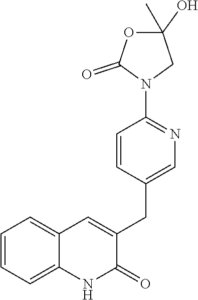 [SMILES: CC1(O)CN(c2ccc(Cc3cc4ccccc4[nH]c3=O)cn2)C(=O)O1] | 352 |
| | rac-5-hydroxy-5-methyl-3-(5-((2- | |
| | oxo-1,2-dihydroquinolin-3-yl)- | |
| | methyl)pyridin-2-yl)oxazolidin- | |
| | 2-one | |
| 52 | 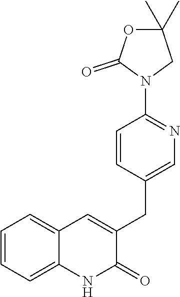 [SMILES: CC1(C)CN(c2ccc(Cc3cc4ccccc4[nH]c3=O)cn2)C(=O)O1] | 350 |
| | 5,5-dimethyl-3-(5-((2-oxo-1,2- | |
| | dihydroquinolin-3-yl)methyl)- | |
| | pyridin-2-yl)oxazolidin-2-one | |
| 53 | 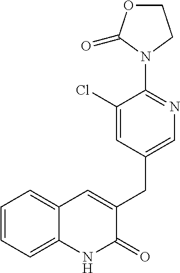 [SMILES: O=C1OCCN1c1ncc(Cc2cc3ccccc3[nH]c2=O)cc1Cl] | 356 |
| | 5,5-dimethyl-3-(5-((2-oxo-1,2- | |
| | dihydroquinolin-3-yl)methyl)- | |
| | pyridin-2-yl)oxazolidin-2-one | |

Examples shown in Table 5 below, were prepared according to procedures analogous to those outlined in Example 46 above using the appropriate starting materials, described in the Preparations or Intermediates above, or as obtained from commercial sources. Also, the products were resolved into their single enantiomers by chiral SFC resolution.

TABLE 5

| | | LCMS | |
|---|---|---|---|
| | Structure | (m/z) | |
| Example | Name | (M + H)+ | SFC conditions |
| 54A (peak 1) and 54B (peak 2) | 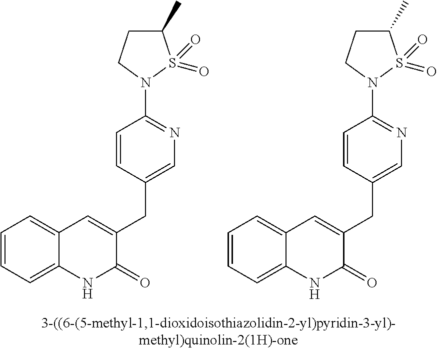 [SMILES: C[C@@H]1CCN(c2ccc(Cc3cc4ccccc4[nH]c3=O)cn2)S1(=O)=O ; C[C@H]1CCN(c2ccc(Cc3cc4ccccc4[nH]c3=O)cn2)S1(=O)=O] | 370 | IB-N 21 × 250 mm column; 30% MeOH (w/ 0.1% NH₄OH) modifier |
| 55A (peak 1) and 55B (peak 2) | 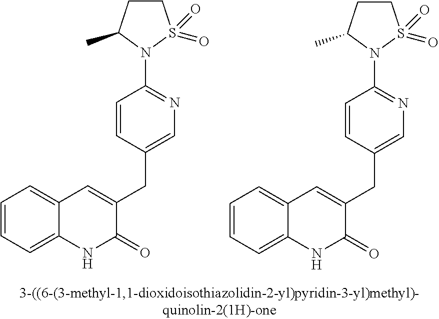 [SMILES: C[C@H]1CCS(=O)(=O)N1c1ccc(Cc2cc3ccccc3[nH]c2=O)cn1 ; C[C@@H]1CCS(=O)(=O)N1c1ccc(Cc2cc3ccccc3[nH]c2=O)cn1] | 370 | OJ-H 21 × 250 mm column; 20% MeOH (w/ 0.1% NH₄OH) modifier |
| 56A (peak 1) and 56B (peak 2) | 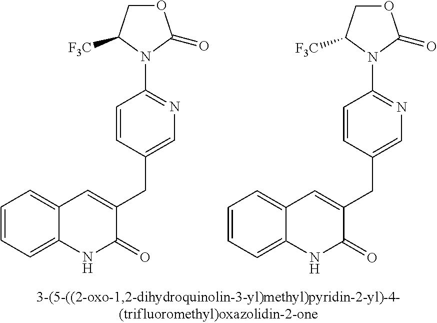 [SMILES: O=C1OC[C@H](C(F)(F)F)N1c1ccc(Cc2cc3ccccc3[nH]c2=O)cn1 ; O=C1OC[C@@H](C(F)(F)F)N1c1ccc(Cc2cc3ccccc3[nH]c2=O)cn1] | 390 | CCA-F4 21 × 250 mm column; 25% MeOH (w/ 0.1% NH₄OH) modifier |
| 57A (peak 1) and 57B (peak 2) | 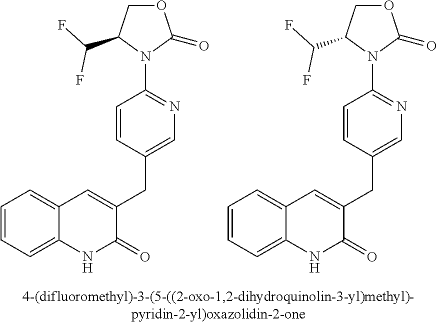 [SMILES: O=C1OC[C@H](C(F)F)N1c1ccc(Cc2cc3ccccc3[nH]c2=O)cn1 ; O=C1OC[C@@H](C(F)F)N1c1ccc(Cc2cc3ccccc3[nH]c2=O)cn1] | 372 | IB 21 × 250 mm column; 25% MeOH (w/ 0.1% NH₄OH) modifier |

### Example 58

### Preparation of 3-(4-((3-methylazetidin-3-yl)amino)benzyl)quinolin-2(1H)-one

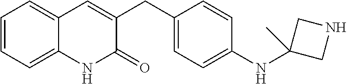 [SMILES: CC1(Nc2ccc(Cc3cc4ccccc4[nH]c3=O)cc2)CNC1]

Toluene (3 ml) was added to an 8 mL vial containing tert-butyl 3-amino-3-methylazetidine-1-carboxylate, oxalic acid (83.2 mg, 0.301 mmol), Ruphos-Pd G4 (16 mg, 0.018 mmol), cesium carbonate (119 mg, 0.366 mmol), and 3-(4-bromobenzyl)-2-methoxyquinoline (60.0 mg, 0.183 mmol). The headspace was purged with argon, and the reaction was stirred at 100° C. for 16 h. Additional Ruphos-Pd G4 (45 mg, 0.040 mmol) was added, as well as another portion of cesium carbonate (119 mg, 0.366 mmol), and the vial was purged with argon and heated at 100° C. for 7 h. The mixture was cooled to room temperature. The reaction was then filtered and concentrated under reduced pressure. Sodium iodide (110 mg, 0.731 mmol) was added to the crude residue, followed by MeCN (3 ml). Then, chlorotrimethylsilane (0.100 ml, 0.788 mmol) was added, and the mixture was stirred at room temperature for 3.5 h. The mixture was filtered and concentrated under reduced pressure. The residue was dissolved in DMSO, filtered, and purified by reverse phase chromatography (MeCN/water w/0.1% TFA) to afford 3-(4-((3-methylazetidin-3-yl)amino)benzyl)quinolin-2(1H)-one. ¹H NMR (600 MHz, DMSO) δ 11.74 (s, 1H), 8.89 (d, J=19.8 Hz, 2H), 7.61 (s, 1H), 7.55 (d, J=7.7 Hz, 1H), 7.44-7.40 (m, 1H), 7.28 (d, J=8.2 Hz, 1H), 7.15-7.11 (m, 1H), 7.05 (d, J=8.4 Hz, 2H), 6.39 (d, J=8.5 Hz, 2H), 3.95 (t, J=6.3 Hz, 4H), 3.68 (s, 2H), 1.52 (s, 3H). LC/MS (m/z): 320 (M+H)+.

### Example 59

### Preparation of 3-(4-(piperazin-1-yl)benzyl)quinolin-2(1H)-one

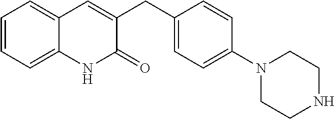 [SMILES: O=c1[nH]c2ccccc2cc1Cc1ccc(N2CCNCC2)cc1]

3-(4-bromobenzyl)quinolin-2(1H)-one (63.8 mg, 0.203 mmol), tert-butyl piperazine-1-carboxylate (76.0 mg, 0.406 mmol), and Ruphos-Pd G4 (34.5 mg, 0.0410 mmol) were added to a 8 mL argon-purged vial, followed by THF (2 ml). The solution was sparged with argon for ˜1 min. A 0.5 M solution of sodium tert-butoxide (0.400 ml, 0.800 mmol) was added. The mixture was stirred at room temperature for 16 h. The mixture was filtered and concentrated. The residue was dissolved in DCM (3 ml), and then TFA (0.600 ml, 7.79 mmol) was added. The mixture was stirred at room temperature for 4 h. The mixture was filtered and concentrated. The residue was dissolved in DMSO (4 mL), filtered, and purified by reversed phase HPLC (MeCN/water w/0.1% TFA) to afford 3-(4-(piperazin-1-yl)benzyl)quinolin-2(1H)-one. ¹H NMR (600 MHz, DMSO-d₆) δ 11.77 (s, 1H), 8.72 (s, 2H), 7.65 (s, 1H), 7.57 (d, J=7.5 Hz, 1H), 7.47-7.41 (m, 1H), 7.29 (d, J=8.2 Hz, 1H), 7.20 (d, J=8.6 Hz, 2H), 7.17-7.11 (m, 1H), 6.93 (d, J=8.7 Hz, 2H), 3.75 (s, 2H), 3.31-3.26 (m, 4H), 3.24 (s, 4H). LC/MS (m/z): 320 (M+H)+.

Examples shown in Table 6 below, were prepared according to procedures analogous to those outlined in Example 59 above using the appropriate starting materials, described in the Preparations or Intermediates above, or as obtained from commercial sources.

TABLE 6

| | Structure | LCMS (m/z) |
|---|---|---|
| Example | Name | (M + H)+ |
| 60 | 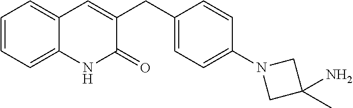 [SMILES: CC1(N)CN(c2ccc(Cc3cc4ccccc4[nH]c3=O)cc2)C1] | 320 |
| | 3-(4-(3-amino-3-methylazetidin-1-yl)benzyl)quinolin- | |
| | 2(1H)-one | |

### Example 61

### Preparation of 3-(4-((2-oxo-1,2-dihydro-1,6-naphthyridin-3-yl)methyl)phenyl)oxazolidin-2-one

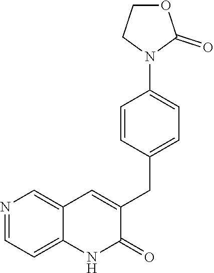 [SMILES: O=C1OCCN1c1ccc(Cc2cc3cnccc3[nH]c2=O)cc1]

3-(4-bromobenzyl)-2-methoxy-1,6-naphthyridine (33 mg, 0.10 mmol) and oxazolidin-2-one (17.6 mg, 0.200 mmol), copper iodide (5.0 mg, 0.025 mmol), potassium carbonate (29.0 mg, 0.21 mmol) and trans-1,2-diaminocyclohexane (3.0 mg, 0.025 mmol) were added to a 8 mL vial under nitrogen. To this was added 1,4-dioxane (1 mL), and the mixture was purged with nitrogen. The reaction mixture was stirred and heated at 90° C. for 15 hours. The mixture was filtered. To the filtrate was added 4 M HCl in dioxane (1 mL) and 1 M aqueous HCl (0.25 mL). The mixture was stirred and heated at 50° C. for 45 minutes. The mixture was concentrated, and the residue was purified by reversed phase HPLC (MeCN/water w/0.1% NH₄OH). ¹H NMR (500 MHz, DMSO-d₆) δ 12.09 (s, 1H), 8.77 (s, 1H), 8.42 (d, J=5.6 Hz, 1H), 7.75 (s, 1H), 7.50 (d, J=8.6 Hz, 2H), 7.32 (d, J=8.5 Hz, 2H), 7.19 (d, J=5.7 Hz, 1H), 4.46-4.40 (m, 2H), 4.07-4.01 (m, 2H), 3.82 (s, 2H). LC/MS (m/z): 322 (M+H)+.

Examples shown in Table 7 below, were prepared according to procedures analogous to those outlined in Example 61 above using the appropriate starting materials, described in the Preparations or Intermediates above, or as obtained from commercial sources.

TABLE 7

| | Structure | LCMS (m/z) |
|---|---|---|
| Example | Name | (M + H)+ |
| 62 | 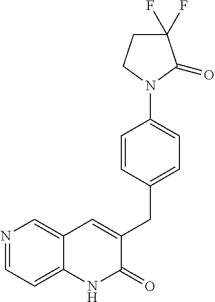 [SMILES: O=C1N(c2ccc(Cc3cc4cnccc4[nH]c3=O)cc2)CCC1(F)F] | 356 |
| | 3-(4-(3,3-difluoro-2-oxopyrrolidin- | |
| | 1-yl)benzyl)-1,6-naphthyridin- | |
| | 2(1H)-one | |

### Example 63

### Preparation of 3-(4-(azetidin-3-yl)benzyl)quinolin-2(1H)-one

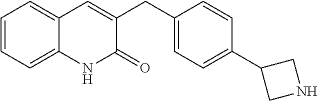 [SMILES: O=c1[nH]c2ccccc2cc1Cc1ccc(C2CNC2)cc1]

A solution of tert-butyl 3-iodoazetidine-1-carboxylate (105 mg, 0.372 mmol) in DMA (3 ml) was added to an argon-purged 8 ml vial containing 3-(4-bromobenzyl)quinolin-2(1H)-one (90 mg, 0.286 mmol), [Ni(dtbbpy)(H₂O)₄]Cl₂ (150 mg, 0.286 mmol), and zinc (37.5 mg, 0.573 mmol). The solution was sparged with argon for ˜1 min. The reaction was then stirred at room temperature over three nights. The reaction was filtered and concentrated under reduced pressure. The resulting residue was then dissolved in DCM (3 ml), and trifluoroacetic acid (0.5 ml, 6.49 mmol) was added. The reaction was stirred at room temperature for ˜2 hrs. Then, additional TFA (0.2 ml) was added, and the reaction was stirred for another ˜30 minutes after which stirring was stopped. The reaction was concentrated under reduced pressure. The resulting residue was dissolved in DMSO, filtered, and purified by reverse phase HPLC (MeCN/water w/0.1% TFA) to afford 3-(4-(azetidin-3-yl)benzyl)quinolin-2(1H)-one. ¹H NMR (600 MHz, DMSO) δ 11.78 (s, 1H), 8.89 (s, 1H), 8.56 (s, 1H), 7.71 (s, 1H), 7.57 (d, J=7.6 Hz, 1H), 7.46-7.42 (m, 1H), 7.34-7.31 (m, 4H), 7.28 (d, J=8.2 Hz, 1H), 7.16-7.12 (m, 1H), 4.26-4.21 (m, 2H), 4.12-4.01 (m, 3H), 3.83 (s, 2H). LC/MS (m/z): 291 (M+H)+.

Examples shown in Table 8 below, were prepared according to procedures analogous to those outlined in Example 63 above using the appropriate starting materials, described in the Preparations or Intermediates above, or as obtained from commercial sources.

TABLE 8

| | Structure | LCMS (m/z) |
|---|---|---|
| Example | Name | (M + H)+ |
| 64 | 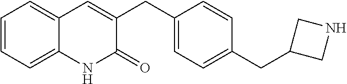 [SMILES: O=c1[nH]c2ccccc2cc1Cc1ccc(CC2CNC2)cc1] | 305 |
| | 3-(4-(azetidin-3-ylmethyl)benzyl)quinolin-2(1H)-one | |

Examples shown in Table 9 below, were prepared according to procedures analogous to those outlined in Example 63 above using the appropriate starting materials, described in the Preparations or Intermediates above, or as obtained from commercial sources. Also, the products were resolved into their single enantiomers by chiral SFC resolution.

TABLE 9

| | | LCMS | |
|---|---|---|---|
| | Structure | (m/z) | |
| Example | IUPAC name | (M + H)+ | SFC conditions |
| 65A (peak 1) and 65B (peak 2) | 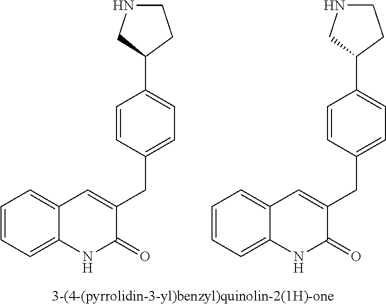 [SMILES: O=c1[nH]c2ccccc2cc1Cc1ccc([C@@H]2CCNC2)cc1 ; O=c1[nH]c2ccccc2cc1Cc1ccc([C@H]2CCNC2)cc1] | 305 | CCA 21 × 250 mm column; 40% MeOH (w/ 0.1% NH₄OH) modifier |

### Example 66: Preparation of N-(2-chloro-4-((2-oxo-1,2-dihydroquinolin-3-yl)methyl)benzyl)acetamide

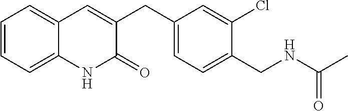 [SMILES: CC(=O)NCc1ccc(Cc2cc3ccccc3[nH]c2=O)cc1Cl]

3-(chloromethyl)-2-methoxyquinoline (42 mg, 0.20 mmol), N-(4-bromo-2-chlorobenzyl)acetamide (79 mg, 0.30 mmol), tetrabutylammonium iodide (18 mg, 0.050 mmol), zinc (26 mg, 0.40 mmol) and [Ni(dtbbpy)(H₂O)₄]Cl₂ (1 mg, 0.02 mmol) were added to a 8 mL vial. DMA (1 mL) was added, and the mixture was purged with argon and stirred at room temperature for 16 h. The mixture was filtered and concentrated. DCM (2 mL) was added, and the mixture was cooled at 0° C. and placed under nitrogen. Boron tribromide (1 M in DCM, 0.90 mL, 0.90 mmol) was added dropwise, and the mixture was allowed to warm to room temperature and stir for 18 hours. Saturated aqueous sodium bicarbonate (1 mL) was added, and the mixture was stirred for 2 minutes. The organic layer was collected with a phase separator and passed through a SAX ion exchange cartridge to remove the tetrabutylammonium iodide. The filtrate was concentrated. The residue was purified reversed phase HPLC (MeCN/water w/0.1% TFA). ¹H NMR (600 MHz, DMSO-d₆) δ 11.82 (s, 1H), 8.31 (t, J=5.5 Hz, 1H), 7.76 (s, 1H), 7.60 (d, J=7.7 Hz, 1H), 7.45 (t, J=7.6 Hz, 1H), 7.37 (s, 1H), 7.29 (d, J=8.2 Hz, 1H), 7.25 (s, 2H), 7.15 (t, J=7.5 Hz, 1H), 4.27 (d, J=5.8 Hz, 2H), 3.82 (s, 2H), 1.88 (s, 3H). LC/MS (m/z): 341 (M+H)+.

Examples shown in Table 10 below, were prepared according to procedures analogous to those outlined in Example 66 above using the appropriate starting materials, described in the Preparations or Intermediates above, or as obtained from commercial sources.

TABLE 10

| | Structure | LCMS (m/z) |
|---|---|---|
| Example | Name | (M + H)+ |
| 67 | 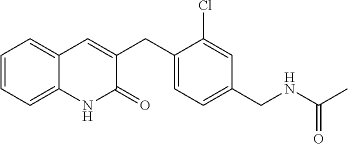 [SMILES: CC(=O)NCc1ccc(Cc2cc3ccccc3[nH]c2=O)c(Cl)c1] | 341 |
| | N-(3-chloro-4-((2-oxo-1,2-dihydroquinolin-3-yl)- | |
| | methyl)benzyl)acetamide | |

### Example 68

### Preparation of N-((3-chloro-5-((2-oxo-1,2-dihydroquinolin-3-yl)methyl)pyridin-2-yl)methyl)acetamide

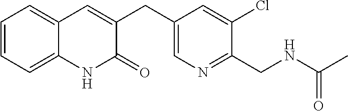 [SMILES: CC(=O)NCc1ncc(Cc2cc3ccccc3[nH]c2=O)cc1Cl]

DMA (3 ml) was added to an argon-purged 8 ml vial containing 3-(chloromethyl)-2-methoxyquinoline (60 mg, 0.289 mmol), N-((5-bromo-3-chloropyridin-2-yl)methyl)acetamide (103 mg, 0.391 mmol), [Ni(dtbbpy)(H₂O)₄]Cl₂ (15.1 mg, 0.0290 mmol), zinc (37.8 mg, 0.578 mmol), and sodium iodide (12 mg, 0.080 mmol). The solution was sparged with argon for ˜1 min. The reaction was then stirred at room temperature for 16 h. The mixture was filtered and concentrated under reduced pressure. The resulting material was suspended in HCl (3 ml, 4 M in dioxane) and concentrated HCl (1.5 ml), and heated at 70° C. for 30 minutes before cooling to room temperature. The sample was then diluted with saturated sodium bicarbonate and 3:1 CHCl₃:IPA. The mixture was passed through a phase separator and the organic layer was concentrated under reduced pressure. The crude material was dissolved in DMSO and purified by reverse phase HPLC (MeCN/water w/0.1% NH₄OH) to afford N-((3-chloro-5-((2-oxo-1,2-dihydroquinolin-3-yl)methyl)pyridin-2-yl)methyl)acetamide. ¹H NMR (600 MHz, DMSO) δ 11.83 (s, 1H), 8.48 (d, J=1.7 Hz, 1H), 8.22 (t, J=5.0 Hz, 1H), 7.85-7.82 (m, 2H), 7.61 (d, J=7.3 Hz, 1H), 7.47-7.43 (m, 1H), 7.29 (d, J=8.2 Hz, 1H), 7.16 (t, J=7.5 Hz, 1H), 4.42 (d, J=5.4 Hz, 2H), 3.86 (s, 2H), 1.86 (s, 3H). LC/MS (m/z): 342 (M+H)+.

Examples shown in Table 11 below, were prepared according to procedures analogous to those outlined in Example 68 above using the appropriate starting materials, described in the Preparations or Intermediates above, or as obtained from commercial sources.

TABLE 11

| | | LCMS |
|---|---|---|
| Ex- | Structure | (m/z) |
| ample | Name | (M + H)+ |
| 69 | 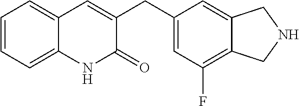 [SMILES: O=c1[nH]c2ccccc2cc1Cc1cc(F)c2c(c1)CNC2] | 295 |
| | 3-((7-fluoroisoindolin-5-yl)methyl)quinolin- | |
| | 2(1H)-one | |
| 70 | 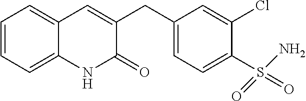 [SMILES: NS(=O)(=O)c1ccc(Cc2cc3ccccc3[nH]c2=O)cc1Cl] | 349 |
| | 2-chloro-4-((2-oxo-1,2-dihydroquinolin-3- | |
| | yl)methyl)benzenesulfonamide | |
| 71 | 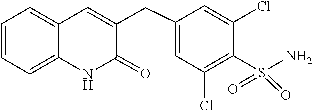 [SMILES: NS(=O)(=O)c1c(Cl)cc(Cc2cc3ccccc3[nH]c2=O)cc1Cl] | 383, 385 |
| | 2,6-dichloro-4-((2-oxo-1,2-dihydroquinolin-3- | |
| | yl)methyl)benzenesulfonamide | |
| 72 | 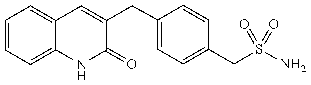 [SMILES: NS(=O)(=O)Cc1ccc(Cc2cc3ccccc3[nH]c2=O)cc1] | 329 |
| | (4-((2-oxo-1,2-dihydroquinolin-3- | |
| | yl)methyl)phenyl)methanesulfonamide | |
| 73 | 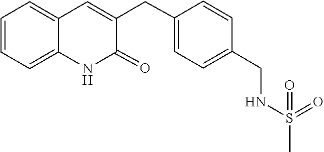 [SMILES: CS(=O)(=O)NCc1ccc(Cc2cc3ccccc3[nH]c2=O)cc1] | 343 |
| | N-(4-((2-oxo-1,2-dihydroquinolin-3- | |
| | yl)methyl)benzyl)methanesulfonamide | |
| 74 | 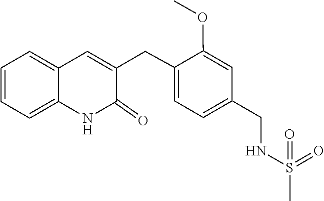 [SMILES: COc1cc(CNS(C)(=O)=O)ccc1Cc1cc2ccccc2[nH]c1=O] | 373 |
| | N-(3-methoxy-4-((2-oxo-1,2-dihydroquinolin- | |
| | 3-yl)methyl)benzyl)methanesulfonamide | |

### Example 75

### Preparation of 3-((3-oxo-2,3-dihydro-1H-inden-5-yl)methyl)quinolin-2(1H)-one

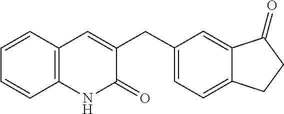 [SMILES: O=C1CCc2ccc(Cc3cc4ccccc4[nH]c3=O)cc21]

### Step A: 6-((2-methoxyquinolin-3-yl)methyl)-2,3-dihydro-1H-inden-1-one

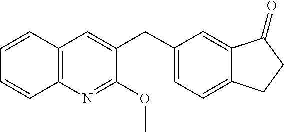 [SMILES: COc1nc2ccccc2cc1Cc1ccc2c(c1)C(=O)CC2]

DMA (4.82 mL) was added to an argon-purged 8 mL vial containing 3-(chloromethyl)-2-methoxyquinoline (100 mg, 0.482 mmol), 6-bromo-2,3-dihydro-1H-inden-1-one (122 mg, 0.578 mmol), [Ni(dtbbpy)(H₂O)₄]Cl₂ (25.2 mg, 0.048 mmol), zinc (63.0 mg, 0.963 mmol) and sodium iodide (28.9 mg, 0.193 mmol). The solution was purged with argon for ˜1 min. The reaction was then stirred at room temperature for 16 h. The crude was filtered, concentrated and taken to the next step without further purification. LC/MS (m/z): 304 (M+H)⁺

### Step B: 3-((3-oxo-2,3-dihydro-1H-inden-5-yl)methyl)quinolin-2(1H)-one

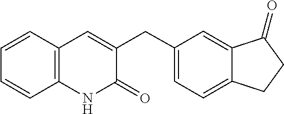 [SMILES: O=C1CCc2ccc(Cc3cc4ccccc4[nH]c3=O)cc21]

Sodium iodide (198 mg, 1.319 mmol) and TMS-Cl (169 μl, 1.319 mmol) at 0° C. was added to a solution of 6-((2-methoxyquinolin-3-yl)methyl)-2,3-dihydro-1H-inden-1-one (100 mg, 0.330 mmol) in MeCN. The reaction was stirred at room temperature for 16 h. The mixture was filtered and concentrated under reduced pressure. The solids were dissolved in DMSO (3 mL). The crude reaction mixture was filtered and purified by reversed phase HPLC (MeCN/water w/0.1% TFA) and lyophilized to afford 3-((3-oxo-2,3-dihydro-1H-inden-5-yl)methyl)quinolin-2(1H)-one. ¹H NMR (600 MHz, DMSO-d₆) δ 11.80 (s, 1H), 7.77 (s, 1H), 7.64 (dd, J=7.8, 1.4 Hz, 1H), 7.59 (d, J=7.7 Hz, 1H), 7.55 (s, 1H), 7.51 (d, J=7.9 Hz, 1H), 7.48-7.40 (m, 1H), 7.29 (d, J=8.2 Hz, 1H), 7.15 (t, J=7.2 Hz, 1H), 3.91 (s, 2H), 3.10-2.99 (m, 2H), 2.62 (dd, J=6.7, 4.9 Hz, 2H). LC/MS (m/z): 290 (M+H)+.

Examples shown in Table 12 below, were prepared according to procedures analogous to those outlined in Example 75 above using the appropriate starting materials, described in the Preparations or Intermediates above, or as obtained from commercial sources.

TABLE 12

| | | LCMS |
|---|---|---|
| Ex- | Structure | (m/z) |
| ample | Name | (M + H)+ |
| 76 | 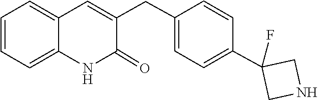 [SMILES: O=c1[nH]c2ccccc2cc1Cc1ccc(C2(F)CNC2)cc1] | 310 |
| | 3-(4-(3-fluoroazetidin-3-yl)benzyl)quinolin- | |
| | 2(1H)-one | |
| 77 | 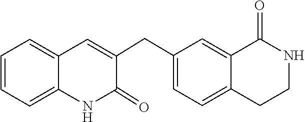 [SMILES: O=C1NCCc2ccc(Cc3cc4ccccc4[nH]c3=O)cc21] | 305 |
| | 3-((1-oxo-1,2,3,4-tetrahydroisoquinolin-7- | |
| | yl)methyl)quinazolin-2(1H)-one | |
| 78 | 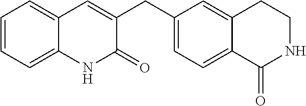 [SMILES: O=C1NCCc2cc(Cc3cc4ccccc4[nH]c3=O)ccc21] | 305 |
| | 3-((1-oxo-1,2,3,4-tetrahydroisoquinolin-6- | |
| | yl)methyl)quinolin-2(1H)-one | |
| 79 | 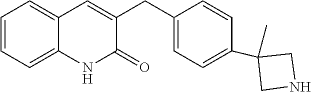 [SMILES: CC1(c2ccc(Cc3cc4ccccc4[nH]c3=O)cc2)CNC1] | 305 |
| | 3-(4-(3-methylazetidin-3-yl)benzyl)quinolin- | |
| | 2(1H)-one | |
| 80 | 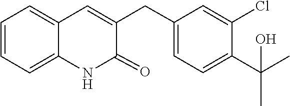 [SMILES: CC(C)(O)c1ccc(Cc2cc3ccccc3[nH]c2=O)cc1Cl] | 328 |
| | 3-(3-chloro-4-(2-hydroxypropan-2- | |
| | yl)benzyl)quinolin-2(1H)-one | |
| 81 | 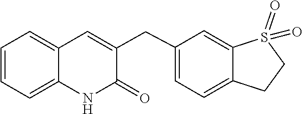 [SMILES: O=c1[nH]c2ccccc2cc1Cc1ccc2c(c1)S(=O)(=O)CC2] | 326 |
| | 3-((1,1-dioxido-2,3-dihydrobenzo[b]thiophen- | |
| | 6-yl)methyl)quinolin-2(1H)-one | |
| 82 | 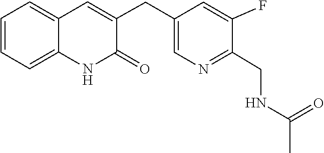 [SMILES: CC(=O)NCc1ncc(Cc2cc3ccccc3[nH]c2=O)cc1F] | 326 |
| | N-((3-fluoro-5-((1-oxo-1,2-dihydroquinolin-3- | |
| | yl)methyl)pyridin-2-yl)methyl)acetamide | |
| 83 | 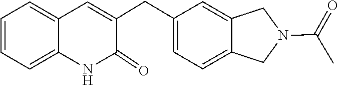 [SMILES: CC(=O)N1Cc2ccc(Cc3cc4ccccc4[nH]c3=O)cc2C1] | 319 |
| | 3-((2-acetylisoindolin-5-yl)methyl)quinolin- | |
| | 2(1H)-one | |
| 84 | 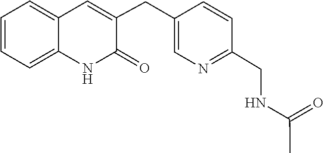 [SMILES: CC(=O)NCc1ccc(Cc2cc3ccccc3[nH]c2=O)cn1] | 308 |
| | N-((5-((2-oxo-1,2-dihydroquinolin-3- | |
| | yl)methyl)pyridin-2-yl)methyl)acetamide | |
| 85 | 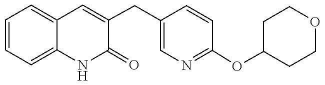 [SMILES: O=c1[nH]c2ccccc2cc1Cc1ccc(OC2CCOCC2)nc1] | 337 |
| | 3-((6-((tetrahydro-2H-pyran-4-yl)oxy)pyridin- | |
| | 3-yl)methyl)quinolin-2(1H)-one | |
| 86 | 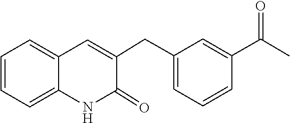 [SMILES: CC(=O)c1cccc(Cc2cc3ccccc3[nH]c2=O)c1] | 278 |
| | 3-(3-acetylbenzyl)quinolin-2(1H)-one | |
| 87 | 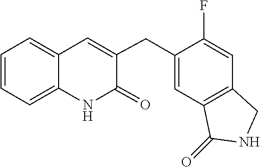 [SMILES: O=C1NCc2cc(F)c(Cc3cc4ccccc4[nH]c3=O)cc21] | 309 |
| | 3-((6-fluoro-3-ixoisoindolin-5- | |
| | yl)methyl)quinolin-2(1H)-one | |
| 88 | 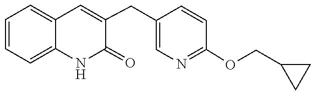 [SMILES: O=c1[nH]c2ccccc2cc1Cc1ccc(OCC2CC2)nc1] | 307 |
| | 3-((6-(cyclopropylmethoxy)pyridin-3- | |
| | yl)methyl)quinolin-2(1H)-one | |
| 89 | 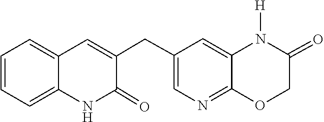 [SMILES: O=C1COc2ncc(Cc3cc4ccccc4[nH]c3=O)cc2N1] | 308 |
| | 7-((2-oxo-1,2-dihydroquinolin-3-yl)methyl)- | |
| | 1H-pyrido[2,3-b][1,4]oxazin-2(3H)-one | |

Examples shown in Table 13 below, were prepared according to procedures analogous to those outlined in Example 75 above using the appropriate starting materials, described in the Preparations or Intermediates above, or as obtained from commercial sources. Also, the products were resolved into their single enantiomers by chiral SFC resolution.

TABLE 13

| | | | LCMS | |
|---|---|---|---|---|
| | Structure | | (m/z) | |
| Example | Name | | (M + H )+ | SFC conditions |
| 90A (peak 1) and 90B (peak 2) | 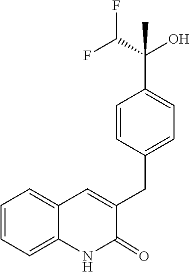 [SMILES: C[C@@](O)(c1ccc(Cc2cc3ccccc3[nH]c2=O)cc1)C(F)F] | 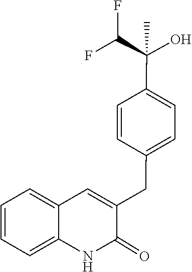 [SMILES: C[C@](O)(c1ccc(Cc2cc3ccccc3[nH]c2=O)cc1)C(F)F] | 330 | Lux-4 21 × 250 mm column; 35% MeOH (w/ 0.1% NH₄OH) modifier |

3-(4-(1,1-difluoro-2-hydroxypropan-2-
yl)benzyl)quinolin-2(1H)-one

### Example 91

### Preparation of N-((3-chloro-5-((2-oxo-1,2-dihydroquinolin-3-yl)methyl)pyridin-2-yl)methyl)methanesulfonamide

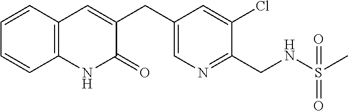 [SMILES: CS(=O)(=O)NCc1ncc(Cc2cc3ccccc3[nH]c2=O)cc1Cl]

### Step A: tert-butyl ((3-chloro-5-((2-methoxyquinolin-3-yl)methyl)pyridin-2-yl)methyl)carbamate

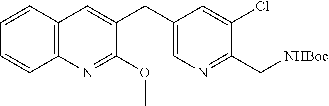 [SMILES: COc1nc2ccccc2cc1Cc1cnc(CNC(=O)OC(C)(C)C)c(Cl)c1]

DMA (4.82 mL) was added to an argon-purged 8 mL vial containing 3-(chloromethyl)-2-methoxyquinoline (100 mg, 0.482 mmol), tert-butyl ((5-bromo-3-chloropyridin-2-yl)methyl)carbamate (201 mg, 0.626 mmol), [Ni(dtbbpy)(H₂O)₄]Cl₂ (252 mg, 0.482 mmol), zinc (63.0 mg, 0.963 mmol) and sodium iodide (18.05 mg, 0.120 mmol). The solution was sparged with argon for ˜1 min. The reaction was then stirred at room temperature for 16 h. The mixture was then filtered, concentrated and used as is in the next step. LC/MS (m/z): 414 (M+H)+.

### Step B: 3-((6-(aminomethyl)-5-chloropyridin-3-yl)methyl)quinolin-2(1H)-one

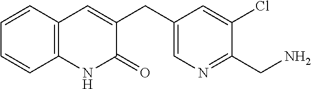 [SMILES: NCc1ncc(Cc2cc3ccccc3[nH]c2=O)cc1Cl]

Sodium iodide (72.4 mg, 0.483 mmol) was added to a 20 mL vial containing tert-butyl ((3-chloro-5-((2-methoxyquinolin-3-yl)methyl)pyridin-2-yl)methyl)carbamate (50 mg, 0.121 mmol), followed by MeCN (1.4 mL). Then, TMS-Cl (61.3 μL, 0.483 mmol) was added to the solution. The mixture was stirred at room temperature for 16 h. The sample was then transferred to a round bottom flask with MeOH and concentrated. The crude material was taken to next step without further purification. LC/MS (m/z): 300 (M+H)+.

### Step C: N-((3-chloro-5-((2-oxo-1,2-dihydroquinolin-3-yl)methyl)pyridin-2-yl)methyl)methanesulfonamide

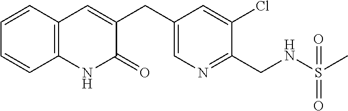 [SMILES: CS(=O)(=O)NCc1ncc(Cc2cc3ccccc3[nH]c2=O)cc1Cl]

Triethylamine (24 μl, 0.17 mmol) at 0° C. was added to a solution of 3-((6-(aminomethyl)-5-chloropyridin-3-yl)methyl)quinolin-2(1H)-one (26 mg, 0.087 mmol) in DCM (1 mL). The mixture was stirred at room temperature for 30 min. The mixture was diluted with DCM (5 mL), and then saturated aqueous solution of NH₄Cl (5 mL) was added. The organic layer was separated and collected. The aqueous layer was further washed with DCM (5 mL). The combined organic layer was dried by passing through phase separator and concentrated under reduced pressure. The crude was purified by flash column chromatography using (10-50%) 3:1 EtOAc:EtOH in hexane. ¹H NMR (600 MHz, DMSO-d₆) δ 11.85 (s, 1H), 8.52 (s, 1H), 7.88 (s, 1H), 7.85 (s, 1H), 7.62 (d, J=7.4 Hz, 1H), 7.49-7.45 (m, 1H), 7.43 (s, 1H), 7.30 (d, J=8.2 Hz, 1H), 7.23-7.12 (m, 1H), 4.37 (s, 2H), 3.87 (s, 2H), 2.94 (s, 3H). LC/MS (m/z): 378 (M+H)+.

### Example 92

### Preparation of 5-((2-oxo-1,2-dihydroquinolin-3-yl)methyl)pyrazolo[1,5-a]pyridine-3-carbonitrile

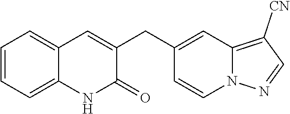 [SMILES: N#Cc1cnn2ccc(Cc3cc4ccccc4[nH]c3=O)cc12]

### Step A: 5-((2-methoxyquinolin-3-yl)methyl)pyrazolo[1,5-a]pyridine-3-carbonitrile

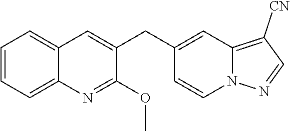 [SMILES: COc1nc2ccccc2cc1Cc1ccn2ncc(C#N)c2c1]

3-(chloromethyl)-2-methoxyquinoline (30 mg, 0.144 mmol), NiCl₂-DME (15.87 mg, 0.072 mmol), picolinimidamide hydrochloride (11.38 mg, 0.072 mmol), zinc (18.89 mg, 0.289 mmol), tetrabutylammonium iodide (80 mg, 0.217 mmol), 5-bromopyrazolo[1,5-a]pyridine-3-carbonitrile (41.7 mg, 0.188 mmol), and DMA (1.5 ml) were added to a 20 mL vial. The mixture was degassed with nitrogen for 1 min. The mixture was stirred for 1 hour. The mixture was poured into a 100 mL flask containing diethyl ether (20 mL), and Celite (5 grams), and the resulting reaction mixture was stirred for 5 minutes. The mixture was filtered through Celite. The filtrate was washed with water (2×20 mL), and the organic layer was dried over magnesium sulfate, filtered, and the solvents were evaporated to afford crude 5-((2-methoxyquinolin-3-yl)methyl)pyrazolo[1,5-a]pyridine-3-carbonitrile, which was taken forward without further purification. LC/MS (m/z): 315 (M+H)+.

### Step B: 5-((2-oxo-1,2-dihydroquinolin-3-yl)methyl)pyrazolo[1,5-a]pyridine-3-carbonitrile

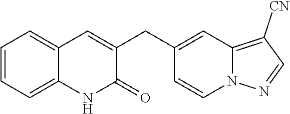 [SMILES: N#Cc1cnn2ccc(Cc3cc4ccccc4[nH]c3=O)cc12]

5-((2-methoxyquinolin-3-yl)methyl)pyrazolo[1,5-a]pyridine-3-carbonitrile (45.4 mg, 0.144 mmol), sodium iodide (43.3 mg, 0.289 mmol), and MeCN (1 ml) were added to a 20 mL vial. The mixture was stirred. TMS-Cl (0.0740 ml, 0.578 mmol) was added to the mixture. The mixture was stirred for 3 hours. The mixture was quenched with saturated aqueous sodium bicarbonate (3 mL) and extracted with DCM (10 mL), using a phase separator. The solvents of the organic layer were evaporated, and the residue was purified silica gel chromatography with 0-70% EtOAc:EtOH (3:1) in hexanes as eluent to afford 5-((2-oxo-1,2-dihydroquinolin-3-yl)methyl)pyrazolo[1,5-a]pyridine-3-carbonitrile. ¹H NMR (499 MHz, DMSO-d₆) δ 11.88 (s, 1H), 8.86 (d, J=7.1 Hz, 1H), 8.60 (s, 1H), 7.88 (s, 1H), 7.81 (s, 1H), 7.62 (d, J=7.7 Hz, 1H), 7.51-7.44 (m, 1H), 7.31 (d, J=8.2 Hz, 1H), 7.22-7.13 (m, 2H), 4.01 (s, 2H). LC/MS (m/z): 301 (M+H)+.

Examples shown in Table 14 below, were prepared according to procedures analogous to those outlined in Example 92 above using the appropriate starting materials, described in the Preparations or Intermediates above, or as obtained from commercial sources. Table 14

| | | LCMS |
|---|---|---|
| Ex- | Structure | (m/z) |
| ample | Name | (M + H)+ |
| 93 | 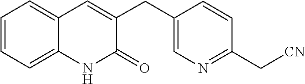 [SMILES: N#CCc1ccc(Cc2cc3ccccc3[nH]c2=O)cn1] | 278 |
| | 2-(5-((2-oxo-1,2-dihydroquinolin-3- | |
| | yl)methyl)pyridin-2-yl)acetonitrile | |
| 94 | 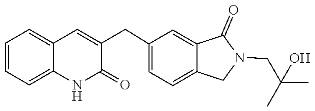 [SMILES: CC(C)(O)CN1Cc2ccc(Cc3cc4ccccc4[nH]c3=O)cc2C1=O] | 363 |
| | 3-((2-(2-hydroxy-2-methylpropyl)-3- | |
| | oxoisoindolin-5-yl)methyl)quinolin-2(1H)-one | |
| 95 | 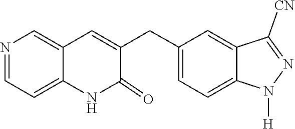 [SMILES: N#Cc1n[nH]c2ccc(Cc3cc4cnccc4[nH]c3=O)cc12] | 302 |
| | 5-((2-oxo-1,2-dihydro-1,6-naphthyridin-3- | |
| | yl)methyl)-1H-indazole-3-carbonitrile | |
| 96 | 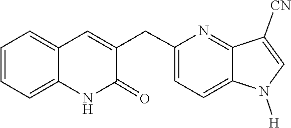 [SMILES: N#Cc1c[nH]c2ccc(Cc3cc4ccccc4[nH]c3=O)nc12] | 301 |
| | 5-((2-oxo-1,2-dihydroquinolin-3-yl)methyl)- | |
| | 1H-pyrrolo[3,2-b]pyridine-3-carbonitrile | |
| 97 | 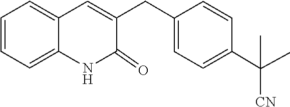 [SMILES: CC(C)(C#N)c1ccc(Cc2cc3ccccc3[nH]c2=O)cc1] | 303 |
| | 2-methyl-2-(4-((2-oxo-1,2-dihydroquinolin-3- | |
| | yl)methyl)phenyl)propanenitrile | |
| 98 | 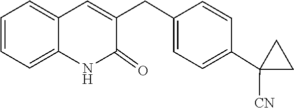 [SMILES: N#CC1(c2ccc(Cc3cc4ccccc4[nH]c3=O)cc2)CC1] | 301 |
| | 1-(4-((2-oxo-1,2-dihydroquinolin-3- | |
| | yl)methyl)phenyl)cyclopropane-1-carbonitrile | |
| 99 | 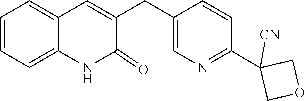 [SMILES: N#CC1(c2ccc(Cc3cc4ccccc4[nH]c3=O)cn2)COC1] | 318 |
| | 3-(5-((2-oxo-1,2-dihydroquinolin-3- | |
| | yl)methyl)pyridin-2-yl)oxetane-3-carbonitrile | |
| 100 | 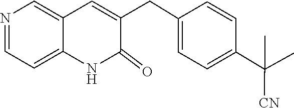 [SMILES: CC(C)(C#N)c1ccc(Cc2cc3cnccc3[nH]c2=O)cc1] | 304 |
| | 2-methyl-2-(4-((2-oxo-1,2-dihydro-1,6- | |
| | naphthyridin-3-yl)methyl)phenyl)propanenitrile | |

### Example 101A and 101B

### Preparation of two isomers of 3-hydroxy-1-(4-((2-oxo-1,2-dihydroquinolin-3-yl)methyl)phenyl)cyclobutane-1-carbonitrile

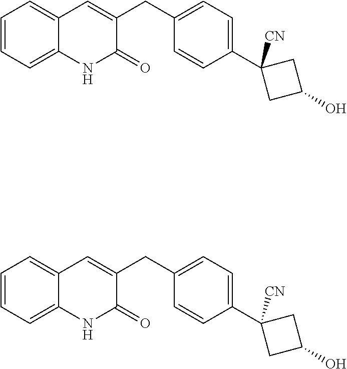 [SMILES: N#C[C@]1(c2ccc(Cc3cc4ccccc4[nH]c3=O)cc2)C[C@@H](O)C1 ; N#C[C@]1(c2ccc(Cc3cc4ccccc4[nH]c3=O)cc2)C[C@H](O)C1]

### Step A: 1-(4-((2-methoxyquinolin-3-yl)methyl)phenyl)-3-oxocyclobutane-1-carbonitrile

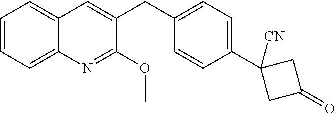 [SMILES: COc1nc2ccccc2cc1Cc1ccc(C2(C#N)CC(=O)C2)cc1]

3-(chloromethyl)-2-methoxyquinoline (100 mg, 0.482 mmol), NiCl₂-DME (52.9 mg, 0.241 mmol), picolinimidamide hydrochloride (37.9 mg, 0.241 mmol), zinc (63.0 mg, 0.963 mmol), tetrabutylammonium iodide (267 mg, 0.722 mmol), 1-(4-bromophenyl)-3-oxocyclobutane-1-carbonitrile (157 mg, 0.626 mmol), and DMA (3 ml) were added to a 20 mL vial. The mixture was degassed with nitrogen for 1 minute. The mixture was stirred for 1 hour. The mixture was poured into a 100 mL flask containing diethyl ether (20 mL) and Celite (10 grams), and the mixture was stirred for 5 minutes. The mixture was filtered through Celite. The filtrate was washed with water (2×20 mL), and the organic layer was dried over magnesium sulfate, filtered, and the solvents were evaporated. The residue was purified by silica gel chromatography with 0-50% EtOAc:EtOH (3:1) in hexanes as eluent to afford 1-(4-((2-methoxyquinolin-3-yl)methyl)phenyl)-3-oxocyclobutane-1-carbonitrile. LC/MS (m/z): 343 (M+H)+.

### Step B: 3-oxo-1-(4-((2-oxo-1,2-dihydroquinolin-3-yl)methyl)phenyl)cyclobutane-1-carbonitrile

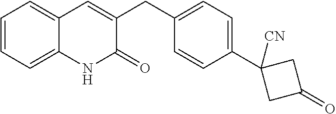 [SMILES: N#CC1(c2ccc(Cc3cc4ccccc4[nH]c3=O)cc2)CC(=O)C1]

Sodium iodide (75 mg, 0.502 mmol), 1-(4-((2-methoxyquinolin-3-yl)methyl)phenyl)-3-oxocyclobutane-1-carbonitrile (86 mg, 0.251 mmol), and MeCN (2 ml) were added to a 20 mL vial. The mixture was stirred. TMS-Cl (0.128 ml, 1.005 mmol) was added to the mixture. The mixture was stirred for 2 hours. The mixture was quenched with saturated aqueous sodium bicarbonate (3 mL) and extracted with DCM (10 mL), using a phase separator. The solvents of the organic layer were evaporated to afford crude 3-oxo-1-(4-((2-oxo-1,2-dihydroquinolin-3-yl)methyl)phenyl) cyclobutane-1-carbonitrile, which was used without further purification. LC/MS (m/z): 329 (M+H)+.

### Step C: Two isomers of 3-hydroxy-1-(4-((2-oxo-1,2-dihydroquinolin-3-yl)methyl)phenyl)cyclobutane-1-carbonitrile

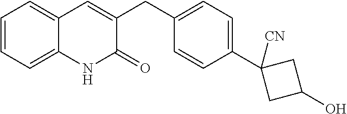 [SMILES: N#CC1(c2ccc(Cc3cc4ccccc4[nH]c3=O)cc2)CC(O)C1]

3-oxo-1-(4-((2-oxo-1,2-dihydroquinolin-3-yl)methyl)phenyl)cyclobutane-1-carbonitrile (82 mg, 0.250 mmol) and MeOH (3 ml) were added. Sodium borohydride (37.8 mg, 0.999 mmol) was added to the mixture. The mixture was stirred for 30 minutes. The mixture was quenched with saturated ammonium chloride (3 mL), and the mixture was extracted with DCM (2×5 mL). The organic layer was collected with a phase separator. The solvents were evaporated, and the residue was purified by silica gel chromatography with 0-80% EtOAc:EtOH (3:1) in hexanes as eluent to afford a ˜2:1 mixture of a diastereomers. The mixture was resolved by SFC (OJ-H 21×250 mm column with 25% MeOH (w/0.1% NH₄OH) as modifier) to afford an isomer of 3-hydroxy-1-(4-((2-oxo-1,2-dihydroquinolin-3-yl)methyl)phenyl)cyclobutane-1-carbonitrile Example 101A (first eluting): ¹H NMR (499 MHz, DMSO-d₆) δ 11.80 (s, 1H), 7.74 (s, 1H), 7.60 (d, J=7.4 Hz, 1H), 7.48-7.41 (m, 1H), 7.36 (s, 4H), 7.29 (d, J=8.2 Hz, 1H), 7.15 (t, J=7.5 Hz, 1H), 5.57 (d, J=7.1 Hz, 1H), 4.40 (h, J=7.1 Hz, 1H), 3.85 (s, 2H), 3.04 (ddd, J=9.5, 7.0, 2.8 Hz, 2H), 2.39-2.34 (m, 2H)) LC/MS (m/z): 331 (M+H)+ and a second isomer of 3-hydroxy-1-(4-((2-oxo-1,2-dihydroquinolin-3-yl)methyl)phenyl)cyclobutane-1-carbonitrile Example 101B (second eluting): ¹H NMR (499 MHz, DMSO-d₆) δ 11.80 (s, 1H), 7.77 (s, 1H), 7.60 (d, J=7.7 Hz, 1H), 7.44 (dd, J=16.9, 7.8 Hz, 3H), 7.37 (d, J=8.4 Hz, 2H), 7.29 (d, J=8.1 Hz, 1H), 7.15 (t, J=7.6 Hz, 1H), 5.57 (d, J=6.6 Hz, 1H), 4.18 (q, J=7.0 Hz, 1H), 3.85 (s, 2H), 2.92 (ddd, J=9.8, 7.1, 2.5 Hz, 2H), 2.70-2.65 (m, 2H). LC/MS (m/z): 331 (M+H)+.

### Example 102A, 102B, 102C and 102D

### Preparation of four isomers of 3-hydroxy-2-(4-((2-oxo-1,2-dihydroquinolin-3-yl)methyl)phenyl)butanenitrile

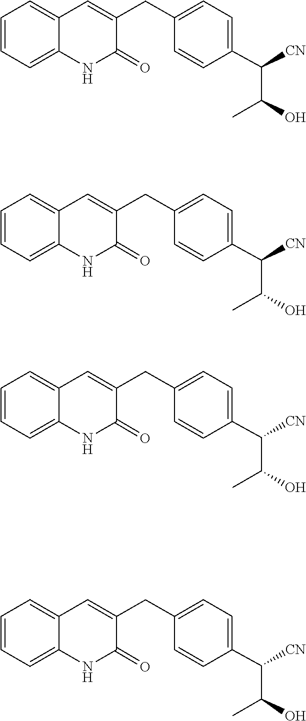 [SMILES: C[C@H](O)[C@H](C#N)c1ccc(Cc2cc3ccccc3[nH]c2=O)cc1 ; C[C@@H](O)[C@H](C#N)c1ccc(Cc2cc3ccccc3[nH]c2=O)cc1 ; C[C@@H](O)[C@@H](C#N)c1ccc(Cc2cc3ccccc3[nH]c2=O)cc1 ; C[C@H](O)[C@@H](C#N)c1ccc(Cc2cc3ccccc3[nH]c2=O)cc1]

### Step A: 2-(4-((2-methoxyquinolin-3-yl)methyl)phenyl)-3-oxobutanenitrile

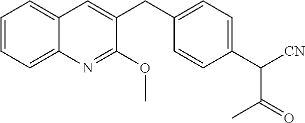 [SMILES: COc1nc2ccccc2cc1Cc1ccc(C(C#N)C(C)=O)cc1]

3-(chloromethyl)-2-methoxyquinoline (500 mg, 2.408 mmol), NiCl₂-DME (185 mg, 0.843 mmol), picolinimidamide hydrochloride (133 mg, 0.843 mmol), zinc (315 mg, 4.82 mmol), tetrabutylammonium iodide (1334 mg, 3.61 mmol), 2-(4-bromophenyl)-3-oxobutanenitrile (745 mg, 3.13 mmol), and DMA (8 ml) were added to a 20 mL vial. The mixture was degassed with nitrogen for 1 minute. The mixture was stirred for 1 h. The mixture was poured into a 250 mL flask containing diethyl ether (40 mL) and Celite (20 g), and the mixture was stirred for 5 minutes. The mixture was filtered through Celite. The filtrate was washed with water (2×40 mL), and the organic layer was dried over magnesium sulfate, filtered, and the solvents were evaporated to afford 2-(4-((2-methoxyquinolin-3-yl)methyl)phenyl)-3-oxobutanenitrile, which was taken forward without further purification/MS (m/z): 331 (M+H)+.

### Step B: 3-hydroxy-2-(4-((2-methoxyquinolin-3-yl)methyl)phenyl)butanenitrile

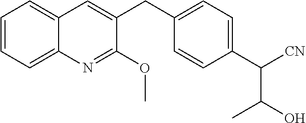 [SMILES: COc1nc2ccccc2cc1Cc1ccc(C(C#N)C(C)O)cc1]

2-(4-((2-methoxyquinolin-3-yl)methyl)phenyl)-3-oxobutanenitrile (386 mg, 1.17 mmol) and MeOH (4 ml) were added to a 20 mL vial and stirred. Sodium borohydride (442 mg, 11.7 mmol) was added to the stirring mixture over 30 minutes in 5 portions. Saturated aqueous ammonium chloride (2 mL) was added to the mixture. The mixture was extracted with DCM (2×10 mL). The organic layer was dried over sodium sulfate, filtered, and the solvents were evaporated to afford 3-hydroxy-2-(4-((2-methoxyquinolin-3-yl)methyl)phenyl)butanenitrile, which was taken forward without further purification. LC/MS (m/z): 333 (M+H)+.

### Step C: Four isomers of 3-hydroxy-2-(4-((2-methoxyquinolin-3-yl)methyl)phenyl)butanenitrile

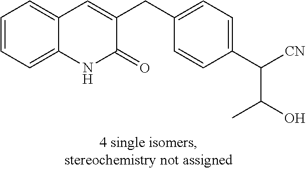

3-hydroxy-2-(4-((2-methoxyquinolin-3-yl)methyl)phenyl)butanenitrile (390 mg, 1.173 mmol), sodium iodide (352 mg, 2.347 mmol), and MeCN (4 ml) were added to a 20 mL vial. The mixture was stirred at room temperature. Saturated sodium bicarbonate (5 mL) was added to the mixture, and the mixture was extracted with DCM (3×10 mL). The organic layer was dried over sodium sulfate, filtered, and the solvents of the filtrate were evaporated. The residue was purified by silica gel chromatography with 0-10% MeOH in DCM as eluent to afford a mixture of isomers. The mixture was resolved by chiral SFC (AS-H 21×250 mm column with 20% MeOH (w/0.1% NH₄OH) as modifier) to afford a mixture of 2 isomers (first eluting) an isomer of 3-hydroxy-2-(4-((2-methoxyquinolin-3-yl)methyl)phenyl)butanenitrile Example 102C (second eluting): ¹H NMR (499 MHz, DMSO-d₆) δ 11.79 (s, 1H), 7.72 (s, 1H), 7.59 (d, J=8.0 Hz, 1H), 7.44 (t, J=7.7 Hz, 1H), 7.31 (q, J=8.1 Hz, 5H), 7.15 (t, J=7.2 Hz, 1H), 5.36 (d, J=5.3 Hz, 1H), 4.08 (d, J=5.3 Hz, 1H), 3.90 (q, J=5.6 Hz, 1H), 3.84 (s, 2H), 1.12 (d, J=6.1 Hz, 3H) LC/MS (m/z): 319 (M+H)+ and a second isomer of 3-hydroxy-2-(4-((2-methoxyquinolin-3-yl)methyl)phenyl)butanenitrile Example 102D (third eluting): ¹H NMR (499 MHz, DMSO-d₆) δ 11.79 (s, 1H), 7.72 (s, 1H), 7.59 (d, J=7.9 Hz, 1H), 7.44 (t, J=7.1 Hz, 1H), 7.31 (q, J=8.2 Hz, 5H), 7.15 (t, J=7.5 Hz, 1H), 5.36 (d, J=5.3 Hz, 1H), 4.08 (d, J=5.3 Hz, 1H), 3.90 (q, J=5.5 Hz, 1H), 3.84 (s, 2H), 1.12 (d, J=6.2 Hz, 3H). LC/MS (m/z): 319 (M+H)+. The mixture of 2 isomers obtained during the first SFC resolution was further resolved by chiral SFC (CCAF4 21×250 mm column with 35% MeOH (w/0.1% NH₄OH) as modifier) to afford a third isomer of 3-hydroxy-2-(4-((2-methoxyquinolin-3-yl)methyl)phenyl)butanenitrile Example 102A (first eluting): ¹H NMR (499 MHz, DMSO-d₆) δ 11.79 (s, 1H), 7.72 (s, 1H), 7.59 (d, J=7.6 Hz, 1H), 7.45 (t, J=7.1 Hz, 1H), 7.33 (d, J=8.2 Hz, 2H), 7.28 (dd, J=12.3, 8.1 Hz, 3H), 7.15 (t, J=7.5 Hz, 1H), 5.41 (d, J=4.6 Hz, 1H), 4.21 (d, J=5.1 Hz, 1H), 4.01-3.91 (m, 1H), 3.84 (s, 2H), 1.05 (d, J=6.2 Hz, 3H). LC/MS (m/z): 319 (M+H)+ and a fourth isomer of 3-hydroxy-2-(4-((2-methoxyquinolin-3-yl)methyl)phenyl)butanenitrile Example 102B (second eluting): ¹H NMR (499 MHz, DMSO-d₆) δ 11.79 (s, 1H), 7.72 (s, 1H), 7.59 (d, J=7.7 Hz, 1H), 7.45 (t, J=7.7 Hz, 1H), 7.33 (d, J=8.1 Hz, 2H), 7.28 (dd, J=12.2, 8.2 Hz, 3H), 7.15 (t, J=7.0 Hz, 1H), 5.41 (d, J=4.6 Hz, 1H), 4.21 (d, J=5.1 Hz, 1H), 4.03-3.91 (m, 1H), 3.84 (s, 2H), 1.05 (d, J=6.2 Hz, 3H). LC/MS (m/z): 319 (M+H)+.

### Example 103

### Preparation of 3-(3-(trifluoromethoxy)benzyl)-1,7-naphthyridin-2(1H)-one

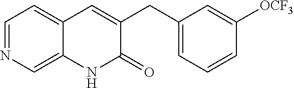 [SMILES: O=c1[nH]c2cnccc2cc1Cc1cccc(OC(F)(F)F)c1]

DMA (0.5 ml) was added to a vial containing 3-bromo-1,7-naphthyridin-2(1H)-one (45.0 mg, 0.2 mmol) and Xphos-Pd G3 (33.9 mg, 0.0400 mmol). (3-(trifluoromethoxy)benzyl)zinc(II) bromide 0.5 M in THF (1.60 ml, 0.800 mmol) was then added, and the mixture was heated at 75° C. for 3 h. The mixture was then diluted with MeCN and a small amount of MP-TMT (200 mg, 0.36 mmol/g) was added, and the reaction was heated again at 75° C. for 20 minutes, filtered, washed with MeCN/MeOH, and concentrated. The residue was then purified by reversed phase HPLC (MeCN/water w/0.1% TFA) to afford 3-(3-(trifluoromethoxy)benzyl)-1,7-naphthyridin-one. ¹H NMR (600 MHz, DMSO-d₆) δ 12.19 (s, 1H), 8.65 (s, 1H), 8.34 (d, J=5.3 Hz, 1H), 7.83 (s, 1H), 7.67 (d, J=5.3 Hz, 1H), 7.45 (t, J=7.9 Hz, 1H), 7.37-7.29 (m, 2H), 7.23 (d, J=8.1 Hz, 1H), 3.95 (s, 2H). LC/MS (m/z): 321 (M+H)+.

Examples shown in Table 15 below, were prepared according to procedures analogous to those outlined in Example 103 above using the appropriate starting materials, described in the Preparations or Intermediates above, or as obtained from commercial sources.

TABLE 15

| | Structure | LCMS (m/z) |
|---|---|---|
| Example | Name | (M + H)+ |
| 104 | 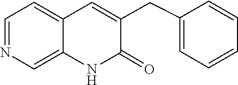 [SMILES: O=c1[nH]c2cnccc2cc1Cc1ccccc1] | 237 |
| | 3-benzyl-1,7-naphthyridin-2(1H)-one | |
| 105 | 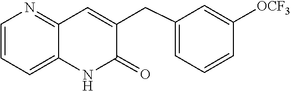 [SMILES: O=c1[nH]c2cccnc2cc1Cc1cccc(OC(F)(F)F)c1] | 321 |
| | 3-(3-(trifluoromethoxy)benzyl)-1,5- | |
| | naphthyridin-2(1H)-one | |
| 106 | 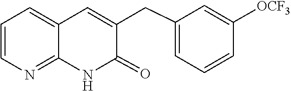 [SMILES: O=c1[nH]c2ncccc2cc1Cc1cccc(OC(F)(F)F)c1] | 321 |
| | 3-(3-(trifluoromethoxy)benzyl)-1,8- | |
| | naphthyridin-2(1H)-one | |

### Example 107

### Preparation of 4-(4-((2-oxo-1,2-dihydroquinolin-3-yl)methyl)phenyl)tetrahydro-2H-pyran-4-carbonitrile

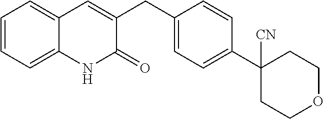 [SMILES: N#CC1(c2ccc(Cc3cc4ccccc4[nH]c3=O)cc2)CCOCC1]

3-(chloromethyl)-2-methoxyquinoline (26 mg, 0.13 mmol), 4-(4-(4,4,5,5-tetramethyl-1,3,2-dioxaborolan-2-yl)phenyl)tetrahydro-2H-pyran-4-carbonitrile (63 mg, 0.20 mmol), Pd(dppf)Cl₂ (9.2 mg, 0.013 mmol) and cesium fluoride (76 mg, 0.50 mmol) were added to a 8 mL vial. Then 10:1 dioxane:water (2 mL) was added under nitrogen. The mixture was stirred and heated at 80° C. for 15 hours. The mixture was filtered and concentrated. To the residue was added MeCN, TMS-Cl (55 mg, 0.50 mmol), and sodium iodide (75 mg, 0.50 mmol). The mixture was stirred for 3 hours, filtered and purified by reversed phase HPLC (MeCN/water w/0.1% NH₄OH). ¹H NMR (600 MHz, DMSO-d₆) δ 11.79 (s, 1H), 7.77 (s, 1H), 7.60 (d, J=7.8 Hz, 1H), 7.48-7.43 (m, 3H), 7.38 (d, J=8.3 Hz, 2H), 7.30 (d, J=8.2 Hz, 1H), 7.15 (t, J=7.5 Hz, 1H), 4.03-3.98 (m, 2H), 3.86 (s, 2H), 3.69-3.61 (m, 2H), 2.09-2.01 (m, 4H). LC/MS (m/z): 345 (M+H)+.

### Example 108

### Preparation of 3-(5-((2-oxo-1,2-dihydroquinolin-3-yl)methyl)pyridin-2-yl)oxazolidin-2-one

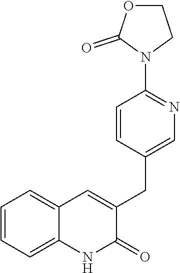 [SMILES: O=C1OCCN1c1ccc(Cc2cc3ccccc3[nH]c2=O)cn1]

### Step A: 3-(5-((2-methoxyquinolin-3-yl)methyl)pyridin-2-yl)oxazolidin-2-one

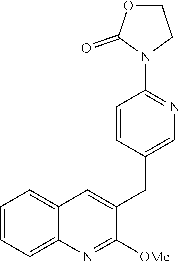 [SMILES: COc1nc2ccccc2cc1Cc1ccc(N2CCOC2=O)nc1]

3-(5-(4,4,5,5-tetramethyl-1,3,2-dioxaborolan-2-yl)pyridin-2-yl)oxazolidin-2-one (58.0 mg, 0.200 mmol), 3-(chloromethyl)-2-methoxyquinoline (35.0 mg, 0.169 mmol), bis(triphenylphosphine) (II) dichloride (11.8 mg, 0.0170 mmol), and potassium fluoride (29.4 mg, 0.506 mmol) were added to a 2 mL microwave vial. The vial was purged with argon followed by addition of MeOH (1.1 mL). The mixture was stirred and heated at 120° C. in a microwave reactor for 5 min. The mixture was diluted with water and extracted with DCM. The organic layer was passed through 500 mg of thiol Si plug, and the solution was concentrated and used for next step. LC/MS (m/z): 336 (M+H)+

### Step B: 3-(5-((2-oxo-1,2-dihydroquinolin-3-yl)methyl)pyridin-2-yl)oxazolidin-2-one

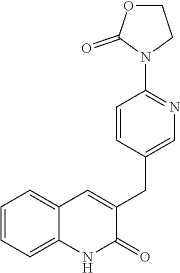 [SMILES: O=C1OCCN1c1ccc(Cc2cc3ccccc3[nH]c2=O)cn1]

NaI (102 mg, 0.680 mmol) and TMSCl (0.090 mL, 0.68 mmol) in 1 mL of MeCN were added to the crude reaction mixture from Step A. The mixture was stirred at room temperature for 16 h. The mixture was filtered and purified by reversed phase HPLC (MeCN/water w/0.10% TFA). ¹H NMR (500 MHz, DMSO-d₆) δ 11.82 (s, 1H), 8.32 (d, J=1.9 Hz, 1H), 7.98 (d, J=8.6 Hz, 1H), 7.80-7.73 (m, 2H), 7.60 (d, J=7.6 Hz, 1H), 7.51-7.39 (m, 1H), 7.30 (d, J=8.2 Hz, 1H), 7.16 (t, J=7.5 Hz, 1H), 4.44 (t, J=8.1 Hz, 3H), 4.14 (t, J=8.1 Hz, 3H), 3.81 (s, 3H). LC/MS (m/z): 322 (M+H)⁺

Examples shown in Table 16 below, were prepared according to procedures analogous to those outlined in Example 108 above using the appropriate starting materials, described in the Preparations or Intermediates above, or as obtained from commercial sources.

TABLE 16

| | Structure | LCMS (m/z) |
|---|---|---|
| Example | Name | (M + H)+ |
| 109 | 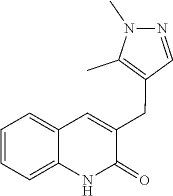 [SMILES: Cc1c(Cc2cc3ccccc3[nH]c2=O)cnn1C] | 254 |
| | 3-((1,5-dimethyl-1H-pyrazol-4- | |
| | yl)methyl)quinolin-2(1H)-one | |
| 110 | 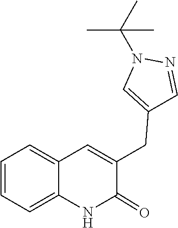 [SMILES: CC(C)(C)n1cc(Cc2cc3ccccc3[nH]c2=O)cn1] | 282 |
| | 3-((1-(tert-butyl)-1H-pyrazol-4- | |
| | yl)methyl)quinolin-2(1H)-one | |
| 111 | 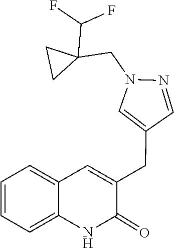 [SMILES: O=c1[nH]c2ccccc2cc1Cc1cnn(CC2(C(F)F)CC2)c1] | 330 |
| | 3-((1-((1-(difluoromethyl)cyclopropyl)methyl)- | |
| | 1H-pyrazol-4-yl)methyl)quinolin-2(1H)-one | |
| 112 | 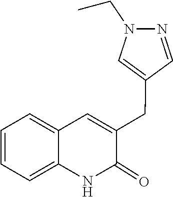 [SMILES: CCn1cc(Cc2cc3ccccc3[nH]c2=O)cn1] | 254 |
| | 3-((1-ethyl-1H-pyrazol-4-yl)methyl)quinolin- | |
| | 2(1H)-one | |

### Example 113

### Preparation of 3-(4-(1-hydroxycyclobutyl)benzyl)-1,6-naphthyridin-2(1H)-one

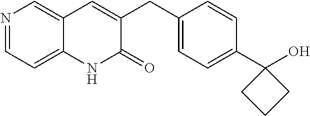 [SMILES: O=c1[nH]c2ccncc2cc1Cc1ccc(C2(O)CCC2)cc1]

### Step A: 1-(4-((2-methoxy-1,6-naphthyridin-3-yl)methyl)phenyl)cyclobutan-1-ol

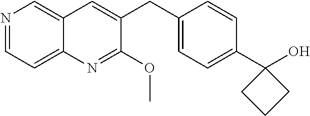 [SMILES: COc1nc2ccncc2cc1Cc1ccc(C2(O)CCC2)cc1]

Sodium carbonate (74.7 mg, 0.705 mmol), Pd(Ph₃P)₄ (78 mg, 0.067 mmol), 3-(chloromethyl)-2-methoxy-1,6-naphthyridine (70 mg, 0.335 mmol) and 1-(4-(4,4,5,5-tetramethyl-1,3,2-dioxaborolan-2-yl)phenyl)cyclobutan-1-ol (110 mg, 0.403 mmol) were added into a 20 mL vial containing a stir bar. Dioxane (2.52 mL) and water (0.84 mL) were then added to the mixture. Then the mixture was capped and sparged with argon for 5 minutes. The reaction was stirred at 100° C. for 3 hours. The reaction was cooled to room temperature. Then, the mixture was diluted with EtOAc (20 mL), filtered through Celite topped with sodium sulfate, and the solvents of the filtrate were evaporated to afford 1-(4-((2-methoxy-1,6-naphthyridin-3-yl)methyl)phenyl)cyclobutan-1-ol. The crude material was used as is without further purification. MS (ESI) m/z: 321 [M+H⁺].

### Step B: 3-(4-(1-hydroxycyclobutyl)benzyl)-1,6-naphthyridin-2(1H)-one

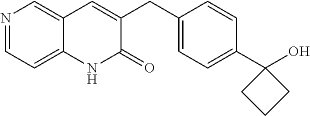 [SMILES: O=c1[nH]c2ccncc2cc1Cc1ccc(C2(O)CCC2)cc1]

Sodium iodide (94 mg, 0.624 mmol) was added to a 40 mL vial containing 1-(4-((2-methoxy-1,6-naphthyridin-3-yl)methyl)phenyl)cyclobutan-1-ol (50 mg, 0.156 mmol), followed by acetonitrile (1.5 mL). Then, chlorotrimethylsilane (198 μL, 1.561 mmol) was added slowly to the solution. The reaction was stirred at room temperature for 16 h. The sample was then filtered by washing with acetonitrile (containing 20% water) (3 mL), transferred into a 20 mL vial and concentrated under reduced pressure. The crude material was dissolved in DMSO (3 mL), filtered and purified by reversed phase HPLC (acetonitrile/water gradient w/0.1% TFA) and lyophilized to afford 3-(4-(1-hydroxycyclobutyl)benzyl)-1,6-naphthyridin-2(1H)-one. ¹H NMR (600 MHz, DMSO-d₆) δ 12.10 (s, 1H), 8.79 (s, 1H), 8.42 (d, J=5.7 Hz, 1H), 7.79 (s, 1H), 7.41 (d, J=8.2 Hz, 2H), 7.26 (d, J=8.2 Hz, 2H), 7.20 (d, J=5.7 Hz, 1H), 5.41 (s, 1H), 3.83 (s, 2H), 2.36 (ddd, J=12.2, 8.8, 5.1 Hz, 2H), 2.29-2.20 (m, 2H), 1.89 (ddq, J=14.6, 9.8, 5.0 Hz, 1H), 1.61 (ddt, J=16.5, 10.9, 8.5 Hz, 1H). LC/MS (m/z): 307 [M+H⁺].

### Example 114

### Preparation of methyl 3-(benzo[d]thiazol-5-ylmethyl)-1,6-naphthyridin-2(1H)-one

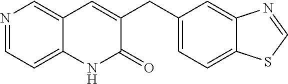 [SMILES: O=c1[nH]c2ccncc2cc1Cc1ccc2scnc2c1]

### Step A: 5-((2-methoxy-1,6-naphthyridin-3-yl)methyl)benzo[d]thiazole

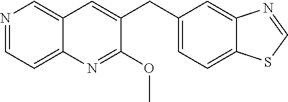 [SMILES: COc1nc2ccncc2cc1Cc1ccc2scnc2c1]

Dioxane (1 ml) and cesium fluoride 3.3M in water (0.232 ml, 0.767 mmol) was added to a vial containing 3-(chloromethyl)-2-methoxy-1,6-naphthyridine (16 mg, 0.077 mmol), Si-DPP-Pd (77 mg, 0.02 mmol, 0.26 mmol/g), and 5-(4,4,5,5-tetramethyl-1,3,2-dioxaborolan-2-yl)benzo[d]thiazole (40.1 mg, 0.153 mmol), and the reaction was heated at reflux for 3 hours. The reaction mixture was filtered and evaporated to dryness. Crude material was used as is in next step.

### Step B: methyl 3-(benzo[d]thiazol-5-ylmethyl)-1,6-naphthyridin-2(1H)-one

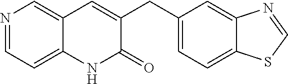 [SMILES: O=c1[nH]c2ccncc2cc1Cc1ccc2scnc2c1]

Dioxane (1.5 ml) and HCl 6 N (0.6 ml, 3.60 mmol) were added to a vial containing 5-((2-methoxy-1,6-naphthyridin-3-yl)methyl)benzo[d]thiazole and the reaction was heated at 80° C. for 30 minutes. The reaction was cooled to room temperature and the solvent was evaporated under reduced pressure. The residue was then purified via prep-HPLC (MeCN/H₂O with 0.1% TFA) to afford methyl (4-((2-oxo-1,2-dihydroquinolin-3-yl)methyl)phenyl)carbamate. ¹H NMR (600 MHz, DMSO-d₆) δ 12.11 (s, 1H), 9.38 (s, 1H), 8.78 (s, 1H), 8.42 (d, J=5.7 Hz, 1H), 8.10 (d, J=8.2 Hz, 1H), 8.06-8.02 (m, 1H), 7.84 (s, 1H), 7.46 (dd, J=8.3, 1.4 Hz, 1H), 7.20 (d, J=5.7 Hz, 1H), 4.03 (s, 2H). LC/MS (m/z): 294 (M+H)+

Examples shown in Table 17 below, were prepared according to procedures analogous to those outlined in Example 114 above using the appropriate starting materials, described in the Preparations or Intermediates above, or as obtained from commercial sources.

TABLE 17

| | | LCMS |
|---|---|---|
| Ex- | Structure | (m/z) |
| ample | Name | (M + H)+ |
| 115 | 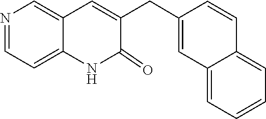 [SMILES: O=c1[nH]c2ccncc2cc1Cc1ccc2ccccc2c1] | 287 |
| | 3-(naphthalen-2-ylmethyl)-1,6-naphthyridin-2(1H)-one | |
| 116 | 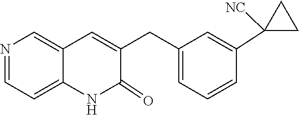 [SMILES: N#CC1(c2cccc(Cc3cc4cnccc4[nH]c3=O)c2)CC1] | 302 |
| | 1-(3-((2-oxo-1,2-dihydro-1,6-naphthyridin-3- | |
| | yl)methyl)phenyl)cyclopropane-1-carbonitrile | |
| 117 | 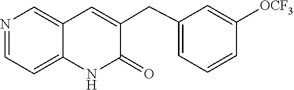 [SMILES: O=c1[nH]c2ccncc2cc1Cc1cccc(OC(F)(F)F)c1] | 321 |
| | 3-(3-(trifluoromethoxy)benzyl)-1,6-naphthyridin-2(1H)-one | |
| 118 | 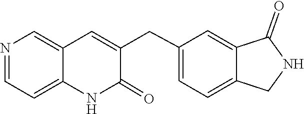 [SMILES: O=C1NCc2ccc(Cc3cc4cnccc4[nH]c3=O)cc21] | 292 |
| | 3-((3-oxoisoindolin-5-yl)methyl)-1,6-naphthyridin-2(1H)- | |
| | one | |

### Example 119

### Preparation of methyl (4-((2-oxo-1,2-dihydroquinolin-3-yl)methyl)phenyl)carbamate

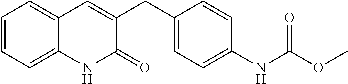 [SMILES: COC(=O)Nc1ccc(Cc2cc3ccccc3[nH]c2=O)cc1]

### Step A: methyl (4-((2-methoxyquinolin-3-yl)methyl)phenyl)carbamate

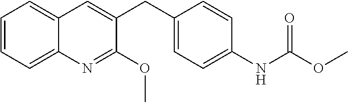 [SMILES: COC(=O)Nc1ccc(Cc2cc3ccccc3nc2OC)cc1]

Methyl (4-(4,4,5,5-tetramethyl-1,3,2-dioxaborolan-2-yl)phenyl)carbamate (0.0550 g, 0.200 mmol), PdCl₂(dppf)(CH₂Cl₂) (0.016 g, 0.020 mmol) and 1,4-dioxane (2 ml) were added to a vial containing 3-(chloromethyl)-2-methoxyquinoline (0.021 g, 0.10 mmol). 5 M aqueous cesium fluoride (0.100 ml, 0.500 mmol) was then added, and the mixture was heated at reflux for 2.5 hours. The mixture was filtered and concentrated. The residue was used as is in the next step.

### Step B: methyl (4-((2-oxo-1,2-dihydroquinolin-3-yl)methyl)phenyl)carbamate

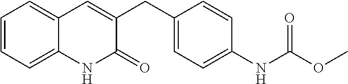 [SMILES: COC(=O)Nc1ccc(Cc2cc3ccccc3[nH]c2=O)cc1]

Dioxane (1.5 ml) and HCl 6 N (0.6 ml, 3.60 mmol) were added to a vial containing methyl (4-((2-oxo-1,2-dihydroquinolin-3-yl)methyl)phenyl)carbamate, and the mixture was heated at 80° C. for 30 minutes. The mixture was cooled to room temperature, and the solvent was evaporated under reduced pressure. The residue was purified by reversed phase HPLC (MeCN/water w/0.100 TFA) to afford methyl (4-((2-oxo-1,2-dihydroquinolin-3-yl)methyl)phenyl)carbamate. ¹H NMR (500 MHz, DMSO-d₆) δ 11.78 (s, 1H), 9.57 (s, 1H), 7.62 (s, 1H), 7.59-7.48 (m, 3H), 7.43 (t, J=7.2 Hz, 1H), 7.37 (d, J=8.2 Hz, 2H), 7.29 (d, J=8.2 Hz, 1H), 7.20 (d, J=8.4 Hz, 2H), 7.14 (t, J=7.4 Hz, 1H), 3.64 (s, 3H). LC/MS (m/z): 309 (M+H)+

Examples shown in Table 18 below, were prepared according to procedures analogous to those outlined in Example 119 above using the appropriate starting materials, described in the Preparations or Intermediates above, or as obtained from commercial sources.

TABLE 18

| | Structure | LCMS (m/z) |
|---|---|---|
| Example | Name | (M + H)+ |
| 120 | 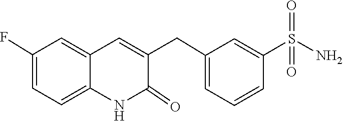 [SMILES: NS(=O)(=O)c1cccc(Cc2cc3cc(F)ccc3[nH]c2=O)c1] | 333 |
| | 3-((6-fluoro-2-oxo-1,2-dihydroquinolin-3- | |
| | yl)mehtyl)benzenesulfonamide | |
| 121 | 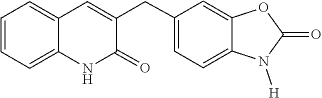 [SMILES: O=c1[nH]c2ccc(Cc3cc4ccccc4[nH]c3=O)cc2o1] | 293 |
| | 6-((2-oxo-1,2-dihydroquinolin-3-yl)methyl)benzo[d]oxazol- | |
| | 2(3H)-one | |
| 122 | 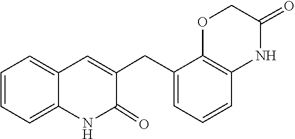 [SMILES: O=C1COc2c(Cc3cc4ccccc4[nH]c3=O)cccc2N1] | 307 |
| | 8-((2-oxo-1,2-dihydroquinolin-3-yl)methyl)-2H- | |
| | benzo[b][1,4]oxazin-3(4H)-one | |
| 120 | 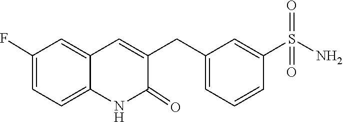 [SMILES: NS(=O)(=O)c1cccc(Cc2cc3cc(F)ccc3[nH]c2=O)c1] | 333 |
| | 3-((6-fluoro-2-oxo-1,2-dihydroquinolin-3- | |
| | yl)methyl)benzenesulfonamide | |
| 123 | 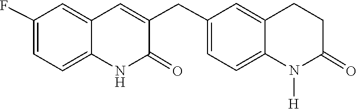 [SMILES: O=C1CCc2cc(Cc3cc4cc(F)ccc4[nH]c3=O)ccc2N1] | 323 |
| | 6-fluoro-3-((2-oxo-1,2,3,4-tetrahydroquinolin-6- | |
| | yl)methyl)quinolin-2(1H)-one | |
| 124 | 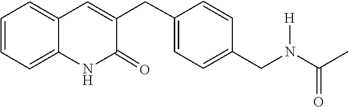 [SMILES: CC(=O)NCc1ccc(Cc2cc3ccccc3[nH]c2=O)cc1] | 307 |
| | N-(4-((2-oxo-1,2-dihydroquinolin-3- | |
| | yl)methyl)benzyl)acetamide | |
| 125 | 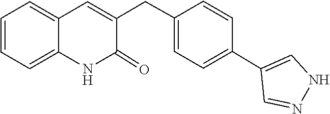 [SMILES: O=c1[nH]c2ccccc2cc1Cc1ccc(-c2cn[nH]c2)cc1] | 302 |
| | 3-(4-(1H-pyrazol-4-yl)benzyl)quinolin-2(1H)-one | |
| 126 | 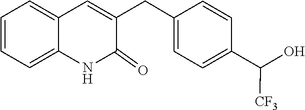 [SMILES: O=c1[nH]c2ccccc2cc1Cc1ccc(C(O)C(F)(F)F)cc1] | 334 |
| | rac-3-(4-(2,2,2-trifluoro-1-hydroxyethyl)benzyl)quinolin- | |
| | 2(1H)-one | |
| 127 | 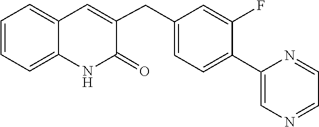 [SMILES: O=c1[nH]c2ccccc2cc1Cc1ccc(-c2cnccn2)c(F)c1] | 332 |
| | 3-(3-fluoro-4-(pyrazin-2-yl)benzyl)quinolin-2(1H)-one | |
| 120 | 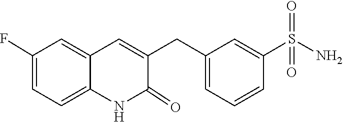 [SMILES: NS(=O)(=O)c1cccc(Cc2cc3cc(F)ccc3[nH]c2=O)c1] | 333 |
| | 3-((6-fluoro-2-oxo-1,2-dihydroquinolin-3- | |
| | yl)methyl)benzenesulfonamide | |
| 128 | 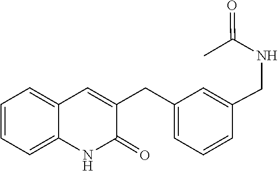 [SMILES: CC(=O)NCc1cccc(Cc2cc3ccccc3[nH]c2=O)c1] | 307 |
| | N-(3-((2-oxo-1,2-dihydroquinolin-3- | |
| | yl)methyl)benzyl)acetamide | |
| 129 | 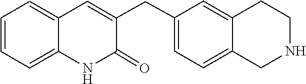 [SMILES: O=c1[nH]c2ccccc2cc1Cc1ccc2c(c1)CCNC2] | 291 |
| | 3-((1,2,3,4-tetrahydroisoquinolin-6-yl)methyl)quinolin-2(1H)- | |
| | one | |
| 130 | 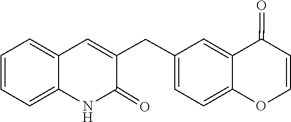 [SMILES: O=c1[nH]c2ccccc2cc1Cc1ccc2occc(=O)c2c1] | 304 |
| | 3-((4-oxo-4H-chromen-6-yl)methyl)quinolin-2(1H)-one | |
| 131 | 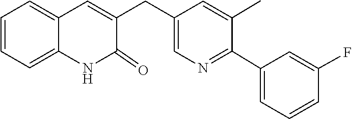 [SMILES: Cc1cc(Cc2cc3ccccc3[nH]c2=O)cnc1-c1cccc(F)c1] | 345 |
| | 3-((6-(3-fluorophenyl)-5-methylpyridin-3-yl)methyl)quinolin- | |
| | 2(1H)-one | |
| 132 | 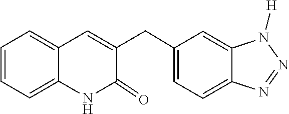 [SMILES: O=c1[nH]c2ccccc2cc1Cc1ccc2nn[nH]c2c1] | 277 |
| | 3-((1H-benzo[d][1,2,3]triazol-6-yl)methyl)quinolin-2(1H)-one | |
| 120 | 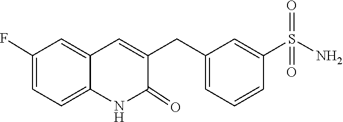 [SMILES: NS(=O)(=O)c1cccc(Cc2cc3cc(F)ccc3[nH]c2=O)c1] | 333 |
| | 3-((6-fluoro-2-oxo-1,2-dihydroquinolin-3- | |
| | yl)methyl)benzenesulfonamide | |
| 133 | 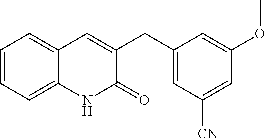 [SMILES: COc1cc(C#N)cc(Cc2cc3ccccc3[nH]c2=O)c1] | 291 |
| | 3-methoxy-5-((2-oxo-1,2-dihydroquinolin-3- | |
| | yl)methyl)benzonitrile | |
| 134 | 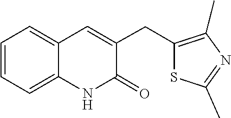 [SMILES: Cc1nc(C)c(Cc2cc3ccccc3[nH]c2=O)s1] | 271 |
| | 3-((2,4-dimethylthiazol-5-yl)methyl)quinolin-2(1H)-one | |
| 135 | 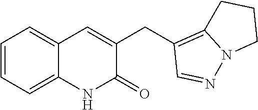 [SMILES: O=c1[nH]c2ccccc2cc1Cc1cnn2c1CCC2] | 266 |
| | 3-((5,6-dihydro-4H-pyrrolo[1,2-b]pyrazol-3- | |
| | yl)methyl)quinolin-2(1H)-one | |
| 136 | 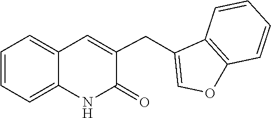 [SMILES: O=c1[nH]c2ccccc2cc1Cc1coc2ccccc12] | 276 |
| | 3-(benzofuran-3-ylmethyl)quinolin-2(1H)-one | |
| 137 | 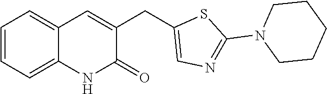 [SMILES: O=c1[nH]c2ccccc2cc1Cc1cnc(N2CCCCC2)s1] | 326 |
| | 3-((2-(piperidin-1-yl)thiazol-5-yl)methyl)quinolin-2(1H)-one | |
| 138 | 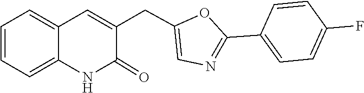 [SMILES: O=c1[nH]c2ccccc2cc1Cc1cnc(-c2ccc(F)cc2)o1] | 321 |
| 120 | 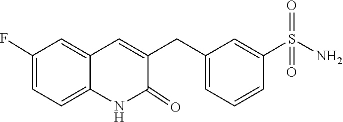 [SMILES: NS(=O)(=O)c1cccc(Cc2cc3cc(F)ccc3[nH]c2=O)c1] | 333 |
| | 3-((6-fluoro-2-oxo-1,2-dihydroquinolin-3- | |
| | yl)methyl)benzenesulfonamide | |
| | 3-((2-(4-fluorophenyl)oxazol-5-yl)methyl)quinolin-2(1H)-one | |
| 139 | 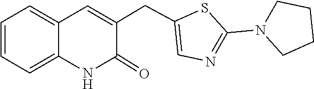 [SMILES: O=c1[nH]c2ccccc2cc1Cc1cnc(N2CCCC2)s1] | 312 |
| | 3-((2-(pyrrolidin-1-yl)thiazol-5-yl)methyl)quinolin-2(1H)-one | |
| 140 | 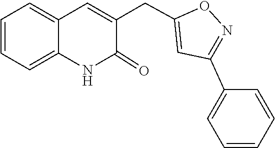 [SMILES: O=c1[nH]c2ccccc2cc1Cc1cc(-c2ccccc2)no1] | 303 |
| | 3-((3-phenylisoxazol-5-yl)methyl)quinolin-2(1H)-one | |
| 141 | 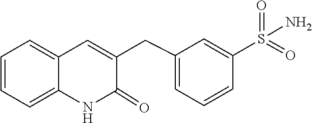 [SMILES: NS(=O)(=O)c1cccc(Cc2cc3ccccc3[nH]c2=O)c1] | 315 |
| | 3-((2-oxo-1,2-dihydroquinolin-3- | |
| | yl)methyl)benzenesulfonamide | |

Examples shown in Table 19 below, were prepared according to procedures analogous to those outlined in Example 119 above using the appropriate starting materials, described in the Preparations or Intermediates above, or as obtained from commercial sources. Also, the products were resolved into their single enantiomers by chiral SFC resolution.

TABLE 19

| | | LCMS | |
|---|---|---|---|
| | Structure | (m/z) | |
| Example | Name | (M + H)+ | SFC conditions |
| 142A (peak 1) and 142B (peak 2) | 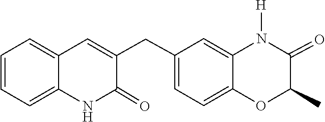 [SMILES: C[C@H]1Oc2ccc(Cc3cc4ccccc4[nH]c3=O)cc2NC1=O] | 321 | SJ 21 × 250 mm column; 25% MeOH (w/ 0.1% NH₄OH) modifier |
| | 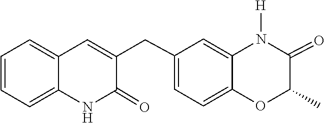 [SMILES: C[C@@H]1Oc2ccc(Cc3cc4ccccc4[nH]c3=O)cc2NC1=O] | | |
| | 2-methyl-6-((2-oxo-1,2-dihydroquinolin-3- | | |
| | yl)methyl)-2H-benzo[b][1,4]oxazin-3(4H)- | | |
| | one | | |

### Example 143

### Preparation of 3-(4-(2-hydroxypropan-2-yl)benzyl)-1-methylquinolin-2(1H)-one

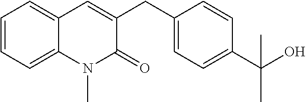 [SMILES: Cn1c(=O)c(Cc2ccc(C(C)(C)O)cc2)cc2ccccc21]

(4-(2-hydroxypropan-2-yl)phenyl)boronic acid (72.0 mg, 0.400 mmol), PdCl₂(dppf)(CH₂Cl₂) (32.7 mg, 0.0400 mmol), potassium fluoride (58.1 mg, 1.00 mmol), and isopropanol (1.5 ml) were added to a vial containing 3-(chloromethyl)-1-methylquinolin-2(1H)-one (41.5 mg, 0.200 mmol). The mixture was then heated at 65° C. overnight. The mixture was cooled to room temperature, filtered, and concentrated. The residue was then purified by reversed phase HPLC (MeCN/water w/0.1% TFA) to afford 3-(4-(2-hydroxypropan-2-yl)benzyl)-1-methylquinolin-2(1H)-one. ¹H NMR (600 MHz, DMSO-d₆) δ 7.75 (s, 1H), 7.67-7.63 (m, 1H), 7.62-7.53 (m, 1H), 7.50 (d, J=8.5 Hz, 1H), 7.36 (d, J=8.2 Hz, 2H), 7.30-7.19 (m, 3H), 4.91 (s, 1H), 3.84 (s, 2H), 3.64 (s, 3H), 1.39 (s, 6H). LC/MS (m/z): 308 (M+H)+

Examples shown in Table 20 below, were prepared according to procedures analogous to those outlined in Example 143 above using the appropriate starting materials, described in the Preparations or Intermediates above, or as obtained from commercial sources.

TABLE 20

| | Structure | LCMS (m/z) |
|---|---|---|
| Example | Name | (M + H)+ |
| 144 | 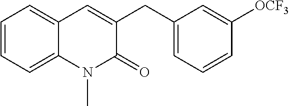 [SMILES: Cn1c(=O)c(Cc2cccc(OC(F)(F)F)c2)cc2ccccc21] | 334 |
| | 1-methyl-3-(3-(trifluoromethoxy)benzyl)quinolin-2(1H)-one | |
| 145 | 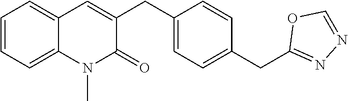 [SMILES: Cn1c(=O)c(Cc2ccc(Cc3nnco3)cc2)cc2ccccc21] | 372 |
| | 3-(4-((1,3,4-oxadiazol-2-yl)methyl)benzyl)-1- | |
| | methylquinolin-2(1H)-one | |
| 146 | 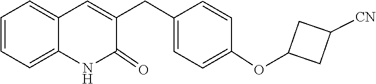 [SMILES: N#CC1CC(Oc2ccc(Cc3cc4ccccc4[nH]c3=O)cc2)C1] | 331 |
| | 3-(4-((2-oxo-1,2-dihydroquinolin-3- | |
| | yl)methyl)phenoxy)cyclobutane-1-carbonitrile | |
| 147 | 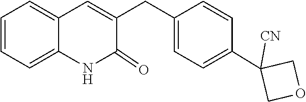 [SMILES: N#CC1(c2ccc(Cc3cc4ccccc4[nH]c3=O)cc2)COC1] | 317 |
| | 3-(4-((2-oxo-1,2-dihydroquinolin-3- | |
| | yl)methyl)phenyl)oxetane-3-carbonitrile | |
| 148 | 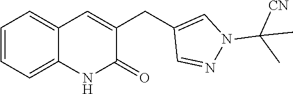 [SMILES: CC(C)(C#N)n1cc(Cc2cc3ccccc3[nH]c2=O)cn1] | 293 |
| | 2-methyl-2-(4-((2-oxo-1,2-dihydroquinolin-3-yl)methyl)-1H- | |
| | pyrazol-1-yl)propanenitrile | |

### Example 149

### Preparation of 8-((5-oxo-2,3-dihydro-1H,5H-pyrido[3,2,1-ij]quinolin-6-yl)methyl)-2H-benzo[b][1,4]oxazin-3(4H)-one

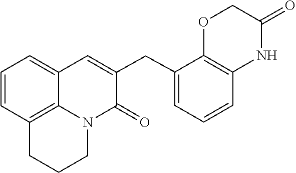 [SMILES: O=C1COc2c(Cc3cc4cccc5c4n(c3=O)CCC5)cccc2N1]

8-(4,4,5,5-tetramethyl-1,3,2-dioxaborolan-2-yl)-2H-benzo[b][1,4]oxazin-3(4H)-one (68.6 mg, 0.250 mmol) was added to a vial containing 6-(chloromethyl)-2,3-dihydro-1H,5H-pyrido[3,2,1-ij]quinolin-5-one (28 mg, 0.12 mmol) and Si-DPP-Pd (138 mg, 0.0360 mmol, 0.260 mmol/g), followed by 1,4-dioxane (1 ml) and a 3.3 M aqueous solution of cesium fluoride (0.207 ml, 0.685 mmol). The mixture was heated at reflux for 3 hours, filtered and concentrated. The residue was then purified by reversed phase HPLC (MeCN/water w/0.1% TFA) to afford 8-((5-oxo-2,3-dihydro-1H,5H-pyrido[3,2,1-ij]quinolin-6-yl)methyl)-2H-benzo[b][1,4]oxazin-3(4H)-one. ¹H NMR (500 MHz, DMSO-d₆) δ 10.70 (s, 1H), 7.46 (d, J=7.7 Hz, 1H), 7.43 (s, 1H), 7.32 (d, J=7.1 Hz, 1H), 7.11 (t, J=7.5 Hz, 1H), 6.90 (t, J=7.6 Hz, 1H), 6.82 (t, J=7.0 Hz, 2H), 4.55 (s, 2H), 4.11-4.05 (m, 2H), 3.81 (s, 2H), 2.93 (t, J=6.0 Hz, 2H), 2.06-1.98 (m, 2H). LC/MS (m/z): 347 (M+H)⁺

### Example 150

### Preparation of N-(5-((2-oxo-1,2-dihydroquinolin-3-yl)methyl)pyridin-2-yl)acetamide

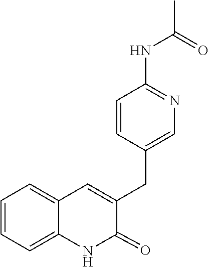 [SMILES: CC(=O)Nc1ccc(Cc2cc3ccccc3[nH]c2=O)cn1]

### Step A: N-(5-((2-methoxyquinolin-3-yl)methyl)pyridin-2-yl)acetamide

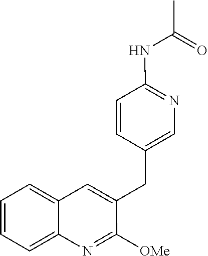 [SMILES: COc1nc2ccccc2cc1Cc1ccc(NC(C)=O)nc1]

3-(chloromethyl)-2-methoxyquinoline (35.3 mg, 0.170 mmol), potassium fluoride (29.4 mg, 0.510 mmol), bis(tirphenylphosphine)palladium(II) dichloride (11.8 mg, 0.0170 mmol) in MeOH (1.1 mL) were added to a 0.5 mL-2 mL microwave vial. The mixture was purged with Ar. The reaction mixture was heated in a microwave reactor at 125° C. for 10 mins. Then the reaction mixture was treated with water and extracted with DCM. The mixture was concentrated, and the residue was used for next step. LC/MS (m/z): 308 (M+H)⁺

### Step B: N-(5-((2-oxo-1,2-dihydroquinolin-3-yl)methyl)pyridin-2-yl)acetamide

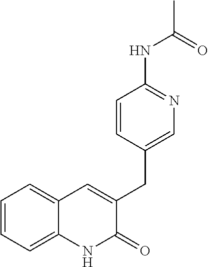 [SMILES: CC(=O)Nc1ccc(Cc2cc3ccccc3[nH]c2=O)cn1]

NaI (101.9 mg, 0.68 mmol) and TMSCl (0.086 mL, 0.68 mmol) in MeCN (1 mL) were added to the crude reaction mixture from Step A. The reaction mixture was stirred at room temperature for 16 h. The mixture was filtered and purified by reversed phase HPLC (MeCN/water w/0.1% TFA) to afford N-(5-((2-oxo-1,2-dihydroquinolin-3-yl)methyl)pyridin-2-yl)acetamide. ¹H NMR (500 MHz, DMSO-d₆) δ 11.82 (s, 1H), 10.53 (s, 1H), 8.25 (d, J=2.0 Hz, 1H), 7.92 (d, J=8.5 Hz, 1H), 7.78-7.69 (m, 2H), 7.60 (d, J=7.8 Hz, 1H), 7.48-7.42 (m, 1H), 7.30 (d, J=8.2 Hz, 1H), 7.20-7.13 (m, 1H), 3.80 (s, 2H), 2.08 (s, 3H). LC/MS (m/z): 294 (M+H)+

### Example 151

### Preparation of N-(4-((1-Methyl-2-oxo-1,2-dihydroquinolin-3-yl)methyl)benzyl)acetamide

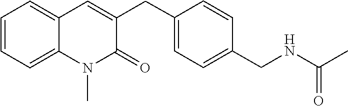 [SMILES: CC(=O)NCc1ccc(Cc2cc3ccccc3n(C)c2=O)cc1]

3-(Chloromethyl)-1-methylquinolin-2(1H)-one (75 mg, 0.363 mmol), Pd(PPh₃)₄ (21 mg, 0.018 mmol) and K₂CO₃ (201 mg, 1.45 mmol) were added to a stirred mixture of N-(4-(4,4,5,5-tetramethyl-1,3,2-dioxaborolan-2-yl)benzyl)acetamide (100 mg, 0.363 mmol) in 1,4-dioxane (5 mL) and water (1 mL) at 30° C. The mixture was stirred and heated at 95° C. for 1 hour. The mixture was cooled to room temperature and then concentrated under reduced pressure. The residue was purified by reverse phase HPLC (MeCN/water w/0.1% TFA) to afford N-(4-((1-methyl-2-oxo-1,2-dihydroquinolin-3-yl)methyl)benzyl)acetamide. ¹H NMR (500 MHz, methanol-d₄) δ 7.59-7.58 (m, 3H), 7.56-7.52 (m, 2H), 7.28-7.25 (m, 3H), 7.24-7.22 (m, 2H), 4.33 (s, 2H), 3.92 (s, 2H), 3.75 (s, 3H), 1.97 (s, 3H). LC/MS (m/z): 321 (M+H)⁺

### Example 152

### Preparation of 3-(Quinolin-6-ylmethyl)quinolin-2(1H)-one

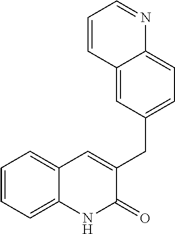 [SMILES: O=c1[nH]c2ccccc2cc1Cc1ccc2ncccc2c1]

### Step A: 2-Methoxy-3-(quinolin-6-ylmethyl)quinoline

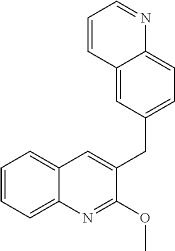 [SMILES: COc1nc2ccccc2cc1Cc1ccc2ncccc2c1]

A mixture of 3-(chloromethyl)-2-methoxyquinoline (24 mg, 50 μmol), 6-(4,4,5,5-tetramethyl-1,3,2-dioxaborolan-2-yl)quinoline (15 mg, 60 μmol), potassium phosphate (32 mg, 150.0 μmol) and Pd(dtbpf)Cl₂ (0.26 mg, 0.500 μmol) was dissolved in dioxane (0.50 mL) and water (0.05 mL) under a nitrogen atmosphere. The reaction mixture was shaken and heated to 100° C. for 16 hrs. The reaction mixture was then concentrated under reduced pressure to afford the crude residue. The residue was then diluted with ethyl acetate (1 mL) and washed with water (1 mL). The organic layer was separated and the aqueous was then washed with additional ethyl acetate (2×1 mL). The organic layers were combined, dried over sodium sulfate, filtered, and concentrated under reduced pressure. The product was used without purification in the next step.

### Step B: 3-(Quinolin-6-ylmethyl)quinolin-2(1H)-one

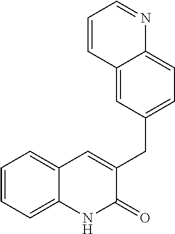 [SMILES: O=c1[nH]c2ccccc2cc1Cc1ccc2ncccc2c1]

A mixture of THF and 37% HCl (2:1 mixture, 1 mL) was added to 2-methoxy-3-(quinolin-6-ylmethyl)quinoline (crude from previous reaction). The reaction mixture was heated to 80° C. and stirred for 2 hours. The reaction mixture was cooled to room temperature and then concentrated under reduced pressure. The residue was purified by preparative HPLC (eluting acetonitrile in water with TFA modifier) to afford 6-((2-oxo-1,2-dihydroquinolin-3-yl)methyl)quinolin-1-ium 2,2,2-trifluoroacetate. ¹H NMR (500 MHz, DMSO-d₆) δ 8.96 (d, J=5 Hz, 1H), 8.54 (d, J=5 Hz, 1H), 8.03 (d, J=10 Hz, 1H), 7.95 (s, 1H), 7.85-7.81 (m, 2H), 7.67-7.60 (m, 2H), 7.48-7.44 (m, 1H), 7.31 (d, J=10 Hz, 1H), 7.17-7.14 (m, 1H), 4.08 (s, 2H). LC/MS (m/z): 287 (M+H)

Examples shown in Table 21 below, were prepared according to procedures analogous to those outlined in Example 21 above using the appropriate starting materials, described in the Preparations or Intermediates above, or as obtained from commercial sources.

TABLE 21

| | | LCMS |
|---|---|---|
| Ex- | Structure | (m/z) |
| ample | Name | (M + H)+ |
| 153 | 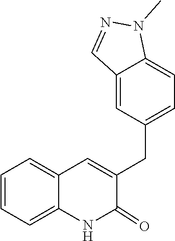 [SMILES: Cn1ncc2cc(Cc3cc4ccccc4[nH]c3=O)ccc21] | 290 |
| | 3-((1-methyl-1H-indazol-5-yl)methyl)quinolin-2(1H)-one | |
| 154 | 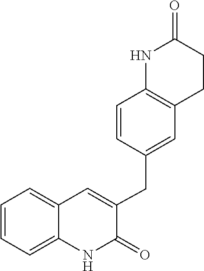 [SMILES: O=C1CCc2cc(Cc3cc4ccccc4[nH]c3=O)ccc2N1] | 305 |
| | 3-((2-oxo-1,2,3,4-tetrahydroquinolin-6-yl)methyl)quinolin- | |
| | 2(1H)-one | |
| 155 | 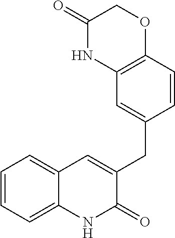 [SMILES: O=C1COc2ccc(Cc3cc4ccccc4[nH]c3=O)cc2N1] | 307 |
| | 6-((2-oxo-1,2-dihydroquinolin-3-yl)methyl)-2H- | |
| | benzo[b][1,4]oxazin-3(4H)-one | |
| 156 | 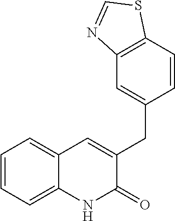 [SMILES: O=c1[nH]c2ccccc2cc1Cc1ccc2scnc2c1] | 293 |
| | 3-(benzo[d]thiazol-5-ylmethyl)quinolin-2(1H)-one | |
| 157 | 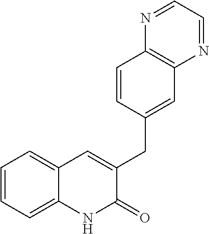 [SMILES: O=c1[nH]c2ccccc2cc1Cc1ccc2nccnc2c1] | 288 |
| | 3-(quinoxalin-6-ylmethyl)quinolin-2(1H)-one | |
| 158 | 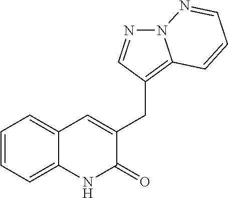 [SMILES: O=c1[nH]c2ccccc2cc1Cc1cnn2ncccc12] | 277 |
| | 3-(pyrazolo[1,5-b]pyridazin-3-ylmethyl)quinolin-2(1H)- | |
| | one | |

### Example 159

### Preparation of (S)-3-(4-(5-oxopyrrolidin-2-yl)benzyl)quinolin-2(1H)-one

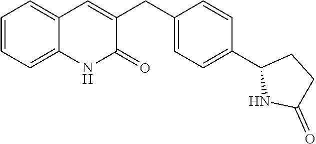 [SMILES: O=C1CC[C@@H](c2ccc(Cc3cc4ccccc4[nH]c3=O)cc2)N1]

### Step A: (S)-5-(4-((2-methoxyquinolin-3-yl)methyl)phenyl)pyrrolidin-2-one

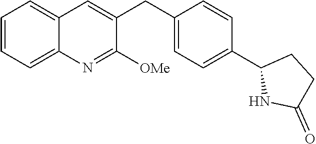 [SMILES: COc1nc2ccccc2cc1Cc1ccc([C@@H]2CCC(=O)N2)cc1]

3-(chloromethyl)-2-methoxyquinoline (17.4 g, 92.4 mmol), (S)-5-(4-(4,4,5,5-tetramethyl-1,3,2-dioxaborolan-2-yl)phenyl)pyrrolidin-2-one (24.0 g, 84.0 mmol), potassium phosphate (53.2 g, 251 mmol), [1,1′-Bis(di-tert-butylphosphino)ferrocene]dichloropalladium(II) (2.72 g, 4.20 mmol), dioxane (192 ml) and water (72.0 ml) were added to 1 L three-necked round bottom flask with stir bar. The mixture was evacuated and back-filled with N2 three times and heated to 75° C. for 14 h. The mixture was then cooled to room temperature and diluted with water. The mixture was then extracted three times with ethyl acetate and the organics were combined, dried with magnesium sulfate, filtered and evaporated under reduced pressure. The crude material was purified on silica gel with methanol in dichloromethane as the eluent.

LC/MS (m/z): 333 (M+H)+

### Step B: (S)-3-(4-(5-oxopyrrolidin-2-yl)benzyl)quinolin-2(1H)-one

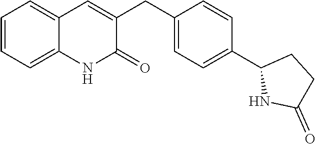 [SMILES: O=C1CC[C@@H](c2ccc(Cc3cc4ccccc4[nH]c3=O)cc2)N1]

(S)-5-(4-((2-methoxyquinolin-3-yl)methyl)phenyl)pyrrolidin-2-one (13.0 g, 39.1 mmol), TMS-Cl (55.0 g, 508 mmol) and acetonitrile (39.0 mL) were added to a 1 L three-necked round bottom flask with a stir bar. The mixture was heated to 70° C. for 12 h. It was then cooled to room temperature and filtered. The cake was collected then slurried with 10 volumes MTBE for 1 h, then the mixture was filtered. The cake was slurried with 12 volumes H₂O:acetonitrile (4:1) for 2 h. the mixture was filtered, and the cake was dried at 60° C. for 5 h. ¹H NMR (600 MHz, DMSO-d₆) δ 11.78 (s, 1H), 8.04 (s, 1H), 7.71 (s, 1H), 7.57 (d, J=7.8 Hz, 1H), 7.44 (t, J=7.7 Hz, 1H), 7.29 (d, J=8.1 Hz, 2H), 7.22 (d, J=8.0 Hz, 2H), 7.14 (t, J=7.5 Hz, 1H), 4.62 (t, J=7.0 Hz, 1H), 3.82 (s, 2H), 2.43 (dq, J=14.0, 7.6 Hz, 1H), 2.22 (t, J=8.2 Hz, 2H), 1.78-1.69 (m, 1H).

LC/MS (m/z): 319 (M+H)+

### Example 160

### Preparation of 3-(4-(2-Hydroxypropan-2-yl)benzyl)quinolin-2(1H)-one

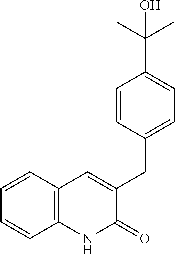 [SMILES: CC(C)(O)c1ccc(Cc2cc3ccccc3[nH]c2=O)cc1]

### Step A: Methyl 4-((2-methoxyquinolin-3-yl)methyl)benzoate

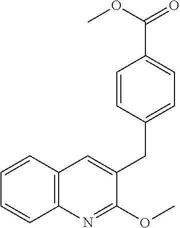 [SMILES: COC(=O)c1ccc(Cc2cc3ccccc3nc2OC)cc1]

A mixture of 3-(chloromethyl)-2-methoxyquinoline (30 mg, 0.14 mmol), (4-(methoxycarbonyl)phenyl)boronic acid (34 mg, 0.20 mmol), PdCl₂(dppf)-CH₂Cl₂ adduct (12 mg, 0.014 mmol), and tripotassium phosphate (92 mg, 0.43 mmol) in dioxane (2 mL) and H₂O (0.4 mL) was stirred at 100° C. for 2 hours. The reaction mixture was poured out into H₂O (20 mL) and extracted with EtOAc (20 mL). The organic layer was washed with brine, dried over sodium sulfate, filtered, and concentrated under reduced pressure. The residue was purified by silica gel chromatography (eluting ethyl acetate in hexanes) to afford methyl 4-((2-methoxyquinolin-3-yl)methyl)benzoate. ¹H NMR (500 MHz, chloroform-d) δ 8.00-7.96 (m, 2H), 7.85 (d, J=8.2 Hz, 1H), 7.66-7.55 (m, 3H), 7.37-7.29 (m, 3H), 4.09 (s, 2H), 4.08 (s, 3H), 3.91 (s, 3H). LC/MS (m/z): 308 (M+H)⁺

### Step B: Methyl 4-((2-oxo-1,2-dihydroquinolin-3-yl)methyl)benzoate

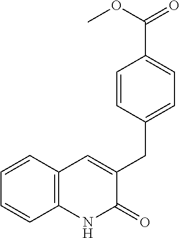 [SMILES: COC(=O)c1ccc(Cc2cc3ccccc3[nH]c2=O)cc1]

Iodotrimethylsilane (117 mg, 0.585 mmol) was added to a mixture of methyl 4-((2-methoxyquinolin-3-yl)methyl)benzoate (30 mg, 0.10 mmol) in acetonitrile (2 mL) at room temperature. The reaction mixture was stirred under an inert atmosphere for 12 hours. Saturated aqueous Na₂SO₃ (2 mL) was added to the reaction mixture. The mixture was diluted with EtOAc (10 mL) and the organic layer was separated. The organic layer was dried over sodium sulfate, filtered, and concentrated under reduced pressure to afford methyl 4-((2-oxo-1,2-dihydroquinolin-3-yl)methyl)benzoate, which was used in the next step without further purification. ¹H NMR (500 MHz, DMSO-d₆) δ 11.87-11.80 (m, 1H), 7.93-7.88 (m, 2H), 7.76 (s, 1H), 7.60 (d, J=7.0 Hz, 1H), 7.47-7.44 (m, 3H), 7.32-7.29 (m, 1H), 7.18-7.14 (m, 1H), 3.93 (s, 2H), 3.84 (s, 3H). LC/MS (m/z): 294 (M+H)⁺

### Step C: 3-(4-(2-Hydroxypropan-2-yl)benzyl)quinolin-2(1H)-one

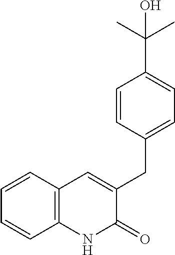 [SMILES: CC(C)(O)c1ccc(Cc2cc3ccccc3[nH]c2=O)cc1]

Methylmagnesium bromide (0.34 mL, 0.68 mmol, 2.0 M in Me-THF) was added dropwise to a solution of methyl 4-((2-oxo-1,2-dihydroquinolin-3-yl)methyl)benzoate (40 mg, 0.14 mmol) in dry THF (2 mL) under nitrogen at 0° C. The reaction mixture was allowed to warm to room temperature and then stirred for 14 hours. Saturated aqueous NH₄Cl (20 mL) was added to the reaction mixture and the mixture was then diluted with CH₂Cl₂ (20 mL). The mixture was washed with water. The organic layer was separated, dried over sodium sulfate, filtered, and concentrated under reduced pressure. The residue was purified by reverse phase HPLC (eluting acetonitrile in water with ammonium bicarbonate modifier) to afford 3-(4-(2-hydroxypropan-2-yl)benzyl)quinolin-2(1H)-one. ¹H NMR (500 MHz, methanol-d₄) δ 7.64 (s, 1H), 7.56 (d, J=7.9 Hz, 1H), 7.52-7.44 (m, 3H), 7.35 (d, J=8.2 Hz, 1H), 7.28 (d, J=8.4 Hz, 2H), 7.25-7.20 (m, 1H), 3.92 (s, 2H), 1.54 (s, 6H). LC/MS (m/z): 294 (M+H)⁺

### Example 161

### Preparation of 3-(4-(3-hydroxy-3-methylcyclobutyl)benzyl)quinolin-2(1H)-one

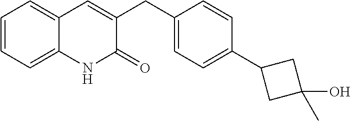 [SMILES: CC1(O)CC(c2ccc(Cc3cc4ccccc4[nH]c3=O)cc2)C1]

### Step A: 3-(4-((2-methoxyquinolin-3-yl)methyl)phenyl)cyclobutan-1-one

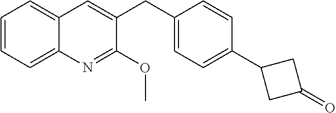 [SMILES: COc1nc2ccccc2cc1Cc1ccc(C2CC(=O)C2)cc1]

Dioxane (5 ml) and water (0.5 ml) was added to a 20 ml vial containing 3-(4-(4,4,5,5-tetramethyl-1,3,2-dioxaborolan-2-yl)phenyl)cyclobutan-1-one (136 mg, 0.500 mmol), 3-(chloromethyl)-2-methoxyquinoline (80 mg, 0.385 mmol), cesium fluoride (234 mg, 1.541 mmol), and 1,1′-bis(diphenylphosphino)ferrocene-palladium(II)dichloride dichloromethane complex (47.2 mg, 0.058 mmol). The vial head space was purged with argon, and the reaction was then heated to 80° C. for ˜4 hours before cooling to room temperature overnight. The reaction was then filtered and concentrated under reduced pressure. The resulting crude material was carried to the next step. LC/MS (m/z): 318 (M+H)+

### Step B: 3-(4-(3-hydroxy-3-methylcyclobutyl)benzyl)quinolin-2(1H)-one

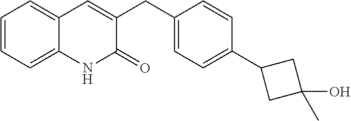 [SMILES: CC1(O)CC(c2ccc(Cc3cc4ccccc4[nH]c3=O)cc2)C1]

THF (6 ml) was added to an argon-purged vial containing crude 3-(4-((2-methoxyquinolin-3-yl)methyl)phenyl)cyclobutan-1-one (122 mg, assumed 0.385 mmol). The solution was cooled to 0° C., and then methylmagnesium bromide (0.16 ml, 0.480 mmol, 3M in diethyl ether) was added dropwise under argon. After addition, the reaction was allowed to warm to room temperature, stirring for ˜1 hr. The reaction was then briefly cooled again before more methylmagnesium bromide (0.5 ml) was added, and the reaction was stirred at room temperature for ˜30 min. After additional monitoring by LCMS, the reaction was cooled again, and a final portion of methylmagnesium bromide (0.5 ml) was added. The reaction was stirred at room temperature for ˜35 min. The reaction was then slowly quenched with MeOH (0.3 ml). The reaction was filtered and concentrated under reduced pressure. Then, sodium iodide (289 mg, 1.925 mmol) was added, followed by acetonitrile (6 ml). Finally, chlorotrimethylsilane (0.25 ml, 1.970 mmol) was added. The reaction was stirred at room temperature overnight. Then, more chlorotrimethylsilane (0.5 ml) was added, and the reaction was stirred at room temperature for ˜3.25 hrs. Stirring was stopped, and then the reaction was filtered and concentrated under reduced pressure. The resulting residue was washed 3× with hexanes, and concentrated under reduced pressure again. The final crude residue was dissolved in DMSO, filtered, and purified by reverse phase chromatography (with NH₄OH modifier) to afford 3-(4-(3-hydroxy-3-methylcyclobutyl)benzyl)quinolin-2(1H)-one as a solid. ¹H NMR (600 MHz, DMSO) δ 11.76 (s, 1H), 7.65 (s, 1H), 7.57 (d, J=7.6 Hz, 1H), 7.44-7.41 (m, 1H), 7.28 (d, J=8.2 Hz, 1H), 7.22-7.16 (m, 4H), 7.13 (t, J=7.2 Hz, 1H), 4.97 (s, 1H), 3.78 (s, 2H), 2.97-2.89 (m, 1H), 2.33-2.27 (m, 2H), 2.09-2.03 (m, 2H), 1.31 (s, 3H). LC/MS (m/z): 320 (M+H)+.

Examples shown in Table 22 below, were prepared according to procedures analogous to those outlined in Example 161 above using the appropriate starting materials, described in the Preparations or Intermediates above, or as obtained from commercial sources.

TABLE 22

| Ex- | | LCMS |
|---|---|---|
| am- | Structure | (m/z) |
| ple | Name | (M + H)+ |
| 162 | 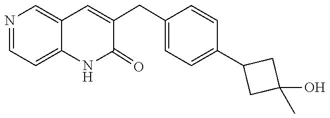 [SMILES: CC1(O)CC(c2ccc(Cc3cc4cnccc4[nH]c3=O)cc2)C1] | 321 |
| | 3-(4-(3-hydroxy-3-methylcyclobutyl)benzyl)- | |
| | 1,6-naphthyridin-2(1H)-one | |

### Example 163

### Preparation of 6-fluoro-3-((2-methyl-2H-indazol-4-yl)methyl)quinolin-2(1H)-one

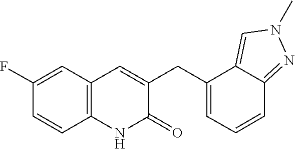 [SMILES: Cn1cc2c(Cc3cc4cc(F)ccc4[nH]c3=O)cccc2n1]

### Step A: (E)-Ethyl 3-(2-methyl-2H-indazol-4-yl)acrylate

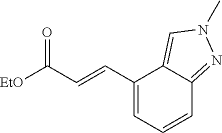 [SMILES: CCOC(=O)/C=C/c1cccc2nn(C)cc12]

A mixture of 4-bromo-2-methyl-2H-indazole (500 mg, 2.4 mmol), ethyl acrylate (0.38 mL, 3.6 mmol), Pd(OAc)₂ (53 mg, 0.24 mmol), tri-o-tolylphosphine (144 mg, 0.47 mmol) and triethylamine (0.99 mL, 7.1 mmol) in DMF (10 mL) was stirred at 100° C. under a nitrogen atmosphere for 6 hours. The reaction mixture cooled to room temperature, quenched with saturated aqueous NH₄Cl (20 mL) and extracted with EtOAc (3×15 mL). The combined organic layers were washed with brine (50 mL), dried over anhydrous sodium sulfate, filtered, and concentrated under reduced pressure to afford (E)-ethyl 3-(2-methyl-2H-indazol-4-yl)acrylate, which was used in the next step without further purification. LC/MS (m/z): 231 (M+H)⁺

### Step B: Ethyl 3-(2-methyl-2H-indazol-4-yl)propanoate

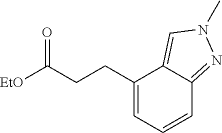 [SMILES: CCOC(=O)CCc1cccc2nn(C)cc12]

Sodium borohydride (238 mg, 6.28 mmol) was added portion-wise to a solution of (E)-ethyl 3-(2-methyl-2H-indazol-4-yl)acrylate (723 mg, 3.14 mmol) and nickel(II) chloride hexahydrate (75 mg, 0.31 mmol) in methanol (15 mL) at 0° C. The reaction mixture was stirred at 0° C. for 1 hour. The reaction mixture was quenched with saturated ammonium chloride (50 mL) and extracted with EtOAc (3×30 mL). The combined organic phases were washed with brine (50 mL), dried over anhydrous sodium sulfate, filtered, and concentrated under reduced pressure. The residue was purified by silica gel column chromatography (EtOAc in hexanes) to afford ethyl 3-(2-methyl-2H-indazol-4-yl)propanoate. ¹H NMR (500 MHz, chloroform-d) δ 7.96 (s, 1H), 7.56 (d, J=8.7 Hz, 1H), 7.22 (dd, J=6.8, 8.6 Hz, 1H), 6.88 (d, J=6.3 Hz, 1H), 4.24 (s, 3H), 4.14 (q, J=7.2 Hz, 2H), 3.19 (t, J=7.8 Hz, 2H), 2.77-2.72 (m, 2H), 1.24 (t, J=7.2 Hz, 3H). LC/MS (m/z): 233 (M+H)⁺

### Step C: 3-(2-Methyl-2H-indazol-4-yl)propanoic acid

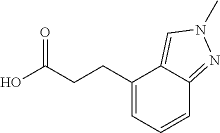

A mixture of ethyl 3-(2-methyl-2H-indazol-4-yl)propanoate (242 mg, 1.04 mmol) and lithium hydroxide hydrate (175 mg, 4.17 mmol) in THF (2 mL) and water (2 mL) was stirred at room temperature for 15 hours. The reaction mixture was diluted with water (10 mL) and then concentrated under reduced pressure to remove the THF. The resulting aqueous mixture was extracted with EtOAc (10 mL). The pH of the aqueous layer was adjusted to 4-5 by the dropwise addition of 12 N HCl, which resulted in the precipitation of solids. The solids were filtered and dried under vacuum to afford 3-(2-methyl-2H-indazol-4-yl)propanoic acid, which was used without further purification. ¹H NMR (500 MHz, DMSO-d₆) δ 12.17 (br s, 1H), 8.43 (s, 1H), 7.41 (d, J=8.7 Hz, 1H), 7.15-7.09 (m, 1H), 6.82 (d, J=6.7 Hz, 1H), 4.15 (s, 3H), 3.05 (br t, J=7.7 Hz, 2H), 2.64 (br t, J=7.7 Hz, 2H). LC/MS (m/z): 205 (M+H)⁺

### Step D: 3-(2-Methyl-2H-indazol-4-yl)propanamide

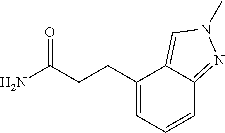

A mixture of 3-(2-methyl-2H-indazol-4-yl)propanoic acid (240 mg, 1.18 mmol), HATU (492 mg, 1.29 mmol), ammonium chloride (126 mg, 2.35 mmol) and TEA (0.490 mL, 3.53 mmol) in DCM (15 mL) was stirred at room temperature for 16 hours. The reaction mixture was concentrated under reduced pressure. The residue was purified by reverse phase HPLC (eluting acetonitrile in water with ammonium bicarbonate modifier) to afford 3-(2-methyl-2H-indazol-4-yl)propanamide. ¹H NMR (500 MHz, DMSO-d₆) δ 8.40 (s, 1H), 7.39 (d, J=8.7 Hz, 1H), 7.31 (br s, 1H), 7.15-7.09 (m, 1H), 6.82-6.76 (m, 2H), 4.15 (s, 3H), 3.02 (t, J=7.8 Hz, 2H), 2.47-2.43 (m, 2H). LC/MS (m/z): 204 (M+H)⁺

### Step E: 6-Fluoro-3-((2-methyl-2H-indazol-4-yl)methyl)quinolin-2(1H)-one

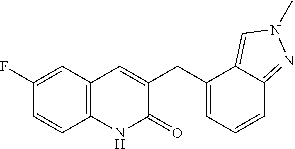 [SMILES: Cn1cc2c(Cc3cc4cc(F)ccc4[nH]c3=O)cccc2n1]

A mixture of 2-bromo-5-fluorobenzaldehyde (30 mg, 0.15 mmol), 3-(2-methyl-2H-indazol-4-yl)propanamide (33 mg, 0.16 mmol), Pd₂(dba)₃ (7 mg, 7 μmol), Xantphos (26 mg, 0.044 mmol) and Cs₂CO₃ (106 mg, 0.325 mmol) in toluene (2 mL) was stirred under a nitrogen atmosphere and heated to 110° C. for 18 hours. The reaction mixture was quenched with saturated aqueous NH₄Cl (15 mL) and extracted with EtOAc (3×15 mL). The combined organic layers were washed with brine (50 mL), dried over anhydrous sodium sulfate, filtered, and concentrated under reduced pressure. The residue was dissolved in toluene (3 mL) and sodium tert-butoxide (43 mg, 0.44 mmol) was added to the mixture. The reaction mixture was stirred and heated to 40° C. for 12 hours. The reaction mixture was then quenched with saturated aqueous NH₄Cl (20 mL) and extracted with EtOAc (3×15 mL). The combined organic phases were washed with brine (50 mL), dried over anhydrous sodium sulfate, filtered, and concentrated under reduced pressure. The residue was purified by reverse phase HPLC (eluting acetonitrile in water with ammonium bicarbonate modifier) to afford 6-fluoro-3-((2-methyl-2H-indazol-4-yl)methyl)quinolin-2(1H)-one. ¹H NMR (500 MHz, methanol-d₄) δ 8.22 (s, 1H), 7.56 (s, 1H), 7.49 (d, J=8.7 Hz, 1H), 7.37-7.31 (m, 1H), 7.29-7.22 (m, 3H), 6.99 (d, J=6.9 Hz, 1H), 4.18-4.16 (m, 5H). LC/MS (m/z): 308 (M+H)⁺

### Example 164

### Preparation 3-Benzyl-4-methylquinolin-2(1H)-one

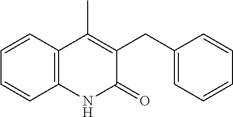 [SMILES: Cc1c(Cc2ccccc2)c(=O)[nH]c2ccccc12]

1-(2-bromophenyl)ethanone (133 mg, 670 μmol), Cs₂CO₃ (655 mg, 2.01 mmol) and methanesulfonato[9,9-dimethyl-4,5-bis(diphenylphosphino)xanthene][2′-amino-1,1′-biphenyl]palladium(ii) dichloromethane adduct (XantPhos Pd G3) (69 mg, 67 μmol) was added to a stirred solution of 3-phenylpropanamide (100 mg, 670 μmol) in 1,4-dioxane (1 mL). The reaction mixture was stirred and heated to 90° C. for 12 hours under a nitrogen atmosphere. The reaction mixture was cooled to room temperature, quenched with water (5 mL), and extracted with EtOAc (3×5 mL). The combined organic layers were washed with brine (10 mL), dried over anhydrous sodium sulfate, filtered, and concentrated under reduced pressure. The residue was purified by reverse phase HPLC (eluting acetonitrile in water, with TFA modifier) to afford 3-benzyl-4-methylquinolin-2(1H)-one. ¹H NMR (400 MHz, methanol-d₄) δ 7.83 (d, J=8.1 Hz, 1H), 7.55-7.47 (m, 1H), 7.35 (d, J=8.1 Hz, 1H), 7.31-7.18 (m, 5H), 7.16-7.10 (m, 1H), 4.16 (s, 2H), 2.50 (s, 3H). LC/MS (m/z): 250 (M+H)⁺

### Example 165

### Preparation of N-(2-methyl-4-((2-oxo-1,2-dihydroquinolin-3-yl)methyl)benzyl)methanesulfonamide

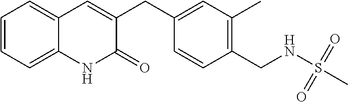 [SMILES: Cc1cc(Cc2cc3ccccc3[nH]c2=O)ccc1CNS(C)(=O)=O]

### Step A: N-(4-bromo-2-methylbenzyl)methanesulfonamide

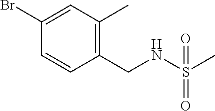

Methanesulfonyl chloride (0.110 ml, 1.42 mmol) in DCM (4.50 ml) was added to a 20 mL vial containing (4-bromo-2-methylphenyl) methanamine (0.270 g, 1.35 mmol), followed by triethylamine (0.565 ml, 4.05 mmol) at 0° C. The reaction mixture was stirred at room temperature for 0.5 h. The mixture was quenched with sat. NaHCO₃aq. and extracted with DCM. The combined organics were concentrated and used directly for the next step. LC/MS (m/z): 300 (M+Na)+

### Step B: N-(2-methyl-4-(4,4,5,5-tetramethyl-1,3,2-dioxaborolan-2-yl)benzyl)methanesulfonamide

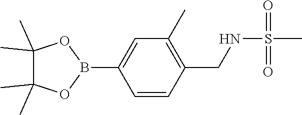

Bis(pinacolato)diboron (44.0 mg, 0.173 mmol), tert-butyl (3-(chloromethyl)quinolin-2-yl) carbonate (39.2 mg, 0.133 mmol) and potassium acetate (39.2 mg, 0.400 mmol) were added to a 0.5-2 mL microwave vial containing N-(4-bromo-2-methylbenzyl)methanesulfonamide (37.0 mg, 0.133 mmol), followed by [1,1′-bis(diphenylphosphino)ferrocene] dichloropalladium (II) (9.8 mg, 0.013 mmol) in 1,4-dioxane (0.7 mL). The reaction mixture was purged with argon and heated in a microwave reactor at 125° C. for 7 min. The mixture was quenched with water and extracted with DCM. The organic layer was dried, filtered, and concentrated. The residue was dissolved in 2 mL of a MeOH/DCM mixture and passed through a small Si-thiol plug. The filtrate was concentrated and used for next step directly. LC/MS (m/z): 348.1 (M+Na)+

### Step C: N-(2-methyl-4-((2-oxo-1,2-dihydroquinolin-3-yl)methyl)benzyl)methanesulfonamide

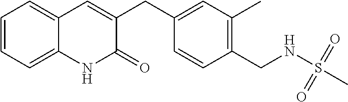 [SMILES: Cc1cc(Cc2cc3ccccc3[nH]c2=O)ccc1CNS(C)(=O)=O]

Bis(triphenylphosphine)palladium (II) dichloride (9.4 mg, 0.013 mmol) and potassium fluoride (38.7 mg, 0.666 mmol) in 0.8 mL of MeOH was added to a 0.5-2 mL microwave vial containing the crude product from step B. The reaction mixture was purged with argon and heated in a microwave reactor at 125° C. for 6 min. The reaction mixture was cooled, diluted with water, extracted with DCM, and the organic layer was concentrated. The mixture was diluted with MeOH and DMSO, passed through Si-thiol plug and purified by reversed phase HPLC (MeCN/water w/0.1% TFA as modifier) to afford N-(2-methyl-4-((2-oxo-1,2-dihydroquinolin-3-yl)methyl)benzyl) methane sulfonamide. ¹H NMR (600 MHz, DMSO-d₆) δ 11.78 (s, 1H), 7.66 (s, 1H), 7.57 (d, J=7.7 Hz, 1H), 7.44 (t, J=7.7 Hz, 1H), 7.33 (t, J=6.1 Hz, 1H), 7.29 (d, J=8.2 Hz, 1H), 7.25-7.22 (m, 1H), 7.14 (t, J=7.5 Hz, 1H), 7.10 (s, 2H), 4.10 (d, J=6.1 Hz, 2H), 3.78 (s, 2H), 2.88 (s, 3H), 2.28 (s, 3H). LC/MS (m/z): 357 (M+H)+

Examples shown in Table 23 below, were prepared according to procedures analogous to those outlined in Example 165 above using the appropriate starting materials, described in the Preparations or Intermediates above, or as obtained from commercial sources.

TABLE 23

| Ex- | Structure | LCMS (m/z) |
|---|---|---|
| ample | Name | (M + H)+ |
| 166 | 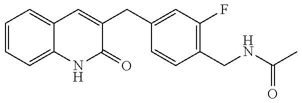 [SMILES: CC(=O)NCc1ccc(Cc2cc3ccccc3[nH]c2=O)cc1F] | 325 |
| | N-(2-fluoro-4-((2-oxo-1,2-dihydroquinolin-3- | |
| | yl)methyl)benzyl)acetamide | |
| 167 | 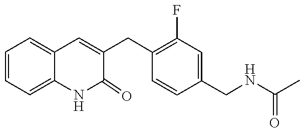 [SMILES: CC(=O)NCc1ccc(Cc2cc3ccccc3[nH]c2=O)c(F)c1] | 325 |
| | N-(3-fluoro-4-((2-oxo-1,2-dihydroquinolin-3- | |
| | yl)methyl)benzyl)acetamide | |
| 168 | 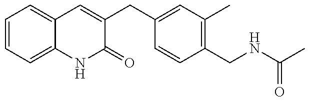 [SMILES: CC(=O)NCc1ccc(Cc2cc3ccccc3[nH]c2=O)cc1C] | 321 |
| | N-(2-methyl-4-((2-oxo-1,2-dihydroquinolin-3- | |
| | yl)methyl)benzyl)acetamide | |

### Example 169

### Preparation of 4-Methyl-6-((2-oxo-1,2-dihydroquinolin-3-yl)methyl)-2H-benzo[b][1,4]oxazin-3(4H)-one

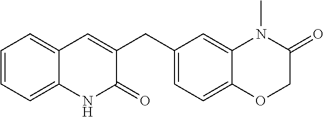 [SMILES: CN1C(=O)COc2ccc(Cc3cc4ccccc4[nH]c3=O)cc21]

### Step A: 4-Methyl-6-(4,4,5,5-tetramethyl-1,3,2-dioxaborolan-2-yl)-2H-benzo[b][1,4]oxazin-3(4H)-one

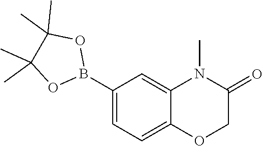 [SMILES: CN1C(=O)COc2ccc(B3OC(C)(C)C(C)(C)O3)cc21]

Potassium acetate (81 mg, 0.83 mmol) and Pd(dppf)Cl₂ (30 mg, 0.041 mmol) were added to a mixture of 6-bromo-4-methyl-2H-benzo[b][1,4]oxazin-3(4H)-one (100 mg, 0.413 mmol) and 4,4,4′,4′,5,5,5′,5′-octamethyl-2,2′-bi(1,3,2-dioxaborolane) (126 mg, 0.496 mmol) in 1,4-dioxane (5 mL) at room temperature. The resulting mixture was stirred and heated to 100° C. for 15 hours. The reaction mixture was concentrated under reduced pressure. The residue was purified by silica gel chromatography (eluting ethyl acetate in petroleum ether) to afford 4-methyl-6-(4,4,5,5-tetramethyl-1,3,2-dioxaborolan-2-yl)-2H-benzo[b][1,4]oxazin-3(4H)-one. ¹H NMR (500 MHz, chloroform-d) (7.49 (d, J=7.9 Hz, 1H), 7.40 (s, 1H), 6.98 (d, J=7.9 Hz, 1H), 4.64 (s, 2H), 3.42 (s, 3H), 1.35 (s, 12H). LC/MS (m/z): 250 (M+H)⁺

### Step B: 4-Methyl-6-((2-oxo-1,2-dihydroquinolin-3-yl)methyl)-2H-benzo[b][1,4]oxazin-3(4H)-one

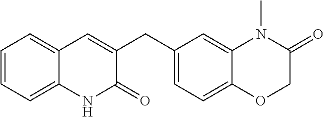 [SMILES: CN1C(=O)COc2ccc(Cc3cc4ccccc4[nH]c3=O)cc21]

Potassium phosphate (tribasic) (110 mg, 0.519 mmol) and Pd(dppf)Cl₂ (2 mg, 3 μmol) were added to a mixture of 4-methyl-6-(4,4,5,5-tetramethyl-1,3,2-dioxaborolan-2-yl)-2H-benzo[b][1,4]oxazin-3(4H)-one (50 mg, 0.17 mmol) and 3-(chloromethyl)quinolin-2(1H)-one (37 mg, 0.19 mmol) in 1,4-dioxane (3 mL) and water (0.6 mL) at room temperature. The reaction mixture was stirred and heated at 80° C. for 15 hours. The reaction mixture was cooled to room temperature and then concentrated under reduced pressure. The residue was purified by reverse phase HPLC (eluting acetonitrile in water, with ammonium bicarbonate modifier) to afford 4-methyl-6-((2-oxo-1,2-dihydroquinolin-3-yl)methyl)-2H-benzo[b][1,4]oxazin-3(4H)-one. ¹H NMR (500 MHz, chloroform-d) (11.80 (br s, 1H), 7.55-7.42 (m, 3H), 7.33 (d, J=8.1 Hz, 1H), 7.20 (t, J=7.5 Hz, 1H), 7.02 (s, 1H), 7.01-6.97 (m, 1H), 6.97-6.93 (m, 1H), 4.60 (s, 2H), 4.00 (s, 2H), 3.38-3.32 (m, 3H). LC/MS (m/z): 321 (M+H)⁺

### Example 170

### Preparation of 8-Chloro-6-((2-oxo-1,2-dihydroquinolin-3-yl)methyl)-2H-benzo[b][1,4]oxazin-3(4H)-one

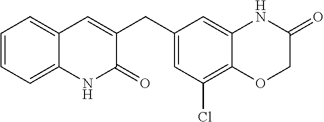 [SMILES: O=C1COc2c(Cl)cc(Cc3cc4ccccc4[nH]c3=O)cc2N1]

### Step A: 8-Chloro-6-(4,4,5,5-tetramethyl-1,3,2-dioxaborolan-2-yl)-2H-benzo[b][1,4]oxazin-3(4H)-one

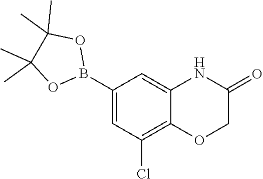 [SMILES: CC1(C)OB(c2cc(Cl)c3c(c2)NC(=O)CO3)OC1(C)C]

A mixture of 6-bromo-8-chloro-2H-benzo[b][1,4]oxazin-3(4H)-one (100 mg, 0.381 mmol), 4,4,4′,4′,5,5,5′,5′-octamethyl-2,2′-bi(1,3,2-dioxaborolane) (106 mg, 0.419 mmol), Pd(dppf)Cl₂ (17 mg, 0.023 mmol), potassium acetate (112 mg, 1.14 mmol) in 1,4-dioxane (2 mL) was sparged with nitrogen at 15° C. The reaction mixture was then stirred and heated to 40° C. for 16 hours. After cooling to room temperature, the reaction mixture was filtered, and the filtrate was concentrated under reduced pressure. The residue was purified by silica gel chromatography (ethyl acetate in petroleum ether) to afford 8-chloro-6-(4,4,5,5-tetramethyl-1,3,2-dioxaborolan-2-yl)-2H-benzo[b][1,4]oxazin-3(4H)-one. ¹H NMR (500 MHz, chloroform-d) δ 8.11 (br s, 1H) 7.51 (s, 1H) 7.11 (s, 1H) 4.75 (s, 2H) 1.33 (s, 12H). LC/MS (m/z): 310 (M+H)⁺

### Step B: 8-Chloro-6-((2-methoxyquinolin-3-yl)methyl)-2H-benzo[b][1,4]oxazin-3(4H)-one

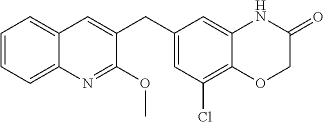 [SMILES: COc1nc2ccccc2cc1Cc1cc(Cl)c2c(c1)NC(=O)CO2]

A mixture of 8-chloro-6-(4,4,5,5-tetramethyl-1,3,2-dioxaborolan-2-yl)-2H-benzo[b][1,4]oxazin-3(4H)-one (80 mg, 0.26 mmol), 3-(chloromethyl)-2-methoxyquinoline (59 mg, 0.28 mmol), K₃PO₄ (110 mg, 0.517 mmol), Pd(dppf)Cl₂ (8 mg, 11 μmol) in 1,4-dioxane (1 mL) and water (0.2 mL) was sparged with nitrogen for 2 minutes. The reaction mixture was stirred and heated to 80° C. for 16 hours. After cooling to room temperature, the reaction mixture was filtered and the filtrate was concentrated under reduced pressure. The residue was purified by silica gel chromatography (ethyl acetate in petroleum ether) to afford 8-chloro-6-((2-methoxyquinolin-3-yl)methyl)-2H-benzo[b][1,4]oxazin-3(4H)-one. ¹H NMR (500 MHz, methanol-d₄) δ 7.84 (s, 1H) 7.79 (d, J=8.54 Hz, 1H) 7.71 (d, J=8.24 Hz, 1H) 7.58 (td, J=7.71, 1.37 Hz, 1H) 7.33-7.40 (m, 1H) 7.11 (d, J=7.78 Hz, 1H) 6.98 (d, J=7.63 Hz, 1H) 6.78 (s, 1H) 5.29 (s, 2H) 4.05 (s, 3H) 4.03 (s, 2H). LC/MS (m/z): 355 (M+H)⁺

### Step C: 8-Chloro-6-((2-oxo-1,2-dihydroquinolin-3-yl)methyl)-2H-benzo[b][1,4]oxazin-3(4H)-one

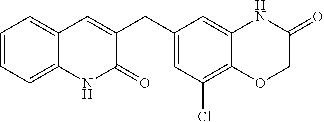 [SMILES: O=C1COc2c(Cl)cc(Cc3cc4ccccc4[nH]c3=O)cc2N1]

HCl (12M in water, 0.5 mL, 6.00 mmol) was added to a mixture of 8-chloro-6-((2-methoxyquinolin-3-yl)methyl)-2H-benzo[b][1,4]oxazin-3(4H)-one (65 mg, 0.18 mmol) in THF (1 mL) at 15° C. The reaction mixture was stirred and heated to 80° C. for 16 hours. The reaction mixture was cooled to room temperature and concentrated under reduced pressure. The residue was purified by reverse phase HPLC (eluting acetonitrile in water, with TFA modifier) to afford 8-chloro-6-((2-oxo-1,2-dihydroquinolin-3-yl)methyl)-2H-benzo[b][1,4]oxazin-3(4H)-one. ¹H NMR (400 MHz, DMSO-d₆) δ 11.78 (s, 1H), 10.76 (br s, 1H), 7.72 (s, 1H), 7.58 (d, J=7.6 Hz, 1H), 7.42-7.40 (m, 1H), 7.26 (d, J=8.0 Hz, 1H), 7.16-7.07 (m, 1H), 6.96 (d, J=2.0 Hz, 1H), 6.74 (d, J=2.0 Hz, 1H), 4.60 (s, 2H), 3.70 (s, 2H). LC/MS (m/z): 341 (M+H)⁺

### Example 171

### Preparation of 5-Fluoro-6-((2-oxo-1,2-dihydroquinolin-3-yl)methyl)-2H-benzo[b][1,4]oxazin-3(4H)-one

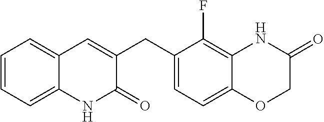 [SMILES: O=C1COc2ccc(Cc3cc4ccccc4[nH]c3=O)c(F)c2N1]

### Step A: 5-Fluoro-6-(4,4,5,5-tetramethyl-1,3,2-dioxaborolan-2-yl)-2H-benzo[b][1,4]oxazin-3(4H)-one

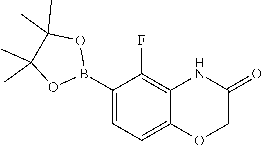 [SMILES: CC1(C)OB(c2ccc3c(c2F)NC(=O)CO3)OC1(C)C]

A mixture of 6-bromo-5-fluoro-2H-benzo[b][1,4]oxazin-3(4H)-one (50 mg, 0.20 mmol), 4,4,4′,4′,5,5,5′,5′-octamethyl-2,2′-bi(1,3,2-dioxaborolane) (77 mg, 0.31 mmol), Pd(dppf)Cl₂ (17 mg, 0.023 mmol), potassium acetate (65 mg, 0.66 mmol) in 1,4-dioxane (2 mL) was sparged with nitrogen at room temperature for 2 minutes. The reaction mixture was stirred and heated to 80° C. for 16 hours. After cooling to room temperature, the reaction mixture was filtered, and the filtrate was concentrated under reduced pressure. The residue was purified by silica gel chromatography (ethyl acetate in petroleum ether) to afford 5-fluoro-6-(4,4,5,5-tetramethyl-1,3,2-dioxaborolan-2-yl)-2H-benzo[b][1,4]oxazin-3(4H)-one. ¹H NMR (500 MHz, chloroform-d) δ 8.18 (br s, 1H) 7.33 (t, J=7.32 Hz, 1H) 6.77 (d, J=8.24 Hz, 1H) 4.66 (s, 2H) 1.34 (s, 12H). LC/MS (m/z): 294 (M+H)⁺

### Step B: 5-Fluoro-6-((2-methoxyquinolin-3-yl)methyl)-2H-benzo[b][1,4]oxazin-3(4H)-one

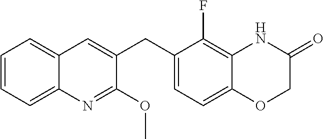 [SMILES: COc1nc2ccccc2cc1Cc1ccc2c(c1F)NC(=O)CO2]

A mixture of 5-fluoro-6-(4,4,5,5-tetramethyl-1,3,2-dioxaborolan-2-yl)-2H-benzo[b][1,4]oxazin-3(4H)-one (30 mg, 0.10 mmol), 3-(chloromethyl)-2-methoxyquinoline (33 mg, 0.16 mmol), K₃PO₄ (67 mg, 0.32 mmol), Pd(dppf)Cl₂ (8 mg, 10 μmol) in 1,4-dioxane (1 mL) and water (0.2 mL) at room temperature was sparged with nitrogen for 2 minutes. The reaction mixture was stirred and heated to 80° C. for 16 hours. After cooling to room temperature, the reaction mixture was filtered, and the filtrate was concentrated under reduced pressure. The residue was purified by silica gel chromatography (ethyl acetate in petroleum ether) to afford 5-fluoro-6-((2-methoxyquinolin-3-yl)methyl)-2H-benzo[b][1,4]oxazin-3(4H)-one. LC/MS (m/z): 339 (M+H)⁺

### Step C: 5-Fluoro-6-((2-oxo-1,2-dihydroquinolin-3-yl)methyl)-2H-benzo[b][1,4]oxazin-3(4H)-one

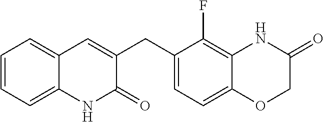 [SMILES: O=C1COc2ccc(Cc3cc4ccccc4[nH]c3=O)c(F)c2N1]

HCl (37% in water, 0.5 mL) was added to a mixture of 5-fluoro-6-((2-methoxyquinolin-3-yl)methyl)-2H-benzo[b][1,4]oxazin-3(4H)-one (25 mg, 0.074 mmol) in THF (1 mL) at room temperature. The reaction mixture was stirred and heated to 80° C. for 16 hours. The reaction mixture was cooled to room temperature and concentrated under reduced pressure. The residue was purified by reverse phase HPLC (eluting acetonitrile in water, with TFA modifier) to afford 5-fluoro-6-((2-oxo-1,2-dihydroquinolin-3-yl)methyl)-2H-benzo[b][1,4]oxazin-3(4H)-one. ¹H NMR (500 MHz, DMSO-d₆) δ=11.85 (s, 1H), 10.89 (s, 1H), 7.60 (d, J=7.6 Hz, 1H), 7.57 (s, 1H), 7.50-7.42 (m, 1H), 7.30 (d, J=8.2 Hz, 1H), 7.15 (t, J=7.2 Hz, 1H), 6.90-6.84 (m, 1H), 6.78 (d, J=8.4 Hz, 1H), 4.60 (s, 2H), 3.79 (s, 2H). LC/MS (m/z): 325 (M+H)⁺

### Example 172

### Preparation of 3-benzyl-6-methoxyquinolin-2(1H)-one

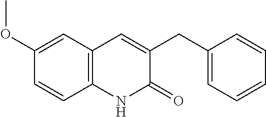 [SMILES: COc1ccc2[nH]c(=O)c(Cc3ccccc3)cc2c1]

Cesium carbonate (328 mg, 1.01 mmol) and methanesulfonato[9,9-dimethyl-4,5-bis(diphenylphosphino)xanthene][2′-amino-1,1′-biphenyl]palladium(ii) dichloromethane adduct (Xantphos-Pd G3, 35 mg, 0.034 mmol) were added to a mixture of 3-phenylpropanamide (50 mg, 0.34 mmol) and 2-bromo-5-methoxybenzaldehyde (72 mg, 0.34 mmol) in 1,4-dioxane (1.2 mL) at 10° C. The reaction mixture was sparged with a stream of nitrogen for 1 minute. The resulting mixture was stirred and heated to 95° C. for 15 hours. The reaction mixture was concentrated under reduced pressure. The residue was purified by reverse phase HPLC (eluting acetonitrile in water, with TFA modifier) to afford 3-benzyl-6-methoxyquinolin-2(1H)-one. ¹H NMR (500 MHz, methanol-d₄) (7.59 (s, 1H), 7.35-7.31 (m, 4H), 7.30-7.27 (m, 1H), 7.26-7.21 (m, 1H), 7.14 (dd, J=2.7, 8.9 Hz, 1H), 7.07 (d, J=2.4 Hz, 1H), 3.94 (s, 2H), 3.83 (s, 3H). LC/MS (m/z): 266 (M+H)⁺

### Example 173

### Preparation of 3-Benzyl-6-(2-hydroxypropan-2-yl)quinolin-2(1H)-one

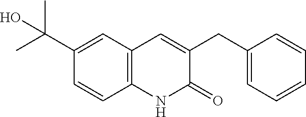 [SMILES: CC(C)(O)c1ccc2[nH]c(=O)c(Cc3ccccc3)cc2c1]

### Step A: 3-Benzyl-6-bromo-2-methoxyquinoline

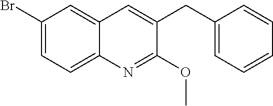 [SMILES: COc1nc2ccc(Br)cc2cc1Cc1ccccc1]

A mixture of 3-benzyl-6-bromo-2-chloroquinoline (300 mg, 0.902 mmol) and sodium methoxide (54 mg, 1.0 mmol) in MeOH (10 mL) was stirred and heated to 80° C. for 16 hours. The reaction mixture was cooled to room temperature and quenched with water (50 mL). The mixture was filtered and the solids were washed with additional water (3×20 mL). The isolated solids were dried under vacuum to afford 3-benzyl-6-bromo-2-methoxyquinoline, which was used in the next step without purification. ¹H NMR (500 MHz, chloroform-d) (7.75 (d, J=2.0 Hz, 1H), 7.71-7.66 (m, 1H), 7.64-7.59 (m, 1H), 7.48 (s, 1H), 7.37-7.29 (m, 2H), 7.26-7.21 (m, 3H), 4.08 (s, 3H), 4.03 (s, 2H). LC/MS (m/z): 328, 330 (M+H)⁺

### Step B: 3-benzyl-6-bromoquinolin-2(1H)-one

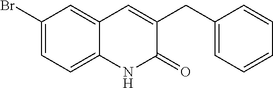 [SMILES: O=c1[nH]c2ccc(Br)cc2cc1Cc1ccccc1]

HCl (37% in water, 5 mL) was added to a solution of 3-benzyl-6-bromo-2-methoxyquinoline (110 mg, 0.335 mmol) in THF (10 mL), at room temperature. The reaction mixture was stirred and heated to 80° C. for 16 hours. The reaction mixture was cooled to room temperature, filtered, and the solids were washed with water (2×10 mL) and ethanol (3×5 mL). The isolated solids were dried under vacuum to give 3-benzyl-6-bromoquinolin-2(1H)-one, which was used in the next step without purification. LC/MS (m/z): 314, 316 (M+H)⁺

### Step C: 3-Benzyl-6-(2-hydroxypropan-2-yl)quinolin-2(1H)-one

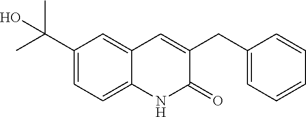 [SMILES: CC(C)(O)c1ccc2[nH]c(=O)c(Cc3ccccc3)cc2c1]

n-BuLi (2.5M in hexane, 0.24 mL, 0.60 mmol) was added to a mixture of 3-benzyl-6-bromoquinolin-2(1H)-one (75 mg, 0.24 mmol) in THF (1.0 mL) at −70° C. over a period of 1 minute. The reaction mixture was stirred at −70° C. for 1 hour, and then acetone (0.020 mL, 0.27 mmol) was added to the mixture. The resulting mixture was stirred for an additional 1 hour at −70° C. The reaction mixture was warmed to room temperature, quenched with saturated aqueous NH₄Cl (0.5 mL), and concentrated under reduced pressure. The residue was diluted with methanol (3 mL) and purified by reverse phase HPLC (eluting acetonitrile in water, with ammonium bicarbonate modifier) to afford 3-benzyl-6-(2-hydroxypropan-2-yl)quinolin-2(1H)-one. ¹H NMR (500 MHz, methanol-d₄) δ 7.69-7.61 (m, 3H), 7.37-7.29 (m, 5H), 7.28-7.20 (m, 1H), 3.94 (s, 2H), 1.57 (s, 6H). LC/MS (m/z): 294 (M+H)⁺

### Example 174

### Preparation of 3-Benzyl-2-oxo-1,2-dihydroquinoline-6-carbonitrile

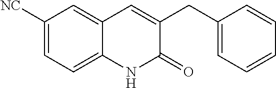 [SMILES: N#Cc1ccc2[nH]c(=O)c(Cc3ccccc3)cc2c1]

### Step A: 3-Benzyl-2-methoxyquinoline-6-carbonitrile

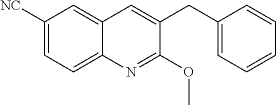 [SMILES: COc1nc2ccc(C#N)cc2cc1Cc1ccccc1]

A mixture of 3-benzyl-6-bromo-2-methoxyquinoline (100 mg, 0.305 mmol), copper (I) cyanide (55 mg, 0.61 mmol) and Pd(PPh₃)₄ (35 mg, 0.030 mmol) in DMF (3 mL) was stirred and heated to 120° C. for 16 hours. The reaction mixture was cooled to room temperature and filtered. The mixture purified by reverse phase HPLC (eluting acetonitrile in water, with TFA modifier) to afford 3-benzyl-2-methoxyquinoline-6-carbonitrile. ¹H NMR (500 MHz, chloroform-d) (7.98 (d, J=1.5 Hz, 1H), 7.87 (d, J=8.5 Hz, 1H), 7.71 (dd, J=2.0, 8.5 Hz, 1H), 7.59 (s, 1H), 7.37-7.31 (m, 2H), 7.27 (br s, 1H), 7.25-7.22 (m, 2H), 4.12 (s, 3H), 4.05 (s, 2H). LC/MS (m/z): 275 (M+H)⁺

### Step B: 3-benzyl-2-oxo-1,2-dihydroquinoline-6-carbonitrile

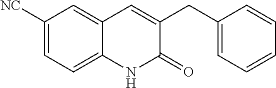 [SMILES: N#Cc1ccc2[nH]c(=O)c(Cc3ccccc3)cc2c1]

A mixture of 3-benzyl-2-methoxyquinoline-6-carbonitrile (60 mg, 0.22 mmol) and HCl (37% in water, 1 mL) in THF (2 mL) was stirred and heated to 80° C. for 2 hours. The reaction mixture was cooled to room temperature, quenched with saturated aqueous NaHCO₃ (5 mL) and extracted with DCM (3×5 mL). The organic layers were combined, washed with brine (10 mL), dried over anhydrous sodium sulfate, filtered, and concentrated under reduced pressure. The residue was purified by reverse phase HPLC (eluting acetonitrile in water, with TFA modifier) to afford 3-benzyl-2-oxo-1,2-dihydroquinoline-6-carbonitrile. ¹H NMR (500 MHz, methanol-d₄) (7.79 (s, 1H), 7.66 (dd, J=1.5, 8.5 Hz, 1H), 7.43-7.27 (m, 7H), 4.00 (s, 2H). LC/MS (m/z): 261 (M+H)⁺

### Example 175

### Preparation of 1-Methyl-3-((2-phenylcyclopropyl)methyl)quinolin-2(1H)-one

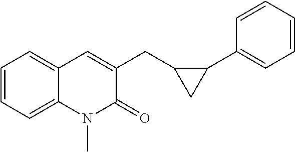 [SMILES: Cn1c(=O)c(CC2CC2c2ccccc2)cc2ccccc21]

### Step A: (E)-1-Methyl-3-(3-phenylallyl)quinolin-2(1H)-one

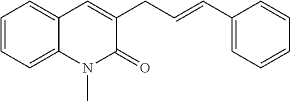 [SMILES: Cn1c(=O)c(C/C=C/c2ccccc2)cc2ccccc21]

A mixture of (E)-styrylboronic acid (103 mg, 0.693 mmol), 3-(chloromethyl)-1-methylquinolin-2(1H)-one (120 mg, 0.578 mmol), Pd(dtbpf)Cl₂ (38 mg, 0.058 mmol) and K₃PO₄ (368 mg, 1.73 mmol) in acetonitrile (10 mL) and water (2 mL) was degassed with nitrogen. The mixture was stirred and heated to 105° C. for 16 hours. After cooling to room temperature, the reaction mixture was extracted with EtOAc (3×20 mL). The combined organic phases were washed with brine (10 mL), dried over anhydrous sodium sulfate, filtered, and concentrated under reduced pressure. The residue was purified by silica gel chromatography (petroleum ether) to afford (E)-1-methyl-3-(3-phenylallyl)quinolin-2(1H)-one. ¹H NMR (400 MHz, methanol-d₄) δ 7.79 (s, 1H), 7.54-7.69 (m, 3H), 7.40 (d, J=7.04 Hz, 2H), 7.25-7.33 (m, 3H), 7.16-7.22 (m, 1H), 6.53-6.60 (m, 1H), 6.42-6.52 (m, 1H), 3.79 (s, 3H), 3.52 (d, J=6.65 Hz, 2H). LC/MS (m/z): 276 (M+H)⁺

### Step B: 1-Methyl-3-((2-phenylcyclopropyl)methyl)quinolin-2(1H)-one

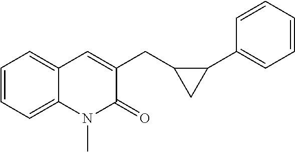 [SMILES: Cn1c(=O)c(CC2CC2c2ccccc2)cc2ccccc21]

1-methyl-1-nitrosourea (449 mg, 4.36 mmol) was added to an Erlenmeyer flask containing Et₂O (30 mL) and 40% aqueous potassium hydroxide (6 mL, 42.8 mmol) at 0° C. The mixture was left to stand for 30 minutes at 0° C., carefully shaking it several times. The organic layer containing the generated diazomethane was decanted and dried over KOH pellets at 0° C. for 1 hour. (E)-1-methyl-3-(3-phenylallyl)quinolin-2(1H)-one (120 mg, 0.436 mmol) and Pd(OAc)₂ (10 mg, 0.045 mmol) were dissolved in a separate mixture of Et₂O (10 mL) and MeOH (2 mL), and the mixture was cooled to 0° C. The solution of diazomethane in Et₂O was then added dropwise to the reaction mixture at 0° C. After the addition was finished, the reaction was stirred at 0° C. for 30 min. AcOH (3 mL) was added to the reaction mixture to quench unreacted diazomethane. After stirring for another 15 min, the reaction mixture was washed with saturated aqueous NaHCO₃ (20 mL) and brine (20 mL). The organic layer was separated, dried over sodium sulfate, filtered, and concentrated under reduced pressure. The residue was purified by reverse phase HPLC (eluting acetonitrile in water, with ammonium bicarbonate modifier) to afford 1-methyl-3-((2-phenylcyclopropyl)methyl)quinolin-2(1H)-one. ¹H NMR (500 MHz, methanol-d₄) (7.79 (s, 1H), 7.59-7.49 (m, 3H), 7.27-7.19 (m, 3H), 7.12-7.02 (m, 3H), 3.73 (s, 3H), 2.75-2.61 (m, 2H), 1.81-1.77 (m, 1H), 1.43-1.36 (m, 1H), 1.05-1.01 (m, 1H), 0.97-0.92 (m, 1H). LC/MS (m/z): 290 (M+H)⁺

### Example 176

### Preparation of 8-Fluoro-6-((2-oxo-1,2-dihydroquinolin-3-yl)methyl)-2H-benzo[b][1,4]oxazin-3(4H)-one

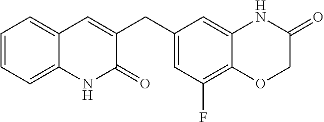 [SMILES: O=C1COc2c(F)cc(Cc3cc4ccccc4[nH]c3=O)cc2N1]

### Step A: 8-Fluoro-6-(4,4,5,5-tetramethyl-1,3,2-dioxaborolan-2-yl)-2H-benzo[b][1,4]oxazin-3(4H)-one

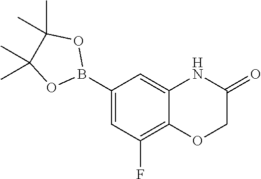 [SMILES: CC1(C)OB(c2cc(F)c3c(c2)NC(=O)CO3)OC1(C)C]

A mixture of 6-bromo-8-fluoro-2H-benzo[b][1,4]oxazin-3(4H)-one (400 mg, 1.63 mmol), 4,4,4′,4′,5,5,5′,5′-octamethyl-2,2′-bi(1,3,2-dioxaborolane) (619 mg, 2.44 mmol), potassium acetate (479 mg, 4.88 mmol) and Pd(dtbpf)Cl₂ (38 mg, 0.052 mmol) in 1,4-dioxane (10 mL) was sparged with nitrogen. The reaction mixture was stirred and heated to 80° C. for 16 hours. After cooling to room temperature, the reaction mixture was concentrated under reduced pressure and the residue was purified by silica gel chromatography (ethyl acetate in petroleum ether) to afford 8-fluoro-6-(4,4,5,5-tetramethyl-1,3,2-dioxaborolan-2-yl)-2H-benzo[b][1,4]oxazin-3(4H)-one. LC/MS (m/z): 294 (M+H)⁺

### Step B: 8-Fluoro-6-((2-methoxyquinolin-3-yl)methyl)-2H-benzo[b][1,4]oxazin-3(4H)-one

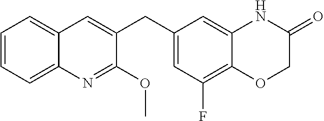 [SMILES: COc1nc2ccccc2cc1Cc1cc(F)c2c(c1)NC(=O)CO2]

A mixture of 8-fluoro-6-(4,4,5,5-tetramethyl-1,3,2-dioxaborolan-2-yl)-2H-benzo[b][1,4]oxazin-3(4H)-one (300 mg, 1.02 mmol), 3-(bromomethyl)-2-methoxyquinoline (310 mg, 1.23 mmol), K₃PO₄ (652 mg, 3.07 mmol), and Pd(dppf)Cl₂ (75 mg, 0.10 mmol) in 1,4-dioxane (10 mL) and water (1 mL) was sparged with nitrogen for 2 minutes at room temperature. The reaction mixture was stirred and heated to 80° C. for 15 hours. The reaction mixture was concentrated under reduced pressure, and the residue was purified by silica gel chromatography (methanol in dichloromethane) to afford 8-fluoro-6-((2-methoxyquinolin-3-yl)methyl)-2H-benzo[b][1,4]oxazin-3(4H)-one. LC/MS (m/z): 339 (M+H)⁺

### Step C: 8-Fluoro-6-((2-oxo-1,2-dihydroquinolin-3-yl)methyl)-2H-benzo[b][1,4]oxazin-3(4H)-one

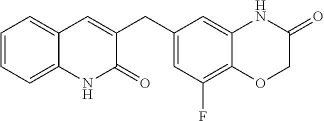 [SMILES: O=C1COc2c(F)cc(Cc3cc4ccccc4[nH]c3=O)cc2N1]

HCl (37% in water, 15 mL) was added to a mixture of 8-fluoro-6-((2-methoxyquinolin-3-yl)methyl)-2H-benzo[b][1,4]oxazin-3(4H)-one (300 mg, 0.887 mmol) in 1,4-dioxane (15 mL). The reaction mixture was stirred and heated to 100° C. for 2 hours. The reaction mixture was cooled to room temperature, concentrated under reduced pressure, and then diluted with MeOH (25 mL). The mixture was stirred for 1 hour at room temperature. The mixture was filtered and the collected solids were dried under vacuum to afford 8-fluoro-6-((2-oxo-1,2-dihydroquinolin-3-yl)methyl)-2H-benzo[b][1,4]oxazin-3(4H)-one. ¹H NMR (400 MHz, DMSO-d₆) (11.78 (s, 1H), 10.76 (s, 1H), 7.70 (s, 1H), 7.57 (d, J=7.6 Hz, 1H), 7.47-7.37 (m, 1H), 7.26 (d, J=8.1 Hz, 1H), 7.17-7.06 (m, 1H), 6.80 (dd, J=1.5, 11.5 Hz, 1H), 6.61 (s, 1H), 4.57 (s, 2H), 3.70 (s, 2H). LC/MS (m/z): 325 (M+H)⁺

### Step B: 7-((2-Methoxyquinolin-3-yl)methyl)-1H-pyrido[2,3-b][1,4]oxazin-2(3H)-one

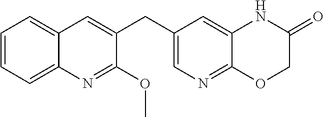 [SMILES: COc1nc2ccccc2cc1Cc1cnc2c(c1)NC(=O)CO2]

A mixture of 7-(4,4,5,5-tetramethyl-1,3,2-dioxaborolan-2-yl)-1H-pyrido[2,3-b][1,4]oxazin-2(3H)-one (720 mg, 2.61 mmol), 3-(chloromethyl)-2-methoxyquinoline (596 mg, 2.87 mmol), K₃PO₄ (1660 mg, 7.82 mmol), and Pd(dppf)Cl₂ (191 mg, 0.261 mmol) in in 1,4-dioxane (1.5 mL) and water (0.3 mL) was sparged with nitrogen for two minutes at room temperature. The reaction mixture was stirred and heated to 80° C. for 15 hours. The reaction mixture was cooled to room temperature and concentrated under reduced pressure. The residue was purified by silica gel chromatography (ethyl acetate in petroleum ether) to afford 7-((2-methoxyquinolin-3-yl)methyl)-1H-pyrido[2,3-b][1,4]oxazin-2(3H)-one. ¹H NMR (400 MHz, chloroform-d) δ 7.89-7.81 (m, 3H), 7.70 (s, 1H), 7.65 (d, J=8.6 Hz, 1H), 7.59 (br t, J=7.0 Hz, 1H), 7.37 (t, J=7.4 Hz, 1H), 6.95 (d, J=2.0 Hz, 1H), 4.80 (s, 2H), 4.08 (s, 3H), 3.98 (s, 2H). LC/MS (m/z): 322 (M+H)⁺

### Step C: 7-((2-Oxo-1,2-dihydroquinolin-3-yl)methyl)-1H-pyrido[2,3-b][1,4]oxazin-2(3H)-one

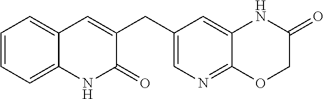 [SMILES: O=C1COc2ncc(Cc3cc4ccccc4[nH]c3=O)cc2N1]

HCl (3.0 M in water, 1.5 mL 4.5 mmol) was added to a mixture of 7-((2-methoxyquinolin-3-yl)methyl)-1H-pyrido[2,3-b][1,4]oxazin-2(3H)-one (340 mg, 1.06 mmol) in 1,4-dioxane (12 mL) and water (4.5 mL) at room temperature under a nitrogen atmosphere. The reaction mixture was stirred and heated to 70° C. for 2 hours. The reaction mixture was cooled to room temperature and concentrated under reduced pressure. The residue was dissolved in DMSO (20 mL) and then triturated with water (20 mL). The suspension was filtered and the collected solids were washed with MeOH (20 mL) and then dried under vacuum to afford 7-((2-oxo-1,2-dihydroquinolin-3-yl)methyl)-1H-pyrido[2,3-b][1,4]oxazin-2(3H)-one. ¹H NMR (400 MHz, DMSO-d₆) δ 11.79 (s, 1H), 10.69 (s, 1H), 7.74-7.71 (m, 2H), 7.57 (d, J=7.8 Hz, 1H), 7.44-7.39 (m, 1H), 7.26 (d, J=8.1 Hz, 1H), 7.14-7.10 (m, 2H), 4.68 (s, 2H), 3.73 (s, 2H). LC/MS (m/z): 308 (M+H)⁺

### Example 177

### Preparation of 8-(hydroxymethyl)-3-(3-(trifluoromethoxy)benzyl)quinolin-2(1H)-one

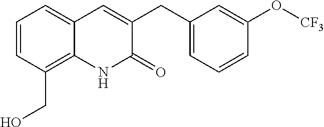 [SMILES: O=c1[nH]c2c(CO)cccc2cc1Cc1cccc(OC(F)(F)F)c1]

### Step A: (8-Bromo-2-chloroquinolin-3-yl)(3-(trifluoromethoxy)phenyl)methanol

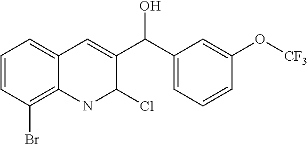 [SMILES: OC(C1=Cc2cccc(Br)c2NC1Cl)c1cccc(OC(F)(F)F)c1]

n-BuLi (2.5M in hexanes, 8.25 mL, 21 mmol) was added to a mixture of diisopropylamine (2.90 mL, 20.6 mmol) in THF (100 mL) at −78° C. over a period of 5 minutes under a nitrogen atmosphere. After stirring for 10 minutes at 0° C., a mixture of 8-bromo-2-chloroquinoline (2.00 g, 8.25 mmol) in THF (2 mL) was added to the reaction mixture at −78° C. The reaction mixture was stirred for another 1 hour at −78° C. A mixture of 3-(trifluoromethoxy)benzaldehyde (2.038 g, 10.72 mmol) in THF (2 mL) was added under a nitrogen atmosphere at −78° C., and the reaction mixture was stirred for another 1 hour. The reaction mixture was quenched with saturated aqueous NH₄Cl (20 mL) and extracted with EtOAc (3×15 mL). The organic layers were combined, washed with brine (15 mL), dried over anhydrous sodium sulfate, filtered, and concentrated under reduced pressure. The residue was purified by silica gel chromatography (ethyl acetate in petroleum ether) to give (8-bromo-2-chloroquinolin-3-yl)(3-(trifluoromethoxy)phenyl)methanol. ¹H NMR (400 MHz, chloroform-d) δ 8.41 (s, 1H), 8.03 (br d, J=7.4 Hz, 1H), 7.84-7.77 (m, 1H), 7.47-7.40 (m, 1H), 7.35 (br d, J=7.8 Hz, 1H), 7.28-7.24 (m, 1H), 7.23-7.11 (m, 2H), 6.27 (s, 1H). LC/MS (m/z): 432, 434 (M+H)⁺

### Step B: 8-Bromo-3-(3-(trifluoromethoxy)benzyl)quinolin-2(1H)-one

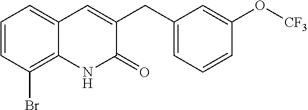 [SMILES: O=c1[nH]c2c(Br)cccc2cc1Cc1cccc(OC(F)(F)F)c1]

A mixture of (8-bromo-2-chloroquinolin-3-yl)(3-(trifluoromethoxy)phenyl)methanol (1.5 g, 3.47 mmol) in TFA (3.0 mL, 39 mmol) was stirred and heated to 100° C. for 12 hours. The reaction mixture was cooled to room temperature. Triethylsilane (2.77 mL, 17.3 mmol) was added, and the reaction mixture was stirred and heated to 80° C. for 16 hours. The reaction mixture was concentrated under reduce pressure. The residue was purified by silica gel chromatography (ethyl acetate in petroleum ether) to afford 8-bromo-3-(3-(trifluoromethoxy)benzyl)quinolin-2(1H)-one. ¹H NMR (400 MHz, DMSO-d₆) δ 10.59 (s, 1H), 7.83 (s, 1H), 7.76 (d, J=7.8 Hz, 1H), 7.64 (d, J=7.0 Hz, 1H), 7.44-7.39 (m, 1H), 7.35-7.30 (m, 2H), 7.19 (br d, J=8.2 Hz, 1H), 7.12 (t, J=7.8 Hz, 1H), 3.91 (s, 2H). LC/MS (m/z): 398, 400 (M+H)⁺

### Step C: 8-(Hydroxymethyl)-3-(3-(trifluoromethoxy)benzyl)quinolin-2(1H)-one

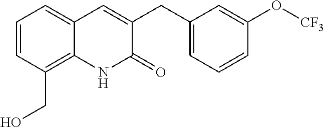 [SMILES: O=c1[nH]c2c(CO)cccc2cc1Cc1cccc(OC(F)(F)F)c1]

A mixture of 8-bromo-3-(3-(trifluoromethoxy)benzyl)quinolin-2(1H)-one (60 mg, 0.15 mmol), (tributylstannyl)methanol (63 mg, 0.20 mmol) and Pd(Ph₃P)₄ (15 mg, 0.013 mmol) in dioxane (2 mL) was degassed and backfilled with nitrogen (three times). The reaction mixture was stirred and heated to 80° C. for 15 hours. The reaction mixture cooled to room temperature, quenched with saturated aqueous KF (10 mL), and stirred for 20 minutes. The mixture was then filtered, and the filtrate was concentrated under reduced pressure. The residue was purified by reverse phase HPLC (eluting acetonitrile in water, with TFA modifier) to afford 8-(hydroxymethyl)-3-(3-(2,2,2-trifluoroethyl)benzyl)quinolin-2(1H)-one. ¹H NMR (400 MHz, DMSO-d₆) (10.67 (s, 1H), 7.80 (s, 1H), 7.50 (d, J=7.6 Hz, 1H), 7.43-7.36 (m, 2H), 7.33-7.28 (m, 2H), 7.16 (br d, J=8.1 Hz, 1H), 7.11 (t, J=7.6 Hz, 1H), 5.49 (t, J=5.4 Hz, 1H), 4.74 (d, J=5.1 Hz, 2H), 3.87 (s, 2H). LC/MS (m/z): 350 (M+H)⁺

### Example 178

### Preparation of 8-(aminomethyl)-3-(3-(trifluoromethoxy)benzyl)quinolin-2(1H)-one

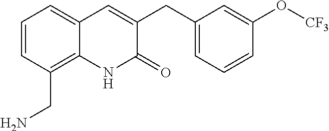 [SMILES: NCc1cccc2cc(Cc3cccc(OC(F)(F)F)c3)c(=O)[nH]c12]

### Step A: tert-butyl ((2-oxo-3-(3-(trifluoromethoxy)benzyl)-1,2-dihydroquinolin-8-yl)methyl)carbamate

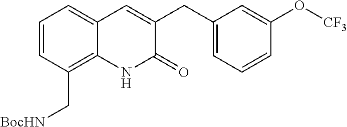 [SMILES: CC(C)(C)OC(=O)NCc1cccc2cc(Cc3cccc(OC(F)(F)F)c3)c(=O)[nH]c12]

A mixture of 8-bromo-3-(3-(trifluoromethoxy)benzyl)quinolin-2(1H)-one (70 mg, 0.18 mmol), potassium (((tert-butoxycarbonyl)amino)methyl)trifluoroborate (48 mg, 0.20 mmol), palladium(II) acetate (8 mg, 0.04 mmol), X-Phos (25 mg, 0.053 mmol), and K₂CO₃ (73 mg, 0.53 mmol) in dioxane (3 mL) and water (0.3 mL) at 20° C. was sparged with nitrogen for 3 minutes. The reaction mixture was stirred and heated to 110° C. for 12 hours. After cooling to room temperature, the reaction mixture was quenched with brine (15 mL) and extracted with EtOAc (3×15 mL). The organic phases were combined, dried over anhydrous sodium sulfate, filtered, and concentrated under reduced pressure. The residue was purified by reverse phase HPLC (eluting acetonitrile in water, with TFA modifier) to afford tert-butyl ((2-oxo-3-(3-(trifluoromethoxy)benzyl)-1,2-dihydroquinolin-8-yl)methyl)carbamate. LC/MS (m/z): 449 (M+H)⁺

### Step B: 8-(Aminomethyl)-3-(3-(trifluoromethoxy)benzyl)quinolin-2(1H)-one

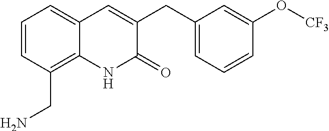 [SMILES: NCc1cccc2cc(Cc3cccc(OC(F)(F)F)c3)c(=O)[nH]c12]

A mixture of tert-butyl ((2-oxo-3-(3-(trifluoromethoxy)benzyl)-1,2-dihydroquinolin-8-yl)methyl)carbamate (25 mg, 0.056 mmol) in HCl (4.0M in dioxane, 3.0 mL, 12 mmol) was stirred at room temperature for 12 hours. The reaction mixture was concentrated under reduced pressure. The residue was purified by reverse phase HPLC (eluting acetonitrile in water, with TFA modifier) to afford 8-(aminomethyl)-3-(3-(trifluoromethoxy)benzyl)quinolin-2(1H)-one. ¹H NMR (400 MHz, DMSO-d₆) δ 11.45 (br s, 1H), 8.05 (br s, 2H), 7.87 (s, 1H), 7.64 (d, J=7.6 Hz, 1H), 7.52 (d, J=6.8 Hz, 1H), 7.44-7.36 (m, 1H), 7.34-7.27 (m, 2H), 7.24-7.12 (m, 2H), 4.30 (br s, 2H), 3.89 (s, 2H). LC/MS (m/z): 349 (M+H)⁺

### Example 179

### Preparation of 3-(4-(2-Hydroxy-2-methylpropyl)benzyl)quinolin-2(1H)-one

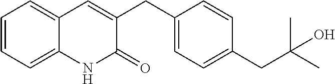 [SMILES: CC(C)(O)Cc1ccc(Cc2cc3ccccc3[nH]c2=O)cc1]

### Step A: Methyl 2-(4-((2-methoxyquinolin-3-yl)methyl)phenyl)acetate

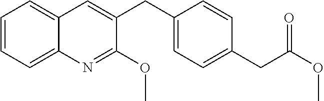 [SMILES: COC(=O)Cc1ccc(Cc2cc3ccccc3nc2OC)cc1]

A mixture of 3-(bromomethyl)-2-methoxyquinoline (240 mg, 0.95 mmol), methyl 2-(4-(4,4,5,5-tetramethyl-1,3,2-dioxaborolan-2-yl)phenyl)acetate (263 mg, 0.952 mmol), Pd(dppf)Cl₂-DCM adduct (78 mg, 0.095 mmol), and tripotassium phosphate (404 mg, 1.90 mmol) in dioxane (6 mL) and water (1.2 mL) was stirred and heated to 80° C. for 2 hours. The reaction mixture was cooled to room temperature, diluted with water (20 mL), and extracted with EtOAc (3×20 mL). The organic layers were combined, dried over sodium sulfate, filtered, and concentrated under reduced pressure to afford methyl 2-(4-((2-methoxyquinolin-3-yl)methyl)phenyl)acetate, which was used in next step without purification. LC/MS (m/z): 322 (M+H)⁺

### Step B: Methyl 2-(4-((2-oxo-1,2-dihydroquinolin-3-yl)methyl)phenyl)acetate

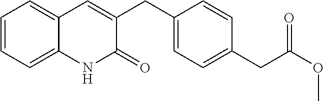 [SMILES: COC(=O)Cc1ccc(Cc2cc3ccccc3[nH]c2=O)cc1]

TMSI (0.64 mL, 4.7 mmol) was added to a mixture of methyl 2-(4-((2-methoxyquinolin-3-yl)methyl)phenyl)acetate (300 mg, 0.934 mmol) in CH₃CN (6 mL) at 0° C. The reaction mixture was warmed to room temperature and stirred for 2 hours. The reaction mixture was quenched with saturated aqueous Na₂SO₃ (30 mL) and was extracted with DCM (50 mL). The organic layer was dried over anhydrous sodium sulfate, filtered, and concentrated under reduced pressure. The residue was purified by reverse phase HPLC (eluting acetonitrile in water, with TFA modifier) to afford methyl 2-(4-((2-oxo-1,2-dihydroquinolin-3-yl)methyl)phenyl)acetate. LC/MS (m/z): 308 (M+H)⁺

### Step C: 3-(4-(2-Hydroxy-2-methylpropyl)benzyl)quinolin-2(1H)-one

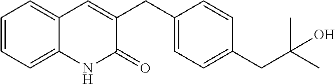 [SMILES: CC(C)(O)Cc1ccc(Cc2cc3ccccc3[nH]c2=O)cc1]

Methylmagnesium bromide (3.0 M in ether, 1.1 mL, 3.3 mmol) was added to a mixture of methyl 2-(4-((2-oxo-1,2-dihydroquinolin-3-yl)methyl)phenyl)acetate (200 mg, 0.651 mmol) in CH₃CN (6 mL) at 0° C. The reaction mixture was stirred at 20° C. for 1.5 hours. The reaction mixture was quenched with saturated aqueous NH₄Cl (20 mL) and extracted with DCM (40 mL). The organic layer was separated, washed with water, dried over anhydrous Na₂SO₄, filtered, and concentrated under reduced pressure. The residue was purified by reverse phase HPLC (eluting acetonitrile in water, with ammonium bicarbonate modifier) to afford 3-(4-(2-hydroxy-2-methylpropyl)benzyl)quinolin-2(1H)-one. ¹H NMR (400 MHz, methanol-d₄) δ 7.51 (s, 1H), 7.44-7.36 (m, 2H), 7.26 (d, J=8.3 Hz, 1H), 7.19-7.07 (m, 5H), 3.84 (s, 2H), 2.68 (s, 2H), 1.12 (s, 6H). LC/MS (m/z): 308 (M+H)⁺

### Example 180

### Preparation of 8-((methylamino)methyl)-3-(3-(trifluoromethoxy)benzyl)quinolin-2(1H)-one

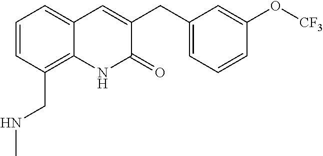 [SMILES: CNCc1cccc2cc(Cc3cccc(OC(F)(F)F)c3)c(=O)[nH]c12]

A mixture of 8-(aminomethyl)-3-(3-(trifluoromethoxy)benzyl)quinolin-2(1H)-one (25 mg, 0.072 mmol), TEA (10 μl, 0.07 mmol), and methyl trifluoromethanesulfonate (18 mg, 0.11 mmol) in hexafluoro-2-propanol (1 mL) was stirred and heated to 80° C. for 12 hours. The reaction mixture was concentrated under reduced pressure. The residue was purified by reverse phase HPLC (eluting acetonitrile in water with ammonia/ammonium bicarbonate modifier) to afford 8-((methylamino)methyl)-3-(3-(trifluoromethoxy)benzyl)quinolin-2(1H)-one. ¹H NMR (400 MHz, methanol-d₄) δ 7.75 (s, 1H), 7.51 (d, J=7.8 Hz, 1H), 7.40-7.34 (m, 2H), 7.33-7.28 (m, 1H), 7.24 (s, 1H), 7.19-7.14 (m, 1H), 7.11 (br d, J=8.2 Hz, 1H), 4.10 (s, 2H), 3.96 (s, 2H), 2.42 (s, 3H). LC/MS (m/z): 363 (M+H)⁺

### Example 181

### Preparation of 3-(3-chloro-4-(5-oxopyrrolidin-2-yl)benzyl)quinolin-2(1H)-one

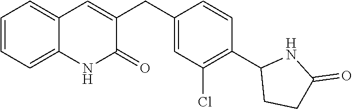 [SMILES: O=C1CCC(c2ccc(Cc3cc4ccccc4[nH]c3=O)cc2Cl)N1]

### Step A: 3-chloro-4-(5-oxopyrrolidin-2-yl)phenyl)boronic acid

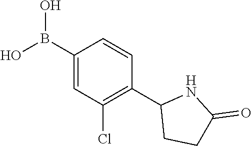 [SMILES: O=C1CCC(c2ccc(B(O)O)cc2Cl)N1]

NCS (223 mg, 1.67 mmol) and gold(III) chloride (8 mg, 0.03 mmol) were added to a stirred solution of 5-(4-(4,4,5,5-tetramethyl-1,3,2-dioxaborolan-2-yl)phenyl)pyrrolidin-2-one (200 mg, 0.7 mmol) in DCE (2 mL) at room temperature. The reaction was stirred and heated to 80° C. for 12 hours, and then heated to 100° C. and stirred for an additional 12 hours. The reaction mixture was cooled to room temperature and then filtered. The filtrate was concentrated under reduced pressure, and the residue was purified by reverse phase HPLC (eluting acetonitrile in water, with TFA modifier) to afford 3-chloro-4-(5-oxopyrrolidin-2-yl)phenyl)boronic acid. LC/MS (m/z): 240 (M+H)⁺

### Step B: 5-(2-Chloro-4-((2-methoxyquinolin-3-yl)methyl)phenyl)pyrrolidin-2-one

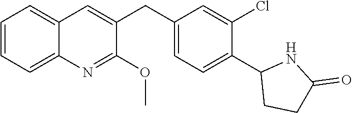 [SMILES: COc1nc2ccccc2cc1Cc1ccc(C2CCC(=O)N2)c(Cl)c1]

A mixture of 3-chloro-4-(5-oxopyrrolidin-2-yl)phenyl)boronic acid (20 mg, 0.083 mmol), 3-(bromomethyl)-2-methoxyquinoline (22 mg, 0.086 mmol), K₃PO₄ (50 mg, 0.23 mmol), and Pd(dppf)Cl₂ (6 mg, 8 μmol) in 1,4-dioxane (1 mL) and water (0.2 mL) was sparged with nitrogen for 3 minutes at room temperature. The reaction mixture was stirred and heated to 80° C. for 15 hours. The reaction mixture was cooled to room temperature and concentrated under reduced pressure. The residue was purified by silica gel chromatography (ethyl acetate in petroleum ether) to afford 5-(2-chloro-4-((2-methoxyquinolin-3-yl)methyl)phenyl)pyrrolidin-2-one. LC/MS (m/z): 367 (M+H)⁺

### Step C: 3-(3-Chloro-4-(5-oxopyrrolidin-2-yl)benzyl)quinolin-2(1H)-one

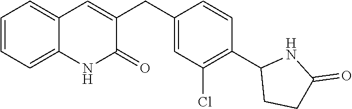 [SMILES: O=C1CCC(c2ccc(Cc3cc4ccccc4[nH]c3=O)cc2Cl)N1]

A solution of 5-(2-chloro-4-((2-methoxyquinolin-3-yl)methyl)phenyl)pyrrolidin-2-one (20 mg, 0.06 mmol) in 1,4-dioxane (1 mL) and HCl (3.0 M in water, 0.50 mL, 1.5 mmol) was stirred and heated to 70° C. for 2 hours. The reaction mixture was concentrated under reduced pressure. The residue was purified by reverse phase HPLC (eluting acetonitrile in water, with TFA modifier) to afford 3-(3-chloro-4-(5-oxopyrrolidin-2-yl)benzyl)quinolin-2(1H)-one. ¹H NMR (400 MHz, methanol-d₄) (7.76 (s, 1H), 7.59 (d, J=7.4 Hz, 1H), 7.51-7.47 (m, 1H), 7.39 (s, 1H), 7.35-7.28 (m, 3H), 7.25-7.20 (m, 1H), 5.17-5.13 (m, 1H), 3.91 (s, 2H), 2.72-2.67 (m, 1H), 2.39 (t, J=8.0 Hz, 2H), 1.92-1.84 (m, 1H). LC/MS (m/z): 353 (M+H)⁺

### Example 182

### Preparation of 3-(4-(4-hydroxytetrahydrofuran-3-yl)benzyl)quinolin-2(1H)-one

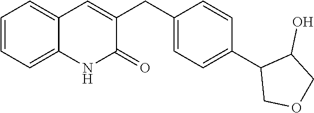 [SMILES: O=c1[nH]c2ccccc2cc1Cc1ccc(C2COCC2O)cc1]

### Step A: 4-Bromotetrahydrofuran-3-ol

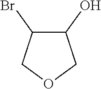

2,5-dihydrofuran (3.0 g, 43 mmol) was added to a stirred solution of NBS (11.4 g, 64.2 mmol) in H₂O (100 mL) at 0° C. The reaction mixture was warmed to room temperature and stirred for 4 hours. The reaction mixture was diluted with EtOAc (25 mL) and washed with brine (20 mL). The organic layer was separated, dried over anhydrous sodium sulfate, filtered, and concentrated under reduced pressure. The residue was purified by silica gel chromatography (ethyl acetate in petroleum ether) to afford 4-bromotetrahydrofuran-3-ol. ¹H NMR (500 MHz, chloroform-d) (4.59-4.55 (m, 1H), 4.40 (dd, J=4.6, 10.7 Hz, 1H), 4.25 (dd, J=4.3, 10.1 Hz, 1H), 4.21-4.17 (m, 1H), 4.07 (dd, J=2.2, 10.6 Hz, 1H), 3.80 (dd, J=1.1, 9.9 Hz, 1H), 2.56 (br s, 1H).

### Step B: (S,R and R,S)-4-(4-((2-methoxyquinolin-3-yl)methyl)phenyl)tetrahydrofuran-3-ol

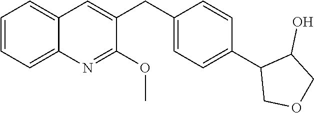 [SMILES: COc1nc2ccccc2cc1Cc1ccc(C2COCC2O)cc1]

A mixture of 4-bromotetrahydrofuran-3-ol (343 mg, 2.06 mmol), 3-(4-bromobenzyl)-2-methoxyquinoline (450 mg, 1.4 mmol), TBAI (506 mg, 1.37 mmol), Mn (226 mg, 4.11 mmol), picolinimidamide hydrochloride (97 mg, 0.62 mmol), and nickel(ii) chloride ethylene glycol dimethyl ether complex (151 mg, 0.686 mmol) in DMA (12 mL) was stirred and heated to 60° C. for 1.5 hour under a nitrogen atmosphere. The reaction mixture was filtered, and the filtrate was concentrated under reduced pressure. The residue was purified by reverse phase HPLC (eluting acetonitrile in water, with TFA modifier) to afford 4-(4-((2-methoxyquinolin-3-yl)methyl)phenyl)tetrahydrofuran-3-ol as a mixture with 5-(4-((2-methoxyquinolin-3-yl)methyl)phenyl)tetrahydrofuran-3-ol. LC/MS (m/z): 336 (M+H)⁺

### Step C: (S,R or R,S)-4-(4-((2-methoxyquinolin-3-yl)methyl)phenyl)tetrahydrofuran-3-ol

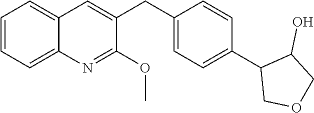 [SMILES: COc1nc2ccccc2cc1Cc1ccc(C2COCC2O)cc1]

The mixture of (S,R and R,S)-4-(4-((2-methoxyquinolin-3-yl)methyl)phenyl)tetrahydrofuran-3-ol and (S,R and R,S)-5-(4-((2-methoxyquinolin-3-yl)methyl)phenyl)tetrahydrofuran-3-ol (150 mg, 0.447 mmol) was resolved by Chiral-SFC (Column Daicel Chiralcel OJ-H (250 mm×50 mm, 5 um); eluting 0.1% ammonia in ethanol) to afford (S,R or R,S)-4-(4-((2-methoxyquinolin-3-yl)methyl)phenyl)tetrahydrofuran-3-ol as the third of four eluting peaks. LC/MS (m/z): 336 (M+H)⁺

### Step D: (S,R or R,S)-3-(4-(4-hydroxytetrahydrofuran-3-yl)benzyl)quinolin-2(1H)-one

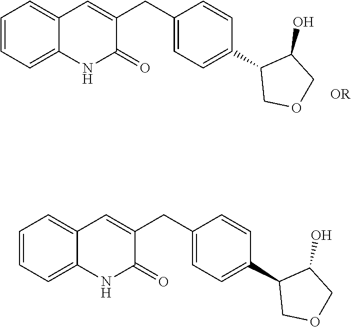 [SMILES: O=c1[nH]c2ccccc2cc1Cc1ccc([C@H]2COC[C@@H]2O)cc1 ; O=c1[nH]c2ccccc2cc1Cc1ccc([C@@H]2COC[C@H]2O)cc1]

HCl (37% in water, 0.15 mL) was added to a solution of (S,R or R,S)-4-(4-((2-methoxyquinolin-3-yl)methyl)phenyl)tetrahydrofuran-3-ol (45 mg, 0.134 mmol) in 1,4-dioxane (1.2 mL) and water (0.45 mL). The reaction mixture was sparged with nitrogen. The reaction mixture was stirred and heated to 70° C. for 2 hours. The reaction mixture was cooled to room temperature and then concentrated under reduced pressure. The residue was purified by reverse phase HPLC (eluting acetonitrile in water, with TFA modifier) to afford 3-(4-(4-hydroxytetrahydrofuran-3-yl)benzyl)quinolin-2-ol. ¹H NMR (400 MHz, methanol-d₄) δ 7.59 (s, 1H), 7.52 (d, J=7.8 Hz, 1H), 7.48-7.42 (m, 1H), 7.38-7.24 (m, 5H), 7.21-7.16 (m, 1H), 5.07 (dd, J=5.5, 10.4 Hz, 1H), 4.55-4.48 (m, 1H), 4.18 (dd, J=4.4, 9.5 Hz, 1H), 3.89 (s, 2H), 3.80 (d, J=10.0 Hz, 1H), 2.24 (dd, J=5.6, 13.2 Hz, 1H), 1.90 (ddd, J=5.3, 10.3, 13.3 Hz, 1H). LC/MS (m/z): 322 (M+H)⁺

### Example 183A and Example 183B

### Preparation of (S or R)-3-(4-(2-methyl-5-oxopyrrolidin-2-yl)benzyl)quinolin-2(1H)-one and (R or S)-3-(4-(2-methyl-5-oxopyrrolidin-2-yl)benzyl)quinolin-2(1H)-one

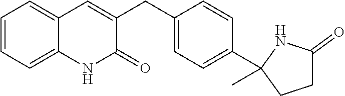 [SMILES: CC1(c2ccc(Cc3cc4ccccc4[nH]c3=O)cc2)CCC(=O)N1]

### Step A: tert-Butyl 2-(4-bromophenyl)pyrrolidine-1-carboxylate

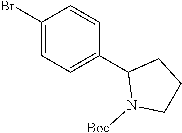 [SMILES: CC(C)(C)OC(=O)N1CCCC1c1ccc(Br)cc1]

(Boc)₂O (2.83 mL, 12.2 mmol) was added to a mixture of 2-(4-bromophenyl)pyrrolidine (2.3 g, 10 mmol) and Et₃N (2.8 mL, 20 mmol) in DCM (20 mL) at 0° C. The reaction mixture was stirred at 20° C. for 2 hours. The reaction mixture was quenched with water (20 mL) and extracted with DCM (3×10 mL). The organic layers were combined, washed with brine (20 mL), dried over anhydrous sodium sulfate, filtered, and concentrated under reduced pressure. The residue was purified by silica gel chromatography (ethyl acetate in petroleum ether) to afford tert-butyl 2-(4-bromophenyl)pyrrolidine-1-carboxylate. ¹H NMR (400 MHz, chloroform-d) δ 7.41 (d, J=8.6 Hz, 2H), 7.04 (br d, J=8.1 Hz, 2H), 4.93-4.65 (m, 1H), 3.67-3.46 (m, 2H), 2.30 (br d, J=6.8 Hz, 1H), 1.92-1.82 (m, 2H), 1.81-1.73 (m, 1H), 1.28 (s, 9H). LC/MS (m/z): 270 (M+H—C₄H₈)⁺

### Step B: tert-Butyl 2-(4-bromophenyl)-2-methylpyrrolidine-1-carboxylate

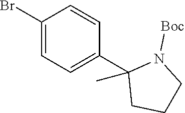 [SMILES: CC(C)(C)OC(=O)N1CCCC1(C)c1ccc(Br)cc1]

A solution of tert-butyl 2-(4-bromophenyl)pyrrolidine-1-carboxylate (2.0 g, 6.1 mmol) in THF (20 mL) was cooled to −78° C. sec-Butyllithium in hexanes (1.0 M in hexanes, 6.0 mL, 6.0 mmol) was added dropwise to the mixture. The reaction mixture was stirred at −78° C. for 2 min, and then methyl iodide (0.77 mL, 12 mmol) was added dropwise. The reaction mixture was stirred at −78° C. for an additional 30 minutes. The reaction mixture was quenched with saturated aqueous NH₄Cl (20 mL) and extracted with EtOAc (3×20 mL). The organic layers were combined, washed with brine (40 mL), dried over anhydrous sodium sulfate, filtered, and concentrated under reduced pressure. The residue was purified by reverse phase HPLC (eluting acetonitrile in water, with ammonium bicarbonate modifier) to afford tert-butyl 2-(4-bromophenyl)-2-methylpyrrolidine-1-carboxylate. ¹H NMR (400 MHz, chloroform-d) δ 7.41 (d, J=8.6 Hz, 2H), 7.11 (br d, J=8.6 Hz, 2H), 3.68 (t, J=6.7 Hz, 2H), 2.06-1.98 (m, 2H), 1.84-1.81 (m, 2H), 1.44 (s, 3H), 1.15 (s, 9H). LC/MS (m/z): 284 (M+H—C₄H₈)⁺

### Step C: tert-Butyl 2-(4-bromophenyl)-2-methyl-5-oxopyrrolidine-1-carboxylate

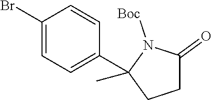 [SMILES: CC(C)(C)OC(=O)N1C(=O)CCC1(C)c1ccc(Br)cc1]

A mixture of ruthenium(III) chloride (4 mg, 20 μmol) and sodium periodate (566 mg, 2.65 mmol) in water (2 mL) was added to a stirred mixture of tert-butyl 2-(4-bromophenyl)-2-methylpyrrolidine-1-carboxylate (180 mg, 530 μmol) in EtOAc (2 mL) at 0° C. The reaction mixture was warmed to room temperature and stirred for 12 hours. The reaction mixture was quenched with water (10 mL) and extracted with EtOAc (3×10 mL). The organic layers were combined, washed with brine (10 mL), dried over anhydrous sodium sulfate, filtered, and concentrated under reduced pressure to afford tert-butyl 2-(4-bromophenyl)-2-methyl-5-oxopyrrolidine-1-carboxylate, which was used without further purification. LC/MS (m/z): 298 (M+H—C₄H₈)⁺

### Step D: tert-Butyl 2-methyl-5-oxo-2-(4-(4,4,5,5-tetramethyl-1,3,2-dioxaborolan-2-yl)phenyl)pyrrolidine-1-carboxylate

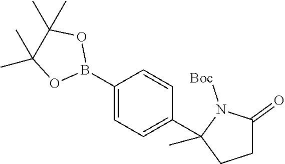 [SMILES: CC(C)(C)OC(=O)N1C(=O)CCC1(C)c1ccc(B2OC(C)(C)C(C)(C)O2)cc1]

A mixture of tert-butyl 2-(4-bromophenyl)-2-methyl-5-oxopyrrolidine-1-carboxylate (187 mg, 528 μmol), bis(pinacolato)diboron (201 mg, 792 μmol), Pd(dppf)Cl₂ (19 mg, 26 μmol), potassium acetate (155 mg, 1.58 mmol) in 1,4-dioxane (1.5 mL) was sparged with nitrogen at room temperature for 5 minutes. The reaction mixture was stirred and heated to 80° C. for 5 hours. The reaction mixture was filtered and the filtrate was concentrated under reduced pressure. The residue was purified by silica gel chromatography (eluting ethyl acetate in petroleum ether) to afford tert-butyl 2-methyl-5-oxo-2-(4-(4,4,5,5-tetramethyl-1,3,2-dioxaborolan-2-yl)phenyl)pyrrolidine-1-carboxylate. LC/MS (m/z): 346 (M+H—C₄H₈)⁺

### Step E: (rac)-tert-Butyl 2-(4-((2-methoxyquinolin-3-yl)methyl)phenyl)-2-methyl-5-oxopyrrolidine-1-carboxylate

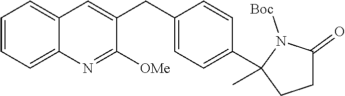 [SMILES: COc1nc2ccccc2cc1Cc1ccc(C2(C)CCC(=O)N2C(=O)OC(C)(C)C)cc1]

A mixture of 3-(chloromethyl)-2-methoxyquinoline (58 mg, 280 μmol), tert-butyl 2-methyl-5-oxo-2-(4-(4,4,5,5-tetramethyl-1,3,2-dioxaborolan-2-yl)phenyl)pyrrolidine-1-carboxylate (112 mg, 279 μmol), K₃PO₄ (119 mg, 559 μmol), and Pd(dppf)Cl₂ (20 mg, 28 μmol) in 1,4-dioxane (2 mL) and water (0.2 mL) was sparged with nitrogen for 5 minutes. The reaction mixture was stirred and heated to 80° C. for 12 hours. The reaction mixture was cooled to room temperature, quenched with water (3 mL), and extracted with EtOAc (3×4 mL). The organic layers were combined, washed with brine (5 mL), dried over anhydrous sodium sulfate, filtered, and concentrated under reduced pressure. The residue was purified by silica gel chromatography (ethyl acetate in petroleum ether) to afford tert-butyl 2-(4-((2-methoxyquinolin-3-yl)methyl)phenyl)-2-methyl-5-oxopyrrolidine-1-carboxylate. LC/MS (m/z): 447 (M+H)⁺

### Step F: (R or S)-5-(4-((2-methoxyquinolin-3-yl)methyl)phenyl)-5-methylpyrrolidin-2-one and (S or R)-5-(4-((2-methoxyquinolin-3-yl)methyl)phenyl)-5-methylpyrrolidin-2-one

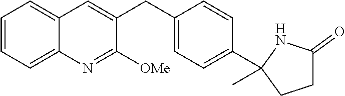 [SMILES: COc1nc2ccccc2cc1Cc1ccc(C2(C)CCC(=O)N2)cc1]

A mixture of (rac)-tert-butyl 2-(4-((2-methoxyquinolin-3-yl)methyl)phenyl)-2-methyl-5-oxopyrrolidine-1-carboxylate (70 mg, 160 μmol) in HCl (4.0 M in dioxane, 5.0 mL, 20 mmol) was stirred at room temperature for 1 hour. The reaction mixture was concentrated under reduced pressure, and the residue was purified by reverse phase HPLC (eluting acetonitrile in water, with TFA modifier) to afford (rac)-5-(4-((2-methoxyquinolin-3-yl)methyl)phenyl)-5-methylpyrrolidin-2-one 2,2,2-trifluoroacetate, which was resolved by Chiral-SFC (Phenomenex-Cellulose-2 30×250 mm column EtOH w/0.1% NH₄OH as cosolvent) to afford (R or S)-5-(4-((2-methoxyquinolin-3-yl)methyl)phenyl)-5-methylpyrrolidin-2-one (first eluting) LC/MS (m/z): 347 (M+H)+ and (S or R)-5-(4-((2-methoxyquinolin-3-yl)methyl)phenyl)-5-methylpyrrolidin-2-one (second eluting) LC/MS (m/z): 347 (M+H)+.

### Step G-1: (R or S)-3-(4-(2-methyl-5-oxopyrrolidin-2-yl)benzyl)quinolin-2(1H)-one

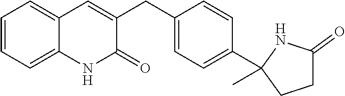 [SMILES: CC1(c2ccc(Cc3cc4ccccc4[nH]c3=O)cc2)CCC(=O)N1]

HCl (3.0 M in water, 0.15 mL, 0.45 mmol) was added to a mixture of (R or S)-5-(4-((2-methoxyquinolin-3-yl)methyl)phenyl)-5-methylpyrrolidin-2-one (first eluting peak from Step F, 22 mg, 64 μmol) in 1,4-dioxane (1 mL) and water (0.45 mL). The reaction mixture was stirred and heated to 80° C. for 1 hour. The reaction mixture was concentrated under reduced pressure. The residue was purified by reverse phase HPLC (eluting acetonitrile in water, with TFA modifier) to afford (R or S)-3-(4-(2-methyl-5-oxopyrrolidin-2-yl)benzyl)quinolin-2(1H)-one. ¹H NMR (400 MHz, methanol-d₄) δ 7.66 (s, 1H), 7.54 (d, J=8.2 Hz, 1H), 7.50-7.44 (m, 1H), 7.35-7.27 (m, 5H), 7.23-7.16 (m, 1H), 3.91 (s, 2H), 2.43-2.34 (m, 1H), 2.33-2.24 (m, 3H), 1.61 (s, 3H). LC/MS (m/z): 333 (M+H)⁺

### Step G-2: (S or R)-3-(4-(2-methyl-5-oxopyrrolidin-2-yl)benzyl)quinolin-2(1H)-one

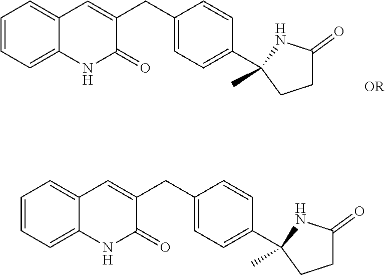 [SMILES: C[C@]1(c2ccc(Cc3cc4ccccc4[nH]c3=O)cc2)CCC(=O)N1 ; C[C@@]1(c2ccc(Cc3cc4ccccc4[nH]c3=O)cc2)CCC(=O)N1]

HCl (3.0 M in water, 0.15 mL, 0.45 mmol) was added to a mixture of (S or R)-5-(4-((2-methoxyquinolin-3-yl)methyl)phenyl)-5-methylpyrrolidin-2-one (second eluting peak from Step F, 22 mg, 64 μmol) in 1,4-dioxane (1 mL) and water (0.45 mL). The reaction mixture was stirred and heated to 80° C. for 1 hour. The reaction mixture was concentrated under reduced pressure. The residue was purified by reverse phase HPLC (eluting acetonitrile in water, with TFA modifier) to afford (S or R)-3-(4-(2-methyl-5-oxopyrrolidin-2-yl)benzyl)quinolin-2(11)-one. ¹H NMR (400 MHz, methanol-d₄) δ 7.65 (s, 1H), 7.53 (d, J=7.8 Hz, 1H), 7.49-7.43 (m, 1H), 7.34-7.27 (m, 5H), 7.22-7.16 (m, 1H), 3.90 (s, 2H), 2.43-2.33 (m, 1H), 2.32-2.24 (m, 3H), 1.60 (s, 3H). LC/MS (m/z): 333 (M+H)⁺

### Example 184

### Preparation of 3-(3-Chloro-4-(2-hydroxypropan-2-yl)benzyl)-1,6-naphthyridin-2(11H)-one

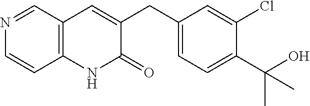 [SMILES: CC(C)(O)c1ccc(Cc2cc3cnccc3[nH]c2=O)cc1Cl]

### Step A: Methyl 2-chloro-4-((2-methoxy-1,6-naphthyridin-3-yl)methyl)benzoate

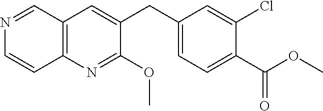 [SMILES: COC(=O)c1ccc(Cc2cc3cnccc3nc2OC)cc1Cl]

A mixture of 3-(chloromethyl)-2-methoxy-1,6-naphthyridine (100 mg, 0.479 mmol), (3-chloro-4-(methoxycarbonyl)phenyl)boronic acid (103 mg, 0.479 mmol), PdCl₂(dppf)-CH₂Cl₂ adduct (39 mg, 0.048 mmol), and tripotassium phosphate (203 mg, 0.959 mmol) in 1,4-dioxane (3 mL) and water (0.5 mL) was sparged with nitrogen and then stirred and heated to 80° C. for 12 hours. The reaction mixture was cooled to room temperature, washed with water (20 mL), and extracted with EtOAc (3×20 mL). The organic layers were combined, dried over sodium sulfate, filtered, and concentrated under reduced pressure. The residue was purified by silica gel chromatography (ethyl acetate in petroleum ether) to afford methyl 2-chloro-4-((2-methoxy-1,6-naphthyridin-3-yl)methyl)benzoate. ¹H NMR (500 MHz, methanol-d₄) δ 9.51 (s, 1H), 8.70 (d, J=6.7 Hz, 1H), 8.35 (s, 1H), 8.23 (d, J=6.7 Hz, 1H), 7.83 (d, J=7.9 Hz, 1H), 7.49 (d, J=1.2 Hz, 1H), 7.36 (dd, J=1.5, 8.1 Hz, 1H), 4.27 (s, 3H), 4.24 (s, 2H), 3.92 (m, 3H). LC/MS (m/z): 343 (M+H)⁺

### Step B: Methyl 2-chloro-4-((2-oxo-1,2-dihydro-1,6-naphthyridin-3-yl)methyl)benzoate

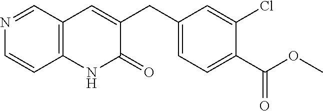 [SMILES: COC(=O)c1ccc(Cc2cc3cnccc3[nH]c2=O)cc1Cl]

Iodotrimethylsilane (0.353 mL, 2.48 mmol) was added to a mixture of methyl 2-chloro-4-((2-methoxy-1,6-naphthyridin-3-yl)methyl)benzoate (170 mg, 0.496 mmol) in CH₃CN (4 mL) under nitrogen atmosphere. The reaction mixture was stirred at room temperature for 2 hours. Saturated aqueous Na₂SO₃ (10 mL) was added to the mixture, and the reaction mixture was then basified with saturated aqueous NaHCO₃ until pH˜8. The mixture was extracted with EtOAc (100 mL). The organic layer was separated, dried over sodium sulfate, filtered, and concentrated under reduced pressure to afford methyl 2-chloro-4-((2-oxo-1,2-dihydro-1,6-naphthyridin-3-yl)methyl)benzoate, which was used in the next step without purification. LC/MS (m/z): 329 (M+H)⁺

### Step C: 3-(3-Chloro-4-(2-hydroxypropan-2-yl)benzyl)-1,6-naphthyridin-2(1H)-one

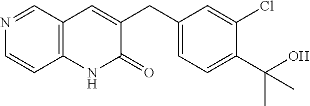 [SMILES: CC(C)(O)c1ccc(Cc2cc3cnccc3[nH]c2=O)cc1Cl]

Methylmagnesium bromide (3.0 M in ether, 0.76 mL, 2.3 mmol) was added to a mixture of methyl 2-chloro-4-((2-oxo-1,2-dihydro-1,6-naphthyridin-3-yl)methyl)benzoate (150 mg, 0.456 mmol) in THF (3 mL) at 0° C. The reaction mixture was warmed to room temperature and then stirred for 1.5 hours. The reaction mixture was quenched with saturated aqueous NH₄Cl (20 mL) and extracted with DCM (40 mL). The organic layer was separated, washed with water (10 mL), dried over sodium sulfate, filtered, and concentrated under reduced pressure. The residue was purified by reverse phase HPLC (eluting acetonitrile in water, with ammonia/ammonium bicarbonate modifier) to afford 3-(3-chloro-4-(2-hydroxypropan-2-yl)benzyl)-1,6-naphthyridin-2(1H)-one. ¹H NMR (500 MHz, methanol-d₄) (9.11 (s, 1H), 8.58 (d, J=6.9 Hz, 1H), 7.90 (s, 1H), 7.77 (d, J=8.2 Hz, 1H), 7.66 (d, J=6.7 Hz, 1H), 7.36 (d, J=1.7 Hz, 1H), 7.27 (dd, J=1.6, 8.2 Hz, 1H), 3.95 (s, 2H), 1.68 (s, 6H). LC/MS (m/z): 329

### Example 185

### Preparation of 5-((2-oxo-1,2-dihydroquinolin-3-yl)methyl)isoindoline-1,3-dione

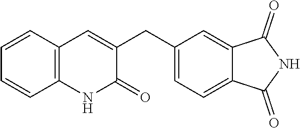 [SMILES: O=C1NC(=O)c2cc(Cc3cc4ccccc4[nH]c3=O)ccc21]

### Step A: 5-(4,4,5,5-Tetramethyl-1,3,2-dioxaborolan-2-yl)isoindoline-1,3-dione

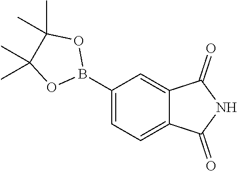 [SMILES: CC1(C)OB(c2ccc3c(c2)C(=O)NC3=O)OC1(C)C]

A mixture of 4-bromophthalimide (500 mg, 2.21 mmol), 4,4,4′,4′,5,5,5′,5′-octamethyl-2,2′-bi(1,3,2-dioxaborolane) (562 mg, 2.21 mmol), Pd(dppf)Cl₂ (162 mg, 0.221 mmol), potassium acetate (434 mg, 4.42 mmol) in 1,4-dioxane (8 mL) was sparged with nitrogen for 3 minutes. The reaction mixture was stirred and heated to 80° C. for 12 hours. The reaction mixture was cooled to room temperature, filtered, and concentrated under reduced pressure. The residue was diluted with water (150 mL) and extracted with EtOAc (150 mL). The organic layer was separated, dried over sodium sulfate, filtered, and concentrated under reduced pressure. The residue was purified by silica gel chromatography (eluting ethyl acetate in petroleum ether) to afford 5-(4,4,5,5-tetramethyl-1,3,2-dioxaborolan-2-yl)isoindoline-1,3-dione. ¹H NMR (400 MHz, methanol-d₄) δ 8.20-8.00 (m, 2H), 7.88-7.71 (m, 1H), 1.44-1.17 (m, 12H). LC/MS (m/z): 274

### Step B: 5-((2-Methoxyquinolin-3-yl)methyl)isoindoline-1,3-dione

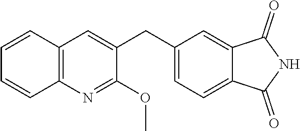 [SMILES: COc1nc2ccccc2cc1Cc1ccc2c(c1)C(=O)NC2=O]

A mixture of 5-(4,4,5,5-tetramethyl-1,3,2-dioxaborolan-2-yl)isoindoline-1,3-dione (440 mg, 1.61 mmol), 3-(chloromethyl)-2-methoxyquinoline (335 mg, 1.61 mmol), K₃PO₄ (684 mg, 3.22 mmol) and Pd(dppf)Cl₂ (118 mg, 0.161 mmol) in 1,4-dioxane (6 mL) and water (1.2 mL) was sparged with nitrogen for 3 minutes. The reaction mixture was stirred and heated to 80° C. for 12 hours. The reaction mixture was cooled to room temperature, diluted with water (50 mL), and extracted with EtOAc (50 mL). The organic layer was separated, dried over sodium sulfate, filtered, and concentrated under reduced pressure. The residue was purified by reverse phase HPLC (eluting acetonitrile in water, with ammonium bicarbonate modifier) to afford 5-((2-methoxyquinolin-3-yl)methyl)isoindoline-1,3-dione. ¹H NMR (500 MHz, chloroform-d) δ 7.84 (d, J=8.4 Hz, 1H), 7.78 (d, J=7.6 Hz, 1H), 7.76 (s, 1H), 7.73 (s, 1H), 7.68-7.64 (m, 2H), 7.60 (t, J=7.7 Hz, 1H), 7.43 (br s, 1H), 7.38 (t, J=7.5 Hz, 1H), 4.19 (s, 2H), 4.05 (s, 3H). LC/MS (m/z): 319

### Step C: 5-((2-Oxo-1,2-dihydroquinolin-3-yl)methyl)isoindoline-1,3-dione

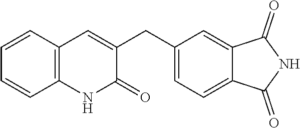 [SMILES: O=C1NC(=O)c2cc(Cc3cc4ccccc4[nH]c3=O)ccc21]

Iodotrimethylsilane (0.107 mL, 0.754 mmol) was added to a mixture of 5-((2-methoxyquinolin-3-yl)methyl)isoindoline-1,3-dione (80 mg, 0.25 mmol) in CH₃CN (3 mL). The reaction mixture was stirred and heated to 40° C. for 2 hours. The reaction mixture was quenched with saturated aqueous Na₂SO₃ (10 mL). The reaction mixture was filtered, and the collected solids were washed with DCM (2×2 mL) and MeOH (2×2 mL) and then dried under vacuum to afford 5-((2-oxo-1,2-dihydroquinolin-3-yl)methyl)isoindoline-1,3-dione. ¹H NMR (500 MHz, DMSO-d₆) (11.85 (s, 1H), 11.26 (s, 1H), 7.87 (s, 1H), 7.79-7.73 (m, 3H), 7.61 (d, J=7.8 Hz, 1H), 7.51-7.41 (m, 1H), 7.30 (d, J=8.1 Hz, 1H), 7.24-7.08 (m, 1H), 4.01 (s, 2H). LC/MS (m/z): 305

### Example 186

### Preparation of 3-(4-(((R or S)-methylsulfonimidoyl)methyl)benzyl)quinolin-2(1H)-one

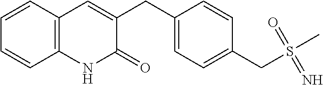 [SMILES: CS(=N)(=O)Cc1ccc(Cc2cc3ccccc3[nH]c2=O)cc1]

### Step A: tert-butyl ((4-((2-methoxyquinolin-3-yl)methyl)benzyl)(methyl)(oxo)-λ⁶-sulfanylidene)carbamate

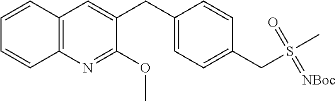

A mixture of 3-(chloromethyl)-2-methoxyquinoline (172 mg, 828 μmol), tert-butyl (methyl(oxo)(4-(4,4,5,5-tetramethyl-1,3,2-dioxaborolan-2-yl)benzyl)-λ⁶-sulfaneylidene)carbamate (337 mg, 0.853 mmol), Pd(dppf)Cl₂ (61 mg, 83 μmol), and K₃PO₄ (352 mg, 1.66 mmol) in 1,4-dioxane (8 mL) and water (0.8 mL) was degassed with a stream of nitrogen for 5 minutes. The mixture was stirred and heated at 80° C. for 12 h. The mixture was quenched with water (10 mL) and extracted with EtOAc (10 mL×3). The organic layers were combined, washed with brine (20 mL), dried over anhydrous Na₂SO₄, filtered, and concentrated under reduced pressure. The residue was purified by silica gel chromatography (eluting ethyl acetate in petroleum ether) to afford tert-butyl ((4-((2-methoxyquinolin-3-yl)methyl)benzyl)(methyl)(oxo)-λ⁶-sulfaneylidene)carbamate as racemic mixture. ¹H NMR (400 MHz, chloroform-d) δ 7.84 (d, J=8.3 Hz, 1H), 7.68 (s, 1H), 7.65 (d, J=8.1 Hz, 1H), 7.59 (dt, J=1.5, 7.7 Hz, 1H), 7.38-7.28 (m, 5H), 4.82-4.67 (m, 2H), 4.08 (s, 3H), 4.07 (s, 2H), 2.92 (s, 3H), 1.25 (s, 9H). LC/MS (m/z): 463 (M+Na)⁺

### Step B: tert-butyl (((R or S)-4-((2-methoxyquinolin-3-yl)methyl)benzyl)(methyl)(oxo)-λ⁶-sulfanylidene)carbamate and tert-butyl (((S or R)-4-((2-methoxyquinolin-3-yl)methyl)benzyl)(methyl)(oxo)-λ⁶-sulfanylidene)carbamate

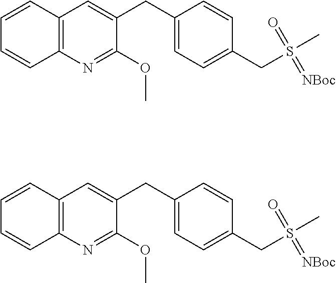

Racemic tert-butyl ((4-((2-methoxyquinolin-3-yl)methyl)benzyl)(methyl)(oxo)-λ⁶-sulfaneylidene)carbamate (0.200 g, 0.454 mmol) was resolved by Chiral-SFC (Column: Daicel Chiralpak AD (250 mm*30 mm, 10 μm); eluting 35% (0.1% NH₃·H₂O in EtOH) in CO₂; flow rate (mL/min): 70) to afford:

tert-butyl (((R or S)-4-((2-methoxyquinolin-3-yl)methyl)benzyl)(methyl)(oxo)-λ⁶-sulfaneylidene)carbamate (tᵣ=5.27 min, ee=100%) as the first eluting peak. LC/MS (m/z): 441 (M+H)⁺

tert-butyl (((S or R)-4-((2-methoxyquinolin-3-yl)methyl)benzyl)(methyl)(oxo)-λ⁶-sulfaneylidene)carbamate (tᵣ=5.77 min, ee=100%) as the second eluting peak. LC/MS (m/z): 441 (M+H)⁺

### Step C: 3-(4-((R or S)-methylsulfonimidoyl)methyl)benzyl)quinolin-2(1H)-one

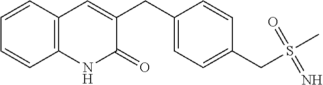 [SMILES: CS(=N)(=O)Cc1ccc(Cc2cc3ccccc3[nH]c2=O)cc1]

Iodotrimethylsilane (74 μL, 52 μmol) was added to a mixture of tert-butyl (((R or S)-4-((2-methoxyquinolin-3-yl)methyl)benzyl)(methyl)(oxo)-λ⁶-sulfaneylidene)carbamate (46 mg, 100 μmol) in acetonitrile (2 mL) at 0° C. The mixture was stirred at 20° C. for 2 h. The mixture was quenched with saturated aqueous Na₂SO₃ (0.5 mL) and diluted with DMF (3 mL). The mixture was directly purified by reverse phase HPLC (eluting acetonitrile in water, with NH₄HCO₃ modifier) to afford 3-(4-(((R or S)-methylsulfonimidoyl)methyl)benzyl)quinolin-2(1H)-one

¹H NMR (500 MHz, methanol-d₄) δ 7.71 (s, 1H), 7.57 (d, J=7.9 Hz, 1H), 7.51-7.46 (m, 1H), 7.43-7.39 (m, 2H), 7.39-7.36 (m, 2H), 7.32 (d, J=8.2 Hz, 1H), 7.25-7.19 (m, 1H), 4.44 (s, 2H), 3.95 (s, 2H), 2.89 (s, 3H). LC/MS (m/z): 349 (M+Na)⁺

### Example 187

### Preparation of 3-(4-(((S or R)-methylsulfonimidoyl)methyl)benzyl)quinolin-2(1H)-one

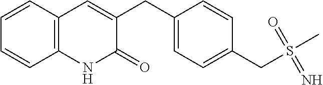 [SMILES: CS(=N)(=O)Cc1ccc(Cc2cc3ccccc3[nH]c2=O)cc1]

Iodotrimethylsilane (78 μL, 55 μmol) was added to a mixture of tert-butyl (((S or R)-4-((2-methoxyquinolin-3-yl)methyl)benzyl)(methyl)(oxo)-λ⁶-sulfaneylidene)carbamate (48 mg, 110 μmol) in acetonitrile (2 mL) at 0° C. The mixture was stirred at 20° C. for 2 h. The mixture was quenched with saturated aqueous Na₂SO₃ (0.5 mL) and diluted with DMF (3 mL). The mixture was purified by reverse phase HPLC (eluting acetonitrile in water, with NH₄HCO₃ modifier) to afford 3-(4-(((S or R)-methylsulfonimidoyl)methyl)benzyl)quinolin-2(1H)-one. ¹H NMR (500 MHz, methanol-d₄) δ 7.71 (s, 1H), 7.57 (dd, J=1.1, 7.9 Hz, 1H), 7.48 (ddd, J=1.4, 7.1, 8.3 Hz, 1H), 7.42-7.39 (m, 2H), 7.39-7.35 (m, 2H), 7.32 (d, J=8.2 Hz, 1H), 7.24-7.19 (m, 1H), 4.44 (s, 2H), 3.95 (s, 2H), 2.89 (s, 3H). LC/MS (m/z): 349 (M+Na)⁺

### Example 188

### Preparation of (R or S)-5-methyl-3-(4-((2-oxo-1,2-dihydro-1,6-naphthyridin-3-yl)methyl)phenyl)oxazolidin-2-one

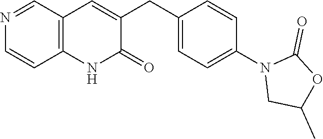 [SMILES: CC1CN(c2ccc(Cc3cc4cnccc4[nH]c3=O)cc2)C(=O)O1]

### Step A: (R and S)-3-(4-((2-methoxy-1,6-naphthyridin-3-yl)methyl)phenyl)-5-methyloxazolidin-2-one

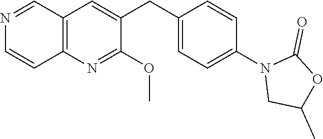 [SMILES: COc1nc2ccncc2cc1Cc1ccc(N2CC(C)OC2=O)cc1]

A mixture of (R and S)-5-methyl-3-(4-(4,4,5,5-tetramethyl-1,3,2-dioxaborolan-2-yl)phenyl)oxazolidin-2-one (368 mg, 1.21 mmol), 3-(chloromethyl)-2-methoxy-1,6-naphthyridine (253 mg, 1.21 mmol), K₃PO₄ (773 mg, 3.64 mmol), and Pd(dppf)Cl₂ (89 mg, 0.12 mmol) in 1,4-dioxane (10 mL) and water (2 mL) was degassed with nitrogen at room temperature for 5 minutes. The mixture was stirred and heated at 80° C. for 4 h. The mixture concentrated under reduced pressure and the residue was partitioned between water (10 mL) and DCM (10 mL). The organic layer was separated and the aqueous layer was re-extracted with DCM (10 mL×3). The organic layers were combined, washed with brine (10 mL), dried over anhydrous Na₂SO₄, filtered, and concentrated under reduced pressure. The residue was purified by silica gel chromatography (eluting ethyl acetate in petroleum ether) to afford (R and S)-3-(4-((2-methoxy-1,6-naphthyridin-3-yl)methyl)phenyl)-5-methyloxazolidin-2-one. ¹H NMR (400 MHz, methanol-d₄) δ 9.02 (s, 1H), 8.52-8.50 (m, 1H), 8.04 (s, 1H), 7.72-7.71 (m, 1H), 7.45-7.29 (m, 4H), 4.66-4.57 (m, 3H), 4.12 (s, 3H), 3.66 (s, 2H), 1.28-1.26 (m, 3H). LC/MS (m/z): 350 (M+H)⁺

### Step B: (R or S)-3-(4-((2-methoxy-1,6-naphthyridin-3-yl)methyl)phenyl)-5-methyloxazolidin-2-one and (S or R)-3-(4-((2-methoxy-1,6-naphthyridin-3-yl)methyl)phenyl)-5-methyloxazolidin-2-one

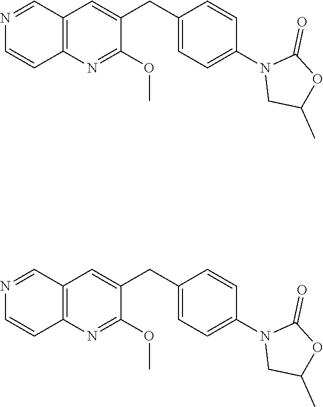 [SMILES: COc1nc2ccncc2cc1Cc1ccc(N2CC(C)OC2=O)cc1 ; COc1nc2ccncc2cc1Cc1ccc(N2CC(C)OC2=O)cc1]

(R and S)-3-(4-((2-methoxy-1,6-naphthyridin-3-yl)methyl)phenyl)-5-methyloxazolidin-2-one (0.400 g, 1.145 mmol) was resolved by Chiral-SFC (Column (S,S)-Whelk-O-1 (250 mm*30 mm, 5 um) eluting 50% (0.1% ammonia in ethanol) in CO₂; Flow Rate (mL/min) 70) to afford:

(R or S)-3-(4-((2-methoxy-1,6-naphthyridin-3-yl)methyl)phenyl)-5-methyloxazolidin-2-one (tᵣ=4.28 min) as the first eluting peak. LC/MS (m/z): 350 (M+H)⁺

(S or R)-3-(4-((2-methoxy-1,6-naphthyridin-3-yl)methyl)phenyl)-5-methyloxazolidin-2-one (tᵣ=6.07 min) as the second eluting peak. LC/MS (m/z): 350 (M+H)⁺

### Step C: (R or S)-5-methyl-3-(4-((2-oxo-1,2-dihydro-1,6-naphthyridin-3-yl)methyl)phenyl)oxazolidin-2-one

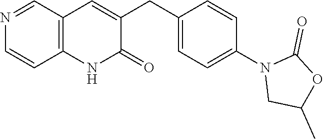 [SMILES: CC1CN(c2ccc(Cc3cc4cnccc4[nH]c3=O)cc2)C(=O)O1]

HCl (12 M in water, 0.050 mL, 0.60 mmol) was added to a mixture of (R or S)-3-(4-((2-methoxy-1,6-naphthyridin-3-yl)methyl)phenyl)-5-methyloxazolidin-2-one (0.050 g, 0.14 mmol) in 1,4-dioxane (1 mL) and water (0.4 mL) at 20° C. The mixture was stirred and heated at 65° C. for 12 h. The mixture was filtered and the filtrate was concentrated under reduced pressure. The residue was purified by reverse phase HPLC (eluting acetonitrile in water, with 0.05% TFA modifier) to afford (R or S)-5-methyl-3-(4-((2-oxo-1,2-dihydro-1,6-naphthyridin-3-yl)methyl)phenyl)oxazolidin-2-one. ¹H NMR (500 MHz, methanol-d₄) δ 9.02 (s, 1H), 8.54-8.52 (m, 1H), 7.83 (s, 1H), 7.60-7.58 (m, 1H), 7.45-7.37 (m, 4H), 4.67-4.59 (m, 2H), 4.11-4.03 (m, 1H), 4.01-3.94 (m, 2H), 1.28-1.27 (m, 3H). LC/MS (m/z): 336 (M+H)⁺

### Example 189

### Preparation of (S or R)-5-methyl-3-(4-((2-oxo-1,2-dihydro-1,6-naphthyridin-3-yl)methyl)phenyl)oxazolidin-2-one

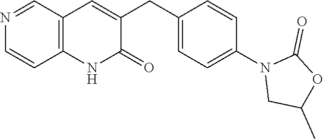 [SMILES: CC1CN(c2ccc(Cc3cc4cnccc4[nH]c3=O)cc2)C(=O)O1]

HCl (12 M in water, 0.050 mL, 0.60 mmol) was added to a mixture of (S or R)-3-(4-((2-methoxy-1,6-naphthyridin-3-yl)methyl)phenyl)-5-methyloxazolidin-2-one (0.050 g, 0.14 mmol) in 1,4-dioxane (1 mL) and water (0.4 mL) at 20° C. The mixture was stirred and heated at 65° C. for 12 h. The mixture was filtered and the filtrate was concentrated under reduced pressure. The residue was purified by reverse phase HPLC (eluting acetonitrile in water, with 0.05% TFA modifier) to afford (S or R)-5-methyl-3-(4-((2-oxo-1,2-dihydro-1,6-naphthyridin-3-yl)methyl)phenyl)oxazolidin-2-one. ¹H NMR (500 MHz, methanol-d₄) δ 9.04 (s, 1H), 8.54 (d, J=6.7 Hz, 1H), 7.83 (s, 1H), 7.61 (d, J=6.7 Hz, 1H), 7.45-7.37 (m, 4H), 4.68-4.58 (m, 2H), 4.10-4.03 (m, 1H), 3.97 (s, 2H), 1.28-1.27 (m, 3H). LC/MS (m/z): 336 (M+H)⁺

### IL4I1 Enzymatic Assay

Interleukin 4 inducible protein 1 (IL4I1) is an L-amino oxidase that catalyzes the oxidation of aromatic residues (Phe, Trp and Tyr): L-amino acid+H₂O+O₂→2-oxo acid+NH₃+H₂O₂. Equal molar of H₂O₂ and the corresponding alpha-ketoacid are produced when IL4I1 and substrate are added. In this assay, the hydrogen peroxide generated by IL4I1 are then detected through a coupled reaction with Amplex Red (10-acetyl-3,7-dihydroxyphenoxazine) and Horse Peroxidase (HRP) to produce Resorufin product that could be detected in the form of fluorescence signals. The assessment of the inhibitory effect of small molecules (EC₅₀) on IL4I1 is measured by the effectiveness of the compounds to inhibit the production of H₂O₂.

Using this assay, the potency (EC₅₀) of each compound was determined from a ten-point (1:3 serial dilution; top compound concentration of 10000 nM) titration curve using the following outlined procedure. To each well of a black flat-bottom Greiner (Cat #781076) 384 well-plate, 25 nL of compound (0.1% DMSO in final assay volume of 25 μL) was dispensed, followed by the addition of 12.5 μL of 1× assay buffer (50 mM Hepes 7.0 and 0.005% Tween20 (Sigma, Cat #P8341; low peroxide grade)) containing 2 nM of recombinant IL4I1 (R&D Systems, Cat #5684-AO-020). Plates were placed in an ambient temperature humidified chamber for a four-hour pre-incubation with compound. Subsequently, each reaction was initiated by the addition of 12.5 μL 1× assay buffer containing 2 mM of each aromatic amino acids (Phe/Tyr/Trp), 0.1 mM Amplex Red and 2 U/mL of HRP. The final reaction in each well of 25 μL consists of 1 nM of IL4I1, 1 mM of each residues (Phe, Tyr and Trp), 0.05 mM Amplex Red and 1 U/mL of HRP. It should be noted that the concentrations of Amplex Red and HRP used here are in excess such that the conversion of H₂O₂ to Resorufin product occurs instantaneously and non-rate limiting. Reactions were allowed to proceed for 120 minutes followed by fluorescence readout on a Spectramax with the following set parameters: 544 nm excitation/590 nm emission, 570 nm cutoff (EnVision is an alternative reader). Dose-response curves were generated by plotting percent effect (% product conversion; Y-axis) vs. Log₁₀ compound concentrations (X-axis). EC₅₀ values were calculated using a non-linear regression, four-parameters sigmoidal dose-response model.

| Example No. | IL4I1 EC₅₀ (nM) |
|---|---|
| 1 | 15.5 |
| 2 | 34.8 |
| 3 | 2.0 |
| 4 | 77.1 |
| 5 | 42.0 |
| 6 | 2.8 |
| 7 | 82.2 |
| 8 | 12.8 |
| 9 | 51.1 |
| 10 | 7.6 |
| 11 | 2.2 |
| 12 | 428.6 |
| 13 | 5.7 |
| 14 | 149.7 |
| 15 | 13.7 |
| 16 | 10.2 |
| 17 | 169.4 |
| 18 | 23.4 |
| 19 | 125.4 |
| 20 | 624.4 |
| 21 | 177.2 |
| 22 | 202.9 |
| 23 | 125.5 |
| 24 | 194.0 |
| 25 | 614.8 |
| 26 | 314.1 |
| 27 | 70.3 |
| 28 | 124.7 |
| 29 | 546.7 |
| 30 | 696.4 |
| 31 | 171.5 |
| 32 | 168.2 |
| 33 | 65.3 |
| 34 | 652.6 |
| 35 | 46.5 |
| 36 | 30.7 |
| 37 | 138.3 |
| 38 | 25.2 |
| 39 | 10.2 |
| 40 | 23.4 |
| 41 | 13.4 |
| 42 | 21.0 |
| 43 | 587.6 |
| 44 | 7.7 |
| 45 | 6.7 |
| 46 | 5.9 |
| 47 | 10.8 |
| 48 | 21.4 |
| 49 | 15.6 |
| 50 | 18.9 |
| 51 | 29.1 |
| 52 | 10.7 |
| 53 | 5.6 |
| 54A | 10.1 |
| 54B | 11.5 |
| 55A | 20.1 |
| 55B | 21.6 |
| 56A | 25.6 |
| 56B | 19.4 |
| 57A | 7.1 |
| 57B | 4.8 |
| 58 | 9.8 |
| 59 | 23.6 |
| 60 | 19.9 |
| 61 | 2.7 |
| 62 | 6.0 |
| 63 | 9.7 |
| 64 | 10.8 |
| 65A | 7.1 |
| 65B | 9.0 |
| 66 | 2.0 |
| 67 | 15.6 |
| 68 | 12.3 |
| 69 | 10.6 |
| 70 | 8.9 |
| 71 | 3.7 |
| 72 | 2.2 |
| 73 | 6.4 |
| 74 | 6.9 |
| 75 | 0.9 |
| 76 | 8.2 |
| 77 | 4.7 |
| 78 | 10.7 |
| 79 | 11.5 |
| 80 | 3.3 |
| 81 | 2.4 |
| 82 | 10.0 |
| 83 | 3.8 |
| 84 | 13.8 |
| 85 | 8.7 |
| 86 | 0.9 |
| 87 | 4.2 |
| 88 | 1.3 |
| 89 | 4.5 |
| 90A | 9.6 |
| 90B | — |
| 91 | 10.9 |
| 92 | 1.7 |
| 93 | 14.4 |
| 94 | 29.6 |
| 95 | 2.5 |
| 96 | 2.0 |
| 97 | 1.4 |
| 98 | 1.0 |
| 99 | 5.9 |
| 100 | 7.3 |
| 101A | 2.6 |
| 101B | — |
| 102A | 5.2 |
| 102B | 7.8 |
| 102C | 11.8 |
| 102D | 10.9 |
| 103 | 466.2 |
| 104 | 9184.0 |
| 105 | 619.2 |
| 106 | 957.8 |
| 107 | 3.2 |
| 108 | 1.9 |
| 109 | 39.9 |
| 110 | 11.5 |
| 111 | 24.6 |
| 112 | 60.5 |
| 113 | 7.0 |
| 114 | 1.5 |
| 115 | 1.4 |
| 116 | 1.7 |
| 117 | 2.1 |
| 118 | 11.1 |
| 119 | 16.0 |
| 120 | 3.6 |
| 121 | 0.9 |
| 122 | 6.6 |
| 123 | 3.7 |
| 124 | 3.5 |
| 125 | 6.1 |
| 126 | 2.7 |
| 127 | 2.1 |
| 128 | 30.4 |
| 129 | 13.6 |
| 130 | 1.8 |
| 131 | 3.7 |
| 132 | 18.6 |
| 133 | 2.0 |
| 134 | 5.1 |
| 135 | 11.3 |
| 136 | 0.5 |
| 137 | 4.5 |
| 138 | 36.9 |
| 139 | 8.5 |
| 140 | 23.4 |
| 141 | 20.2 |
| 142A | 4.4 |
| 142B | 81.4 |
| 143 | 2.6 |
| 144 | 7.2 |
| 145 | 154.5 |
| 146 | 1.7 |
| 147 | 2.3 |
| 148 | 258.4 |
| 149 | 150.3 |
| 150 | 16.9 |
| 151 | 3.3 |
| 152 | 5.3 |
| 153 | 1.2 |
| 154 | 5.4 |
| 155 | 2.0 |
| 156 | 1.4 |
| 157 | 6.2 |
| 158 | 60.2 |
| 159 | 4.1 |
| 160 | 6.3 |
| 161 | 3.2 |
| 162 | 6.8 |
| 163 | 5.4 |
| 164 | 10000.0 |
| 165 | 6.2 |
| 166 | 2.7 |
| 167 | 4.9 |
| 168 | 3.4 |
| 169 | 2.3 |
| 170 | 1.0 |
| 171 | 7.0 |
| 172 | 532.2 |
| 173 | 6843.0 |
| 174 | 10000.0 |
| 175 | 7.7 |
| 176 | 1.4 |
| 177 | 1906.0 |
| 178 | 99.0 |
| 179 | 4.9 |
| 180 | 104.6 |
| 181 | 2.5 |
| 182 | 7.3 |
| 183A | 15.7 |
| 183B | 11.1 |
| 184 | 15.3 |
| 185 | 9.1 |
| 186 | 28.2 |
| 187 | 28.6 |
| 188 | 12.3 |
| 189 | 16.7 |

What is claimed:

1. A compound of Formula I 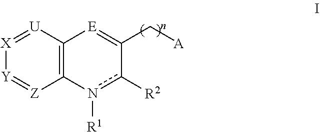
or a pharmaceutically acceptable salt thereof, wherein:
A is aryl, C₃-C₁₀cycloalkyl, heteroaryl or cycloheteroalkyl, wherein the aryl, C₃-C₁₀cycloalkyl, heretoaryl or cycloheteroalkyl is unsubstituted or substituted with 1 to 4 substituents independently selected from the group consisting of:
halogen,
C₁-C₆alkyl,
haloC₁-C₆alkyl,
—OH,
C₁-C₆alkylOH,
haloC₁-C₆alkylOH,
—COC₁-C₆alkyl,
—CN,
C₁-C₆alkylCN,
alkoxy,
haloalkoxy,
C₃-C₁₀cycloalkyl,
C₁-C₆alkylC₃-C₁₀cycloalkyl,
—OC₃-C₁₀cycloalkyl,
—O(C₁-C₆)C₃-C₁₀cycloalkyl,
aryl,
C₁-C₆alkylaryl,
heteroaryl,
C₁-C₆alkylheteroaryl,
C₁-C₆alkylcycloheteroalkyl,
cycloheteroalkyl,
—Ocycloheteroalkyl,
—COcycloheteroalkyl,
—NHcycloheteroalkyl,
—SO₂NH₂,
C₁-C₆alkylSO₂NH₂,
C₁-C₆alkylNHSO₂C₁-C₆alkyl,
—NHSO₂C₁-C₆alkyl,
—NHCOOC₁-C₆alkyl,
—NHCOC₁-C₆alkyl,
C₁-C₆alkylNHCOOC₁-C₆alkyl,
C₁-C₆alkylNHCOC₁-C₆alkyl, and 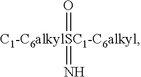
wherein the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, —NHcycloheteroalkyl, cycloheteroalkyl, COcycloheteroalkyl, C₁-C₆alkylcycloheteroalkyl, is unsubstituted or substituted with 1 to 3 substituents independently selected from the group consisting of halogen, C₁-C₆alkyl, C₃-C₁₀cycloalkyl, haloC₁-C₆alkyl, —NH₂, alkoxy, —OH, C₁-C₆alkylOH, —CN, C₁-C₆alkylCN, COC₁-C₆alkyl and oxo;
E is N, O or CR³;
U is N, S or CR⁴, wherein when U is N, X, Y and Z are CR⁵, CR⁶ and CR⁷ respectively;
X is a bond, N or CR⁵, wherein when X is N, U, Y and Z are CR⁴, CR⁶ and CR⁷ respectively;
Y is N or CR⁶, wherein when Y is N, X, U and Z are CR⁵, CR⁴ and CR⁷ respectively;
Z is N, S or CR⁷, wherein when Z is N, X, Y and U are CR⁵, CR⁶ and CR⁴ respectively;
R¹ is a bond between the nitrogen R¹ is attached and the carbon R² is attached, hydrogen or C₁-C₆alkyl, or when taken with R⁷ forms a C₅-C₇cycloalkyl;
R² is oxo or —OH, wherein when R² is oxo R¹ is not a bond between the nitrogen R¹ is attached and the carbon R² is attached;
R³ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁴ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁵ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁶ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁷ is hydrogen, halogen, —CN, —OH, C₁-C₆alkyl, C₁-C₆alkylNH₂, C₁-C₆alkylNHC₁-C₆alkyl, alkoxy or C₁-C₆alkylOH or when taken with R¹ forms a C₅-C₇cycloalkyl; and
n is 1 or 2.

2. The compound of claim 1, having the Formula Ia: 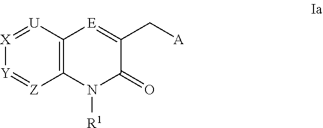
or a pharmaceutically acceptable salt thereof, wherein:
A is aryl, C₃-C₁₀cycloalkyl, heteroaryl or cycloheteroalkyl, wherein the aryl, C₃-C₁₀cycloalkyl, heretoaryl or cycloheteroalkyl is unsubstituted or substituted with 1 to 4 substituents independently selected from the group consisting of halogen, C₁-C₆alkyl, haloC₁-C₆alkyl, —OH, C₁-C₆alkylOH, haloC₁-C₆alkylOH, —COC₁-C₆alkyl, —CN, C₁-C₆alkylCN, alkoxy, haloalkoxy, C₃-C₁₀cycloalkyl, C₁-C₆alkylC₃-C₁₀cycloalkyl, —OC₃-C₁₀cycloalkyl, —O(C₁-C₆)C₃-C₁₀cycloalkyl, aryl, C₁-C₆alkylaryl, heteroaryl, C₁-C₆alkylheteroaryl, C₁-C₆alkylcycloheteroalkyl, cycloheteroalkyl, —Ocycloheteroalkyl, —COcycloheteroalkyl, —NHcycloheteroalkyl, —SO₂NH₂, C₁-C₆alkylSO₂NH₂, C₁-C₆alkylNHSO₂C₁-C₆alkyl, —NHSO₂C₁-C₆alkyl, —NHCOOC₁-C₆alkyl, —NHCOC₁-C₆alkyl, C₁-C₆alkylNHCOOC₁-C₆alkyl, and C₁-C₆alkylNHCOC₁-C₆alkyl, and 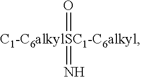
wherein the C₁-C₆alkylCN, haloC₁-C₆alkyl, C₃-C₁₀cycloalkyl, C₁-C₆alkylC₃-C₁₀cycloalkyl, —OC₃-C₁₀cycloalkyl, aryl, C₁-C₆alkylaryl, heteroaryl, —NHcycloheteroalkyl, cycloheteroalkyl, —COcycloheteroalkyl, and C₁-C₆alkylcycloheteroalkyl, is unsubstituted or substituted with 1 to 3 substituents independently selected from the group consisting of halogen, C₁-C₆alkyl, C₃-C₁₀cycloalkyl, haloC₁-C₆alkyl, —NH₂, alkoxy, —OH, C₁-C₆alkylOH, —CN, C₁-C₆alkylCN, COC₁-C₆alkyl and oxo;
E is N, O or CR³;
U is N, S or CR⁴, wherein when U is N, X, Y and Z are CR⁵, CR⁶ and CR⁷ respectively;
X is a bond, N or CR⁵, wherein when X is N, U, Y and Z are CR⁴, CR⁶ and CR⁷ respectively;
Y is N or CR⁶, wherein when Y is N, X, U and Z are CR⁵, CR⁴ and CR⁷ respectively;
Z is N, S or CR⁷, wherein when Z is N, X, Y and U are CR⁵, CR⁶ and CR⁴ respectively;
R¹ is hydrogen or C₁-C₆alkyl, or when taken with R⁷ forms a C₅-C₇cycloalkyl;
R³ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁴ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁵ is hydrogen, halogen, —CN, —OH, C₁-C₆alkyl, C₁-C₆alkylNH₂, C₁-C₆alkylNHC₁-C₆alkyl, alkoxy or C₁-C₆alkylOH;
R⁶ is hydrogen, halogen, —CN, —OH, C₁-C₆alkyl, C₁-C₆alkylNH₂, C₁-C₆alkylNHC₁-C₆alkyl, alkoxy or C₁-C₆alkylOH; and
R⁷ is hydrogen, halogen, —CN, —OH, C₁-C₆alkyl, C₁-C₆alkylNH₂, C₁-C₆alkylNHC₁-C₆alkyl, alkoxy or C₁-C₆alkylOH or when taken with R¹ forms a C₅-C₇cycloalkyl.

3. The compound of claim 1, or a pharmaceutically acceptable salt thereof, wherein E is CR³, wherein R³ is hydrogen or methyl.

4. The compound of claim 1, or a pharmaceutically acceptable salt thereof, wherein U is CR⁴, wherein R⁴ is hydrogen.

5. The compound of claim 1, or a pharmaceutically acceptable salt thereof, wherein X is N or CR⁵, wherein R⁵ is hydrogen, fluorine, methoxy, —CN or isopropanol.

6. The compound of claim 1, or a pharmaceutically acceptable salt thereof, wherein Y is N or CR⁶, wherein R⁶ is hydrogen.

7. The compound of claim 1, or a pharmaceutically acceptable salt thereof, wherein Z is N or CR⁷, wherein R⁷ is hydrogen, ethanol, CH₂NH₂ or CH₂NHCH₃ or when taken with R¹ forms a cyclohexane ring.

8. The compound of claim 1, or a pharmaceutically acceptable salt thereof, wherein n is 1.

9. The compound of claim 1, or a pharmaceutically acceptable salt thereof, wherein A is aryl, wherein the aryl is

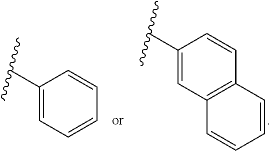 [SMILES: *c1ccccc1 ; *c1ccc2ccccc2c1]

10. The compound of claim 1, or a pharmaceutically acceptable salt thereof, wherein A is aryl, wherein the aryl is 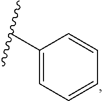 [SMILES: *c1ccccc1]
wherein the aryl is independently unsubstituted or substituted with 1 to 3 substituents selected from the group consisting of chlorine, fluorine, methoxy, methyl, —CN, —CH₂NHCOCH₃, —SO₂NH₂, —CH₂SO₂NH₂, —CH₂NHSO₂CH₃, C(CH₃)₂)OH, —COCH₃, —OCF₃, —NHCOOCH₃, —NHC(O)CH3, —CH2NHCOOCH₃,

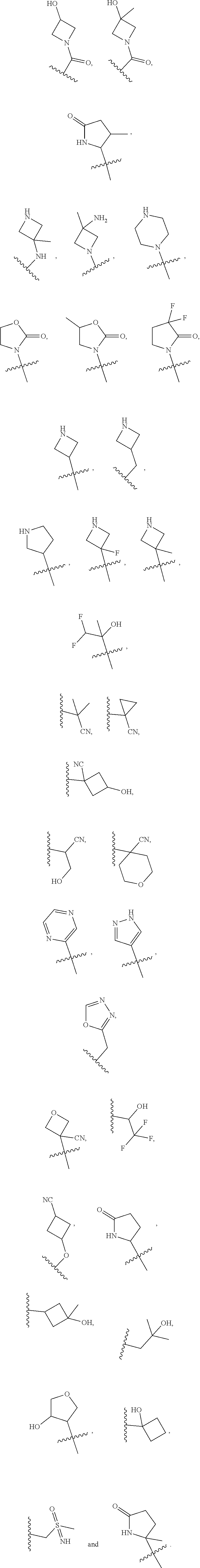

11. The compound of claim 1, or a pharmaceutically acceptable salt thereof, wherein A is C₃-C₁₀cycloalkyl, wherein the C₃-C₁₀cycloalkyl is cyclopropyl substituted with phenyl.

12. The compound of claim 1, or a pharmaceutically acceptable salt thereof, wherein A is heteroaryl, wherein the heteroaryl is

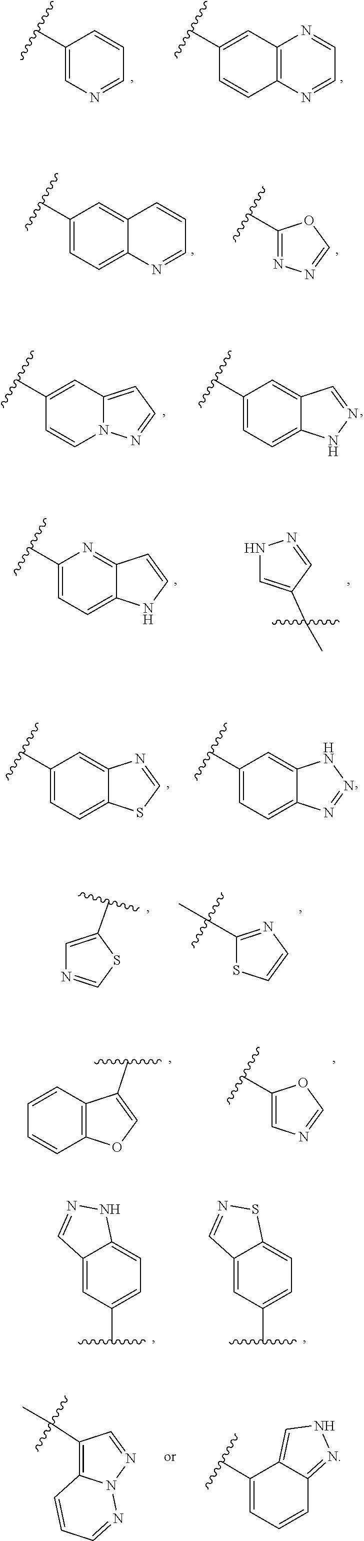

13. The compound of claim 1, or a pharmaceutically acceptable salt thereof, wherein A is heteroaryl, wherein the heteroaryl is 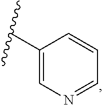 [SMILES: *c1cccnc1]
wherein the heteroaryl is unsubstituted or substituted with 1 to 3 substituents independently selected from the group consisting of chlorine, fluorine, —CH₂CN, —NHSO₂CH₂CH₃, —NHSO₂CH₃, —CH₂NHCOCH₃, —CH₂NHSO₂CH₃, —NHCOCH₃,

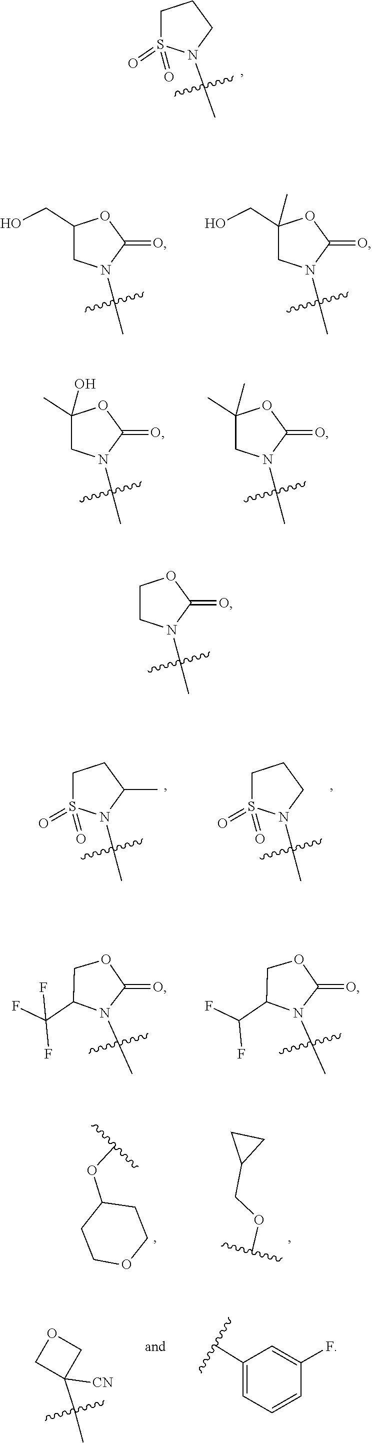

14. The compound of claim 1, or a pharmaceutically acceptable salt thereof, wherein A is cycloheteroalkyl, wherein the cycloheteroalkyl is 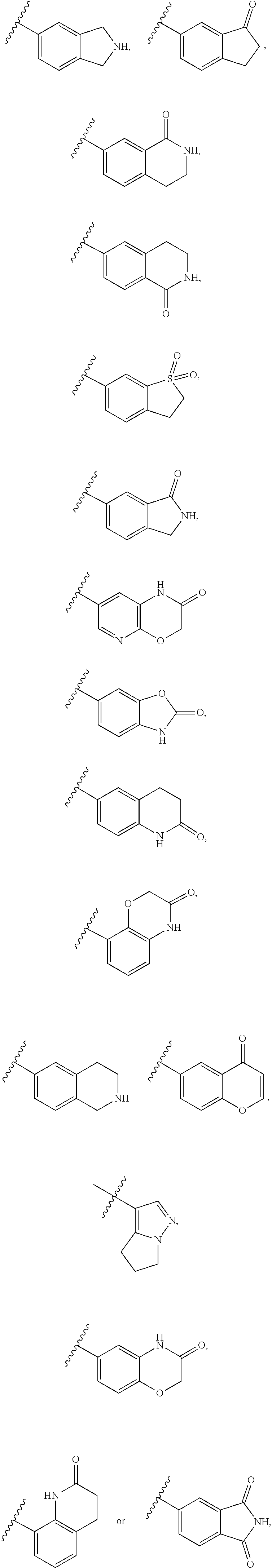
wherein the cycloheteroalkyl is unsubstituted or substituted with 1 to 3 substituents independently selected from the group consisting of fluorine, chlorine, —COCH₃, —CN, methyl, ethyl, isobutyl, —CH₂NHCOOCH₃,

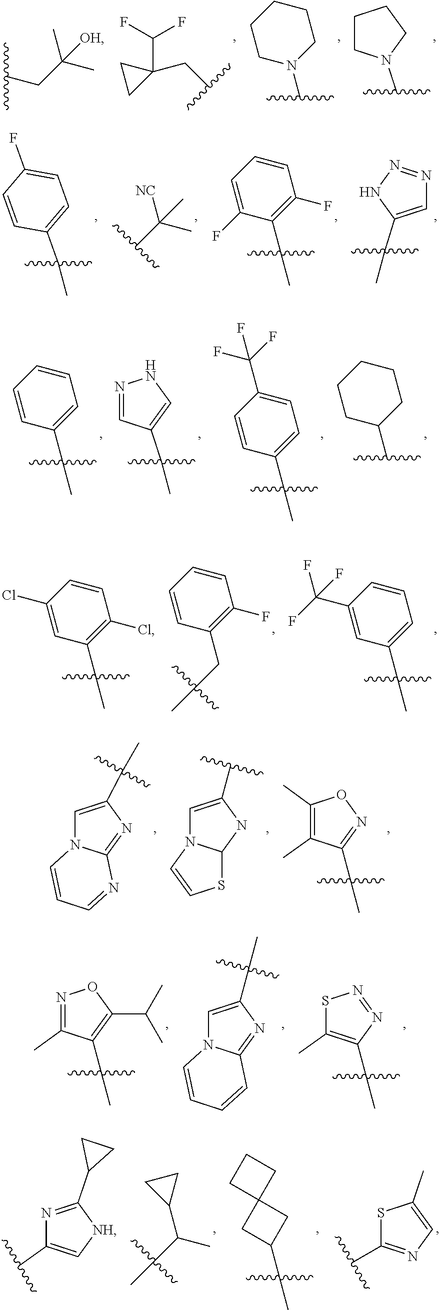

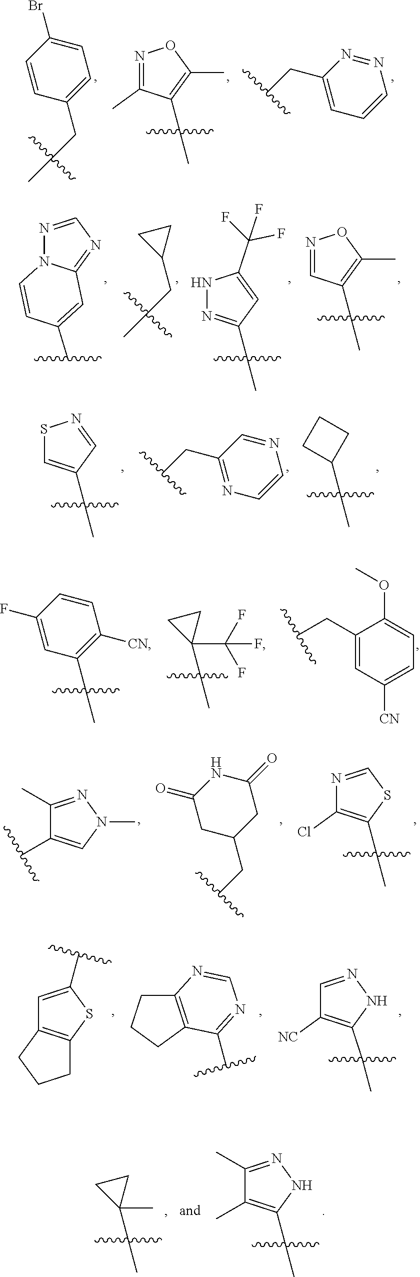

15. The compound of claim 1, or a pharmaceutically acceptable salt thereof, wherein A is 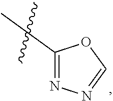 [SMILES: *c1nnco1]
which is unsubstituted or substituted with 1 to 3 substituents independently selected from the group consisting of methyl, —CH₂NHCOOCH₃, —NHCOOCH₃,

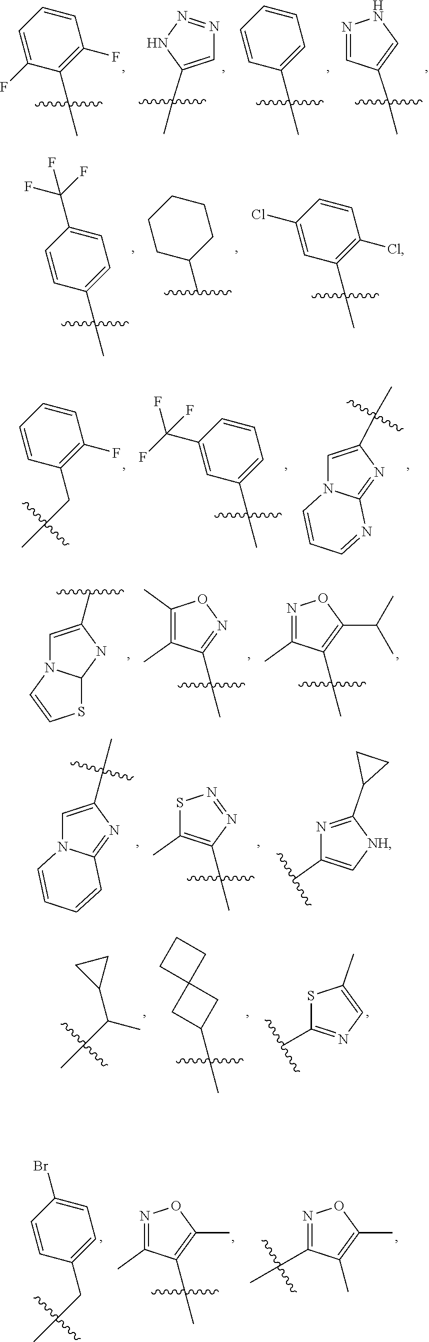

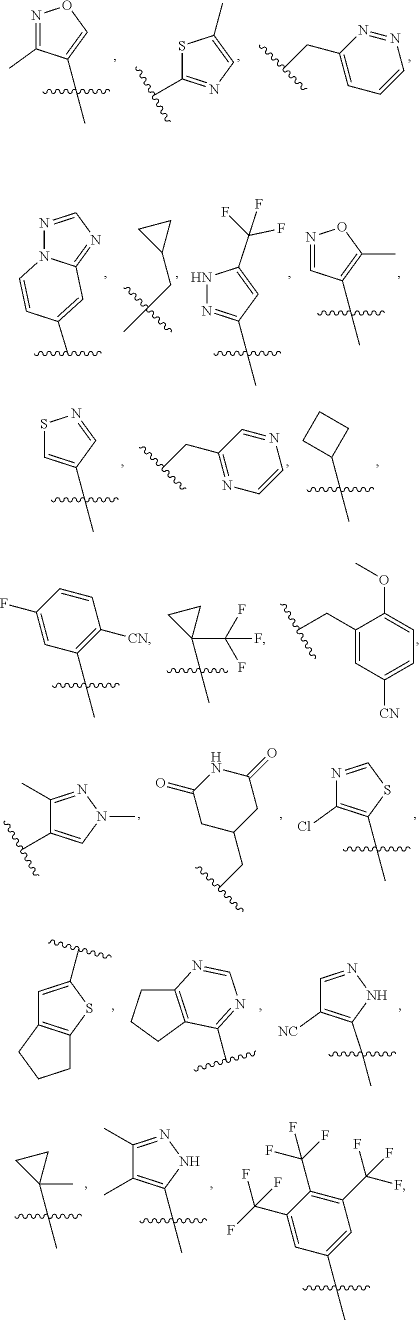

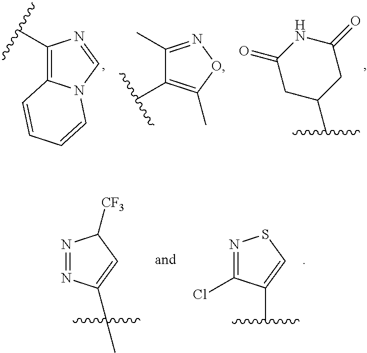

16. A compound, or a pharmaceutically acceptable salt thereof, having the following structure:

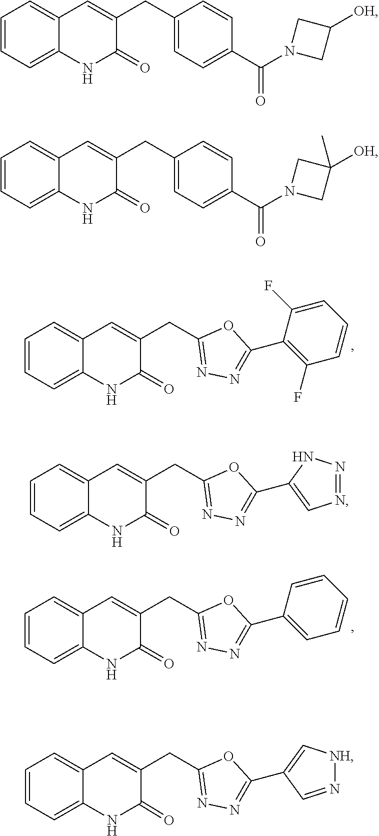

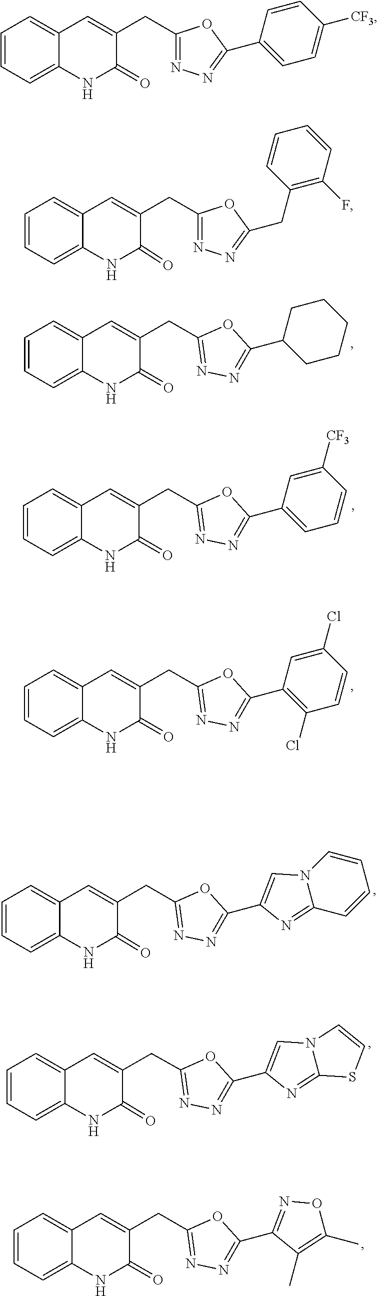

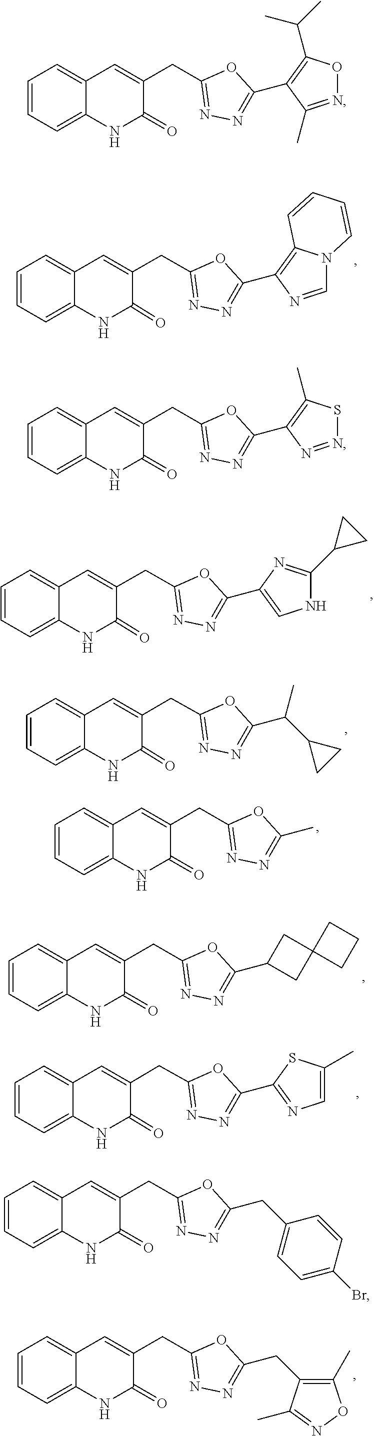

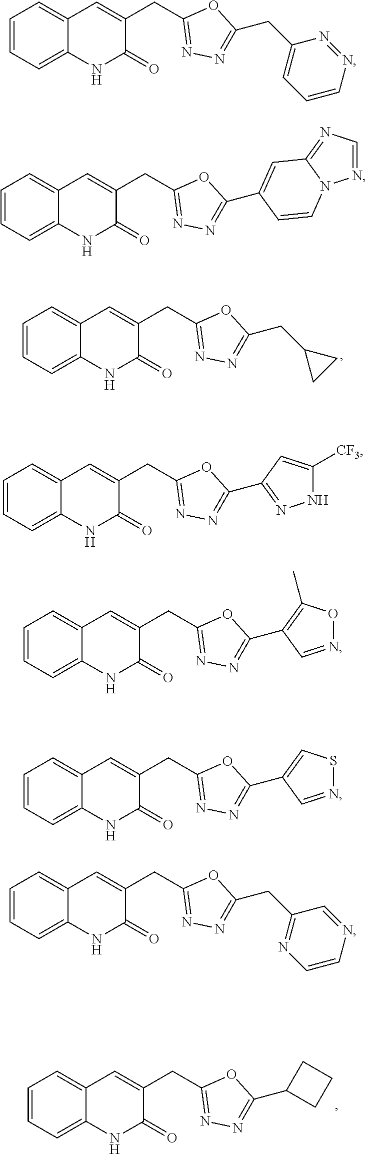

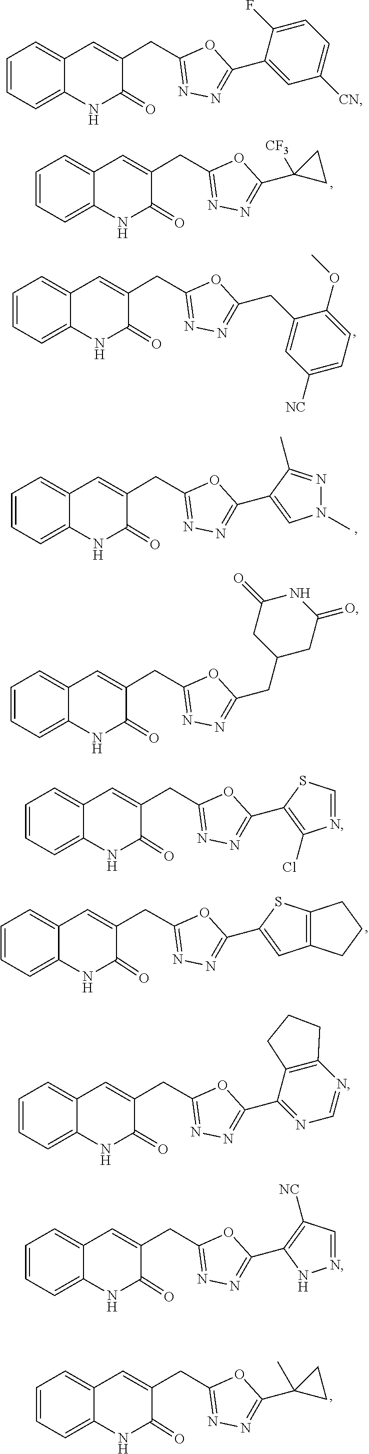

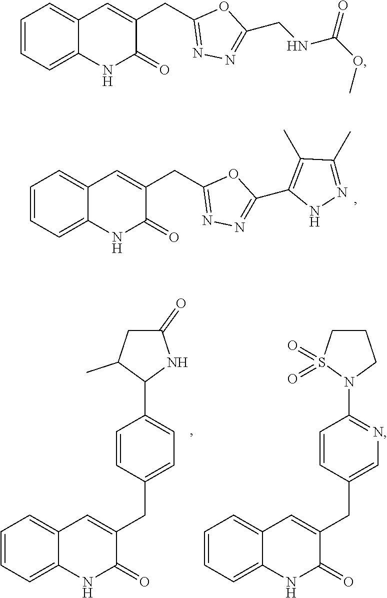

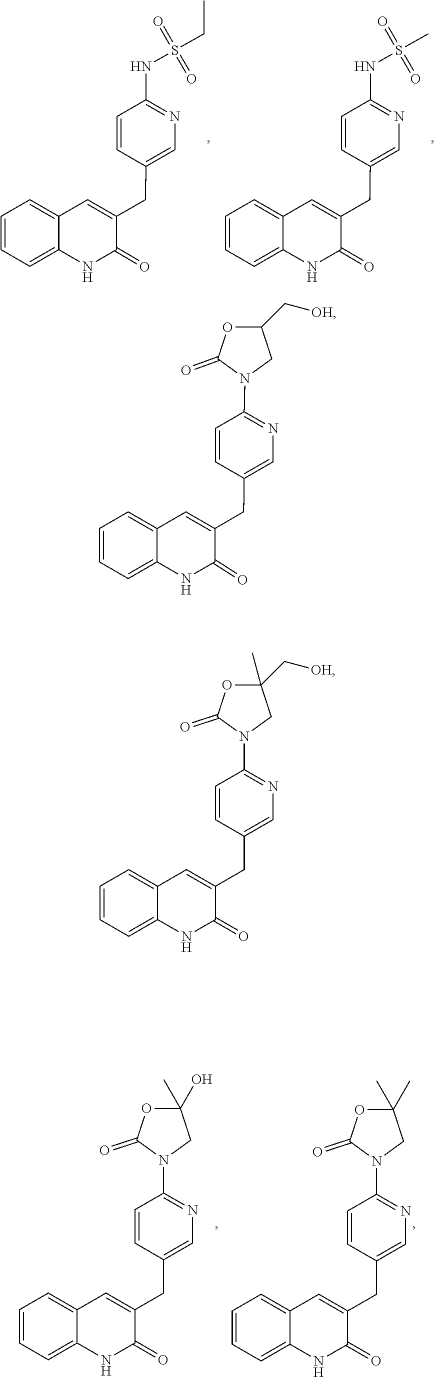

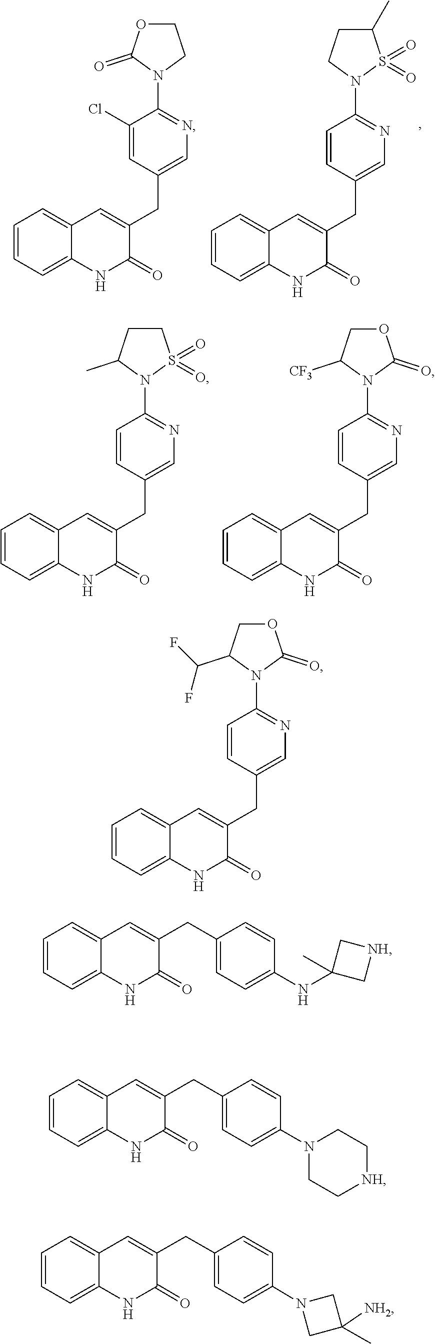

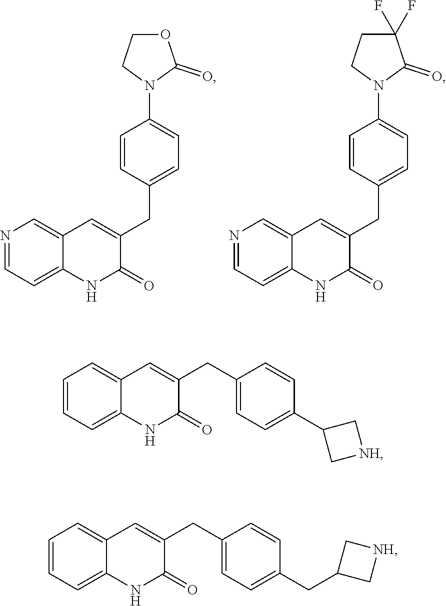 [SMILES: O=C1OCCN1c1ccc(Cc2cc3cnccc3[nH]c2=O)cc1 ; O=C1N(c2ccc(Cc3cc4cnccc4[nH]c3=O)cc2)CCC1(F)F ; O=c1[nH]c2ccccc2cc1Cc1ccc(C2CNC2)cc1 ; O=c1[nH]c2ccccc2cc1Cc1ccc(CC2CNC2)cc1]

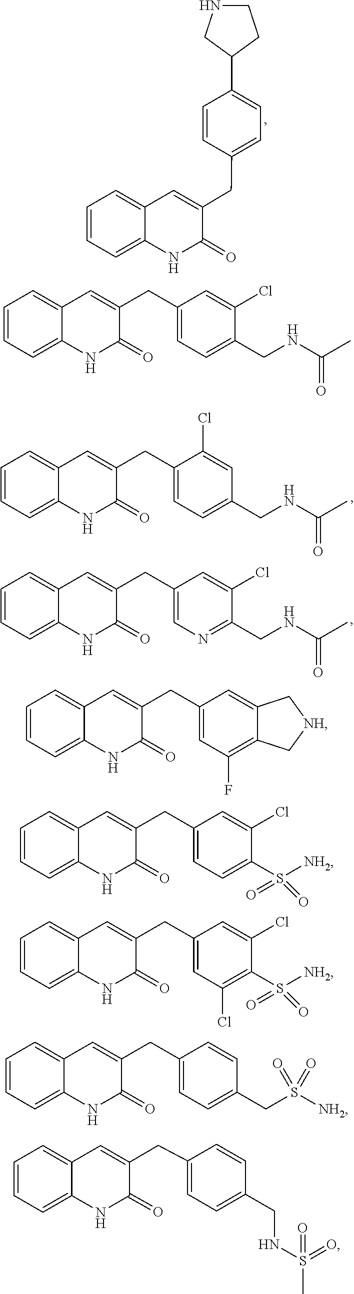

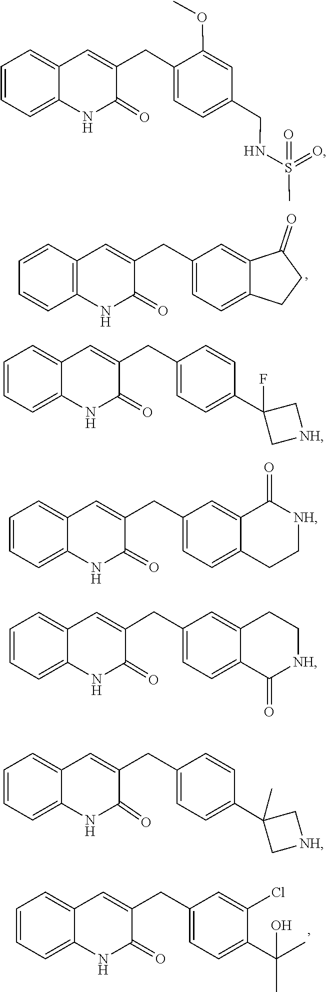

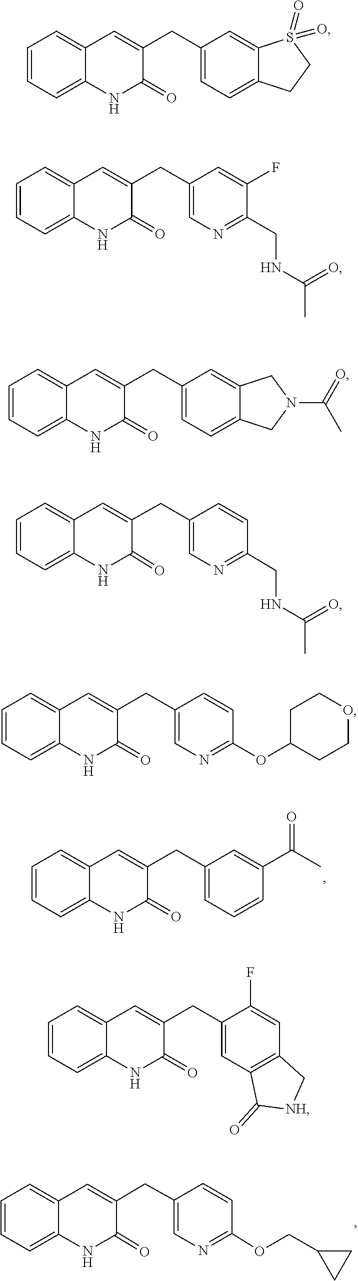

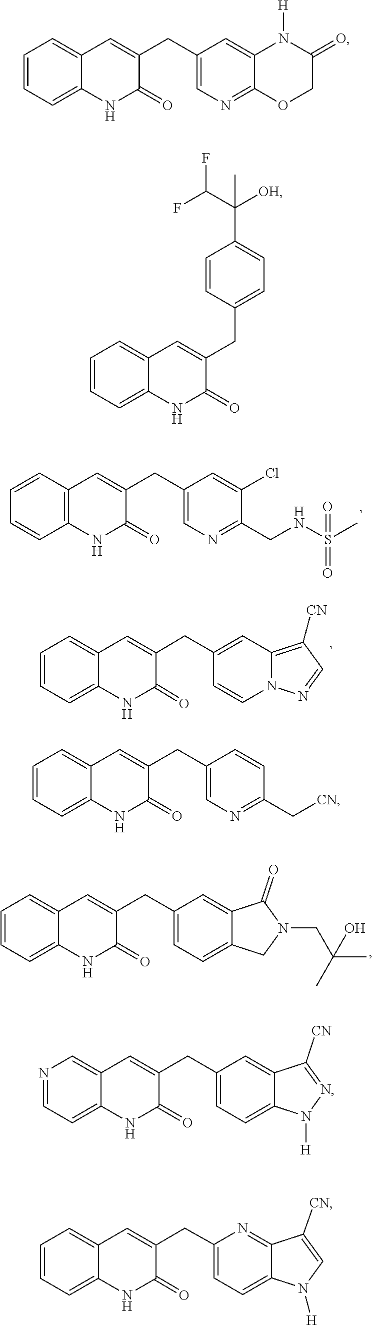

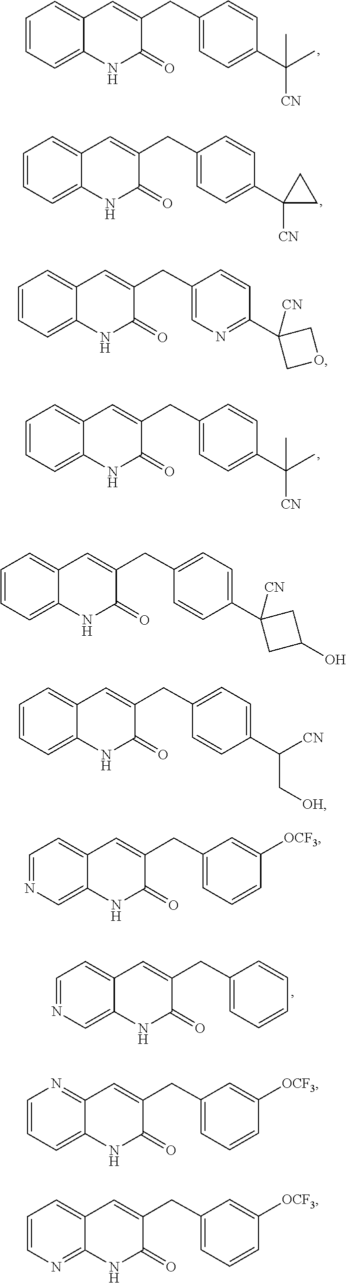

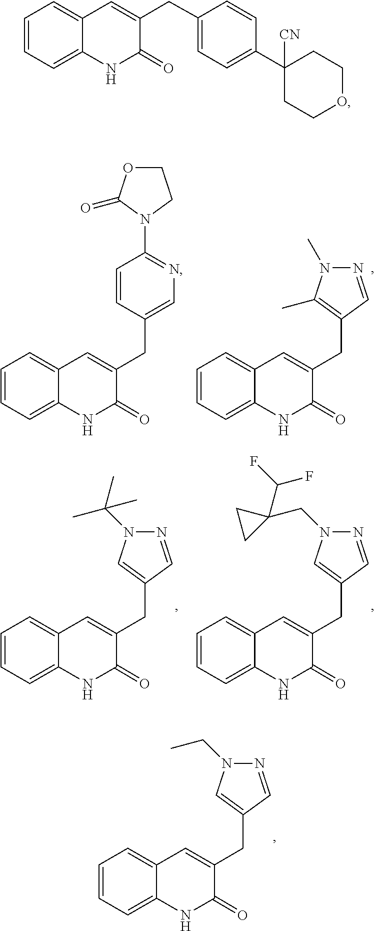 [SMILES: N#CC1(c2ccc(Cc3cc4ccccc4[nH]c3=O)cc2)CCOCC1 ; O=C1OCCN1c1ccc(Cc2cc3ccccc3[nH]c2=O)cn1 ; Cc1c(Cc2cc3ccccc3[nH]c2=O)cnn1C ; CC(C)(C)n1cc(Cc2cc3ccccc3[nH]c2=O)cn1 ; O=c1[nH]c2ccccc2cc1Cc1cnn(CC2(C(F)F)CC2)c1 ; CCn1cc(Cc2cc3ccccc3[nH]c2=O)cn1]

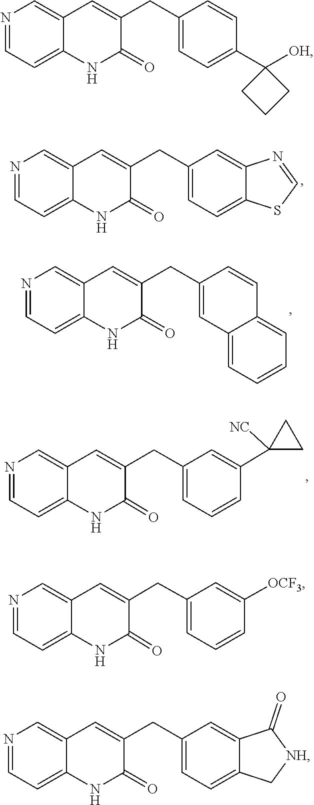

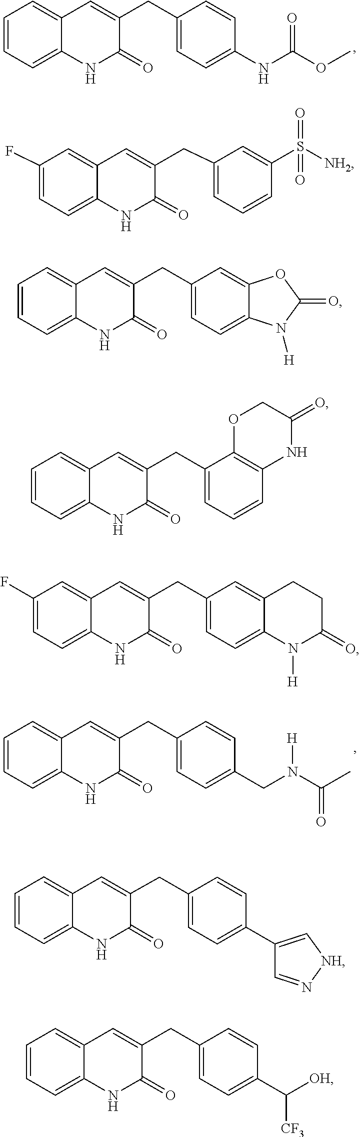

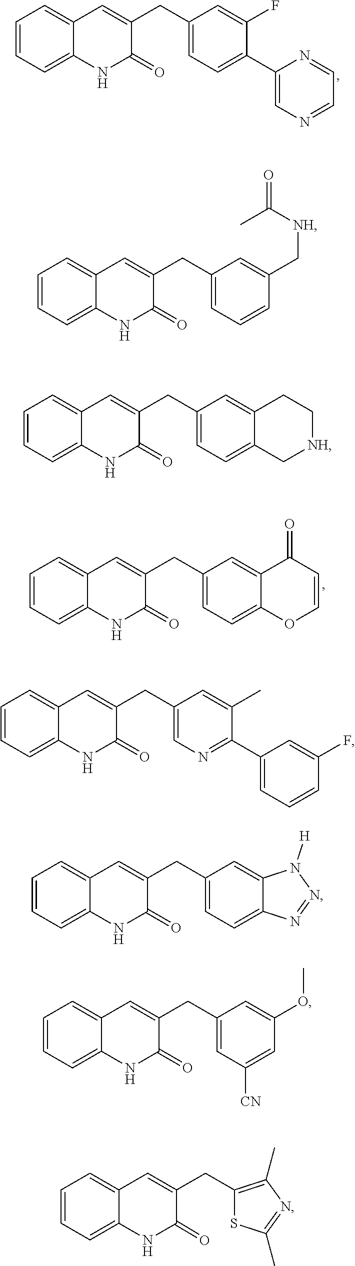

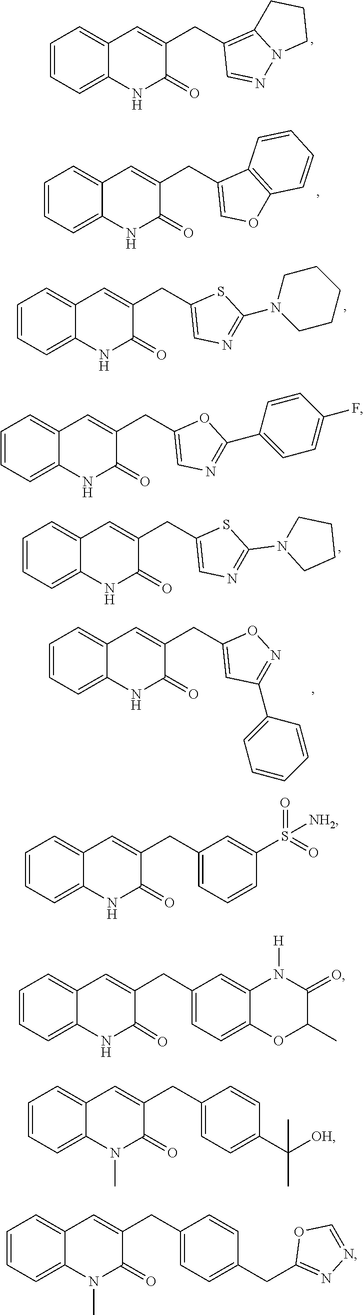

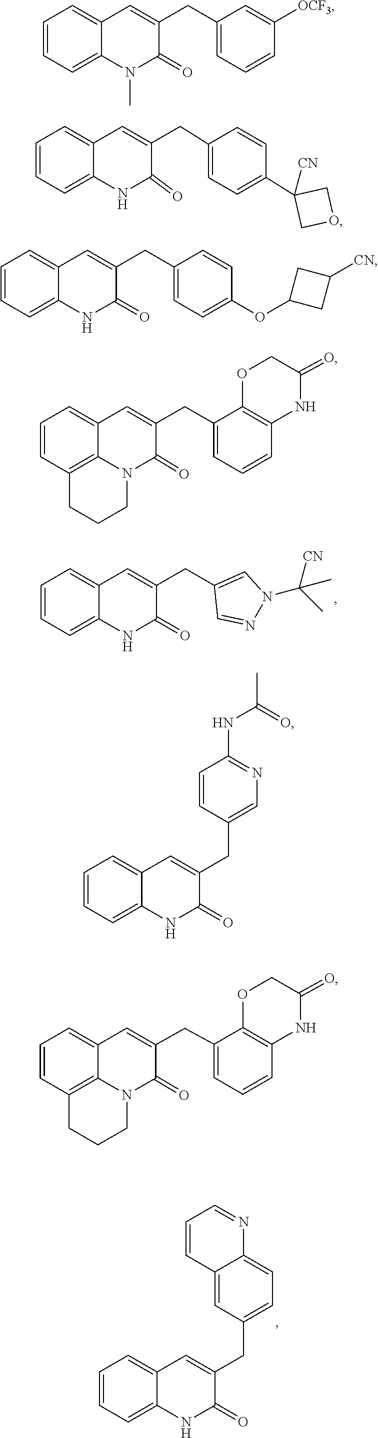

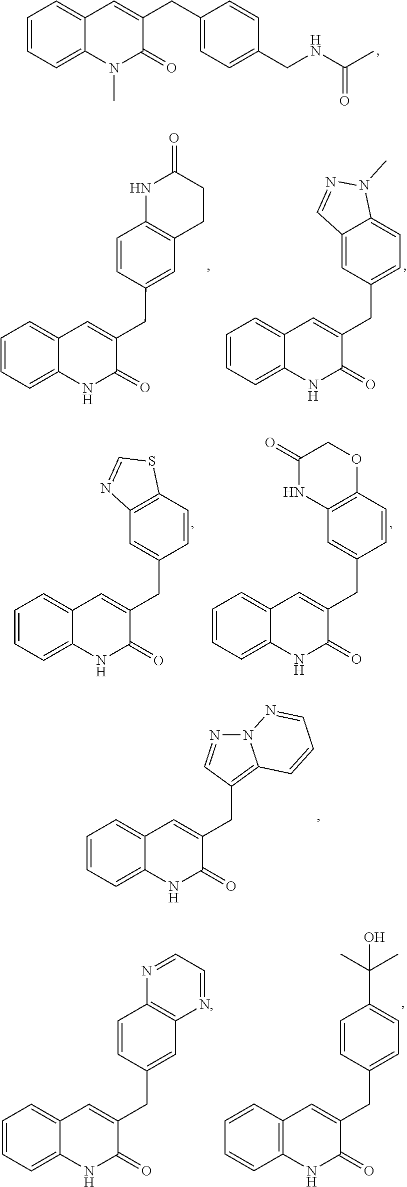

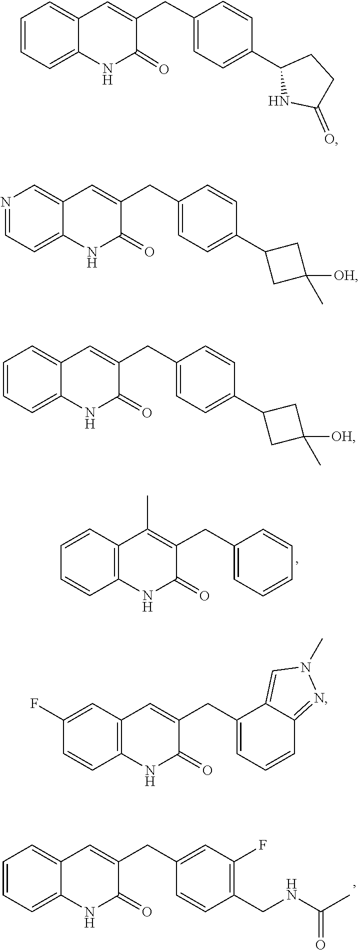 [SMILES: O=C1CC[C@@H](c2ccc(Cc3cc4ccccc4[nH]c3=O)cc2)N1 ; CC1(O)CC(c2ccc(Cc3cc4cnccc4[nH]c3=O)cc2)C1 ; CC1(O)CC(c2ccc(Cc3cc4ccccc4[nH]c3=O)cc2)C1 ; Cc1c(Cc2ccccc2)c(=O)[nH]c2ccccc12 ; Cn1cc2c(Cc3cc4cc(F)ccc4[nH]c3=O)cccc2n1 ; CC(=O)NCc1ccc(Cc2cc3ccccc3[nH]c2=O)cc1F]

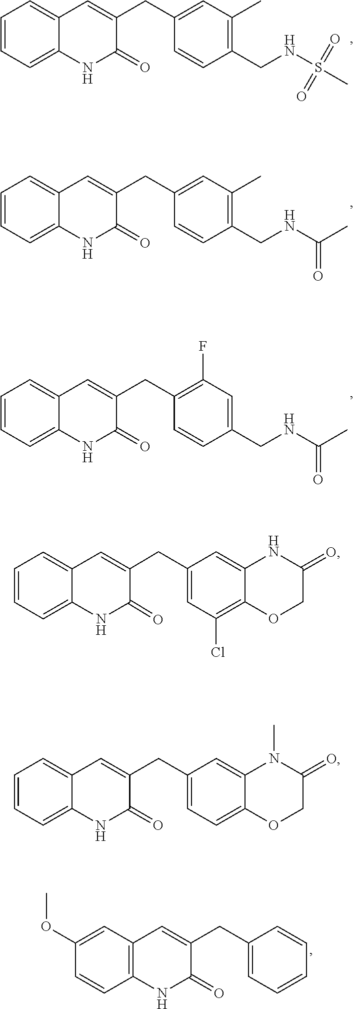 [SMILES: Cc1cc(Cc2cc3ccccc3[nH]c2=O)ccc1CNS(C)(=O)=O ; CC(=O)NCc1ccc(Cc2cc3ccccc3[nH]c2=O)cc1C ; CC(=O)NCc1ccc(Cc2cc3ccccc3[nH]c2=O)c(F)c1 ; O=C1COc2c(Cl)cc(Cc3cc4ccccc4[nH]c3=O)cc2N1 ; CN1C(=O)COc2ccc(Cc3cc4ccccc4[nH]c3=O)cc21 ; COc1ccc2[nH]c(=O)c(Cc3ccccc3)cc2c1]

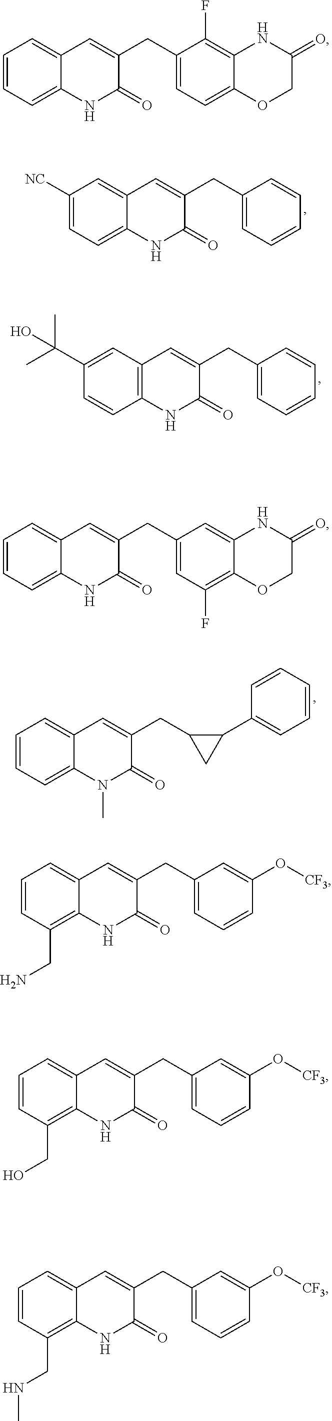

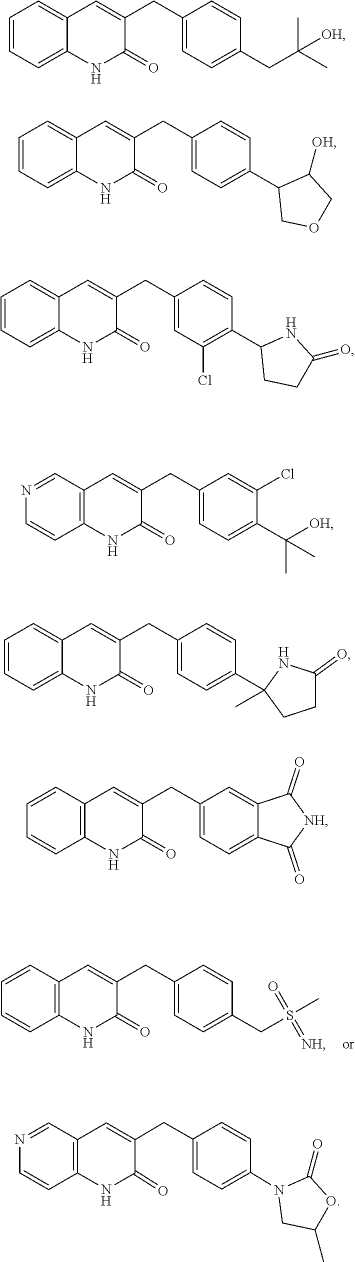

17. A method of treating cancer in a patient in need thereof comprising administering to the patient a therapeutically effective amount of a compound of claim 1, or a pharmaceutically acceptable salt thereof.

18. (canceled)

19. A pharmaceutical composition comprising a compound of claim 1, or a pharmaceutically acceptable salt thereof and a pharmaceutically acceptable carrier.

20. A pharmaceutical composition comprising a compound of claim 1 and a pharmaceutically acceptable carrier.
